SEQUENCE LISTING (15 sequences)

SEQ ID NO: 1 - DNA, 25 bp, Artificial Sequence
  Synthetic
cccgagactcagtgaaattgaactc

SEQ ID NO: 2 - DNA, 17 bp, Artificial Sequence
  Synthetic
aaggttaagcctcacgg

SEQ ID NO: 3 - DNA, 22 bp, Artificial Sequence
  Synthetic
ggtcctctcgtactaggagcag

SEQ ID NO: 4 - DNA, 20 bp, Artificial Sequence
  Synthetic
taatacgactcactataggg

SEQ ID NO: 5 - DNA, 26 bp, Artificial Sequence
  Synthetic
ggttataatgaattttgcttattaac

SEQ ID NO: 6 - DNA, 84 bp, Artificial Sequence
  Synthetic
taatacgactcactatagggcttaagtataaggaggaaaaaatatgttggtattccaaatgcgtaatgtagataaaacatctac

SEQ ID NO: 7 - DNA, 78 bp, Artificial Sequence
  Synthetic
ggttataatgaattttgcttattaacgatagaattctatcacttatttcaaaatagtagatgttttatctacattacg

SEQ ID NO: 8 - DNA, 44 bp, Artificial Sequence
  Synthetic
ggaggaaaaaatatgacacactcaatgagacttcgtattttccc

SEQ ID NO: 9 - DNA, 41 bp, Artificial Sequence
  Synthetic
ctatcacttacaaagttgggaaaatacgaagtctcattgag

SEQ ID NO: 10 - DNA, 58 bp, Artificial Sequence
  Synthetic
taatacgactcactatagggcttaagtataaggaggaaaaaatatgacacactcaatg

SEQ ID NO: 11 - DNA, 60 bp, Artificial Sequence
  Synthetic
ggttataatgaattttgcttattaacgatagaattctatcacttacaaagttgggaaaat

SEQ ID NO: 12 - protein, 17 aa, Artificial Sequence
  Synthetic
MLVFQMRNVDKTSTILK

SEQ ID NO: 13 - RNA, 54 nt, Artificial Sequence
  Synthetic
auguugguauuccaaaugcguaauguagauaaaacaucuacuauuuugaaauaa

SEQ ID NO: 14 - protein, 13 aa, Artificial Sequence
  Synthetic
MTHSMRLRIFPTL

SEQ ID NO: 15 - RNA, 42 nt, Artificial Sequence
  Synthetic
augacacacucaaugagauugcguauuuucccaacuuuguaa

(19) United States
(12) Patent Application Publication
(10) Pub. No.: US 2023/0203081 A1
(43) Pub. Date: Jun. 29, 2023

(54) LINCOSAMIDE ANTIBIOTICS AND USES THEREOF

(71) Applicant: President and Fellows of Harvard College, Cambridge, MA (US)

(72) Inventors: Andrew G. Myers, Cambridge, MA (US); Katherine J. Silvestre, Cambridge, MA (US); Matthew James Mitcheltree, Cambridge, MA (US); Giambattista Testolin, Cambridge, MA (US); Jeremy D. Mason, Cambridge, MA (US); Aditya Pote, Cambridge, MA (US); Daniel Terwilliger, Cambridge, MA (US)

(73) Assignee: President and Fellows of Harvard College, Cambridge, MA (US)

(21) Appl. No.: 17/926,809

(22) PCT Filed: May 20, 2021
(86) PCT No.: PCT/US2021/033371

§ 371 Date: Nov. 21, 2022

Related U.S. Application Data
(60) Provisional application No. 63/167,138, filed on Mar. 29, 2021.
(60) Provisional application No. 63/029,046, filed on May 22, 2020.

(51) Int. Cl. C07H 15/16; A61P 31/04

(52) U.S. Cl. CPC C07H 15/16; A61P 31/04

(57) ABSTRACT

Provided are compounds of Formula (I) for the treatment of infectious and inflammatory diseases. The compounds described herein are lincosamides modified at the amino acid (southern) region. The compounds may have further modification at the C-7 position of the aminooctose (northern) region, thus distinguishing them from lincomycin and clindamycin. Also provided are methods for preparing the compounds, pharmaceutical compositions comprising the compounds, and methods of treating infectious diseases using the disclosed compounds.

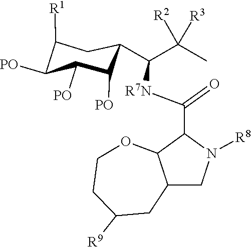

### RELATED APPLICATIONS

This application claims priority under 35 U.S.C. § 119(e) to U.S. Provisional Applications, U.S. Ser. No. 63/029,046, filed May 22, 2020, and U.S. Ser. No. 63/167,138, filed Mar. 29, 2021, each of which is incorporated herein by reference in its entirety.

### BACKGROUND

Emerging resistance to existing antibiotics is rapidly developing as a crisis of global proportions, especially for infections originating from drug-resistant Gram-negative bacteria. Pathogenic bacteria can transmit genes coding for antibiotic resistance both vertically (to their progeny) and horizontally (to neighboring bacteria of different lineages), and as a result antibiotic resistance can evolve quickly, particularly in nosocomial (hospital) settings. See, e.g., Wright, Chem. Commun. (2011) 47:4055-4061. More than 99,000 people die annually in the U.S. from healthcare-associated infections, more than all casualties from car accidents, HIV, and breast cancer combined, creating an estimated burden of up to $45 billion in U.S. healthcare costs. See, e.g., Klevens et al., Public Health Rep (2007) 122:160-166. The current crisis is exacerbated by decreased research in the development of new antibiotics by most major pharmaceutical companies. See, e.g., Projan, Curr. Opin. Microbiol. (2003) 6:427-430. The current rate of introduction of new antibiotics does not adequately address growing resistance, and with the ease of international travel and increasing population densities, the need for innovation in the field has never been higher.

The lincosamides are a class of antibiotics that prevent bacterial growth by interfering with the synthesis of proteins. They bind to the 23s portion of the 50S subunit of bacterial ribosomes and cause premature dissociation of the peptidyl-tRNA from the ribosome. Lincosamides do not interfere with protein synthesis in human cells (or those of other eukaryotes) because human ribosomes are structurally different than those of bacteria.

The first lincosamide to be discovered was lincomycin, but the use of lincomycin as an antibiotic has been largely superseded by clindamycin, which exhibits improved antibacterial activity. Clindamycin also exhibits some activity against parasitic protozoa and has been used to treat toxoplasmosis and malaria. Lincosamides are typically used to treat Staphylococcus and Streptococcus infections but have also proved to be useful in treating Bacteroides fragilis and other anaerobic infections. They are used in the treatment of toxic shock syndrome and thought to directly block M protein production that leads to the severe inflammatory response.

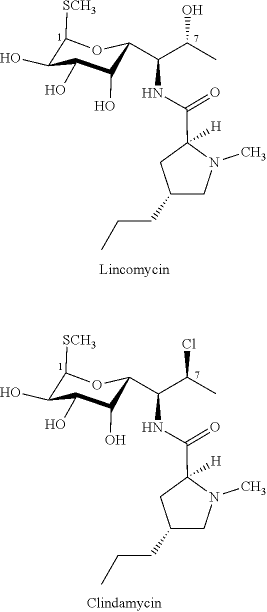

Target bacteria may alter the drug's binding site leading to resistance (similar to resistance found with macrolides and streptogramins). The resistance mechanism is methylation of the 23s binding site. If this occurs, then the bacteria are resistant to both macrolides and lincosamides. In rare instances, enzymatic inactivation of clindamycin has also been reported.

In addition, lincosamide antibiotics are associated with pseudomembranous colitis caused by Clostridium difficile (C. difficile). Pseudomembranous colitis is inflammation of the colon associated with an overgrowth of C. difficile. This overgrowth of C. difficile is most often related to recent lincosamide antibiotic use. For example, clindamycin, currently the only lincosamide in clinical use, carries a black-box warning for its tendency to promote C. difficile-associated diarrhea (CDAD).

Accordingly, the discovery and development of new antibiotics effective against drug-resistant bacteria, particularly lincosamides, represents a currently unmet medical need.

### SUMMARY

The disclosed lincosamides demonstrate potent activity against high-priority, clinically relevant pathogens including clindamycin- and azithromycin-resistant strains of S. aureus, S. pneumoniae, and E. faecalis—strains against which effective new antibiotics are in demand. Moreover, the disclosed lincosamides show potential promise as safer alternatives to clindamycin, owing to a diminished negative impact on commensal gut flora due to increased activity against C. difficile. The disclosed synthetic lincosamides also demonstrate activity against Gram-negative pathogens like E. coli.

In one aspect, the present disclosure provides compounds of Formula (I):

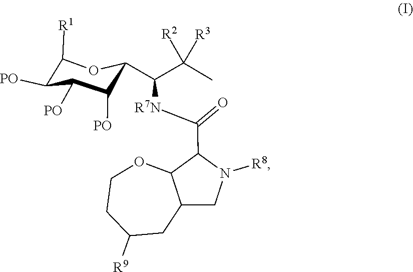

and pharmaceutically acceptable salts, solvates, hydrates, polymorphs, co-crystals, tautomers, stereoisomers, isotopically labeled derivatives, and prodrugs thereof, wherein:

each P is independently hydrogen or an oxygen protecting group;

R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, or —SR^{A};

R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};

R³ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroaliphatic;

R⁷ is hydrogen or unsubstituted alkyl;

R⁸ is hydrogen, substituted or unsubstituted alkyl, substituted or unsubstituted carbocyclyl, substituted or unsubstituted heterocyclyl, substituted or unsubstituted heteroaliphatic, —C(═O)R^{A}, —C(═O)OR^{A}, —C(═O)N(R^{A})₂, —S(O)₂R^{A}, or a nitrogen protecting group;

R⁹ is substituted or unsubstituted alkyl, substituted or unsubstituted alkenyl, substituted or unsubstituted alkynyl, substituted or unsubstituted carbocyclyl, or substituted or unsubstituted heteroaliphatic, provided that R⁹ is not aralkyl; and

each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

In certain embodiments, the present disclosure provides compounds of Formulae (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), and (I-u):

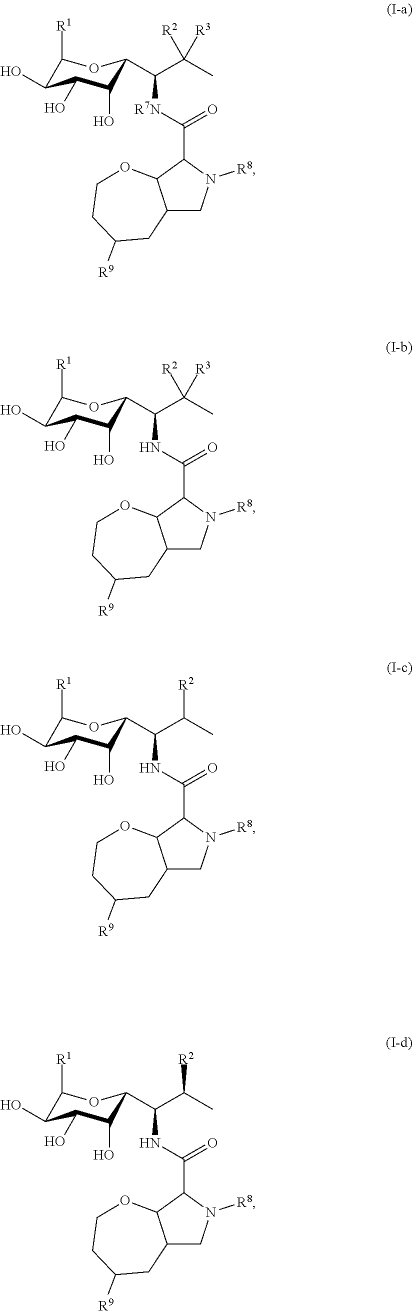

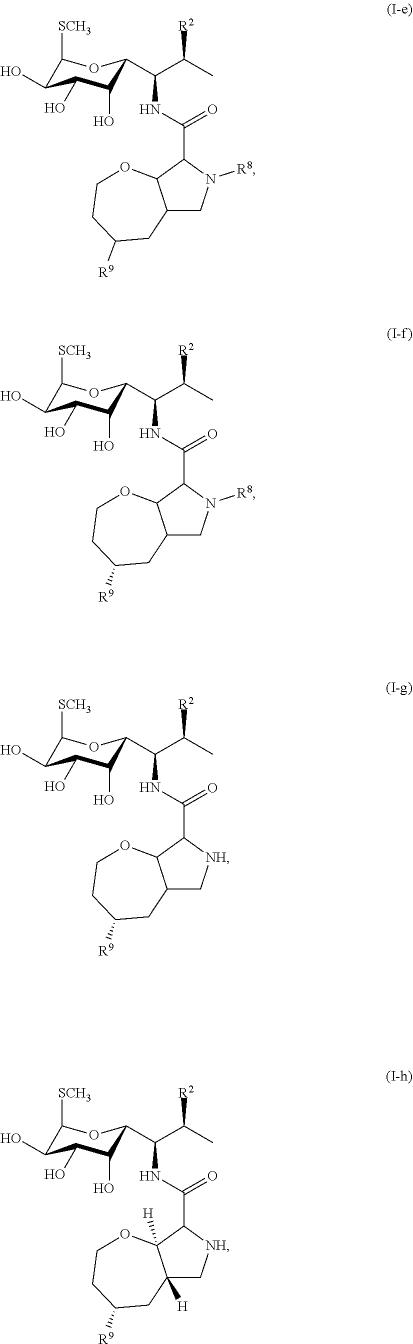

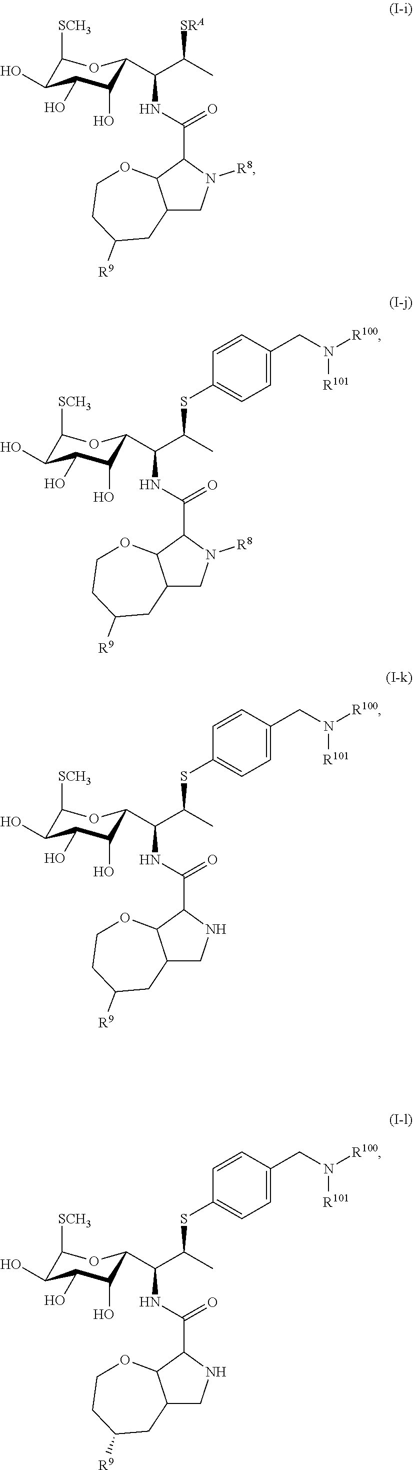

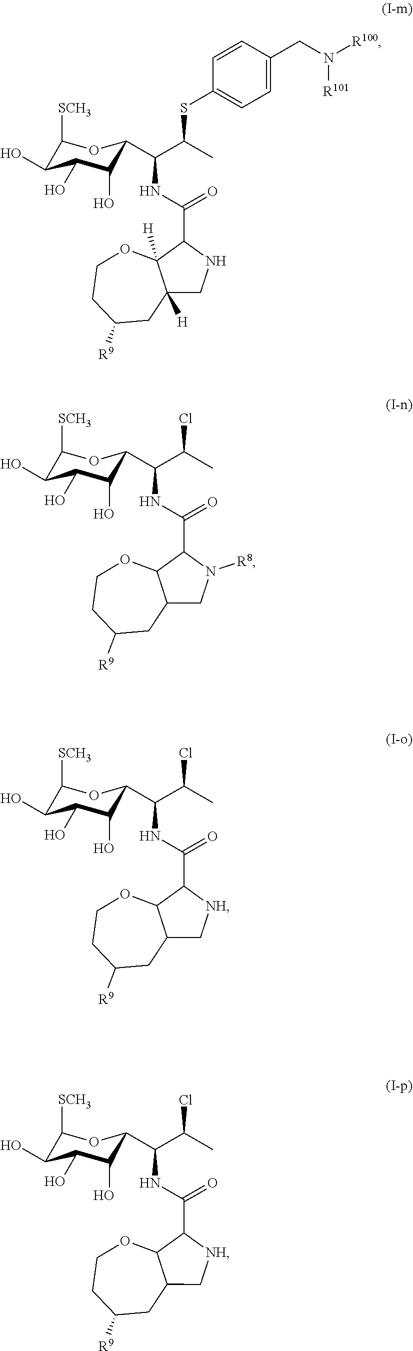

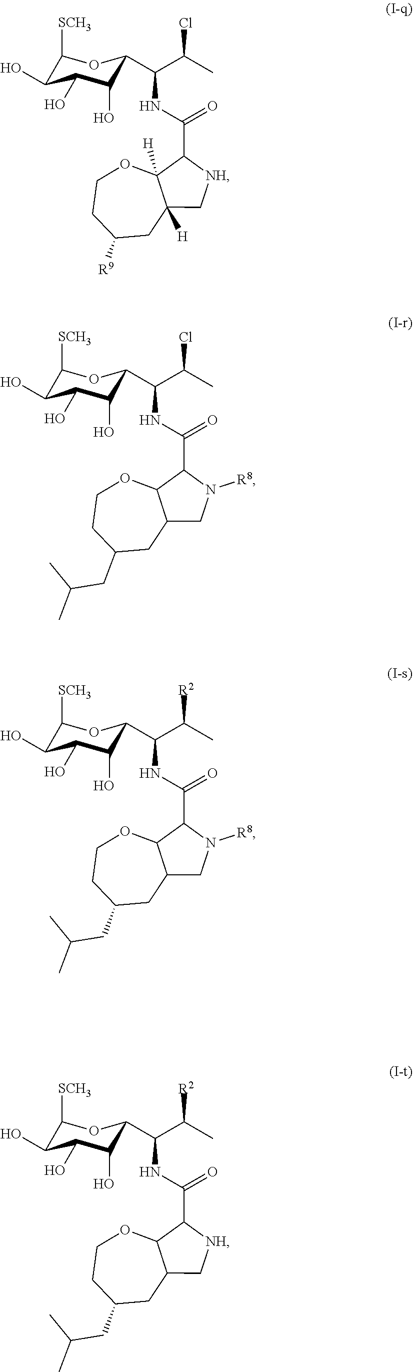

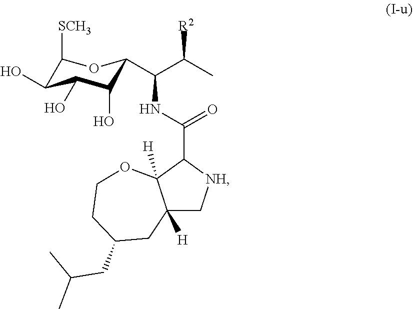

and pharmaceutically acceptable salts thereof.

The disclosed compounds have anti-microbial activity and may be used to treat and/or prevent infectious diseases. Pharmaceutical compositions of the compounds, kits comprising the compounds and/or compositions, and methods of treatment using the compounds or compositions thereof are provided herein. Infectious diseases which may be treated with compounds of the invention include, but are not limited to, bacterial infections caused by Staphylococcus, Streptococcus, Enterococcus, Acinetobacter, Clostridium, Bacteroides, Klebsiella, Escherichia, Pseudomonas, and Haemophilus species.

The details of certain embodiments of the invention are set forth in the Detailed Description of Certain Embodiments, as described below. Other features, objects, and advantages of the invention will be apparent from the Definitions, Examples, and Claims.

### BRIEF DESCRIPTION OF THE DRAWINGS

FIG. 1 is a depiction of the crystal structure of FSA-513018b with perspective views showing 50% probability displacement.

FIG. 2A and FIG. 2B show time-kill kinetics, post-antibiotic effect, and post-antibiotic sub-MIC effect data of FSA-513018b (SA-3 in the figures) against susceptible S. aureus and E. faecium strains. FIG. 2A shows arrayed growth curves for three susceptible strains showing concentration effects on growth inhibition (time-kill), growth kinetics following exposure to antibiotic at 4x MIC (RAE), and growth kinetics under sub-MIC concentrations following exposure to antibiotic at 4× MIC (PA-SME). Points represent the mean of bacterial counts (n=2), while error bars depict standard deviations from the mean. FIG. 2B shows tabulated PAE and PA-SME durations (determined as the difference in time required for bacterial counts to rise 10× between experimental and untreated control arms).

FIGS. 3A-3F shows the effects of FSA-513018b (OPP-3 in the figures) on mammalian cells. FIG. 3A shows normalized hemolysis of human erythrocytes relative to Triton X-100. FIGS. 3B-3C show mitochondrial ToxGlo data showing effects of FSA-513018b on HepG2 cellular membrane integrity and ATP production relative to vehicle-treated control (antimycin serves as a positive control for mitotoxicity). FIGS. 3D-3F show comparison of effects of FSA-513018b, clindamycin, doxycycline, and azithromycin on cell viability (CellTiter-Glo). Where applicable, GI₅₀ values (μM) are reported beside the dose-response curves.

FIGS. 4A-4B show pharmacodynamic effects of FSA-513018b (SA-3 in the figures) in a murine neutropenic thigh infection model employing S. pyogenes ATCC 19615 or S. aureus (MRSA). Points represent mean values across two experiments, and error bars depict standard deviation from the mean (n=4 independent animals per time point). Statistical comparison of 24-h time points was conducted using one-way ANOVA with Tukey's multiple comparison test. *p<0.05, ***p<0.0005, ****p<0.0001. FIG. 4C also shows the pharmacodynamic effects of FSA-513018b in a murine neutropenic thigh infection model employing S. pyogenes ATCC 19615, S. aureus (MRSA, HAV017), A. baumanii ATCC 19606, and E. coli HAV504. Points represent mean values across two experiments, and error bars depict standard deviation from the mean (n=4 independent animals per time point). P-values represent statistical comparison of 12 hour timepoints using Tukey's analysis with Welch's correction. FIG. 4D shows survival over time of mice infected with S. pyogenes ATCC 19615 treated with FSA-513018b (n=8), clindamycin (2, n=8), or vehicle (n=10).

FIG. 5 shows FSA-513018b efficiently arrests translation at the start codon. Toeprinting analysis of sites of FSA-513018b-induced translation arrest within the MLS_{B} resistance genes ermB and ermD. Because all reactions contained mupirocin, an inhibitor of Ile-tRNA synthetase, the ribosomes that escape inhibition by ribosome-targeting antibiotics are trapped at the Ile codon (white arrowheads). Black arrows mark translation arrest at the start codon, while white arrows denote known erythromycin-induced arrest sites D10 (ermBL) and L7 (ermDL). ERY, erythromycin; CLI, clindamycin.

FIGS. 6A-6F show the structure of FSA-513018b (SA-3 in the figures) in complex with the 70S ribosome, mRNA, and tRNAs. FIG. 6A. Unbiased Fₒ-F_{c} electron density map of FSA-513018b in complex with the Tth 70S ribosome (mesh “A”) contoured at 3.0σ. FIG. 6B. Overview of the drug-binding site in the Tth 70S ribosome viewed as a cross-cut section through the nascent peptide exit tunnel. FIG. 6C. Superposition of the ribosome-bound oxepanoprolinamide FSA-513018b (*) with the previous structures of clindamycin bound to the tRNA-free 70S ribosome from eubacterium E. coli (\hat{ }, PDB entry 4V7V³²) or to 50S ribosomal subunit from archaeon H. marismortui harboring G2099A mutation in its 23S rRNA (#, PDB entry 1YJN⁴⁸). All structures were aligned based on domain V of the 23S rRNA. FIGS. 6D-6E. Close-up views of FSA-513018b bound in the PTC, highlighting H-bond interactions (dashed lines) and the intercalation of the 7′-isobutyl substituent within the A-site cleft formed by A2451 and C2452 of the 23S rRNA. FIG. 6F. Superimposed models of the A- and P-site tRNAs from the current FSA-513018b—ribosome complex structure with the previous structure of drug-free ribosome containing the same tRNAs (PDB entry 6XHW⁵²). The binding of FSA-513018b to the ribosome prevents accommodation of the aminoacyl moiety and proper placement of the CCA-end of the incoming A-site tRNA.

FIGS. 7A-7D show the structure of FSA-513018b (SA-3 in the figures) bound to the Erm-methylated 70S ribosome. FIGS. 7A-7B. Electron density map (mesh) contoured at 3.0σ of FSA-513018b in complex with the Erm-modified Tth 70S ribosome containing N⁶-dimethylated A2058 residue in the 23S rRNA. FIGS. 7C-7D. Superposition of the FSA-513018b structure (*) in complex with the WT 70S ribosome, containing unmodified residue A2058, and the structure of FSA-513018b (\hat{ }) in complex with Erm-modified 70S ribosome, containing m₂⁶A2058. Hydrogen-bonds are depicted with dashed lines. Note that the binding positions of FSA-513018b are almost identical in the two structures, while FSA-513018b binding to the Erm-modified ribosome causes substantial movement of m₂⁶A2058 from its canonical position (arrow).

### DEFINITIONS

### Chemical Terms

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). In certain embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In certain embodiments, the haloalkyl moiety has 1 to 8 carbon atoms (“C₁₋₈ haloalkyl”). In certain embodiments, the haloalkyl moiety has 1 to 6 carbon atoms (“C₁₋₆ haloalkyl”). In certain embodiments, the haloalkyl moiety has 1 to 4 carbon atoms (“C₁₋₄ haloalkyl”). In certain embodiments, the haloalkyl moiety has 1 to 3 carbon atoms (“C₁₋₃ haloalkyl”). In certain embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In certain embodiments, a heteroalkyl group is a saturated group having 1 to 9 carbon atoms and 1 or more heteroatoms within the parent chain (“heteroC₁₋₉ alkyl”). In certain embodiments, a heteroalkyl group is a saturated group having 1 to 8 carbon atoms and 1 or more heteroatoms within the parent chain (“heteroC₁₋₈ alkyl”). In certain embodiments, a heteroalkyl group is a saturated group having 1 to 7 carbon atoms and 1 or more heteroatoms within the parent chain (“heteroC₁₋₇ alkyl”). In certain embodiments, a heteroalkyl group is a saturated group having 1 to 6 carbon atoms and 1 or more heteroatoms within the parent chain (“heteroC₁₋₆ alkyl”). In certain embodiments, a heteroalkyl group is a saturated group having 1 to 5 carbon atoms and 1 or 2 heteroatoms within the parent chain (“heteroC₁₋₅ alkyl”). In certain embodiments, a heteroalkyl group is a saturated group having 1 to 4 carbon atoms and 1 or 2 heteroatoms within the parent chain (“heteroC₁₋₄ alkyl”). In certain embodiments, a heteroalkyl group is a saturated group having 1 to 3 carbon atoms and 1 heteroatom within the parent chain (“heteroC₁₋₃ alkyl”). In certain embodiments, a heteroalkyl group is a saturated group having 1 to 2 carbon atoms and 1 heteroatom within the parent chain (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

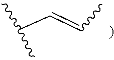

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In certain embodiments, a heteroalkenyl group has 2 to 9 carbon atoms at least one double bond, and 1 or more heteroatoms within the parent chain (“heteroC₂₋₉ alkenyl”). In certain embodiments, a heteroalkenyl group has 2 to 8 carbon atoms, at least one double bond, and 1 or more heteroatoms within the parent chain (“heteroC₂₋₈ alkenyl”). In certain embodiments, a heteroalkenyl group has 2 to 7 carbon atoms, at least one double bond, and 1 or more heteroatoms within the parent chain (“heteroC₂₋₇ alkenyl”). In certain embodiments, a heteroalkenyl group has 2 to 6 carbon atoms, at least one double bond, and 1 or more heteroatoms within the parent chain (“heteroC₂₋₆ alkenyl”). In certain embodiments, a heteroalkenyl group has 2 to 5 carbon atoms, at least one double bond, and 1 or 2 heteroatoms within the parent chain (“heteroC₂₋₅ alkenyl”). In certain embodiments, a heteroalkenyl group has 2 to 4 carbon atoms, at least one double bond, and 1 or 2 heteroatoms within the parent chain (“heteroC₂₋₄ alkenyl”). In certain embodiments, a heteroalkenyl group has 2 to 3 carbon atoms, at least one double bond, and 1 heteroatom within the parent chain (“heteroC₂₋₃ alkenyl”). In certain embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In certain embodiments, a heteroalkynyl group has 2 to 9 carbon atoms, at least one triple bond, and 1 or more heteroatoms within the parent chain (“heteroC₂₋₉ alkynyl”). In certain embodiments, a heteroalkynyl group has 2 to 8 carbon atoms, at least one triple bond, and 1 or more heteroatoms within the parent chain (“heteroC₂₋₈ alkynyl”). In certain embodiments, a heteroalkynyl group has 2 to 7 carbon atoms, at least one triple bond, and 1 or more heteroatoms within the parent chain (“heteroC₂₋₇ alkynyl”). In certain embodiments, a heteroalkynyl group has 2 to 6 carbon atoms, at least one triple bond, and 1 or more heteroatoms within the parent chain (“heteroC₂₋₆ alkynyl”). In certain embodiments, a heteroalkynyl group has 2 to 5 carbon atoms, at least one triple bond, and 1 or 2 heteroatoms within the parent chain (“heteroC₂₋₅ alkynyl”). In certain embodiments, a heteroalkynyl group has 2 to 4 carbon atoms, at least one triple bond, and 1 or 2 heteroatoms within the parent chain (“heteroCheteroC₂₋₄ alkynyl”). In certain embodiments, a heteroalkynyl group has 2 to 3 carbon atoms, at least one triple bond, and 1 heteroatom within the parent chain (“heteroCheteroC₂₋₃ alkynyl”). In certain embodiments, a heteroalkynyl group has 2 to 6 carbon atoms, at least one triple bond, and 1 or 2 heteroatoms within the parent chain (“heteroCheteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 7 ring carbon atoms (“C₃₋₇ carbocyclyl”). In certain embodiments, a carbocyclyl group has 3 to 6 ring carbon atoms (“CC₃₋₆ carbocyclyl”). In certain embodiments, a carbocyclyl group has 4 to 6 ring carbon atoms (“C₄₋₆ carbocyclyl”). In certain embodiments, a carbocyclyl group has 5 to 6 ring carbon atoms (“C₅₋₆ carbocyclyl”). In certain embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In certain embodiments, “carbocyclyl” is a monocyclic, saturated carbocyclyl group having from 3 to 14 ring carbon atoms (“C₃₋₁₄ cycloalkyl”). In certain embodiments, a cycloalkyl group has 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 4 to 6 ring carbon atoms (“C₄₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Carbocyclylalkyl” is a subset of “alkyl” and refers to an alkyl group substituted by a carbocyclyl group, wherein the point of attachment is on the alkyl moiety.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In certain embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In certain embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In certain embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In certain embodiments, the 5-6 membered heterocyclyl has 1-3 ring heteroatoms selected from nitrogen, oxygen, and sulfur. In certain embodiments, the 5-6 membered heterocyclyl has 1-2 ring heteroatoms selected from nitrogen, oxygen, and sulfur. In certain embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Heterocyclylalkyl” is a subset of “alkyl” and refers to an alkyl group substituted by an heterocyclyl group, wherein the point of attachment is on the alkyl moiety.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In certain embodiments, an aryl group has 6 ring carbon atoms (“C₆ aryl”; e.g., phenyl). In certain embodiments, an aryl group has 10 ring carbon atoms (“C₁₀ aryl”; e.g., naphthyl such as 1-naphthyl and 2-naphthyl). In certain embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In certain embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In certain embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In certain embodiments, the 5-6 membered heteroaryl has 1-3 ring heteroatoms selected from nitrogen, oxygen, and sulfur. In certain embodiments, the 5-6 membered heteroaryl has 1-2 ring heteroatoms selected from nitrogen, oxygen, and sulfur. In certain embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X^{-,} —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, —NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —(R^{cc})₂, —CN, —C(═O)R^{aa}, —C(═O)N(R^{cc})₂, —CO₂R^{aa}, —SO₂R^{aa}, —C═NR^{cc})OR^{aa}, —C(—NR^{cc})N(R^{cc})₂, —SO₂N(R^{cc})₂, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X^{-,} —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(—NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋10 carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from ₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X^{-,} —NH(C₁₋₆ alkyl)₂⁺X^{-,} —NH₂(C₁₋₆ alkyl) ⁺X^{-,} —NH₃⁺X^{-,} —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alky), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X^{-,} wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-B OC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo), —OR^{aa} (when the O atom is attached to a carbonyl group, wherein R^{aa} is as defined herein), —O(C═O)R^{LG}, or —O(SO)₂R^{LG} (e.g., tosyl, mesyl, besyl), wherein R^{LG} is optionally substituted alkyl, optionally substituted aryl, or optionally substituted heteroaryl. In certain embodiments, the leaving group is a halogen. In certain embodiments, the leaving group is I.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The invention is not intended to be limited in any manner by the above exemplary listing of substituents.

### Other Definitions

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal “Disease,” “disorder,” and “condition” are used interchangeably herein.

The term “administer,” “administering,” or “administration” refers to implanting, absorbing, ingesting, injecting, inhaling, or otherwise introducing a compound described herein, or a composition thereof, in or on a subject.

As used herein, and unless otherwise specified, the terms “treat,” “treating” and “treatment” contemplate an action that occurs while a subject is suffering from the specified infectious disease or inflammatory condition, which reduces the severity of the infectious disease or inflammatory condition, or retards or slows the progression of the infectious disease or inflammatory condition (“therapeutic treatment”), and also contemplates an action that occurs before a subject begins to suffer from the specified infectious disease or inflammatory condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of an infectious disease or inflammatory condition, or to delay or minimize one or more symptoms associated with the infectious disease or inflammatory condition. A therapeutically effective amount of a compound means an amount of therapeutic agent, alone or in combination with other therapies, which provides a therapeutic benefit in the treatment of the infectious disease or inflammatory condition. The term “therapeutically effective amount” can encompass an amount that improves overall therapy, reduces or avoids symptoms or causes of infectious disease or inflammatory condition, or enhances the therapeutic efficacy of another therapeutic agent.

As used herein, and unless otherwise specified, a “prophylactically effective amount” of a compound is an amount sufficient to prevent an infectious disease or inflammatory condition, or one or more symptoms associated with the infectious disease or inflammatory condition, or prevent its recurrence. A prophylactically effective amount of a compound means an amount of a therapeutic agent, alone or in combination with other agents, which provides a prophylactic benefit in the prevention of the infectious disease or inflammatory condition. The term “prophylactically effective amount” can encompass an amount that improves overall prophylaxis or enhances the prophylactic efficacy of another prophylactic agent.

The term “inflammatory condition” refers to those diseases, disorders, or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent) Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

### DETAILED DESCRIPTION OF CERTAIN EMBODIMENTS

The compounds disclosed herein include lincosamide analogues. The disclosed compounds have increased structural diversity over known lincosamides, such as lincomycin and clindamycin. In particular, the disclosed compounds have structures that include a fused bicyclic ring at the amino-acid (southern) region, and also may be modified at the C-7 position of the aminooctose (northern) region. The disclosed lincosamides provide unexpected and potent activity against various microorganisms, including Gram negative bacteria. For example, the compounds display exceptional activity against enterococcal strains, overcome erm- and cfr-mediated multidrug resistance, and display activity against Gram-negative pathogens. The disclosed lincosamides are non-hemolytic, non-toxic, and possess improved activity profiles relative to clindamycin, such as increased activity against resistant strains of bacteria, including Clostridium difficile. Also disclosed are methods for the preparation of the disclosed compounds, pharmaceutical compositions comprising the compounds, uses of the compounds, and methods of using the compounds (e.g., treatment of an infectious disease, prevention of an infectious disease).

The activity of the disclosed lincosamides against posttranscriptionally modified ribosomes could not have been predicted by prior structural knowledge, just as their expanded spectrum of action resists explanation by established physicochemical-property considerations alone. The exceptional safety and efficacy of the compounds in animal models of infection allows for the development of this class into orally bioavailable, broad-spectrum antibiotics capable of overcoming the threat of rising resistance mechanisms.

In one aspect, provided are compounds of Formula (I):

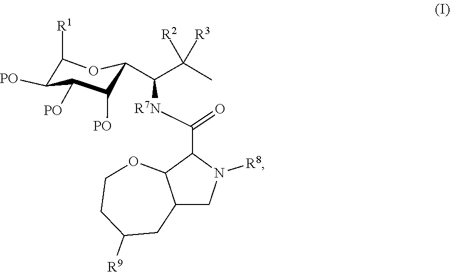

and pharmaceutically acceptable salts, salts, solvates, hydrates, polymorphs, co-crystals, tautomers, stereoisomers, isotopically labeled derivatives, and prodrugs thereof, wherein:

each P is independently hydrogen or an oxygen protecting group;

R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, or —SR^{A};

R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};

R³ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroaliphatic;

R⁷ is hydrogen or unsubstituted alkyl;

R⁸ is hydrogen, substituted or unsubstituted alkyl, substituted or unsubstituted carbocyclyl, substituted or unsubstituted heterocyclyl, substituted or unsubstituted heteroaliphatic, —C(═O)R^{A}, —C(═O)OR^{A}, —C(═O)N(R^{A})₂, —S(O)₂R^{A}, or a nitrogen protecting group;

R⁹ is substituted or unsubstituted alkyl, substituted or unsubstituted alkenyl, substituted or unsubstituted alkynyl, substituted or unsubstituted carbocyclyl, or substituted or unsubstituted heteroaliphatic, provided that R⁹ is not aralkyl; and

each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

In certain embodiments of the compound of Formula (I), each P is hydrogen.

Unless otherwise stated, any formulae described herein are also meant to include salts, solvates, hydrates, polymorphs, co-crystals, tautomers, stereoisomers, and isotopically labeled derivatives thereof. In certain embodiments, the provided compound is a salt of any of the formulae described herein. In certain embodiments, the provided compound is a pharmaceutically acceptable salt of any of the formulae described herein. In certain embodiments, the provided compound is a solvate of any of the formulae described herein. In certain embodiments, the provided compound is a hydrate of any of the formulae described herein. In certain embodiments, the provided compound is a polymorph of any of the formulae described herein. In certain embodiments, the provided compound is a co-crystal of any of the formulae described herein. In certain embodiments, the provided compound is a tautomer of any of the formulae described herein. In certain embodiments, the provided compound is a stereoisomer of any of the formulae described herein. In certain embodiments, the provided compound is of an isotopically labeled form of any of the formulae described herein. For example, compounds having the present structures except for the replacement of hydrogen by deuterium or tritium, replacement of ¹⁹F with ¹⁸F, or the replacement of a ¹²C by a ¹³C or ¹⁴C are within the scope of the disclosure. In certain embodiments, the provided compound is a deuterated form of any of the formulae or compounds described herein.

### Group R¹

As generally defined herein, R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, or —SR^{A}.

In certain embodiments, R¹ is substituted or unsubstituted alkyl, substituted or unsubstituted alkenyl, substituted or unsubstituted carbocyclyl, substituted or unsubstituted heterocyclyl, substituted or unsubstituted heteroaryl, substituted or unsubstituted heteroaralkyl, substituted or unsubstituted heteroalkyl, —OR^{A}, —N(R^{A})₂, or —SR^{A}.

In certain embodiments, R¹ is substituted or unsubstituted alkyl, substituted or unsubstituted carbocyclyl, substituted or unsubstituted heteroaralkyl, or —SR^{A}.

In certain embodiments, R¹ is substituted or unsubstituted alkyl, substituted or unsubstituted carbocyclyl, or —SR^{A}.

In certain embodiments, R¹ is

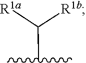

wherein R^{1a} and R^{1b} are each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}, or R^{1a} and R^{1b} are joined to form a substituted or unsubstituted heterocyclic ring, or a substituted or unsubstituted carbocyclic ring.

In certain embodiments, R^{1a} and R^{1b} are each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —OR^{A}, —OS(O)₂R^{A}, —N₃, —N(R^{A})₂, or R^{1a} and R^{1b} are joined to form a substituted or unsubstituted carbocyclic ring.

In certain embodiments, R^{1a} and R^{1b} are each independently hydrogen, halogen, substituted or unsubstituted alkyl, substituted or unsubstituted heteroaryl, or —N(R^{A})₂; or R^{1a} and R^{1b} are joined to form a substituted or unsubstituted carbocyclic ring.

In certain embodiments, R^{1a} and R^{1b} together with the carbon to which they are attached, form an optionally substituted cycloalkyl or optionally substituted heterocyclyl.

In certain embodiments, R^{1a} and R^{1b} together with the carbon to which they are attached, form an optionally substituted cycloalkyl. In certain embodiments, R^{1a} and R^{1b} together with the carbon to which they are attached, form an optionally substituted C₃₋₆ cycloalkyl. In certain embodiments, R^{1a} and R^{1b} together with the carbon to which they are attached, form an optionally substituted C₃₋₅ cycloalkyl. In certain embodiments, R^{1a} and R^{1b} together with the carbon to which they are attached, form an optionally substituted C₃₋₄ cycloalkyl. In certain embodiments, R^{1a} and R^{1b} together with the carbon to which they are attached, form an optionally substituted cyclopentyl. In certain embodiments, R^{1a} and R^{1b} together with the carbon to which they are attached, form an optionally substituted cyclobutyl. In certain embodiments, R^{1a} and R^{1b} together with the carbon to which they are attached, form an optionally substituted cyclopropyl. In certain embodiments, R^{1a} and R^{1b} together with the carbon to which they are attached, form an unsubstituted cyclopropyl.

In certain embodiments, R^{1a} and R^{1b} together with the carbon to which they are attached, form an optionally substituted heterocyclyl. In certain embodiments, R^{1a} and R^{1b} together with the carbon to which they are attached, form an optionally substituted 3-7 membered heterocyclyl. In certain embodiments, R^{1a} and R^{1b} together with the carbon to which they are attached, form an optionally substituted 4-7 membered heterocyclyl. In certain embodiments, R^{1a} and R^{1b} together with the carbon to which they are attached, form an optionally substituted 4-6 membered heterocyclyl. In certain embodiments, R^{1a} and R^{1b} together with the carbon to which they are attached, form an optionally substituted 4-6 membered heterocyclyl with at least one nitrogen atom in the ring. In certain embodiments, R^{1a} and R^{1b} together with the carbon to which they are attached, form an optionally substituted azetidine, pyrrolidine, or piperidine. In certain embodiments, R^{1a} and R^{1b} together with the carbon to which they are attached, form a substituted azetidine, pyrrolidine, or piperidine.

In certain embodiments, R¹ is of the formula:

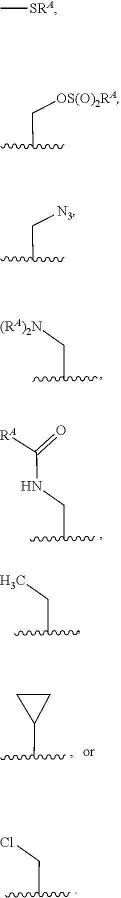

In certain embodiments, R¹ is of the formula:

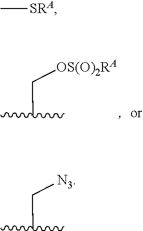

In certain embodiments, R¹ is of the formula:

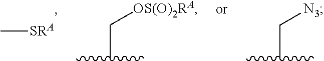

and R^{A} is substituted or unsubstituted aryl or substituted or unsubstituted alkyl.

In certain embodiments, R¹ is —SR^{A}. In certain embodiments, R¹ is —SR^{A}; and R^{A} is a substituted or unsubstituted alkyl. In certain embodiments, R¹ is —SR^{A}; and R^{A} is an unsubstituted alkyl. In certain embodiments, R¹ is —SR^{A}; and R^{A} is an unsubstituted C₁₋₄ alkyl. In certain embodiments, R¹ is —SCH₃.

R² and R³

As generally defined herein, R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)R^{A}, —OC(═O)N(R^{A})₂, —NR^{A}S(O)₂R^{A}, —OS(O)₂R^{A}, or —S(O)₂R^{A}.

In certain embodiments, R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaryl, substituted or unsubstituted heteroalkyl, —OR^{A}, —N₃, —N(R^{A})₂, —SR^{A}, —NR^{A}C(═O)R^{A}, or —OC(═O)N(R^{A})₂.

In certain embodiments, R² is halogen, substituted or unsubstituted alkyl, substituted or unsubstituted alkenyl, substituted or unsubstituted heteroaryl, —OR^{A}, —N₃, —N(R^{A})₂, —SR^{A}, —NR^{A}C(═O)R^{A}, or —OC(═O)N(R^{A})₂. In certain embodiments, R² is substituted or unsubstituted heteroaryl. In certain embodiments, R² is substituted or unsubstituted 5-membered heteroaryl. In certain embodiments, R² is substituted or unsubstituted pyrrolyl, imidazolyl, pyrazolyl, or triazolyl.

In certain embodiments, R² is halogen, substituted or unsubstituted alkyl, —OR^{A}, —N₃, —N(R^{A})₂, or —SR^{A}. In certain embodiments, R² is halogen or —SR^{A}. In certain embodiments, R² is —Cl or —SCH₃. In certain embodiments, R² is halogen. In certain embodiments, R² is —Cl. In certain embodiments, R² is —SR^{A}. In certain embodiments, R² is —SR^{A}; and R^{A} is substituted or unsubstituted heteroaryl or substituted or unsubstituted aryl. In certain embodiments, R² is —SR^{A}; and R^{A} is substituted or unsubstituted heteroaryl. In certain embodiments, R² is —SR^{A}; and R^{A} is substituted or unsubstituted thiadiazole. In certain embodiments, R² is —SR^{A}; and R^{A} is substituted thiadiazole. In certain embodiments, R² is —SR^{A}; and R^{A} is substituted or unsubstituted aryl. In certain embodiments, R² is —SR^{A}; and R^{A} is substituted aryl. In certain embodiments, R² is —SR^{A}; and R^{A} is substituted phenyl. In certain embodiments, R² is

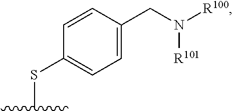

wherein R¹⁰⁰ and R¹⁰¹ are each independently hydrogen or alkyl, or together with the atoms to which they are attached form a substituted or unsubstituted heterocyclyl ring. In certain embodiments, R² is

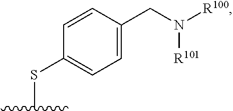

wherein R¹⁰⁰ and R¹⁰¹ are each independently hydrogen or C₁₋₄ alkyl, or together with the atoms to which they are attached form a a substituted or unsubstituted heterocyclyl ring. In certain embodiments, R² is

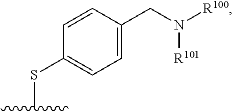

wherein R¹⁰⁰ and R¹⁰¹ are each independently alkyl or together with the atoms to which they are attached form a a substituted or unsubstituted heterocyclyl ring. In certain embodiments, R² is

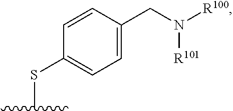

wherein R¹⁰⁰ and R¹⁰¹ are each independently C₁₋₄ alkyl or together with the atoms to which they are attached form a a substituted or unsubstituted heterocyclyl ring. In certain embodiments, R² is

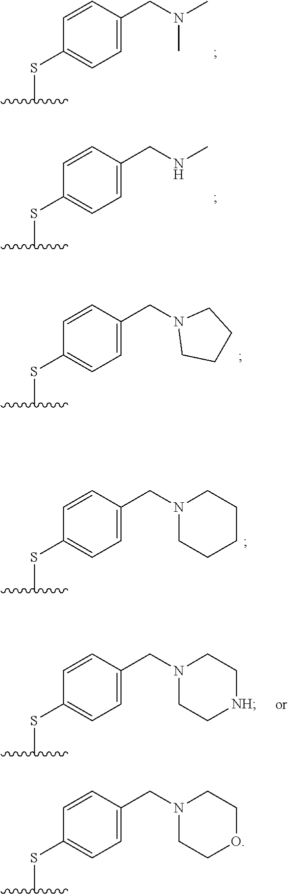 [SMILES: *Sc1ccc(CN(C)C)cc1 ; *Sc1ccc(CNC)cc1 ; *Sc1ccc(CN2CCCC2)cc1 ; *Sc1ccc(CN2CCCCC2)cc1 ; *Sc1ccc(CN2CCNCC2)cc1 ; *Sc1ccc(CN2CCOCC2)cc1]

In certain embodiments, R² is

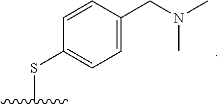 [SMILES: *Sc1ccc(CN(C)C)cc1]

As generally defined herein, R³ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroaliphatic.

In certain embodiments, R³ is hydrogen, halogen, substituted or unsubstituted alkyl, or substituted or unsubstituted alkenyl. In certain embodiments, R³ is hydrogen or substituted or unsubstituted alkyl. In certain embodiments, R³ is hydrogen. In certain embodiments, R³ is substituted or unsubstituted C₁₋₆ alkyl. In certain embodiments, R³ is unsubstituted C₁₋₆ alkyl. In certain embodiments, R³ is unsubstituted C₁₋₃ alkyl. In certain embodiments, R³ is unsubstituted C₁₋₂ alkyl. In certain embodiments, R³ is ethyl. In certain embodiments, R³ is methyl.

In certain embodiments, R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaryl, substituted or unsubstituted heteroalkyl, —OR^{A}, —N₃, —N(R^{A})₂, —SR^{A}, —NR^{A}C(═O)R^{A}, or —OC(═O)N(R^{A})₂; and R³ is hydrogen, halogen, substituted or unsubstituted alkyl, or substituted or unsubstituted alkenyl.

In certain embodiments, R² is halogen, substituted or unsubstituted alkyl, substituted or unsubstituted alkenyl, substituted or unsubstituted heteroaryl, —OR^{A}, —N₃, —N(R^{A})₂, —SR^{A}, —NR^{A}C(═O)R^{A}, or —OC(═O)N(R^{A})₂; and R³ is hydrogen, halogen, substituted or unsubstituted alkyl, or substituted or unsubstituted alkenyl.

In certain embodiments, R² is halogen, substituted or unsubstituted alkyl, —OR^{A}, —N₃, —N(R^{A})₂, or —SR^{A}; and R³ is hydrogen, halogen, substituted or unsubstituted alkyl, or substituted or unsubstituted alkenyl.

In certain embodiments, R² is halogen, substituted or unsubstituted alkyl, —OR^{A}, —N₃, —N(R^{A})₂, or —SR^{A}; and R³ is hydrogen or substituted or unsubstituted alkyl.

In certain embodiments, R² is halogen, —OR^{A}, or —SR^{A}; and R³ is hydrogen or substituted or unsubstituted alkyl. In certain embodiments, R² is halogen or —SR^{A}; and R³ is hydrogen. In certain embodiments, R² is —Cl or —SR^{A}; and R³ is hydrogen.

In certain embodiments, R² is halogen; and R³ is hydrogen. In certain embodiments, R² is is —Cl; and R³ is hydrogen. In certain embodiments, R² is halogen; and R³ is halogen. In certain embodiments, R² is —F; and R³ is —F.

In certain embodiments, R² is —SR^{A}; and R³ is hydrogen. In certain embodiments, R² is

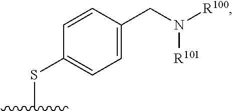

wherein R¹⁰⁰ and R¹⁰¹ are each independently hydrogen or alkyl, or together with the atoms to which they are attached form a substituted or unsubstituted heterocyclyl ring; and R³ is hydrogen. In certain embodiments, R² is

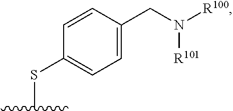

wherein R¹⁰⁰ and R¹⁰¹ are each independently hydrogen or C₁₋₄ alkyl, or together with the atoms to which they are attached form a a substituted or unsubstituted heterocyclyl ring; and R³ is hydrogen. In certain embodiments, R² is

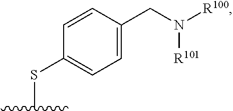

wherein R¹⁰⁰ and R¹⁰¹ are each independently alkyl or together with the atoms to which they are attached form a a substituted or unsubstituted heterocyclyl ring; and R³ is hydrogen. In certain embodiments, R² is

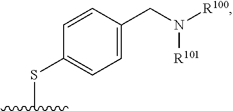

wherein R¹⁰⁰ and R¹⁰¹ are each independently C₁₋₄ alkyl or together with the atoms to which they are attached form a a substituted or unsubstituted heterocyclyl ring; and R³ is hydrogen. In certain embodiments, R² is

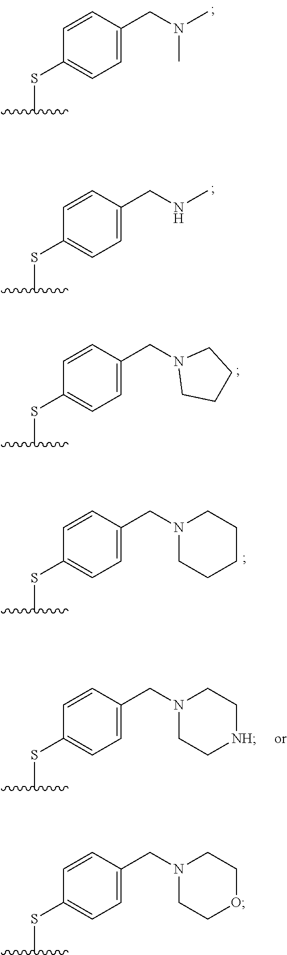 [SMILES: *Sc1ccc(CN(C)C)cc1 ; *Sc1ccc(CNC)cc1 ; *Sc1ccc(CN2CCCC2)cc1 ; *Sc1ccc(CN2CCCCC2)cc1 ; *Sc1ccc(CN2CCNCC2)cc1 ; *Sc1ccc(CN2CCOCC2)cc1]

and R³ is hydrogen. In certain embodiments, R² is

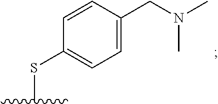 [SMILES: *Sc1ccc(CN(C)C)cc1]

and R³ is hydrogen.

### Group R⁷

As generally defined herein, R⁷ is hydrogen or unsubstituted alkyl.

In certain embodiments, R⁷ is hydrogen or unsubstituted alkyl. In certain embodiments, R⁷ is unsubstituted alkyl. In certain embodiments, R⁷ is unsubstituted C₁₋₆ alkyl. In certain embodiments, R⁷ is unsubstituted C₁₋₄ alkyl. In certain embodiments, R⁷ is unsubstituted C₁₋₃ alkyl. In certain embodiments, R⁷ is unsubstituted C₁₋₂ alkyl. In certain embodiments, R⁷ is ethyl. In certain embodiments, R⁷ is methyl. In certain embodiments, R⁷ is hydrogen.

### Group R⁸

As generally defined herein, R⁸ is hydrogen, substituted or unsubstituted alkyl, substituted or unsubstituted carbocyclyl, substituted or unsubstituted heterocyclyl, substituted or unsubstituted heteroaliphatic, —C(═O)R^{A}, —C(═O)OR^{A}, —C(═O)N(R^{A})₂, —S(O)₂R^{A}, or a nitrogen protecting group.

In certain embodiments, R⁸ is hydrogen, substituted or unsubstituted alkyl, or —C(═O)R^{A}. In certain embodiments, R⁸ is hydrogen or substituted or unsubstituted alkyl. In certain embodiments, R⁸ is hydrogen or unsubstituted alkyl. In certain embodiments, R⁸ is hydrogen or unsubstituted C₁₋₆ alkyl. In certain embodiments, R⁸ is hydrogen or unsubstituted C₁₋₄ alkyl. In certain embodiments, R⁸ is hydrogen or unsubstituted C₁₋₃ alkyl. In certain embodiments, R⁸ is hydrogen or unsubstituted C₁₋₂ alkyl. In certain embodiments, R⁸ is hydrogen or ethyl. In certain embodiments, R⁸ is hydrogen or methyl. In certain embodiments, R⁸ is hydrogen.

In certain embodiments, R⁸ is unsubstituted C₁₋₆ alkyl. In certain embodiments, R⁸ is unsubstituted C₁₋₄ alkyl. In certain embodiments, R⁸ is unsubstituted C₁₋₃ alkyl. In certain embodiments, R⁸ is unsubstituted C₁₋₂ alkyl. In certain embodiments, R⁸ is ethyl. In certain embodiments, R⁸ is methyl.

### R⁹

As generally defined herein, R⁹ is substituted or unsubstituted alkyl, substituted or unsubstituted alkenyl, substituted or unsubstituted alkynyl, substituted or unsubstituted carbocyclyl, or substituted or unsubstituted heteroaliphatic, provided that R⁹ is not aralkyl. In certain embodiments, the R⁹ substituent is the (S) stereocenter. In certain embodiments, the R⁹ substituent is the (R) stereocenter.

In certain embodiments, R⁹ is substituted or unsubstituted alkyl, substituted or unsubstituted alkenyl, substituted or unsubstituted carbocyclyl, or substituted or unsubstituted heteroalkyl. In certain embodiments, R⁹ is substituted or unsubstituted alkyl, substituted or unsubstituted alkenyl, substituted or unsubstituted carbocyclyl, or unsubstituted heteroalkyl. In certain embodiments, R⁹ is substituted or unsubstituted alkyl, substituted or unsubstituted alkenyl, or substituted or unsubstituted carbocyclyl.

In certain embodiments, R⁹ is substituted or unsubstituted alkenyl. In certain embodiments, R⁹ is unsubstituted alkenyl. In certain embodiments, R⁹ is —CH═CH₂.

In certain embodiments, R⁹ is substituted or unsubstituted carbocyclyl. In certain embodiments, R⁹ is unsubstituted carbocyclyl. In certain embodiments, R⁹ is unsubstituted C₃₋₆ cycloalkyl. In certain embodiments, R⁹ is unsubstituted C₅₋₆ cycloalkyl. In certain embodiments, R⁹ is unsubstituted cyclopentyl. In certain embodiments, R⁹ is unsubstituted cyclohexyl.

In certain embodiments, R⁹ is substituted or unsubstituted alkyl. In certain embodiments, R⁹ is substituted or unsubstituted C₁₋₆ alkyl. In certain embodiments, R⁹ is substituted C₁₋₆ alkyl. In certain embodiments, R⁹ is C₁₋₆ alkyl substituted with halogen, alkenyl, C₃₋₆ cycloalkyl, or —OR^{A}. In certain embodiments, R⁹ is C₁₋₆ alkyl substituted with halogen, alkenyl, C₃₋₆ cycloalkyl, heterocyclyl, —N(R^{A})₂, —SO₂R^{A}, or —OR^{A}. In certain embodiments, R⁹ is —CH₂CH═CH₂. In certain embodiments, R⁹ is —CH₂CH₂F. In certain embodiments, R⁹ is —CH₂CHF₂. In certain embodiments, R⁹ is —CH₂CH₂CH₂F. In certain embodiments, R⁹ is —CH₂CH₂CF₂H. In certain embodiments, R⁹ is —CH₂CH₂CH₂Cl. In certain embodiments, R⁹ is

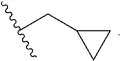 [SMILES: *CC1CC1]

In certain embodiments, R⁹ is —CH₂CH₂CH₂OCH₃. In certain embodiments, R⁹ is —CH₂CH₂CH₂OH. In certain embodiments, R⁹ is —CH₂CH₂CH₂SO₂CH₃. In certain embodiments, R⁹ is

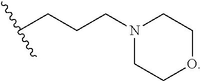 [SMILES: *CCCN1CCOCC1]

In certain embodiments, R⁹ is —CH₂CH₂CH₂NH₂. In certain embodiments, R⁹ is —CH₂CH₂CH₂N(CH₃)₂. In certain embodiments, R⁹ is —CH₂C(CH₃)₂OH. In certain embodiments, R⁹ is —CH₂CF₂CH₃. In certain embodiments R⁹ is —CH₂CH═CH₂, —CH₂CH₂F, —CH₂CHF₂,

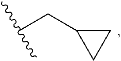 [SMILES: *CC1CC1]

or —CH₂CH₂CH₂OCH₃. In certain embodiments, R⁹ is —CH₂CH═CH₂, —CH₂CH₂F, —CH₂CHF₂, —CH₂CF₂CH₃, —CH₂CH₂CH₂F, —CH₂CH₂CF₂H, —CH₂CH₂CH₂Cl,

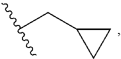 [SMILES: *CC1CC1]

—CH₂CH₂CH₂OCH₃, —CH₂CH₂CH₂OH, —CH₂C(CH₃)₂OH, —CH₂CH₂CH₂SO₂CH₃, CH₂CH₂CH₂NH₂, —CH₂CH₂CH₂N(CH₃)₂, or

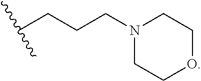 [SMILES: *CCCN1CCOCC1]

In certain embodiments, R⁹ is unsubstituted C₁₋₆ alkyl. In certain embodiments, R⁹ is —CH₃, —CH₂CH₃, —CH₂CH₂CH₃, —CH(CH₃)₂, —CH₂CH(CH₃)₂, or —C(CH₃)₃. In certain embodiments, R⁹ is —CH₃, —CH₂CH₃, —CH₂CH₂CH₃, —CH₂CH₂CH₂CH₃, —CH(CH₃)₂, —CH₂CH(CH₃)₂, —CH₂C(CH₃)₃, or —C(CH₃)₃. In certain embodiments, R⁹ is —CH₃. In certain embodiments, R⁹ is —CH₂CH₃. In certain embodiments, R⁹ is —CH₂CH₂CH₃. In certain embodiments, R⁹ is —CH(CH₃)₂. In certain embodiments, R⁹ is —C(CH₃)₃. In certain embodiments, R⁹ is —CH₂CH(CH₃)₂.

In certain embodiments, R⁹ is —CH₂CH═CH₂, —CH₃, —CH₂CH₃, —CH₂CH₂CH₃, —CH(CH₃)₂, —CH₂CH(CH₃)₂, —CH(CH₃)₃, —CH₂CH₂F, —CH₂CHF₂,

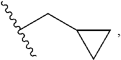 [SMILES: *CC1CC1]

—CH₂CH₂CH₂OCH₃,

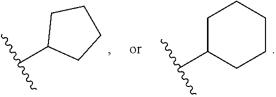 [SMILES: *C1CCCC1 ; *C1CCCCC1]

In certain embodiments, R⁹ is —CH═CH₂, —CH₂CH═CH₂, —CH₃, —CH₂CH₃, —CH₂CH₂CH₃, —CH₂CH₂CH₂CH₃, —CH(CH₃)₂, —CH₂CH(CH₃)₂, —CH₂C(CH₃)₃, —CH₂CH₂F, —CH₂CHF₂, —CH₂CF₂CH₃, —CH₂CH₂CH₂F, —CH₂CH₂CF₂H, —CH₂CH₂CH₂Cl,

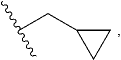 [SMILES: *CC1CC1]

—CH₂CH₂CH₂OCH₃, —CH₂CH₂CH₂OH, —CH₂C(CH₃)₂OH, —CH₂CH₂CH₂SO₂CH₃, CH₂CH₂CH₂NH₂, —CH₂CH₂CH₂N(CH₃)₂,

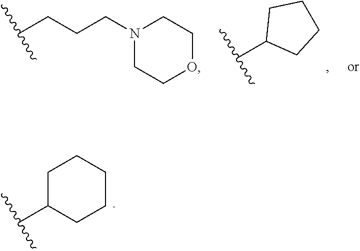

### Embodiments of Formula (I)

In certain embodiments, the compound of Formula (I) is a compound of Formula (I-a):

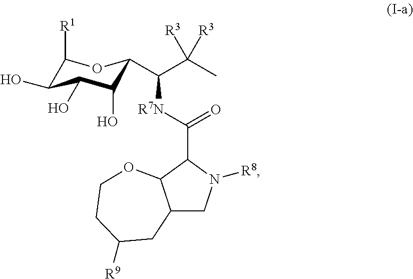

or a pharmaceutically acceptable salt thereof, wherein R¹, R², R³, R⁷, R⁸, and R⁹ are as defined herein.

In certain embodiments of the compound of Formula (I-a), R¹ is —SR^{A}; R⁷ is hydrogen; and R⁸ is hydrogen or methyl. In certain embodiments of the compound of Formula (I-a), R¹ is —SCH₃; R⁷ is hydrogen; and R⁸ is hydrogen or methyl. In certain embodiments of the compound of Formula (I-a), R¹ is —SCH₃; R⁷ is hydrogen; and R⁸ is hydrogen. In certain embodiments of the compound of Formula (I-a), R¹ is —SCH₃; R⁷ is hydrogen; and R⁸ is methyl.

In certain embodiments, the compound of Formula (I) is a compound of Formula (I-b):

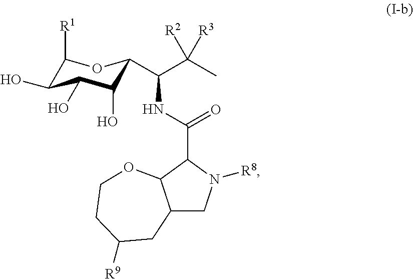

or a pharmaceutically acceptable salt thereof, wherein R¹, R², R³, R⁸, and R⁹ are as defined herein.

In certain embodiments, the compound of Formula (I) is a compound of Formula (I-c):

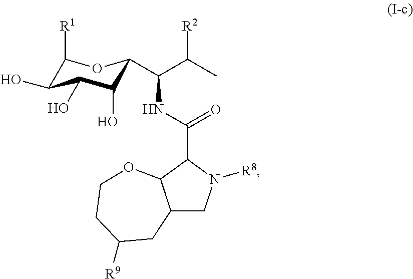

or a pharmaceutically acceptable salt thereof, wherein R¹, R², R⁸, and R⁹ are as defined herein.

In certain embodiments, the compound of Formula (I) is a compound of Formula (I-d):

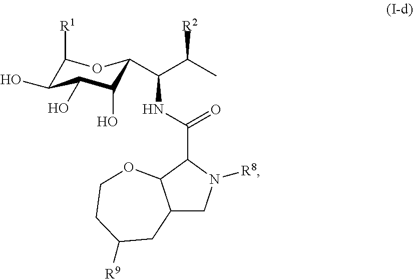

or a pharmaceutically acceptable salt thereof, wherein R¹, R², R⁸, and R⁹ are as defined herein.

In certain embodiments, the compound of Formula (I) is a compound of Formula (I-e):

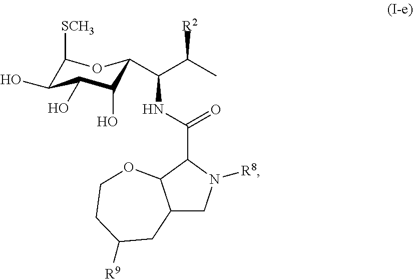

or a pharmaceutically acceptable salt thereof, wherein R², R⁸, and R⁹ are as defined herein.

In certain embodiments, the compound of Formula (I) is a compound of Formula (I-f):

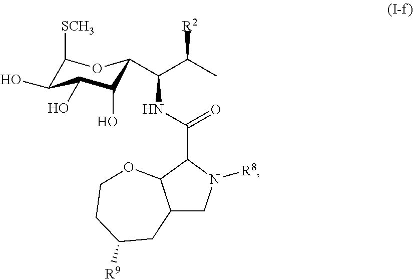

or a pharmaceutically acceptable salt thereof, wherein R², R⁸, and R⁹ are as defined herein.

In certain embodiments, the compound of Formula (I) is a compound of Formula (I-g):

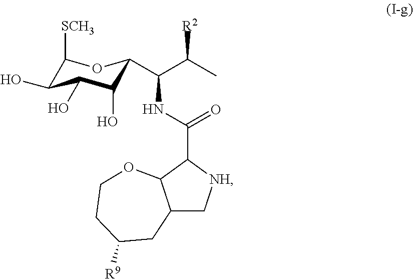

or a pharmaceutically acceptable salt thereof, wherein R² and R⁹ are as defined herein.

In certain embodiments, the compound of Formula (I) is a compound of Formula (I-h):

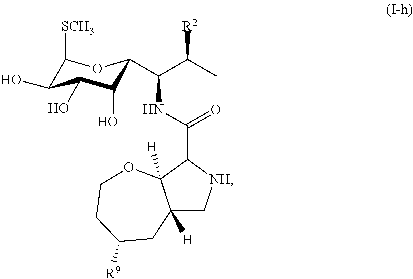

or a pharmaceutically acceptable salt thereof, wherein R² and R⁹ are as defined herein.

In certain embodiments, the compound of Formula (I) is a compound of Formula (I-i):

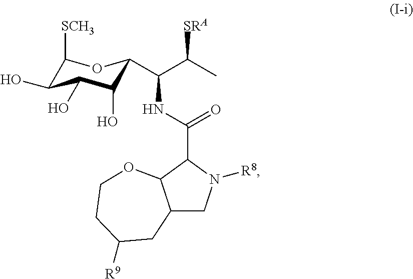

or a pharmaceutically acceptable salt thereof, wherein R^{A}, R⁸, and R⁹ are as defined herein.

In certain embodiments, the compound of Formula (I) is a compound of Formula (I-j):

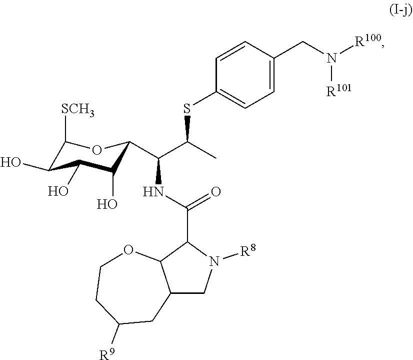

or a pharmaceutically acceptable salt thereof, wherein R¹⁰⁰ and R¹⁰¹ are each independently hydrogen or alkyl, or together with the atoms to which they are attached form a substituted or unsubstituted heterocyclyl ring; and R⁸ and R⁹ are as defined herein.

In certain embodiments, the compound of Formula (I) is a compound of Formula (I-j-1):

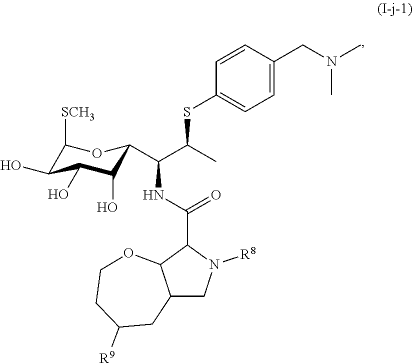

or a pharmaceutically acceptable salt thereof, wherein R⁸ and R⁹ are as defined herein.

In certain embodiments, the compound of Formula (I) is a compound of Formula (I-k):

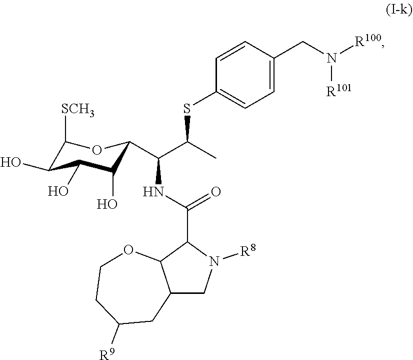

or a pharmaceutically acceptable salt thereof, wherein R¹⁰⁰ and R¹⁰¹ are each independently hydrogen or alkyl, or together with the atoms to which they are attached form a substituted or unsubstituted heterocyclyl ring; and R⁹ is as defined herein.

In certain embodiments, the compound of Formula (I) is a compound of Formula (I-k-1):

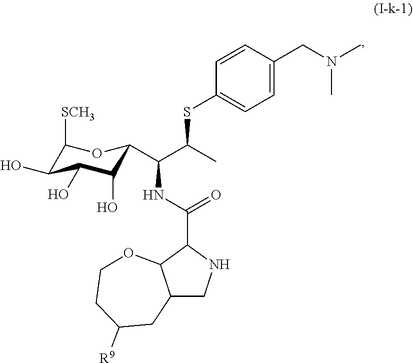

or a pharmaceutically acceptable salt thereof, wherein R⁹ is as defined herein.

In certain embodiments, the compound of Formula (I) is a compound of Formula (I-l):

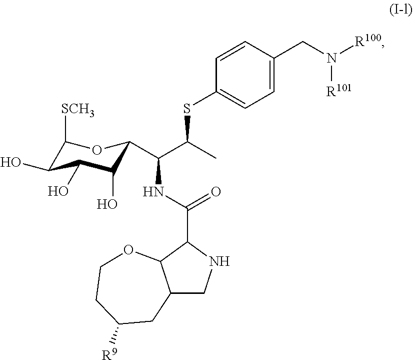

or a pharmaceutically acceptable salt thereof, wherein R¹⁰⁰ and R¹⁰¹ are each independently hydrogen or alkyl, or together with the atoms to which they are attached form a substituted or unsubstituted heterocyclyl ring; and R⁹ is as defined herein.

In certain embodiments, the compound of Formula (I) is a compound of Formula (I-l-1):

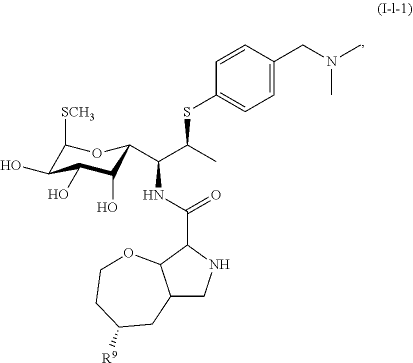

or a pharmaceutically acceptable salt thereof, wherein R⁹ is as defined herein.

In certain embodiments, the compound of Formula (I) is a compound of Formula (I-m):

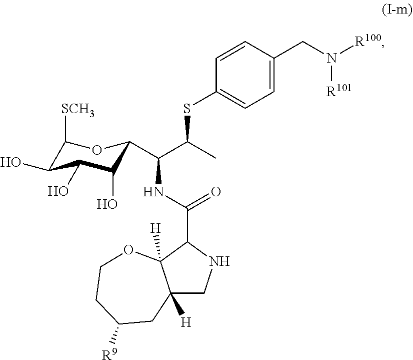

or a pharmaceutically acceptable salt thereof, wherein R¹⁰⁰ and R¹⁰¹ are each independently hydrogen or alkyl, or together with the atoms to which they are attached form a substituted or unsubstituted heterocyclyl ring; and R⁹ is as defined herein.

In certain embodiments, the compound of Formula (I) is a compound of Formula (I-m-1):

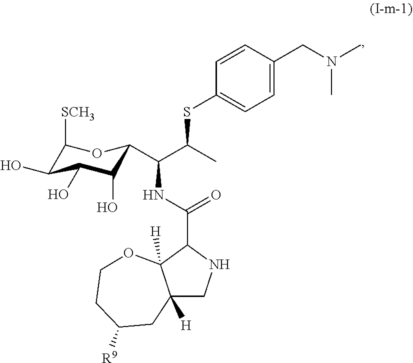

or a pharmaceutically acceptable salt thereof, wherein R⁹ is as defined herein.

In certain embodiments, the compound of Formula (I) is a compound of Formula (I-n):

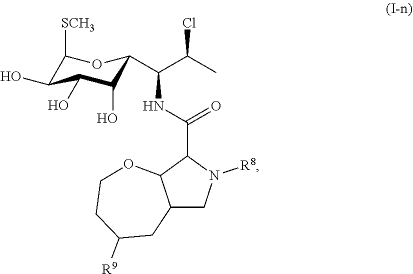

or a pharmaceutically acceptable salt thereof, wherein R⁸ and R⁹ are as defined herein.

In certain embodiments, the compound of Formula (I) is a compound of Formula (I-o):

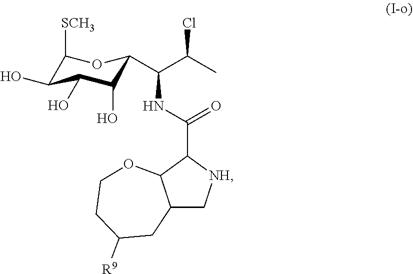

or a pharmaceutically acceptable salt thereof, wherein R⁹ is as defined herein.

In certain embodiments, the compound of Formula (I) is a compound of Formula (I-p):

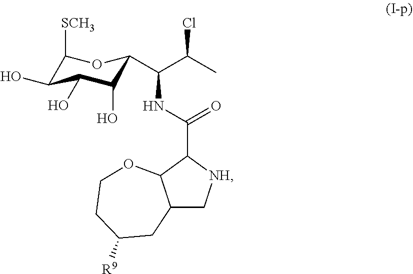

or a pharmaceutically acceptable salt thereof, wherein R⁹ is as defined herein.

In certain embodiments, the compound of Formula (I) is a compound of Formula (I-q):

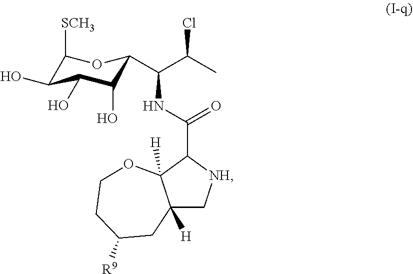

or a pharmaceutically acceptable salt thereof, wherein R⁹ is as defined herein.

In certain embodiments, the compound of Formula (I) is a compound of Formula (I-r):

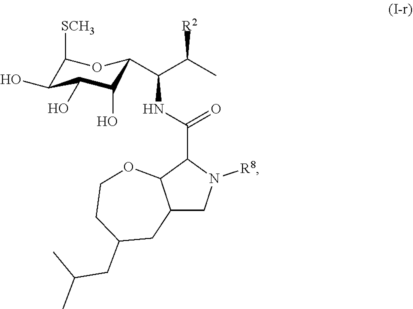

or a pharmaceutically acceptable salt thereof, wherein R² and R⁸ are as defined herein.

In certain embodiments, the compound of Formula (I) is a compound of Formula (I-s):

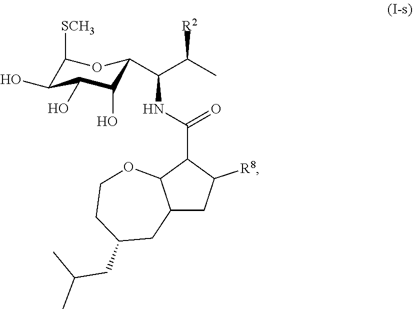

or a pharmaceutically acceptable salt thereof, wherein R² and R⁸ are as defined herein.

In certain embodiments, the compound of Formula (I) is a compound of Formula (I-t):

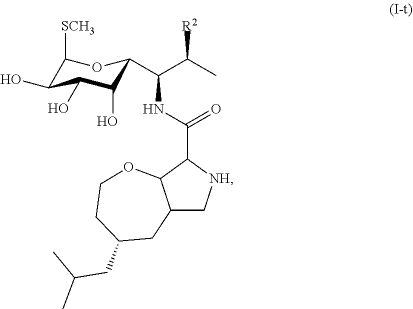

or a pharmaceutically acceptable salt thereof, wherein R² is as defined herein.

In certain embodiments, the compound of Formula (I) is a compound of Formula (I-u):

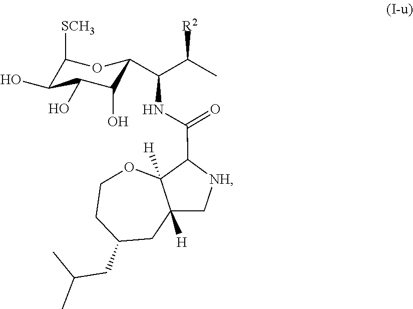

or a pharmaceutically acceptable salt thereof, wherein R² is as defined herein.

### Exemplary Compounds

Exemplary compounds of Formula (I) include, but are not limited to, the compounds listed in Table 1.

TABLE 1
Exemplary compounds of Formula (1)

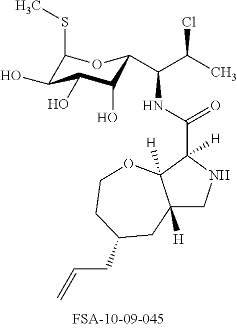

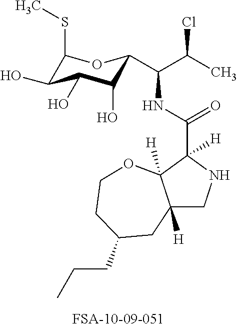

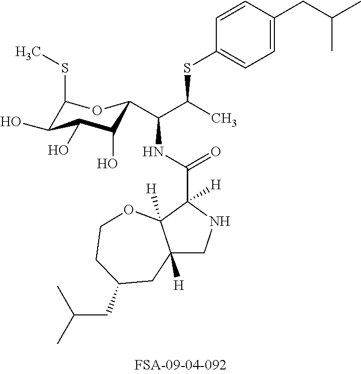

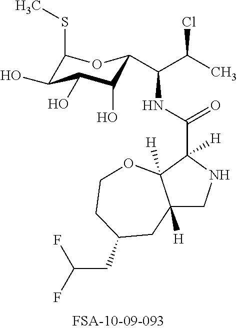

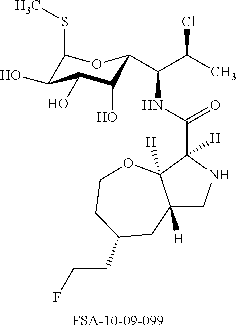

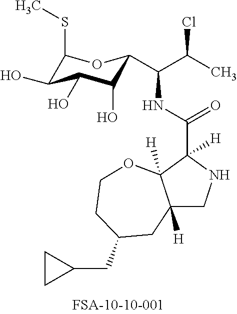

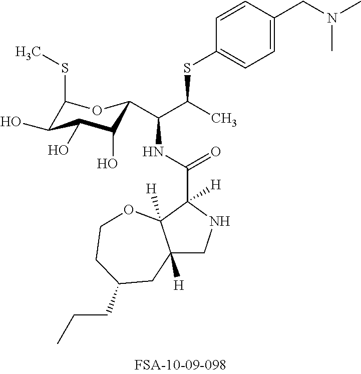

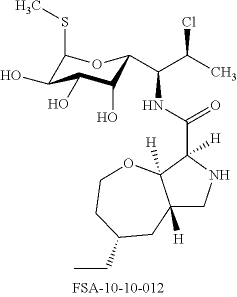

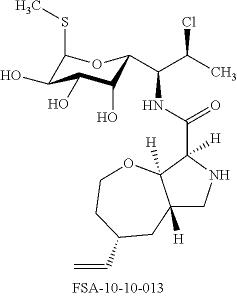

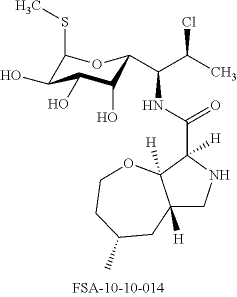

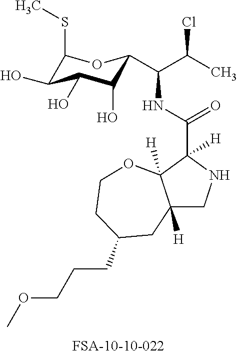

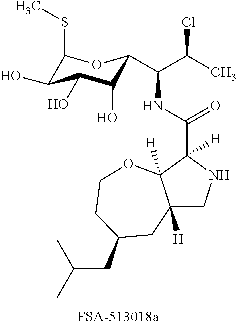

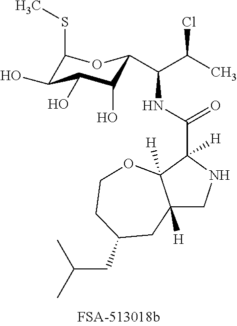

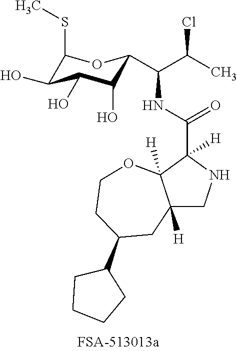

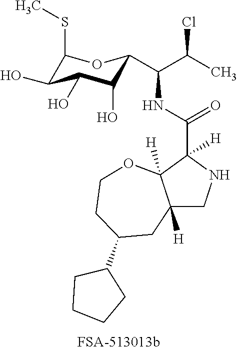

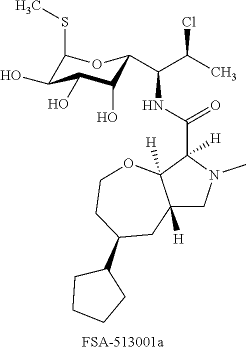

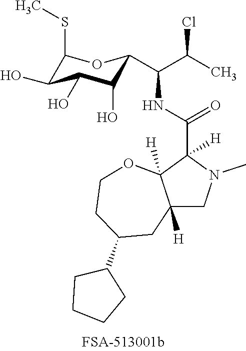

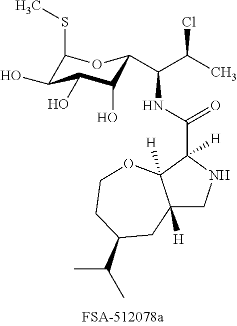

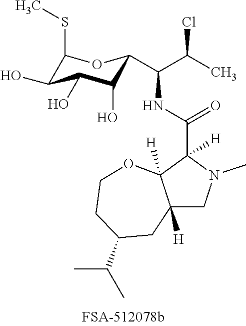

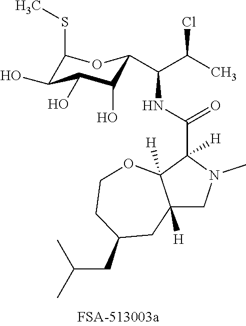

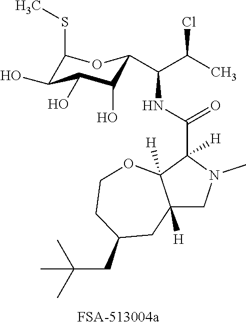

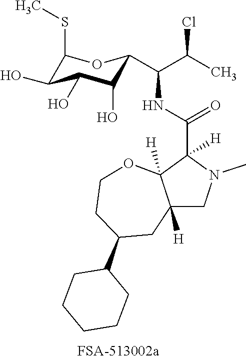

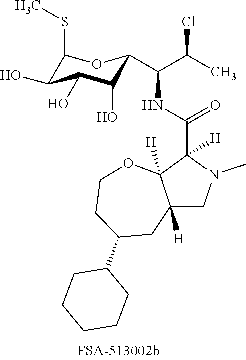

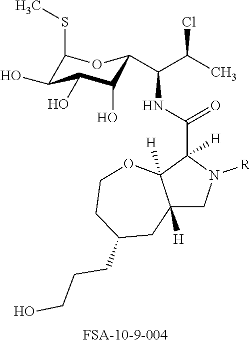

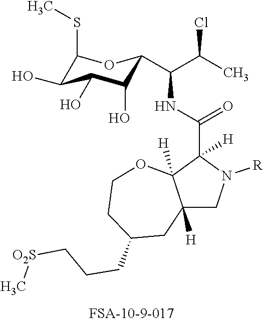

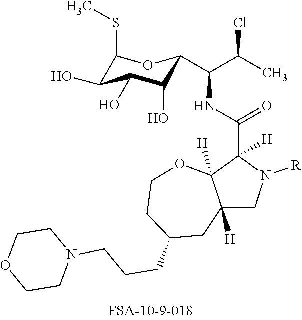

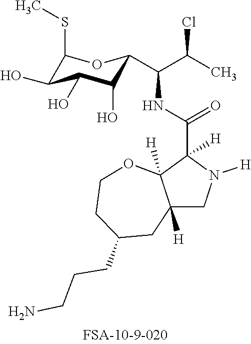

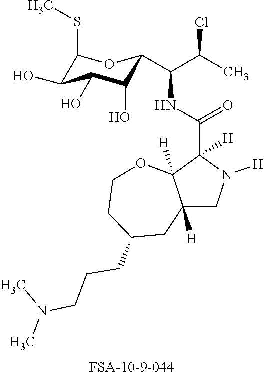

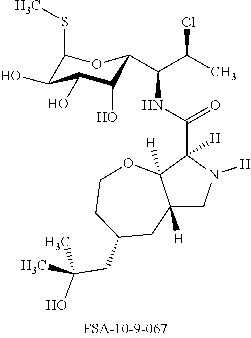

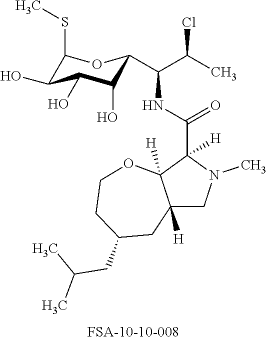

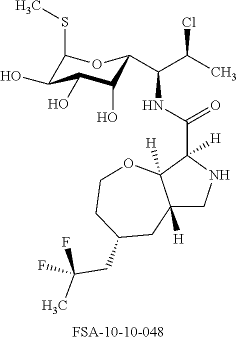

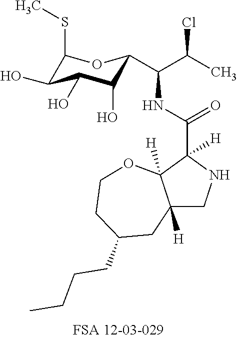

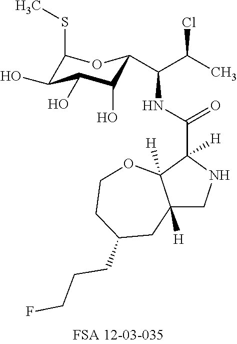

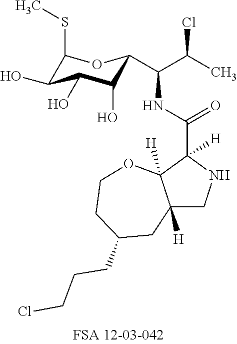

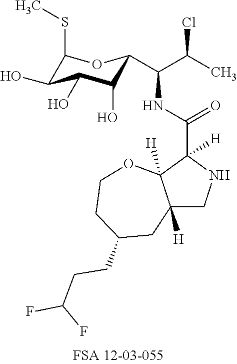

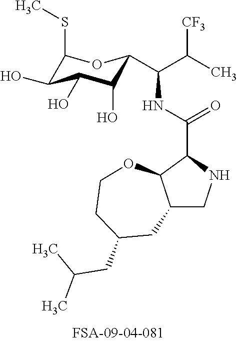

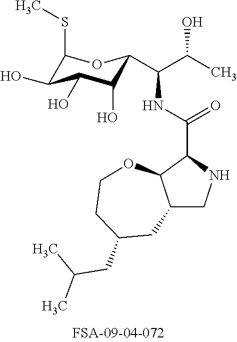

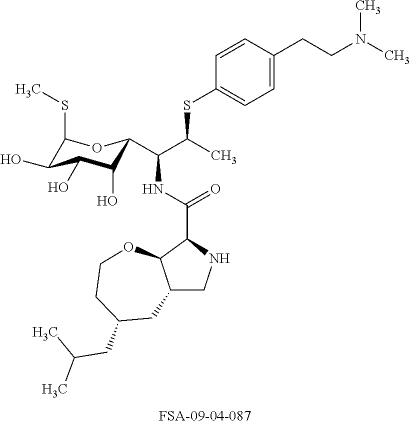

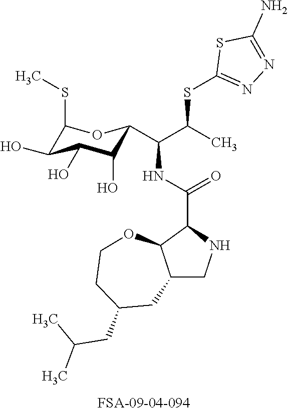

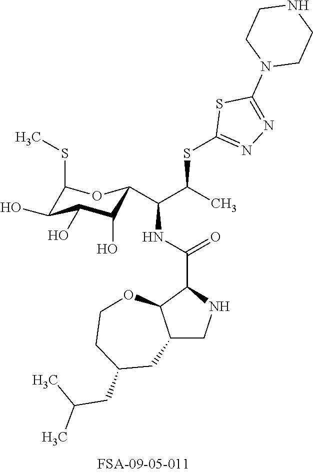

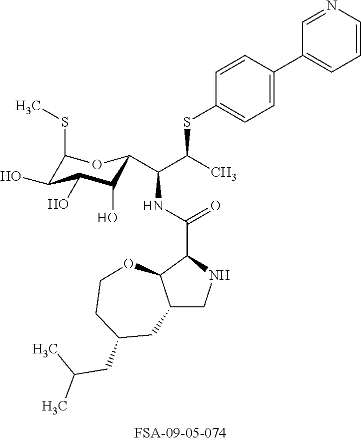

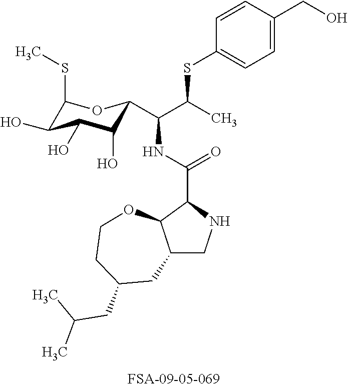

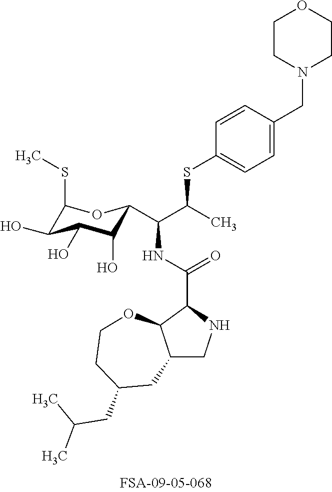

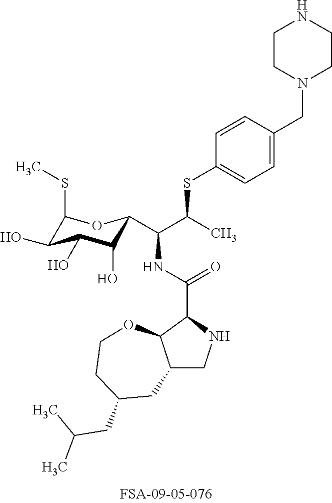

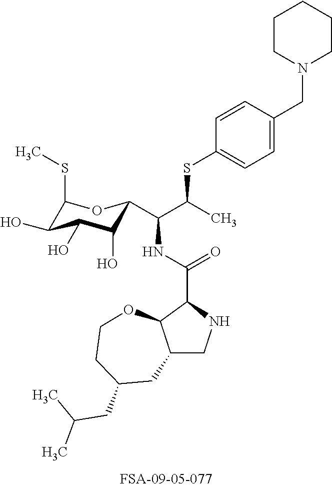

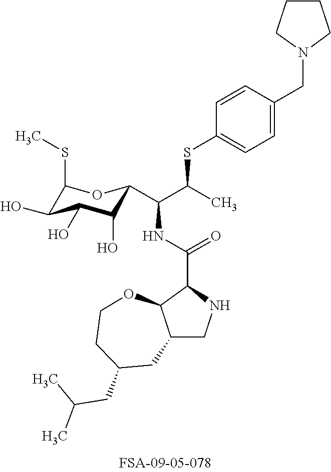

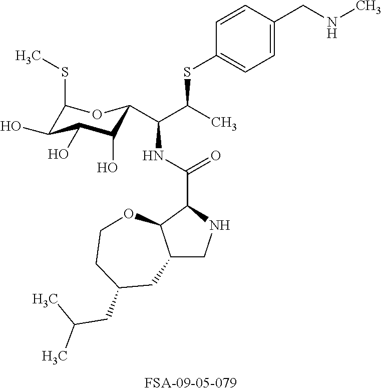

### Preparation of Compounds of Formula (I)

In another aspect, compounds of the present disclosure are prepared by coupling a compound of Formula (A) and a compound of Formula (B) as depicted in Scheme 1.

Exemplary methods that may be used in the preparation of a compound of the present disclosure are described herein, and are not to be construed as limiting. The compounds described herein may be prepared by other methods of synthesis known in the art, and the procedures described herein may be modified or combined with other known methods.

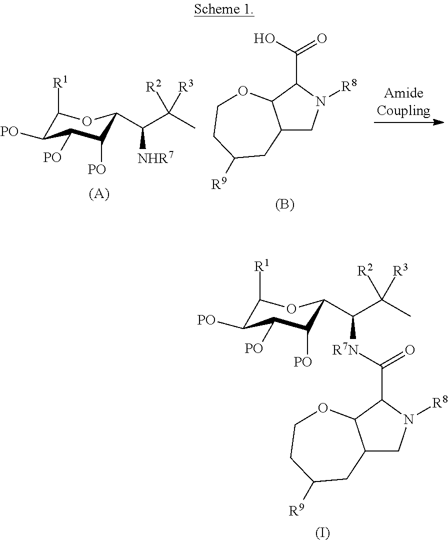

For all intermediates, P, R¹, R², R³, R⁷, R⁸, and R⁹ are as defined herein for a compound of Formula (I), unless otherwise stated.

In certain embodiments, the amide bond formation is promoted by an amide coupling reagent (e.g., 3-(diethoxyphosphoryloxy)-1,2,3-benzotriazin-4(3H)-one (DEPBT), 1-[bis(dimethylamino)methylene]-1H-1,2,3,-triazolo[4,5-b]pyridinium 3-oxid hexafluorophosphate (HATU). 1-ethyl-3-(3-dimethylaminopropyl)carbodiimide (EDC), hydroxybenzotriazole (HOBt)). In certain embodiments, the amide coupling reagent (e.g., DEPBT, HATU, EDC, HOBt) is reacted with the compound of Formula (B). In certain embodiments, the amide coupling reagent (e.g., DEPBT, HATU, EDC, HOBt) is reacted with the compound of Formula (B) prior to reacting with the compound of Formula (A). In certain embodiments, the amide coupling reagent is DEPBT.

In certain embodiments, the method comprises adding up to 1.0, 1.1, 1.2, 1.3, 1.4, 1.5, 1.6, 1.7, 1.8. 1.9, 2.0, 2.1, 2.2, 2.3, 2.4, 2.5, 2.6, 2.7, 2.8. 2.9, or 3.0 equivalents of the amide coupling reagent. In certain embodiments, the method comprises performing the coupling reaction at room temperature, ambient temperature, or elevated temperature. In certain embodiments, the method comprises performing the coupling reaction at 20-60° C., 20-50° C., 20-40° C., 20-30° C., 20-25° C., or 25-30° C.

In certain embodiments, the method further comprises oxidation of a compound of Formula (C):

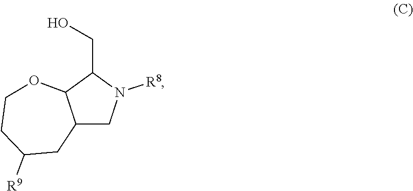

or salt thereof, to provide the compound of Formula (B), wherein R⁸ and R⁹ are as defined herein.

In certain embodiments, the oxidation comprises a multistep transformation of the alcohol to the carboxylic acid. In certain embodiments, the oxidation comprises two reaction steps. In certain embodiments, the oxidation comprises a first oxidation of the alcohol to an aldehyde (e.g., by Dess-Martin oxidation) and a second oxidation step of the aldehyde to the carboxylic acid (e.g., by Pinnick oxidation).

In certain embodiments, the method further comprises formation of a compound of Formula (C), the method comprising cyclizing a compound of Formula (D):

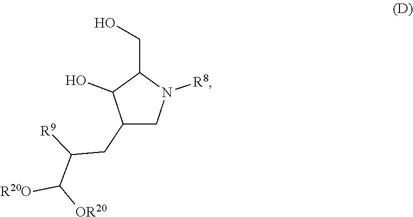

or a salt thereof, wherein R⁸ and R⁹ are as defined herein; and each R²⁰ is independently hydrogen, or substituted or unsubstitued alkyl, or both instances of R²⁰ join together, with the atoms to which they are attached, to form a substituted or unsubstitued heterocycle.

In certain embodiments, the cyclizing comprises addition of an acid (e.g., p-toluenesulfonic acid). In certain embodiments, the step of cyclizing further comprises addition of a silane (e.g., triethylsilane). In certain embodiments, the step of cyclizing comprises addition of 1,1,1,3,3,3-hexafluoroisopropanol. In certain embodiments, the cyclizing comprises addition of 1,1,1,3,3,3-hexafluoroisopropanol and p-toluenesulfonic acid.

In certain embodiments, the compound of Formula (D) is the compound of Formula (D-1):

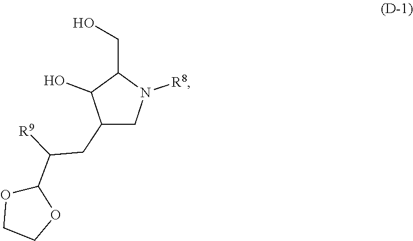

or a salt thereof, wherein R⁸ and R⁹ are as defined herein.

In certain embodiments, the compound of Formula (D) is the compound of Formula (D-2):

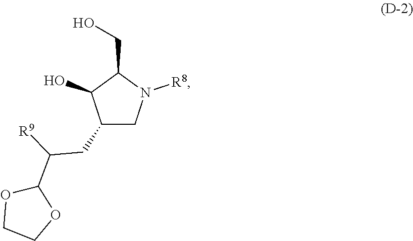

or a salt thereof, wherein R⁸ and R⁹ are as defined herein.

In certain embodiments, the method further comprises formation of a compound of Formula (D), the method comprising oxidizing a compound of Formula (E):

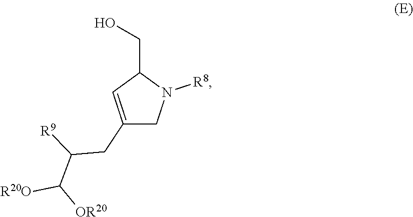

or a salt thereof, wherein each R²⁰ is independently hydrogen, or substituted or unsubstitued alkyl, or both instances of R²⁰ join together, with the atoms to which they are attached, to form a substituted or unsubstitued heterocycle; and R⁸ and R⁹ are as defined herein.

In certain embodiments, the oxidizing further comprises silylation of the primary alcohol to form a compound of Formula (E-a):

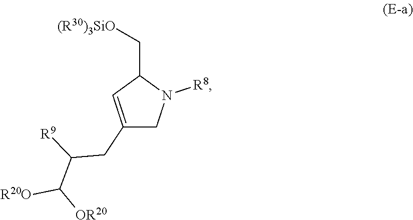

or a salt thereof, wherein each R³⁰ is independently a hydrogen, substituted or unsubstituted alkyl, or substituted or unsubstituted aryl; each R²⁰ is independently hydrogen, or substituted or unsubstitued alkyl, or both instances of R²⁰ join together, with the atoms to which they are attached, to form a substituted or unsubstitued heterocycle; and R⁸ and R⁹ are as defined herein.

In certain embodiments, the compound of Formula (E-a) is the compound of Formula (E-a-1):

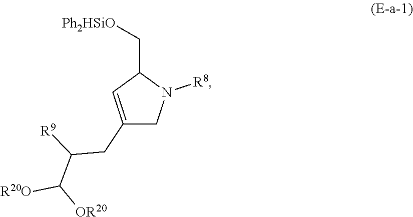

or a salt thereof, wherein each R²⁰ is independently hydrogen, or substituted or unsubstitued alkyl, or both instances of R²⁰ join together, with the atoms to which they are attached, to form a substituted or unsubstitued heterocycle; and R⁸ and R⁹ are as defined herein.

In certain embodiments, the step of oxidizing further comprises addition of an oxidizing agent (e.g., H₂O₂) and a transition metal catalyst (e.g., a platinum metal catalyst, e.g., Pt₂(dvtms)₃ to form the compound of Formula (D).

In certain embodiments, the compound of Formula (E) is the compound of Formula (E-1):

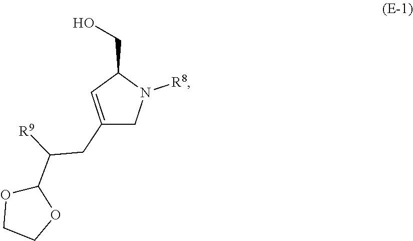

or a salt thereof.

In certain embodiments, the method further comprises formation of a compound of Formula (E), the method comprising coupling a compound of Formula (F):

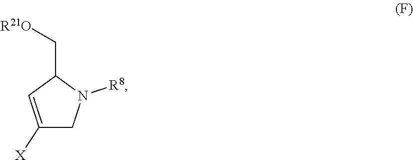

or a salt thereof, with a compound of Formula (G):

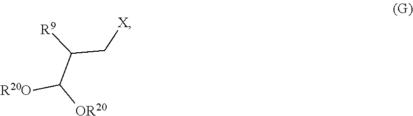

or a salt thereof, wherein R⁸, R⁹, and R²⁰ are as defined herein; R²¹ is an oxygen protecting group or hydrogen; and each X is independently a leaving group. In certain embodiments, each X is independently a halogen or a triflate. In certain embodiments, each X is independently iodide or triflate. In certain embodiments, the compound of Formula (G) forms an organozinc intermediate. In certain embodiments, the coupling further comprises addition of a transition metal catalyst (e.g., a palladium metal catalyst, such as PdCl₂dppf).

In certain embodiments, the compound of Formula (F) is the compound of Formula (F-1):

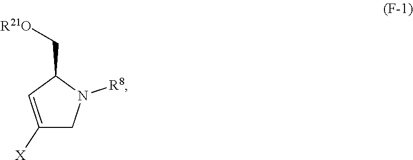

or a salt thereof, wherein X is a leaving group; R²¹ is an oxygen protecting group or hydrogen; and R⁸ is as defined herein.

In certain embodiments, the compound of Formula (G) is the compound of Formula (G-1):

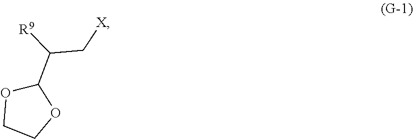

or a salt thereof, wherein X is a leaving group; and R⁹ is as defined herein.

In certain embodiments, the compound of Formula (G) is the compound of Formula (G-2):

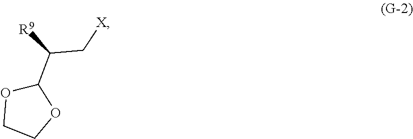

or a salt thereof, wherein X is a leaving group; and R⁹ is as defined herein.

In certain embodiments, the compound of Formula (G) is the compound of Formula (G-3):

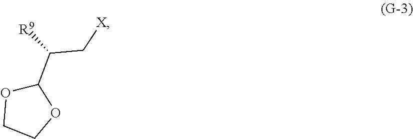

or a salt thereof, wherein X is a leaving group; and R⁹ is as defined herein.

In certain embodiments, the method further comprises coupling a compound of Formula (H):

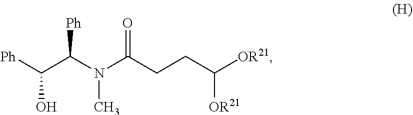

or a salt thereof, with a compound of Formula (J):

R⁹—X (J),

or a salt thereof, wherein R⁹ is as defined herein; R²¹ is an oxygen protecting group or hydrogen; and X is a leaving group. In certain embodiments, X is a halogen.

In certain embodiments, the coupling comprises treating the compound of Formula (H) with a base prior to reacting with a compound of Formula (J). In certain embodiments, the base is an organolithium reagent. In certain embodiments, the base is lithium hexamethyldisilazide, lithium diisopropylamide, or lithium tetramethylpiperidide. In certain embodiments, the base is lithium diisopropylamide.

In certain embodiments, the method comprises reacting the compound of Formula (G) with the compound of Formula (H) at a temperature of less than 0° C., less than −10° C., less than −20° C., less than −30° C., less than −40° C., less than −50° C., less than −60° C., less than −70° C., less than −75° C., less than −80° C., or less than −85° C.

In certain embodiments, the compound of Formula (H) is the compound of Formula (H-1):

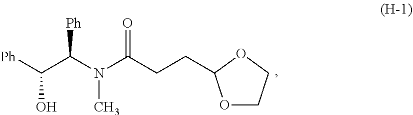 [SMILES: CN(C(=O)CCC1OCCO1)[C@H](c1ccccc1)[C@H](O)c1ccccc1]

or a salt thereof.

In certain embodiments, the compound of Formula (J) is the compound of Formula (J-1):

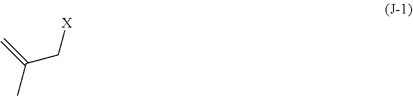

or a salt thereof, wherein X is a leaving group. In certain embodiments, X is a halogen.

### Pharmaceutical Compositions and Administration

The present disclosure provides pharmaceutical compositions comprising a compound as described herein, or a pharmaceutically acceptable salt thereof, and a pharmaceutically acceptable excipient.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the compound of the present disclosure. The amount of the compound is generally equal to the dosage of the compound which would be administered to a subject and/or a convenient fraction of such a dosage such as, for example, one-half or one-third of such a dosage.

Relative amounts of the compound, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) compound.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Liquid dosage forms for oral and parenteral administration include pharmaceutically acceptable emulsions, microemulsions, solutions, suspensions, syrups and elixirs. In addition to the compounds, the liquid dosage forms may comprise inert diluents commonly used in the art such as, for example, water or other solvents, solubilizing agents, and emulsifiers, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium.

Solid dosage forms for oral administration include capsules, tablets, pills, powders, and granules. In such solid dosage forms, the compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Dosage forms for topical and/or transdermal administration of a compound of this invention may include ointments, pastes, creams, lotions, gels, powders, solutions, sprays, inhalants and/or patches. Generally, the compound is admixed under sterile conditions with a pharmaceutically acceptable carrier and/or any needed preservatives and/or buffers as can be required.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily amount of the compound will be decided by the attending physician within the scope of sound medical judgment. The specific therapeutically effective dose level for any particular subject will depend upon a variety of factors including the disease, disorder, or condition being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent, the therapeutic regimen, and/or the condition of the subject. Oral administration is the preferred mode of administration. However, in certain embodiments, the subject may not be in a condition to tolerate oral administration, and thus intravenous, intramuscular, and/or rectal administration are also preferred alternative modes of administration.

An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein.

It will be also appreciated that a compound or composition, as described herein, can be administered in combination with one or more additional therapeutically active agents. The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In certain embodiments, the levels utilized in combination will be lower than those utilized individually.

Exemplary additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins. Therapeutically active agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the additional therapeutically active agent is an antibiotic. Exemplary antibiotics include, but are not limited to, penicillins (e.g., penicillin, amoxicillin), cephalosporins (e.g., cephalexin), compounds (e.g., erythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aureomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, and quinupristin/dalfoprisin (Syndercid™)

Also encompassed by the invention are kits (e.g., pharmaceutical packs). The kits provided may comprise an inventive pharmaceutical composition or compound and a container (e.g., a vial, ampule, bottle, syringe, and/or dispenser package, or other suitable container). In certain embodiments, provided kits may optionally further include a second container comprising a pharmaceutical excipient for dilution or suspension of an inventive pharmaceutical composition or compound. In certain embodiments, the inventive pharmaceutical composition or compound provided in the container and the second container are combined to form one unit dosage form.

### Methods of Treatment and Uses

The present disclosure contemplates using compounds of the present invention for the treatment of infectious diseases, for example, fungal, bacterial, viral, and/or parasitic infections. Lincosamides are generally known to exhibit anti-bacterial activity.

Thus, as generally described herein, provided is a method of treating an infectious disease comprising administering an effective amount of a compound of the present disclosure, or a pharmaceutically acceptable salt thereof, to a subject in need thereof. Such a method can be conducted in vivo (i.e., by administration to a subject). Treating, as used herein, encompasses therapeutic treatment and prophylactic treatment.

In certain embodiments, the effective amount is a therapeutically effective amount. For example, in certain embodiments, the method slows the progress of an infectious disease in the subject. In certain embodiments, the method improves the condition of the subject suffering from an infectious disease. In certain embodiments, the subject has a suspected or confirmed infectious disease.

In certain embodiments, the effective amount is a prophylactically effective amount. For example, in certain embodiments, the method prevents or reduces the likelihood of an infectious disease, e.g., in certain embodiments, the method comprises administering a compound of the present invention to a subject in need thereof in an amount sufficient to prevent or reduce the likelihood of an infectious disease. In certain embodiments, the subject is at risk of an infectious disease (e.g., has been exposed to another subject who has a suspected or confirmed infectious disease or has been exposed or thought to be exposed to a pathogen).

In one aspect, provided is a method of killing a microorganism (e.g., fungus, bacterium, virus, parasite) comprising contacting the microorganism with an effective amount of a compound of the present disclosure. The compound may contact the microorganism in vivo (e.g., in a subject in need thereof) or in vitro.

In another aspect, provided is a method of inhibiting the growth of a microorganism (e.g., fungus, bacterium, virus, parasite) comprising contacting the microorganism with an effective amount of a compound of the present disclosure. The compound may contact the microorganism in vivo (e.g., in a subject in need thereof) or in vitro.

In another aspect, provided is an in vitro method of inhibiting pathogenic growth comprising contacting an effective amount of the compound of the present invention with a pathogen (e.g., a bacteria, virus, fungus, or parasite) in a cell culture.

In another aspect, provided is an in vitro method of inhibiting pathogenic growth comprising contacting a pathogen (e.g., a bacteria, virus, fungus, or parasite) with an effective amount of a compound of the present disclosure. In another aspect, provided is a method of inhibiting protein synthesis (e.g., by interfering with the synthesis of proteins by binding to the 23s portion of the 50S subunit of the bacterial ribosome and causing premature dissociation of the peptidyl-tRNA from the ribosome) with an effective amount of a compound of the present disclosure. In certain embodiments, inhibiting protein synthesis comprises inhibiting the ribosome of bacteria with an effective amount of a compound of the present disclosure. Protein synthesis may be inhibited in vivo or in vitro.

As used herein, “infectious disease” and “microbial infection” are used interchangeably, and refer to an infection with a pathogen, such as a fungus, bacteria, virus, or a parasite. In certain embodiments, the infectious disease is caused by a fungus, bacteria, or a parasite. In certain embodiments, the infectious disease is caused by a pathogen resistant to other treatments. In certain embodiments, the infectious disease is caused by a pathogen that is multi-drug tolerant or resistant, e.g., the infectious disease is caused by a pathogen that neither grows nor dies in the presence of or as a result of other treatments.

In certain embodiments, the infectious disease is a bacterial infection. For example, in certain embodiments, provided is a method of treating a bacterial infection comprising administering an effective amount of a compound of the present invention, or a pharmaceutically acceptable salt thereof, to a subject in need thereof.

In certain embodiments, the compound has a mean inhibitory concentration (MIC), with respect to a particular bacteria, of less than 50 μg/mL, less than 25 μg/mL, less than 20 μg/mL, less than 10 μg/mL, less than 5 μg/mL, or less than 1 μg/mL.

In certain embodiments, the bacteria is susceptible (e.g., responds to) or resistant to known commercial compounds, such as azithromycin, lincomycin, clindamycin, telithromycin, erythromycin, spiramycin, and the like. In certain embodiments, the bacteria is resistant to a known compound. For example, in certain embodiments, the bacteria is lincomycin or clindamycin resistant.

In certain embodiments, the bacterial infection is resistant to other antibiotics (e.g., non-compound) therapy. For example, in certain embodiments, the pathogen is vancomycin resistant (VR). In certain embodiments, the pathogen is methicillin-resistant (MR), e.g., in certain embodiments, the bacterial infection is an methicillin-resistant S. aureus infection (a MRSA infection). In certain embodiments, the pathogen is quinolone resistant (QR). In certain embodiments, the pathogen is fluoroquinolone resistant (FR, FQR).

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacterial infection is an infection with a Gram positive bacterium.

In certain embodiments, the Gram positive bacterium is a bacterium of the phylum Firmicutes.

In certain embodiments, the bacteria is a member of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus.

In certain embodiments, the bacteria is a member of the phylum Firmicutes and the genus Staphylococcus, i.e., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In certain embodiments, the Staphylococcus infection is a S. aureus infection.

In certain embodiments, the S. aureus has an efflux (e.g., mef, msr) genotype. Bacteria of the efflux genotypes actively pump drug out of the cell via efflux pumps.

In certain embodiments, the S. aureus has a methylase (e.g., erm) genotype. In certain embodiments, erm is the bacterial gene class coding for erythromycin ribosomal methylase, which methylates a single adenine in 23S rRNA, itself a component of 50S rRNA.

In certain embodiments, the bacteria is a member of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the Bacillus infection is a B. subtilis infection. In certain embodiments, the B. subtilis has an efflux (e.g., mef, msr) genotype. In certain embodiments, the B. subtilis has a methylase (e.g., erm) genotype.

In certain embodiments, the bacteria is a member of the phylum Firmicutes and the genus Streptococcus, i.e., the bacterial infection is a Strepococcus infection. Exemplary Streptococcus bacteria include, but are not limited to, S. agalactiae, S. anginosus, S. bovis, S. canis, S. constellatus, S. dysgalactiae, S. equinus, S. iniae, S. intermedius, S. mitis, S. mutans, S. oralis, S. parasanguinis, S. peroris, S. pneumoniae, S. pyogenes, S. ratti, S. salivarius, S. thermophilus, S. sanguinis, S. sobrinus, S. suis, S. uberis, S. vestibularis, S. viridans, and S. zooepidemicus. In certain embodiments, the Strepococcus infection is an S. pyogenes infection. In certain embodiments, the Strepococcus infection is an S. pneumoniae infection. In certain embodiments, the S. pneumoniae has an efflux (e.g., mef, msr) genotype. In certain embodiments, the S. pneumoniae has a methylase (e.g., erm) genotype.

In certain embodiments, the bacteria is a member of the phylum Firmicutes and the genus Clostridium, i.e., the bacterial infection is a Clostridium infection. Exemplary Clostridia bacteria include, but are not limited to, C. botulinum, C. difficile, C. perfringens, C. tetani, C. scindens, and C. sordellii.

In certain embodiments, the compounds of the disclosure are a safer alternative to clindamycin, due to reduced incidence of pseudomembranous colitis. In certain embodiments, the compounds of the disclosure have increased activity against Clostridium difficile (C. difficile) in comparison to clindamycin. In certain embodiments, the compounds have a mean inhibitory concentration (MIC), with respect to C. difficile, of less than 50 μg/mL, less than 25 μg/mL, less than 20 μg/mL, less than 10 μg/mL, less than 5 μg/mL, or less than 1 μg/mL.

In certain embodiments, the bacterial infection is an infection with a Gram negative bacteria.

In certain embodiments, the Gram negative bacteria is a bacteria of the phylum Proteobacteria and the genus Escherichia. i.e., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the Escherichia infection is an E. coli infection.

In certain embodiments, the Gram negative bacteria is a bacteria of the phylum Proteobacteria and the genus Haemophilus. i.e., the bacterial infection is an Haemophilus infection. Exemplary Haemophilus bacteria include, but are not limited to, H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus. In certain embodiments, the Haemophilus infection is an H. influenzae infection.

In certain embodiments, the Gram negative bacteria is a bacteria of the phylum Proteobacteria and the genus Acinetobacter. i.e., the bacterial infection is an Acinetobacter infection. Exemplary Acinetobacter bacteria include, but are not limited to, A. baumanii, A. haemolyticus, and A. lwoffii. In certain embodiments, the Acinetobacter infection is an A. baumanii infection.

In certain embodiments, the Gram negative bacteria is a bacteria of the phylum Proteobacteria and the genus Klebsiella. i.e., the bacterial infection is a Klebsiella infection. Exemplary Klebsiella bacteria include, but are not limited to, K. granulomatis, K. oxytoca, K. michiganensis, K. pneumoniae, K. quasipneumoniae, and K. variicola. In certain embodiments, the Klebsiella infection is a K. pneumoniae infection.

In certain embodiments, the Gram negative bacteria is a bacteria of the phylum Proteobacteria and the genus Pseudomonas. i.e., the bacterial infection is a Pseudomonas infection. Exemplary Pseudomonas bacteria include, but are not limited to, P. aeruginosa, P. oryzihabitans, P. plecoglissicida, P. syringae, P. putida, and P. fluoroscens. In certain embodiments, the Pseudomonas infection is a P. aeruginosa infection.

In certain embodiments, the Gram negative bacteria is a bacteria of the phylum Proteobacteria and the genus Neisseria. i.e., the bacterial infection is a Neisseria infection. Exemplary Neisseria bacteria include, but are not limited to, N. cinerea, N. gonorrhoeae, N. polysaccharea, N. lactamica, N. meningitidis, N. mucosa, N. oralis and N. subflava. In certain embodiments, the Neisseria infection is a N. gonorrhoeae infection.

In certain embodiments, the Gram negative bacteria is a bacteria of the phylum Bacteroidetes and the genus Bacteroides. i.e., the bacterial infection is a Bacteroides infection. Exemplary Bacteroides bacteria include, but are not limited to, B. fragilis, B. distasonis, B. ovatus, B. thetaiotaomicron, and B. vulgatus. In certain embodiments, the Bacteroides infection is a B. fragilis infection.

In certain embodiments, the bacteria is an atypical bacteria, i.e., are neither Gram positive nor Gram negative.

In certain embodiments, the infectious disease is an infection with a parasitic infection. Thus, in certain embodiments, provided is a method of treating a parasitic infection comprising administering an effective amount of a compound of the present invention, or a pharmaceutically acceptable salt thereof, to a subject in need thereof.

In certain embodiments, the compound has an IC₅₀ (uM) with respect to a particular parasite, of less than 50 uM, less than 25 uM, less than 20 uM, less than 10 uM, less than 5 uM, or less than 1 uM.

Exemplary parasites include, but are not limited to, Trypanosoma spp. (e.g., Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp. (e.g., P. flaciparum), Crytosporidium spp., Isospora spp., Balantidium spp., Pneumocystis spp., Babesia, Loa Loa, Ascaris lumbricoides, Dirofilaria immitis, and Toxoplasma ssp. (e.g. T. gondii).

As generally described herein, the present disclosure further provides a method of treating an inflammatory condition (e.g., inflammatory disease) comprising administering an effective amount of a compound of the present disclosure, or a pharmaceutically acceptable salt thereof, to a subject in need thereof. Such a method can be conducted in vivo (i.e., by administration to a subject) or in vitro (e.g., upon contact with the pathogen, tissue, or cell culture). Treating, as used herein, encompasses therapeutic treatment and prophylactic treatment.

In certain embodiments, the effective amount is a therapeutically effective amount. For example, in certain embodiments, the method slows the progress of an inflammatory condition in the subject. In certain embodiments, the method improves the condition of the subject suffering from an inflammatory condition. In certain embodiments, the subject has a suspected or confirmed inflammatory condition.

In certain embodiments, the effective amount is a prophylatically effective amount. For example, in certain embodiments, the method prevents or reduces the likelihood of an inflammatory condition, e.g., in certain embodiments, the method comprises administering a compound of the present invention to a subject in need thereof in an amount sufficient to prevent or reduce the likelihood of an inflammatory condition. In certain embodiments, the subject is at risk to an inflammatory condition.

In certain embodiments, the inflammatory condition is an acute inflammatory condition (e.g., for example, inflammation resulting from an infection). In certain embodiments, the inflammatory condition is a chronic inflammatory condition. In certain embodiments, the inflammatory condition is inflammation associated with cancer.

As generally described herein, the present disclosure further provides a method of treating a central nervous system disorder comprising administering an effective amount of a compound of the present disclosure, or a pharmaceutically acceptable salt thereof, to a subject in need thereof. Such a method can be conducted in vivo (i.e., by administration to a subject) or in vitro (e.g., upon contact with a tissue or cell culture). Treating, as used herein, encompasses therapeutic treatment and prophylactic treatment.

In certain embodiments, the effective amount is a therapeutically effective amount. For example, in certain embodiments, the method slows the progress of a central nervous system disorder in the subject. In certain embodiments, the method improves the condition of the subject suffering from a central nervous system disorder. In certain embodiments, the subject has a suspected or confirmed central nervous system disorder.

In certain embodiments, the effective amount is a prophylatically effective amount. For example, in certain embodiments, the method prevents or reduces the likelihood of a central nervous system disorder, e.g., in certain embodiments, the method comprises administering a compound of the present disclosure to a subject in need thereof in an amount sufficient to prevent or reduce the likelihood of a central nervous system disorder. In certain embodiments, the subject is at risk of developing a central nervous system disorder.

In certain embodiments, compounds of the present disclosure may treat a central nervous system disorder by modulating the serotonin 5-HT_{2C} receptor. In certain embodiments, the compounds of the present disclosure are allosteric modulators of the serotonin 5-HT_{2C} receptor, e.g., see Zhou et al. ACS Chemical Neuroscience 2012, 3, 538-545, and Dinh et al. Molecular Pharmacology 2003, 64, 78-84.

In certain embodiments, the central nervous system disorder is addiction, anxiety, depression, obesity, eating disorders, Parkinson's disease, or schizophrenia.

### Examples

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthesis of Lincosamide Analogues

General Experimental Procedures: All reactions were performed in oven- or flame-dried round-bottomed or modified Schlenk flasks fitted with rubber septa under a positive pressure of argon (dried by passage through a column of Drierite calcium sulfate desiccant), unless otherwise noted. Air- and moisture-sensitive liquids and solutions were transferred via syringe or stainless steel cannula. When necessary (so noted), solutions were deoxygenated by three cycles of freezing (liquid nitrogen), evacuation, and thawing under static vacuum. Organic solutions were concentrated by rotary evaporation (house vacuum, ˜60 Torr) at 23-30° C. Flash-column chromatography was performed as described by Still et al., (Still, W. C.; Kahn, M.; Mitra, A., J. Org. Chem. 1978, 43 (14), 2923-2925), employing silica gel (60-Å pore size, 230-400 mesh, Agela Technologies, Chicago, Ill.; or RediSep silica cartridges, Teledyne Isco, Lincoln, Nebr.). Analytical thin-layer chromatography (TLC) was performed using glass plates pre-coated with silica gel (0.25 mm, 60-Å pore size, 230-400 mesh, Merck KGA) impregnated with a fluorescent indicator (254 nm). In special cases (so noted), analytical TLC was performed with aminopropyl-modified silica gel (NH₂ silica gel, 60-Å pore size, Wako Chemicals USA) impregnated with a fluorescent indicator (254 nm). TLC plates were visualized by exposure to ultraviolet light (UV) and/or exposure to iodine vapor (I₂), basic aqueous potassium permanganate solution (KMnO₄), acidic ethanolic para-anisaldehyde solution (PAA), acidic aqueous ceric ammonium molybdate solution (CAM), or ethanolic solution of phosphomolybdic acid (PMA) followed by brief heating on a hot plate as needed (˜200° C., ≤15 s). In some cases, reaction monitoring was carried out by analytical liquid chromatography—mass spectrometry (LCMS), or by flow-injection analysis-high-resolution mass spectrometry (FIA-HRMS).

Instrumentation: Proton nuclear magnetic resonance (¹H NMR) spectra and carbon nuclear magnetic resonance (¹³C NMR) spectra were recorded on Varian Mercury 400 (400 MHz/100 MHz), Varian Inova 500 (500 MHz/125 MHz), or Varian Inova 600 (600 MHz/150 MHz) NMR spectrometers at 23° C. Proton chemical shifts are expressed in parts per million (ppm, δ scale) and are referenced to residual protium in the NMR solvent (CHCl₃, δ 7.26; CHD₃OD, δ 3.31; C₆H₅D, δ 7.16). Carbon chemical shifts are expressed as parts per million (ppm, δ scale) and are referenced to the carbon resonance of the NMR solvent (CDCl₃, δ 77.2; CD₃OD, δ 49.0; C₆D₆, δ 128.1). Data are reported as follows: Chemical shift, multiplicity (s=singlet, d=doublet, t=triplet, q=quartet, qn=quintet, dd=doublet of doublets, td=triplet of doublets, ABq=AB quartet, m=multiplet, br=broad, app=apparent), integration, and coupling constant (J) in Hertz (Hz). Infrared transmittance (IR) spectra were obtained using a Bruker ALPHA FTIR spectrophotometer referenced to a polystyrene standard. Data are represented as follows: Frequency of absorption (cm⁻¹), and intensity (s=strong, m=medium, br=broad). High-resolution mass spectrometry (including FIA-HRMS reaction monitoring) was performed at the Harvard University Mass Spectrometry Facility using a Bruker micrOTOF-QII mass spectrometer. Ultra high-performance liquid chromatography—mass spectrometry (LCMS) was performed using an Agilent Technologies 1260-series analytical HPLC system in tandem with an Agilent Technologies 6120 Quadrupole mass spectrometer; a Zorbax Eclipse Plus reverse-phase C₁₈ column (2.1×50 mm, 1.8 μm pore size, 600 bar rating; Agilent Technologies, Santa Clara, Calif.) was employed as stationary phase. LCMS samples were eluted at a flow rate of 650 μL/min, beginning with 5% acetonitrile-water containing 0.1% formic acid, grading linearly to 100% acetonitrile containing 0.1% formic acid over 3 minutes, followed by 100% acetonitrile containing 0.1% formic acid for 2 minutes (5 minute total run time).

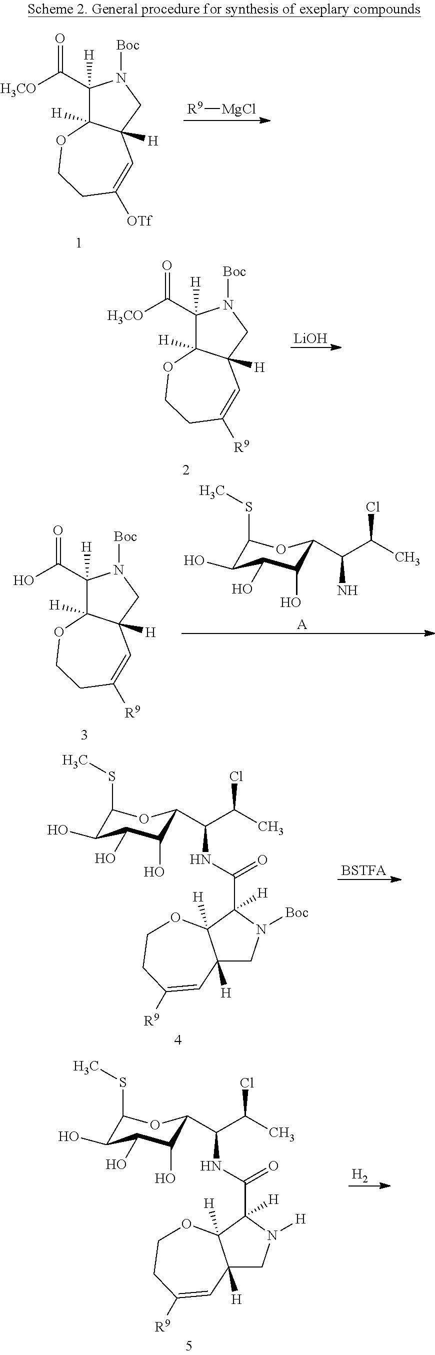

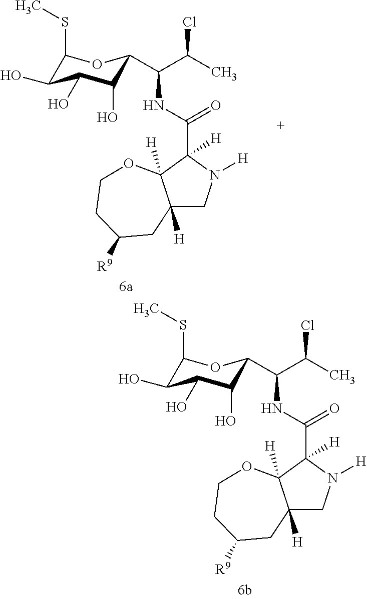

Step 1: A solution of alkyl Grignard reagent (1.2 equiv.) in THF or diethyl ether was added dropwise by syringe to a stirring solution of 1 (1.0 equiv.), iron (III) acetylacetonate (0.05 equiv.), and N-methylpyrrolidone (10.0 equiv.) at −30° C. in THF such that the concentration of 1 was 0.05 M (See PCT/US2018/046167, incorporated herein by reference, for details on the preparation of 1). The resultant solution immediately changed in color from red-orange to dark brown/black. The solution was stirred at −30° C. under argon. Consumption of 1 was monitored by TLC (EtOAc in hexanes). After complete consumption of 1 was observed, the reaction solution was quenched with saturated aqueous ammonium chloride and extracted three times with EtOAc. The combined organic layers were washed with brine, dried over sodium sulfate, filtered and concentrated under reduced pressure. The crude product was purified by column chromatography (EtOAc in hexanes) to yield 2.

Step 2: A 1.0 N aq. solution of lithium hydroxide (2.0 equiv.) was added to a solution of 2 (1.0 equiv.) in methanol and THF in a 1:1 ratio such that the concentration of 2 was 0.2 M. The resultant solution was stirred at ambient temperature until complete consumption of the methyl ester was observed by mass spectrometry. The reaction solution was acidified with 1 N aq. hydrochloric acid until it reached pH 2 and extracted five times with EtOAc. The combined organic layers were dried over sodium sulfate, filtered, and concentrated to yield 3, which was taken forward without further purification.

Step 3: Triethylamine (3.2 equiv.) was added to a solution of 3 (1.0 equiv.) and A (1.1 equiv.) in DMF such that the concentration of 3 was 0.35 M at 0° C. (See PCT/US2018/046167, incorporated herein by reference, for details on the preparation of A). The resultant colorless solution was stirred for 10 min at 0° C., and HATU (1.3 equiv.) was added in a single portion. The resultant bright yellow solution was stirred at ambient temperature under argon. Consumption of 3 was monitored by mass spectrometry. After complete consumption of 3 was observed by mass spectrometry, the reaction solution was concentrated under reduced pressure and directly purified by column chromatography (methanol in DCM) to yield 4. Due to amide and carbamate rotamerism, this intermediate was taken forward to N-Boc deprotection before full characterization.

Step 4: Bis(trimethylsilyl)trifluoroacetamide (3.5 equiv.) was added by syringe to a solution of 4 (1.0 equiv.) in acetonitrile such that the concentration of 4 was 0.1 M at 0° C. The resultant solution was stirred at 0° C. for 5 min, stirred at ambient temperature for 15 min, then cooled back to 0° C. Trimethylsilyl iodide (1.0 equiv.) was added dropwise by syringe to the reaction solution at 0° C. The resultant solution was stirred at 0° C. until complete N-Boc deprotection was observed by mass spectrometry. The reaction solution was quenched with dropwise addition of methanol and concentrated under reduced pressure. The crude product was purified on reverse-phase HPLC (acetonitrile in water) to yield 5.

Step 5: A suspension of 5 (1.0 equiv.) and palladium hydroxide (20% on carbon, 1.0 equiv.) in anhydrous methanol such that the concentration of 5 was 0.1 M was stirred under an atmosphere of hydrogen (1 atm) until complete hydrogenation was observed by mass spectrometry. The reaction solution was filtered through a pad of Celite, washing with methanol, and concentrated. The crude product was purified on reverse-phase HPLC (acetonitrile in water) to yield 6a, either as a single diastereomer or alongside the epimer 6b. In some cases, concomitant N-methylation was observed during hydrogenation. To obtain the desired N-desmethyl versions, the corresponding N-Boc oxepine was prepared and hydrogenation was carried out before N-Boc deprotection. The following compounds were prepared according to the method described in Scheme 2.

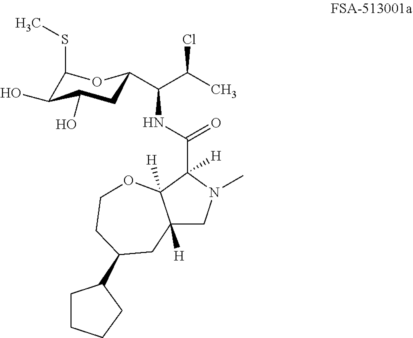

¹H NMR (600 MHz, CD₃OD) δ 5.30 (d, J=5.6 Hz, 1H), 4.62 (q, J=6.1 Hz, 1H), 4.37 (d, J=10.8 Hz, 1H), 4.16 (d, J=9.9 Hz, 1H), 4.11-4.05 (m, 4H), 3.56 (dd, J=10.2, 3.5 Hz, 1H), 3.46 (m, 1H), 3.27 (d, J=9.7 Hz, 1H), 3.20 (dd, J=9.0, 6.4 Hz, 1H), 2.42 (m, 1H), 2.40 (s, 3H), 2.23 (m, 1H), 2.13 (s, 3H), 1.76 (m, 4H), 1.70 (m, 3H), 1.62 (m, 4H), 1.55 (m, 3H), 1.49 (d, J=6.8 Hz, 3H), 1.15 (m, 2H). HRMS (ESI+, m/z): [M+H]+ calc'd for C24H41ClN2O6S, 520.2374; found 520.24.

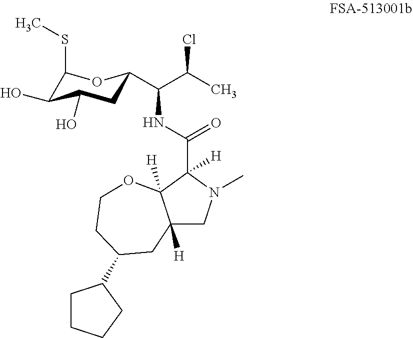

¹H NMR (600 MHz, CD₃OD) δ 5.30 (d, J=5.5 Hz, 1H), 4.63 (m, 1H), 4.36 (d, J=9.9 Hz, 1H), 4.24 (m, 1H), 4.16 (d, J=10.0 Hz, 1H), 4.07 (dd, J=10.2, 5.6 Hz, 1H), 4.02 (d, J=3.2 Hz, 1H), 3.98 (m, 1H), 3.78 (m, 1H), 3.58 (dd, J=10.2, 3.5 Hz, 1H), 2.37 (s, 3H), 2.27 (m, 1H), 2.17 (m, 1H), 2.13 (s, 3H), 2.02 (m, 1H), 1.82-1.77 (m, 4H), 1.73-1.66 (m, 3H), 1.64 (m, 2H), 1.54 (m, 2H), 1.48 (d, J=6.8 Hz, 1H), 1.43 (m, 1H), 1.29 (m, 1H), 1.16 (m, 2H), 0.99 (m, 1H). HRMS (ESI+, rn/z): [M+H]+ calc'd for C24H41ClN2O6S, 520.2374, found 520.2413.

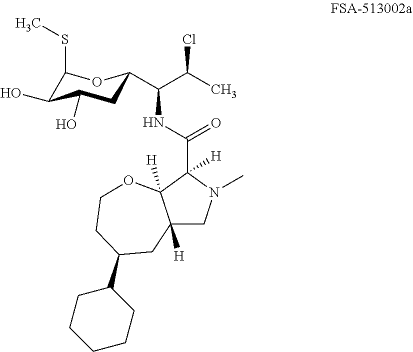

¹H NMR (600 MHz, CD₃OD) δ 5.30 (d, J=5.5 Hz, 1H), 4.62 (q, J=6.3 Hz, 1H), 4.37 (d, J=9.9 Hz, 1H), 4.16 (d, J=9.9 Hz, 1H), 4.11-4.05 (m, 4H), 3.57 (dd, J=10.2, 3.3 Hz, 1H), 3.41 (t, J=11.2 Hz, 1H), 3.24 (d, J=9.5 Hz, 1H), 3.20 (m, 1H), 2.39 (s, 3H), 2.37 (m, 1H), 2.21 (m, 1H), 2.13 (s, 3H), 1.77-1.62 (m, 9H), 1.54 (dd, J=14.6, 4.8 Hz, 1H), 1.49 (d, J=6.7 Hz, 3H), 1.46 (m, 1H), 1.29-1.23 (m, 4H), 1.16 (m, 1H), 1.09 (m, 1H), 1.00 (m, 1H). HRMS (ESI+, m/z): [M+H]+ calc'd for C26H45ClN2O5S, 534.253; found 534.2542.

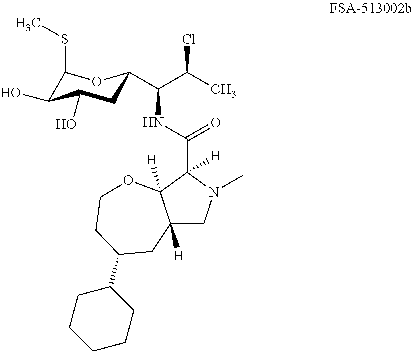

¹H NMR (600 MHz, CD₃OD) δ 5.31 (d, J=5.6 Hz, 1H), 4.62 (m, 1H), 4.37 (m, 1H), 4.21 (d, J=9.9 Hz, 1H), 4.12 (d, J=3.1 Hz, 1H), 4.07 (dd, J=10.2, 5.5 Hz, 1H), 4.02 (m, 1H), 3.77 (t, J=9.5 Hz, 1H), 3.60 (dd, J=10.3, 3.5 Hz, 1H), 3.24 (d, J=10.1 Hz, 1H), 3.20 (m, 1H), 3.01 (m, 1H), 2.60 (m, 1H), 2.41 (s, 3H), 2.13 (s, 3H), 1.86 (m, 1H), 1.76 (m, 3H), 1.69 (m, 4H), 1.48 (d, J=6.7 Hz, 2H), 1.29 (m, 6H), 1.18-1.09 (m, 4H). HRMS (ESI+, m/z): [M+H]+ C25H43ClN2O6S, 535.2603; found 535.2586.

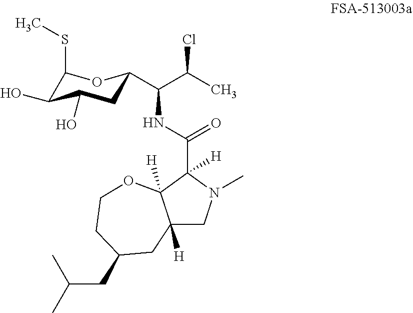

¹H NMR (600 MHz, CD₃OD) δ 5.30 (d, J=5.5 Hz, 1H), 4.63 (q, J=6.8 Hz, 1H), 4.36 (dd, J=9.8 Hz, 1H), 4.15 (d, J=9.9 Hz, 1H), 4.11-4.04 (m, 4H), 3.57 (dd, J=10.2, 3.5 Hz, 1H), 3.47 (m, 1H), 3.22 (d, J=9.7 Hz, 1H), 3.17 (dd, J=9.0, 6.3 Hz, 1H), 2.42 (m, 1H), 2.38 (s, 3H), 2.19 (m, 1H), 2.13 (s, 3H), 1.98 (m, 1H), 1.68 (m, 1H), 1.64-1.57 (m, 3H), 1.55-1.48 (m, 1H), 1.49 (d, J=6.8 Hz, 3H), 1.29 (br, 1H), 1.19 (m, 2H), 0.89 (t, J=7.2 Hz, 6H). HRMS (ESI+, m/z): [M+H]+ calc'd for C23H41ClN2O6S, 508.2374; found 508.241.

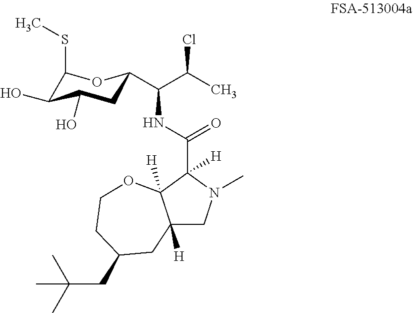

¹H NMR (600 MHz, CD₃OD) δ 5.30 (d, J=5.6 Hz, 1H), 4.63 (q, J=6.8 Hz, 1H), 4.35 (d, J=9.9 Hz, 1H), 4.14 (m, 1H), 4.12 (m, 1H), 4.09 (d, J=3.6 Hz, 1H), 4.06 (dd, J=10.2, 5.6 Hz, 1H), 4.03 (m, 1H), 3.57 (dd, J=10.2, 3.6 Hz, 1H), 3.51 (dd, J=12.0, 10.1 Hz, 1H), 3.19 (d, J=9.8 Hz, 1H), 3.15 (dd, J=9.0, 6.3 Hz, 1H), 2.42 (m, 1H), 2.36 (s, 3H), 2.16 (dd, J=11.5, 9.0 Hz, 1H), 2.13 (s, 3H), 2.02 (m, 1H), 1.80 (m, 1H), 1.62 (m, 3H), 1.49 (d, J=6.8 Hz, 3H), 1.31 (dd, J=14.1, 5.5 Hz, 1H), 1.29 (m, 1H), 1.25 (dd, J=14.0, 4.9 Hz, 1H), 0.92 (s, 9H). HRMS (ESI+, m/z): [M+H]+ calc'd for C24H43ClN2O6S, 522.253; found 522.257.

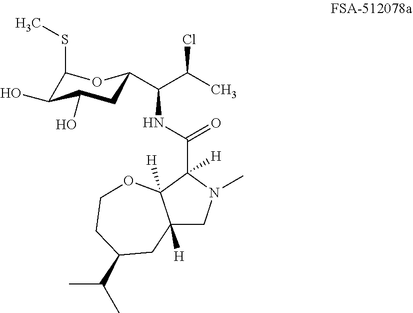

¹H NMR (600 MHz, CD₃OD) δ 5.30 (d, J=5.6 Hz, 1H), 4.58 (q, J=6.9 Hz, 1H), 4.47 (d, J=10.0 Hz, 1H), 4.27 (d, J=9.0 Hz, 1H), 4.24 (d, J=9.9 Hz, 1H), 4.17 (t, J=8.9 Hz, 1H), 4.08 (m, 2H), 3.88 (d, J=2.4 Hz, 1H), 3.57 (dd, J=10.2, 3.4 Hz, 1H), 3.54 (m, 1H), 3.46 (dd, J=10.8, 7.3 Hz, 1H), 2.78 (t, J=11.4 Hz, 1H), 2.31 (m, 1H), 2.15 (s, 3H), 1.81 (m, 2H), 1.73-1.63 (m, 3H), 1.52 (d, J=6.8 Hz, 3H), 1.48 (m, 1H), 0.90 (dd, J=6.8, 3.3 Hz, 6H). HRMS (ESI+, m/z): [M+H]+ calc'd for C21H37ClN2O6S, 480.2061; found 480.2075.

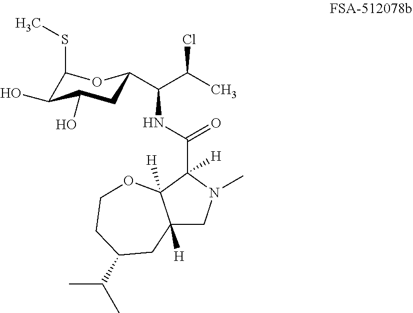

¹H NMR (600 MHz, CD₃OD) δ 5.30 (d, J=5.6 Hz, 1H), 4.58 (q, J=7.0 Hz, 1H), 4.45 (d, J=10.4 Hz, 1H), 4.23 (d, J=10.0 Hz, 1H), 4.18-4.07 (m, 4H), 3.90 (m, 1H), 3.57 (dd, J=10.1, 3.2 Hz, 1H), 3.52 (dd, J=11.9, 9.8 Hz, 1H), 3.40 (m, 1H), 2.71 (t, J=11.2 Hz, 1H), 2.28 (m, 1H), 2.14 (s, 3H), 1.80-1.61 (m, 5H), 1.51 (d, J=6.8 Hz, 3H), 1.48 (m, 2H), 0.90 (dd, J=6.8, 2.9 Hz, 6H). HRMS (ESI+, m/z): [M+H]+ calc'd for C21H37CLN2O6S, 480.2061; found 480.2084.

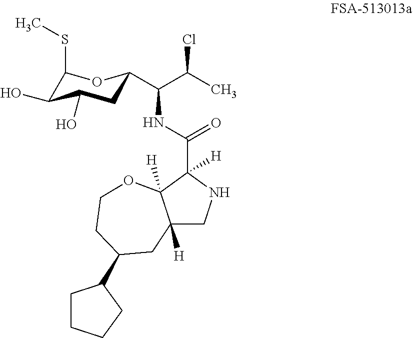

¹H NMR (600 MHz, CD₃OD) δ 5.30 (d, J=5.7 Hz, 1H), 4.57 (q, J=6.8 Hz, 1H), 4.50 (d, J=10.0 Hz, 1H), 4.40 (d, J=9.0 Hz, 1H), 4.27 (d, J=10.0 Hz, 1H), 4.23 (t, J=9.0 Hz, 1H), 4.08 (dd, J=10.2, 5.6 Hz, 1H), 4.05 (m, 1H), 3.85 (d, J=2.5 Hz, 1H), 3.63 (ddd, J=11.7, 8.5, 2.5 Hz, 1H), 3.58 (dd, J=10.2, 3.2 Hz, 1H), 3.52 (dd, J=11.1, 7.4 Hz, 1H), 2.87 (t, J=11.7 Hz, 1H), 2.38 (m, 1H), 2.15 (s, 3H), 1.86-1.77 (m, 6H), 1.73 (m, 1H), 1.65 (m, 2H), 1.60-1.53 (m, 3H), 1.52 (d, J=6.8 Hz, 3H), 1.14 (m, 2H). HRMS (ESI+, m/z): [M+H]+ calc'd for C23H39ClN2O6S, 506.2217; found 506.2216.

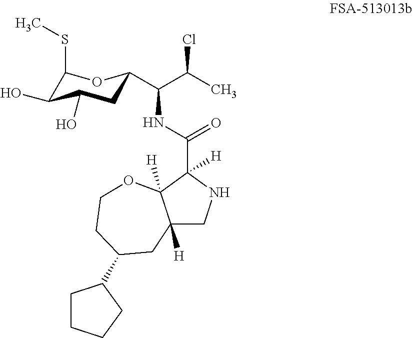

¹H NMR (600 MHz, CD₃OD) δ 5.76 (m, 1H), 5.56 (m, 1H), 5.32 (d, J=5.6 Hz, 1H), 4.60-4.56 (m, 2H), 4.54 (m, 1H), 4.40-4.36 (m, 2H), 4.31 (dq, J=17.2, 2.8 Hz, 1H), 4.25 (d, J=5.7 Hz, 1H), 4.11 (dd, J=10.2, 5.6 Hz, 1H), 3.83 (d, J=3.2 Hz, 1H), 3.60 (m, 2H), 3.10 (dd, J=11.6, 8.3 Hz, 1H), 2.87 (m, 1H), 2.67 (m, 1H), 2.16 (s, 3H), 2.15-2.11 (m, 1H), 1.51 (d, J=6.7 Hz, 3H).

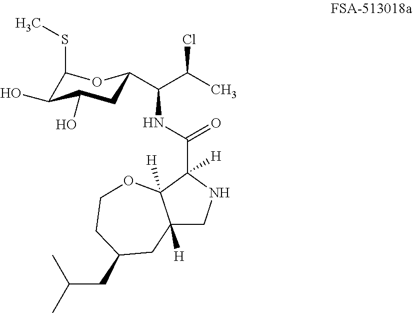

¹H NMR (500 MHz, CD₃OD) δ 8.47 (s, 1H), 5.31 (d, J=5.6 Hz, 1H), 4.57 (m, 1H), 4.52 (d, J=11.1 Hz, 1H), 4.46 (d, J=9.0 Hz, 1H), 4.28 (d, J=9.9 Hz, 1H), 4.26 (m, 1H), 4.09 (dd, J=10.2, 5.6 Hz, 1H), 4.02 (m, 1H), 3.86 (m, 1H), 3.65 (m, 1H), 3.58 (dd, J=10.2, 3.3 Hz, 1H), 3.55 (m, 1H), 2.91 (t, J=11.8 Hz, 1H), 2.40 (m, 1H), 2.15 (s, 3H), 2.07 (m, 1H), 1.76 (m, 1H), 1.64 (m, 4H), 1.53 (d, J=6.8 Hz, 3H), 1.23 (t, J=7.2 Hz, 2H), 0.91 (d, J=6.3 Hz, 3H), 0.89 (d, J=6.3 Hz, 3H). HRMS (ESI-TOF) m/z: Calc'd for [C₂₂H₃₉ClN₂O₆S+H]⁺: 495.2290; Found: 495.2294.

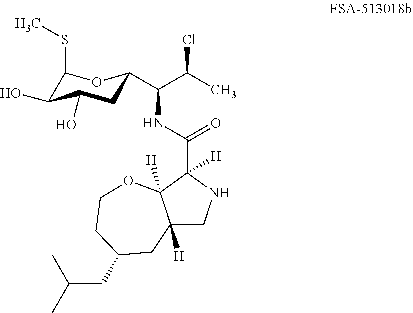

¹H NMR (600 MHz, CD₃OD) δ 8.48 (br s, 1H), 5.32 (d, J=5.7 Hz, 1H), 4.56 (qd, J=1.6, 6.8 Hz, 1H), 4.52 (dd, J=1.5, 10.0 Hz, 1H), 4.49 (d, J=8.8 Hz, 1H), 4.35 (t, J=9.3 Hz, 1H), 4.28 (d, J=10.0 Hz, 1H), 4.09 (dd, J=5.6, 10.3 Hz, 1H), 4.00 (dt, J=3.9, 12.0 Hz, 1H), 3.88 (d, J=3.3 Hz, 1H), 3.80 (td, J=3.7, 11.2 Hz, 1H), 3.63-3.56 (m, 2H), 2.91 (t, J=11.8 Hz, 1H), 2.34-2.26 (m, 1H), 2.15 (s, 3H), 1.99 (dt, J=3.3, 13.3 Hz, 1H), 1.81-1.62 (m, 4H), 1.54 (d, J=6.8 Hz, 3H), 1.23-1.15 (m, 2H), 1.01-0.94 (m, 1H), 0.89 (d, J=6.5 Hz, 6H). HRMS (ESI-TOF) m/z: Calc'd for [C₂₂H₃₉ClN₂O₆S+H]⁺: 495.2290; Found: 495.2293.

The following compounds were prepared according to the procedures below.

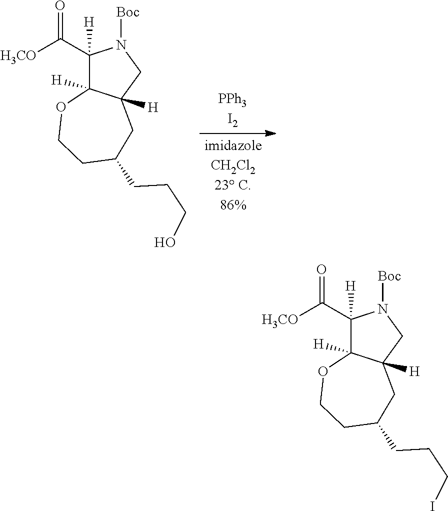 [SMILES: COC(=O)[C@@H]1[C@@H]2OCC[C@@H](CCCO)C[C@H]2CN1C(=O)OC(C)(C)C ; COC(=O)[C@@H]1[C@@H]2OCC[C@@H](CCCI)C[C@H]2CN1C(=O)OC(C)(C)C]

Triphenylphosphine (27.0 mg, 0.103 mmol, 1.3 equiv) was dissolved in dichloromethane (0.80 mL) and the solution was held at 23° C. in a water bath. Solid iodine (26.7 mg, 0.105 mmol, 1.33 equiv) was added to this solution and the resulting dark solution was stirred at 23° C. for 10 min. Solid imidazole (16.2 mg, 0.238 mmol, 3 equiv) was added and the resulting yellow suspension was stirred at 23° C. for a further 10 min. A solution of alcohol ##(28.3 mg, 0.079 mmol) in dichloromethane (0.60 mL) was added via cannula, and dichloromethane (0.20 mL) was used to quantitate the transfer. The solution was stirred at 23° C. for 1 h. The reaction mixture was quenched with a saturated aqueous solution of sodium thiosulfate (25 mL). The biphasic mixture was stirred at 23° C. for 30 min, after which water (20 mL) was added. The mixture was extracted with dichloromethane (3×30 mL) and the combined organic layers were dried over sodium sulfate. The dried solution was concentrated in vacuo and the residue was purified by flash chromatography on silica gel (10%→20% ethyl acetate-hexanes) to provide the iodide (31.8 mg, 86%) as a colorless oil. ¹H NMR (1.5:1 rotamer ratio, “major” and “minor”, respectively, 500 MHz, CDCl₃) δ: 4.44 (d, J=8.1 Hz, 0.4H, minor), 4.35 (d, J=8.2 Hz, 0.6H, major), 4.14-4.03 (m, 1H), 3.95-3.69 (m, 5H), 3.15 (t, J=6.9 Hz, 2H), 2.87 (t, J=10.6 Hz, 0.4H, minor), 2.84 (t, J=10.6 Hz, 0.6H, major), 2.63-2.45 (m, 1H), 1.98-1.56 (m, 6H), 1.44-1.31 (m, 10H), 0.99-0.89 (m, 1H).

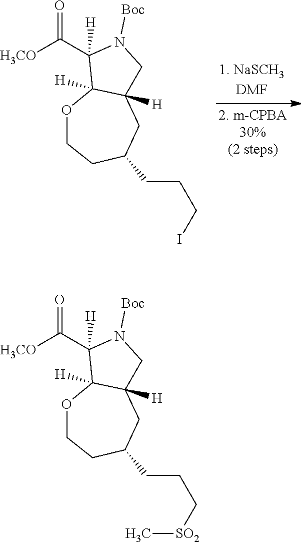 [SMILES: COC(=O)[C@@H]1[C@@H]2OCC[C@@H](CCCI)C[C@H]2CN1C(=O)OC(C)(C)C ; COC(=O)[C@@H]1[C@@H]2OCC[C@@H](CCCS(C)(=O)=O)C[C@H]2CN1C(=O)OC(C)(C)C]

Sodium thiomethoxide (11.0 mg, 0.156 mmol, 5 equiv) was added to a solution of alkyl iodide (14.6 mg, 31 μmol) in N,N-dimethylformamide (0.31 mL) and the resulting suspension was stirred at 23° C. for 5 h. The reaction mixture was quenched with a saturated aqueous solution of sodium bicarbonate (20 mL) and the resulting solution was extracted with dichloromethane (2×20 mL). The combined organic layers were dried over sodium sulfate and the dried solution was concentrated in vacuo to provide the crude sulfide, which was used in the next step without further purification.

m-Chloroperbenzoic acid (73%, 29.5 mg as is, 21.6 mg of m-chloroperbenzoic acid, 0.125 mmol, 4 equiv) was added to a solution of crude sulfide (31 μmol) in dichloromethane (0.62 mL) and the resulting solution was stirred at 23° C. for 18 h. The reaction mixture was diluted with dichloromethane (20 mL) and was quenched with a saturated aqueous solution of sodium thiosulfate (10 mL). The biphasic mixture was stirred at 23° C. for 30 min and the layers were separated. The aqueous layer was further extracted with dichloromethane (20 mL), and the combined organic layers were dried over sodium sulfate. The dried solution was concentrated in vacuo and the residue was purified by flash chromatography on silica gel (20%→40% acetone-hexanes) to provide the sulfone (3.9 mg, 30%) as a colorless oil. ¹H NMR (1.5:1 rotamer ratio, “major” and “minor”, respectively, 500 MHz, CDCl₃) δ: 4.45 (d, J=8.2 Hz, 0.4H, minor), 4.36 (d, J=8.2 Hz, 0.6H, major), 4.13-4.07 (m, 1H), 3.94-3.70 (m, 6H), 2.99 (t, J=8.6 Hz, 2H), 2.91 (s, 3H), 2.89-2.84 (m, 1H), 2.65-2.49 (m, 1H), 2.01-1.58 (m, 7H), 1.47-1.37 (m, 11H), 1.01-0.92 (m, 1H).

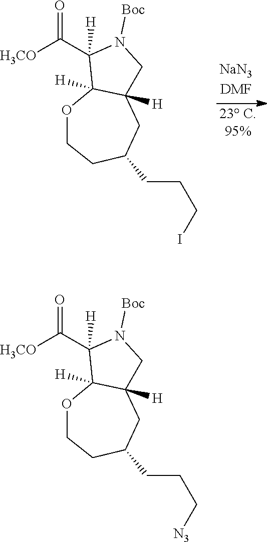 [SMILES: COC(=O)[C@@H]1[C@@H]2OCC[C@@H](CCCI)C[C@H]2CN1C(=O)OC(C)(C)C ; COC(=O)[C@@H]1[C@@H]2OCC[C@@H](CCCN=[N+]=[N-])C[C@H]2CN1C(=O)OC(C)(C)C]

Sodium azide (9.5 mg, 0.146 mmol, 5 equiv) was added to a solution of alkyl iodide (13.6 mg, 29 μmol) in N,N-dimethylformamide (0.29 mL) and the resulting suspension was stirred at 23° C. for 16 h. The reaction mixture was quenched with a saturated aqueous solution of sodium bicarbonate (20 mL) and the resulting solution was extracted with dichloromethane (2×20 mL). The combined organic layers were dried over sodium sulfate and the dried solution was concentrated in vacuo. The residue was purified by flash chromatography on silica gel (10%→25% ethyl acetate-hexanes) to provide the azide (10.6 mg, 95%) as a colorless oil. ¹H NMR (1.5:1 rotamer ratio, “major” and “minor”, respectively, 500 MHz, CDCl₃) δ: 4.45 (d, J=8.1 Hz, 0.4H, minor), 4.36 (d, J=8.2 Hz, 0.6H, major), 4.14-4.05 (m, 1H), 3.95-3.69 (m, 6H), 3.26 (t, J=6.8 Hz, 2H), 2.88 (t, J=10.6 Hz, 0.4H, minor), 2.86 (t, J=10.6 Hz, 0.6H, major), 2.65-2.45 (m, 1H), 2.02-1.89 (m, 1H), 1.81-1.71 (m, 1H), 1.67-1.54 (m, 4H), 1.43 (s, 3.6H, minor), 1.39 (s, 5.4H, major), 1.38-1.30 (m, 2H), 0.99-0.89 (m, 1H).

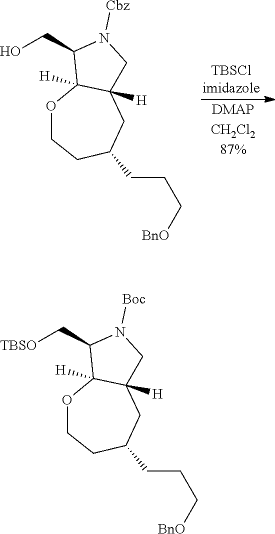 [SMILES: O=C(OCc1ccccc1)N1C[C@@H]2C[C@H](CCCOCc3ccccc3)CCO[C@H]2[C@H]1CO ; CC(C)(C)OC(=O)N1C[C@@H]2C[C@H](CCCOCc3ccccc3)CCO[C@H]2[C@H]1CO[Si](C)(C)C(C)(C)C]

Tert-Butyldimethylsilyl chloride (217 mg, 1.44 mmol, 1.2 equiv) was added to a solution of alcohol (543.4 mg, 1.20 mmol), imidazole (163 mg, 2.40 mmol, 2 equiv), and N,N-dimethylaminopyridine (14.7 mg, 0.12 mmol, 0.10 equiv) in dichloromethane (6.0 mL) at 23° C. The resulting solution was stirred at 23° C. for 3 h and was quenched with a saturated aqueous solution of ammonium chloride (30 mL). The resulting mixture was extracted with dichloromethane (3×30 mL) and the combined organic layers were dried over sodium sulfate. The dried solution was concentrated in vacuo and the residue was purified by flash chromatography on silica gel (10%→15% ethyl acetate-hexanes) to provide silyl ether ##(592.4 mg, 87%) as a colorless oil. ¹H NMR (1:1 rotamer ratio, 600 MHz, CDCl₃) δ: 7.40-7.22 (m, 10H), 5.27-5.08 (m, 2H), 4.51 (s, 2H), 4.04-3.79 (m, 6H), 3.70-3.63 (m, 1H), 3.49-3.43 (m, 2H), 2.96 (t, J=10.3 Hz, 0.5H), 2.92 (t, J=10.2 Hz, 0.5H), 2.82-2.65 (m, 1H), 2.00-1.78 (m, 2H), 1.68-1.51 (m, 4H), 1.43-1.32 (m, 2H), 0.90 (s, 4.5H), 0.89 (s, 4.5H), 0.94-0.86 (m, 1H), 0.05 to −0.03 (4s, 6H).

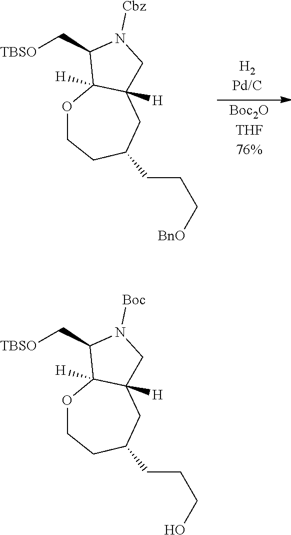 [SMILES: CC(C)(C)[Si](C)(C)OC[C@@H]1[C@@H]2OCC[C@@H](CCCOCc3ccccc3)C[C@H]2CN1C(=O)OCc1ccccc1 ; CC(C)(C)OC(=O)N1C[C@@H]2C[C@H](CCCO)CCO[C@H]2[C@H]1CO[Si](C)(C)C(C)(C)C]

10% Palladium on carbon (222 mg, 22.2 mg of palladium, 0.21 mmol, 0.2 equiv) was added to a solution of benzyl ether (592.4 mg, 1.043 mmol) and di-tert-butyl dicarbonate (342 mg, 1.565 mmol, 1.5 equiv) in tetrahydrofuran (21 mL) under nitrogen atmosphere. The suspension was sparged with hydrogen for 5 min and was then stirred under hydrogen atmosphere at 23° C. for 24 h. The reaction vessel was flushed with nitrogen and the suspension was filtered through a Celite pad. The filter cake was washed sequentially with tetrahydrofuran (20 mL) and methanol (20 mL) and the filtrate was concentrated in vacuo. The residue was purified by flash chromatography on silica gel (20%→40% ethyl acetate-hexanes) to provide the alcohol (349.9 mg, 76%) as a colorless oil. ¹H NMR (1:1 rotamer ratio, 400 MHz, CDCl₃) δ: 3.98-3.67 (m, 6H), 3.63 (t, J=6.6 Hz, 2H), 3.57-3.44 (m, 1H), 2.89-2.59 (m, 2H), 1.98-1.75 (m, 2H), 1.63-1.49 (m, 4H), 1.43 (s, 9H), 1.39-1.27 (m, 2H), 0.95-0.82 (m, 1H), 0.87 (s, 9H), 0.04 to −0.01 (4s, 6H).

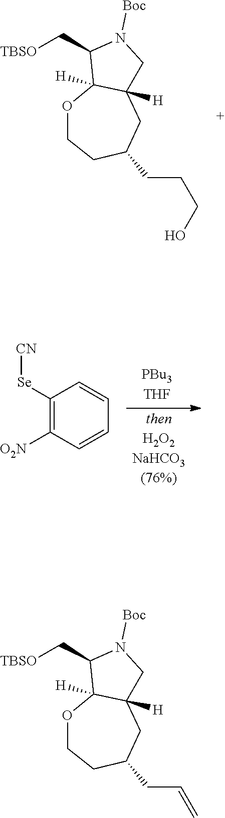 [SMILES: CC(C)(C)OC(=O)N1C[C@@H]2C[C@H](CCCO)CCO[C@H]2[C@H]1CO[Si](C)(C)C(C)(C)C ; N#C[Se]c1ccccc1[N+](=O)[O-] ; C=CC[C@@H]1CCO[C@@H]2[C@@H](C1)CN(C(=O)OC(C)(C)C)[C@@H]2CO[Si](C)(C)C(C)(C)C]

o-Nitrophenylselenocyanate (224 mg, 0.096 mmol, 1.5 equiv) was added to a solution of alcohol (291.6 mg, 0.657 mmol) in tetrahydrofuran (13.0 mL) at 23° C. To the resulting solution was added tributylphosphine (0.24 mL, 199 mg, 0.986 mmol, 1.5 equiv) and the solution color changed from orange to dark brown. The reaction mixture was stirred at 23° C. for 2 h and was then cooled to 0° C. Sodium bicarbonate (1.24 g, 14.8 mmol, 22.5 equiv) and a 30% aqueous solution of hydrogen peroxide (2.0 mL, 19.7 mmol, 30 equiv) were added and the resulting suspension was warmed to 23° C. The reaction mixture was stirred at 23° C. for 2 h and was quenched with a saturated aqueous solution of ammonium chloride (30 mL). The mixture was extracted with dichloromethane (3×30 mL) and the combined organic layers were dried over sodium sulfate. The dried solution was concentrated in vacuo and the residue was purified by flash chromatography on silica gel (2%→7% ethyl acetate-hexanes) to provide the olefin (213.3 mg, 76%) as a pale yellow oil. ¹H NMR (1:1 rotamer ratio, 600 MHz, CDCl₃) δ: 5.80-5.68 (m, 1H), 5.00 (d, J=10.7 Hz, 1H), 4.99 (d, J=17.4 Hz, 1H), 3.97-3.68 (m, 6H), 3.54 (dd, J=8.8, 10.6 Hz, 0.5H), 3.50 (dd, J=8.4, 10.2 Hz, 0.5H), 2.85 (t, J=10.3 Hz, 0.5H), 2.79 (t, J=10.1 Hz, 0.5H), 2.76-2.62 (m, 1H), 2.11-1.78 (m, 4H), 1.64-1.53 (m, 2H), 1.43 (s, 9H), 0.96-0.86 (m, 1H), 0.87 (s, 9H), 0.05 to −0.03 (4s, 6H).

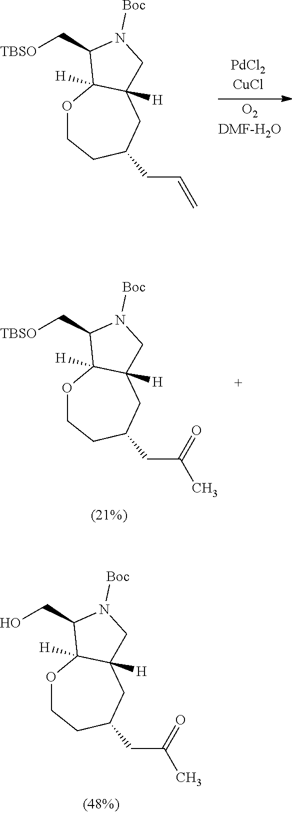 [SMILES: C=CC[C@@H]1CCO[C@@H]2[C@@H](C1)CN(C(=O)OC(C)(C)C)[C@@H]2CO[Si](C)(C)C(C)(C)C ; CC(=O)C[C@@H]1CCO[C@@H]2[C@@H](C1)CN(C(=O)OC(C)(C)C)[C@@H]2CO[Si](C)(C)C(C)(C)C ; CC(=O)C[C@@H]1CCO[C@@H]2[C@@H](C1)CN(C(=O)OC(C)(C)C)[C@@H]2CO]

Copper (I) chloride (20 mg, 202 μmol, 2 equiv) and palladium (II) chloride (7.0 mg, 39 μmol, 0.4 equiv) were added to a solution of olefin (41.6 mg, 98 μmol) in a mixture of N,N-dimethylformamide-water (10:1, 2.0 mL) at 23° C. The resulting suspension was sparged with oxygen for 5 min, after which it was vigorously stirred under oxygen atmosphere for 14 h. The reaction mixture was filtered through a Celite pad, and the filter cake was washed with dichloromethane (10 mL). The filtrate was concentrated in vacuo, and water was removed by repeated azeotroping with toluene. The residue was purified by flash chromatography on silica gel (10%→20%→50%→65% ethyl acetate-hexanes) to provide separately silyl ether-containing ketone (9.2 mg, 21%) as a colorless oil and primary alcohol-containing ketone (15.2 mg, 48%). ¹H NMR (1:1 rotamer ratio, 600 MHz, CDCl₃) δ: 3.97-3.65 (m, 6H), 3.59-3.46 (m, 1H), 2.88-2.67 (m, 2H), 2.48-2.32 (m, 2H), 2.13 (s, 3H), 1.93-1.73 (m, 2H), 1.65-1.54 (m, 1H), 1.50-1.37 (m, 10H), 1.00-0.82 (m, 10H), 0.07-0.00 (4s, 6H).

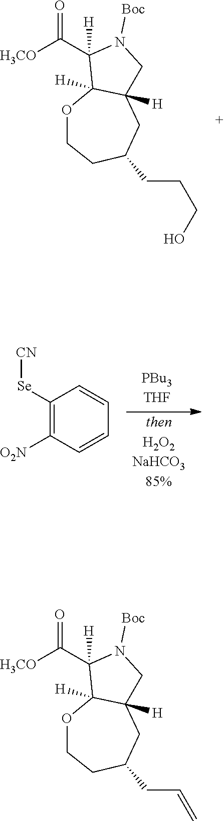 [SMILES: COC(=O)[C@@H]1[C@@H]2OCC[C@@H](CCCO)C[C@H]2CN1C(=O)OC(C)(C)C ; N#C[Se]c1ccccc1[N+](=O)[O-] ; C=CC[C@@H]1CCO[C@@H]2[C@@H](C1)CN(C(=O)OC(C)(C)C)[C@@H]2C(=O)OC]

o-Nitrophenylselenocyanate (323 mg, 1.42 mmol, 1.6 equiv) was added to a solution of alcohol (318.2 mg, 0.890 mmol) in tetrahydrofuran (18.0 mL) at 23° C. To the resulting solution was added tributylphosphine (0.35 mL, 288 mg, 1.42 mmol, 1.6 equiv) and the solution color changed from orange to dark brown. The reaction mixture was stirred at 23° C. for 2 h and was then cooled to 0° C. Sodium bicarbonate (1.68 g, 20 mmol, 22.5 equiv) and a 30% aqueous solution of hydrogen peroxide (2.7 mL, 26.7 mmol, 30 equiv) were added and the resulting suspension was warmed to 23° C. The reaction mixture was stirred at 23° C. for 2 h and was quenched with a saturated aqueous solution of ammonium chloride (30 mL). The mixture was extracted with dichloromethane (3×30 mL) and the combined organic layers were dried over sodium sulfate. The dried solution was concentrated in vacuo and the residue was purified by flash chromatography on silica gel (8%→16% ethyl acetate-hexanes) to provide the olefin (257.5 mg, 85%) as a pale yellow oil. ¹H NMR (1.5:1 rotamer ratio, “major” and “minor”, respectively, 500 MHz, CDCl₃) δ:5.74 (ddt, J=7.1, 10.5, 17.4 Hz, 0.6H, major), 5.73 (ddt, J=7.1, 10.5, 17.4 Hz, 0.4H, minor), 5.04-4.97 (m, 2H), 4.45 (d, J=8.2 Hz, 0.4H, minor), 4.36 (d, J=8.2 Hz, 0.6H, major), 4.15-4.07 (m, 1H), 3.94-3.82 (m, 2H), 3.81-3.71 (m, 4H), 2.88 (t, J=10.6 Hz, 0.4H, minor), 2.87 (t, J=10.6 Hz, 0.6H, major), 2.63-2.47 (m, 1H), 2.12-1.90 (m, 3H), 1.82-1.75 (m, 1H), 1.62-1.55 (m, 2H), 1.43 (s, 3.6H, minor), 1.39 (s, 5.4H, major), 0.99-0.90 (m, 1H).

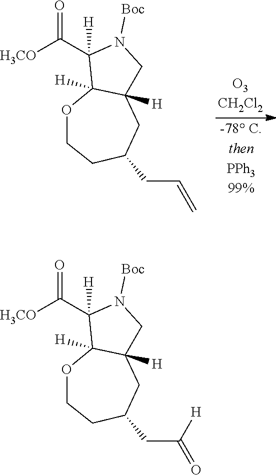 [SMILES: C=CC[C@@H]1CCO[C@@H]2[C@@H](C1)CN(C(=O)OC(C)(C)C)[C@@H]2C(=O)OC ; COC(=O)[C@@H]1[C@@H]2OCC[C@@H](CC=O)C[C@H]2CN1C(=O)OC(C)(C)C]

Olefin (90.3 mg, 0.266 mmol) was dissolved in dichloromethane (8.0 mL) and the resulting solution was cooled to −78° C. The solution was sparged with ozone until a blue color persisted (ca. 2 min), after which it was sparged with oxygen until the solution was colorless. Triphenylphosphine (140 mg, 0.532 mmol, 2 equiv) was added and the reaction mixture was warmed to 23° C. and was stirred at that temperature for 30 min. The volatiles were removed in vacuo and the residue was purified by flash chromatography on silica gel (20%→35% ethyl acetate-hexanes) to provide the aldehyde (90.2 mg, 99%) as a colorless oil. ¹H NMR (1.5:1 rotamer ratio, “major” and “minor”, respectively, 500 MHz, CDCl₃) δ: 9.69 (s, 1H), 4.39 (d, J=8.1 Hz, 0.4H, minor), 4.31 (d, J=8.2 Hz, 0.6H, major), 4.12-4.03 (m, 1H), 3.91-3.63 (m, 6H), 2.89-2.76 (m, 1H), 2.65-2.49 (m, 1H), 2.45-2.32 (m, 2H), 2.29-2.14 (m, 1H), 1.98-1.85 (m, 1H), 1.76-1.59 (m, 2H), 1.38 (s, 3.6H, minor), 1.34 (s, 5.4H, major), 1.01 (app q, J=11.6 Hz, 1H).

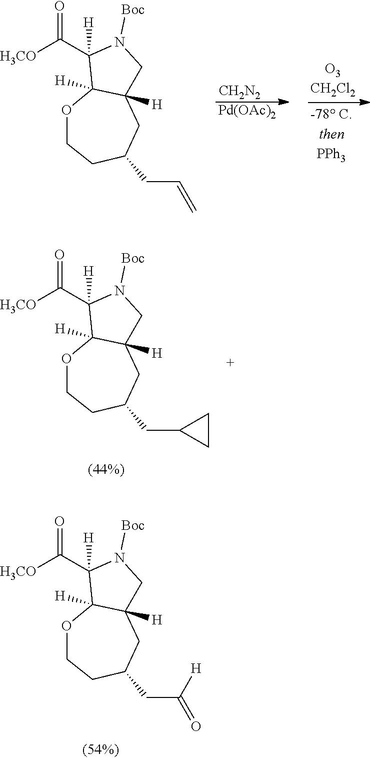 [SMILES: C=CC[C@@H]1CCO[C@@H]2[C@@H](C1)CN(C(=O)OC(C)(C)C)[C@@H]2C(=O)OC ; "CH2N2" ; COC(=O)[C@@H]1[C@@H]2OCC[C@@H](CC3CC3)C[C@H]2CN1C(=O)OC(C)(C)C ; "Pd(OAc)2" ; COC(=O)[C@@H]1[C@@H]2OCC[C@@H](CC=O)C[C@H]2CN1C(=O)OC(C)(C)C]

Olefin (24.9 mg, 73 μmol) was dissolved in a freshly prepared solution of diazomethane in ethyl ether (˜0.18 M, 2.0 mL, 0.37 mmol, 5 equiv) and the solution was cooled to 0° C. Palladium (II) acetate (3.3 mg, 15 μmol, 0.15 equiv) was added (vigorous gas evolution) and the resulting black suspension was stirred at 23° C. for 18 h. The reaction mixture was diluted with ethyl acetate (20 mL) and acetic acid (0.5 mL) was added. The suspension was stirred at 23° C. for 20 min, after which it was filtered through a Celite pad. The filtrate was washed sequentially with a saturated aqueous solution of sodium bicarbonate (20 mL) and a saturated aqueous solution of sodium chloride (20 mL). The organic layer was dried over sodium sulfate and the dried solution was concentrated in vacuo to provide an inseparable mixture of cyclopropane and olefin, which was used in the next step without further purification.

The crude mixture was dissolved in dichloromethane (1.5 mL) and the solution was cooled to −78° C. The solution was sparged with ozone until a blue color persisted (ca. 5 min) and the solution was then sparged with oxygen until the solution became colorless. Triphenylphosphine (58 mg, 0.22 mmol, 3 equiv) was added and the resulting solution was warmed to 23° C. over a 30 min period. The volatiles were removed in vacuo and the residue was purified by flash chromatography on silica gel (10%→15% ethyl acetate-hexanes) to provide separately cyclopropane ##(11.4 mg, 44%) and aldehyde (13.5 mg, 54%) as colorless oils. Cyclopropane: ¹H NMR (1.5:1 rotamer ratio, “major” and “minor”, respectively, 400 MHz, CDCl₃) δ: 4.45 (d, J=8.1 Hz, 0.4H, minor), 4.36 (d, J=8.2 Hz, 0.6H, major), 4.16-4.07 (m, 1H), 3.96-3.70 (m, 6H), 2.88 (t, J=10.7 Hz, 0.4H, minor), 2.86 (t, J=10.7 Hz, 0.6H, major), 2.67-2.48 (m, 1H), 2.10-1.98 (m, 1H), 1.89-1.79 (m, 1H), 1.75-1.58 (m, 2H), 1.44 (s, 3.6H, minor), 1.38 (s, 5.4H, major), 1.23-1.10 (m, 2H), 0.95 (app q, J=12.3 Hz, 1H), 0.70-0.58 (m, 1H), 0.47-0.38 (m, 2H), 0.03 to −0.04 (m, 2H).

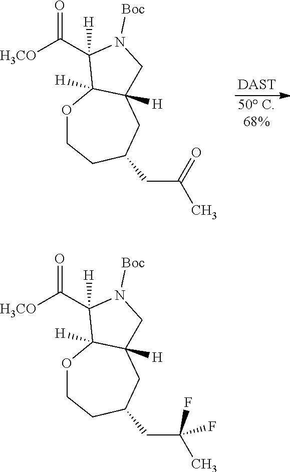

Ketone (11.3 mg, 32 μmol) was dissolved in diethylaminosulfur trifluoride (0.30 mL) and the resulting solution was heated to 50° C. for 48 h. The reaction mixture was diluted with dichloromethane (30 mL) and the organic solution was washed with a saturated aqueous solution of sodium bicarbonate (30 mL). The organic layer was dried over sodium sulfate and the dried solution was concentrated in vacuo. The residue was purified by flash chromatography on silica gel (15%→25% ethyl acetate-hexanes) to provide the difluoride (8.2 mg, 68%) as a colorless oil. ¹H NMR (1.5:1 rotamer ratio, “major” and “minor”, respectively, 500 MHz, CDCl₃) δ: 4.45 (d, J=8.1 Hz, 0.4H, minor), 4.36 (d, J=8.4 Hz, 0.6H, major), 4.16-4.09 (m, 1H), 3.93-3.71 (m, 6H), 2.92-2.83 (m, 1H), 2.69-2.52 (m, 1H), 2.09-1.65 (m, 6H), 1.59 (t, J=18.5 Hz, 3H), 1.44 (s, 3.6H, minor), 1.39 (s, 5.4H, major), 1.09-0.99 (m, 1H)

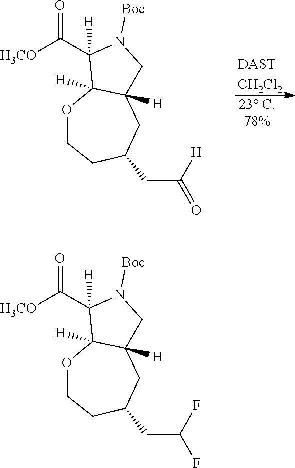 [SMILES: COC(=O)[C@@H]1[C@@H]2OCC[C@@H](CC=O)C[C@H]2CN1C(=O)OC(C)(C)C ; "DASTCH2Cl2" ; "23° C.78%" ; COC(=O)[C@@H]1[C@@H]2OCC[C@@H](CC(F)F)C[C@H]2CN1C(=O)OC(C)(C)C]

Diethylaminosulfur trifluoride (24 μL, 29.5 mg, 183 μmol, 4 equiv) was added to a solution of aldehyde (15.6 mg, 46 μmol) in dichloromethane (0.46 mL) at 23° C. The solution was stirred at 23° C. for 4 h and dichloromethane (20 mL) was added. The reaction mixture was quenched with a saturated aqueous solution of sodium carbonate (10 mL) and water (10 mL) and the mixture was vigorously stirred for 10 min. The layers were separated and the aqueous layer was further extracted with dichloromethane (20 mL). The combined organic layers were dried over sodium sulfate and the dried solution was concentrated in vacuo. The residue was purified by flash chromatography on silica gel (10%→15% ethyl acetate-hexanes) to provide the gem-difluoride (12.9 mg, 78%) as a colorless oil. ¹H NMR (1.5:1 rotamer ratio, “major” and “minor”, respectively, 500 MHz, CDCl₃) δ: 5.85 (app tq, J=4.4, 56.5 Hz, 1H), 4.46 (d, J=8.1 Hz, 0.4H, minor), 4.36 (d, J=8.2 Hz, 0.6H, major), 4.15-4.08 (m, 1H), 3.94-3.71 (m, 6H), 2.89 (t, J=10.6 Hz, 0.4H, minor), 2.87 (t, J=10.6 Hz, 0.6H, major), 2.71-2.52 (m, 1H), 2.06-1.66 (m, 6H), 1.43 (s, 3.6H, minor), 1.39 (s, 5.4H, major), 1.06 (app dt, J=11.6, 13.4 Hz, 1H).

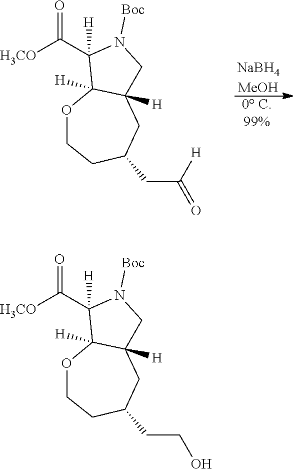 [SMILES: COC(=O)[C@@H]1[C@@H]2OCC[C@@H](CC=O)C[C@H]2CN1C(=O)OC(C)(C)C ; "0° C.99%" ; "NaBH4MeOH" ; COC(=O)[C@@H]1[C@@H]2OCC[C@@H](CCO)C[C@H]2CN1C(=O)OC(C)(C)C]

Sodium borohydride (16 mg, 0.41 mmol, 3 equiv) was added to an ice-cooled solution of aldehyde (47.1 mg, 0.138 mmol) in methanol (2.8 mL). The resulting mixture was stirred at 0° C. for 30 min, after which water (10 mL) and a saturated aqueous solution of ammonium chloride (10 mL) were added. The resulting solution was stirred for 12 h at 23° C. and the mixture was extracted with dichloromethane (2×30 mL). The combined organic phases were dried over sodium sulfate and the dried solution was concentrated in vacuo. The residue was purified by flash chromatography on silica gel (40%→50% ethyl acetate-hexanes) to provide the alcohol (46.7 mg, 99%) as a colorless oil. ¹H NMR (1.5:1 rotamer ratio, “major” and “minor”, respectively, 600 MHz, CDCl₃) δ: 4.42 (d, J=8.1 Hz, 0.4H, minor), 4.33 (d, J=8.2 Hz, 0.6H, major), 4.12-4.04 (m, 1H), 3.90-3.68 (m, 6H), 3.64 (td, J=1.9, 6.6 Hz, 2H), 2.85 (t, J=10.6 Hz, 0.4H, minor), 2.83 (t, J=10.6 Hz, 0.6H, major), 2.62-2.48 (m, 1H), 2.00-1.91 (m, 1H), 1.80-1.72 (m, 2H), 1.66-1.58 (m, 1H), 1.56-1.49 (m, 2H), 1.40 (s, 3.6H, minor), 1.36 (s, 5.4H, major), 0.99-0.89 (m, 1H);

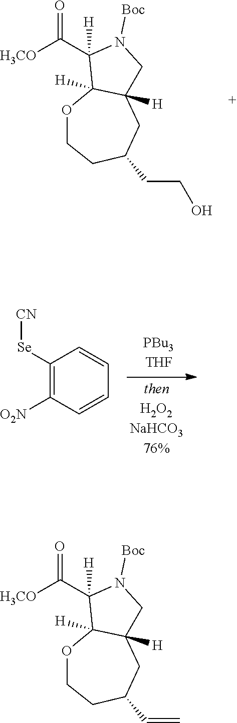 [SMILES: COC(=O)[C@@H]1[C@@H]2OCC[C@@H](CCO)C[C@H]2CN1C(=O)OC(C)(C)C ; N#C[Se]c1ccccc1[N+](=O)[O-] ; C=C[C@@H]1CCO[C@@H]2[C@@H](C1)CN(C(=O)OC(C)(C)C)[C@@H]2C(=O)OC]

o-Nitrophenylselenocyanate (34 mg, 0.15 mmol, 1.5 equiv) was added to a solution of alcohol (34.3 mg, 0.10 mmol) in tetrahydrofuran (2.0 mL) at 23° C. To the resulting solution was added tributylphosphine (0.04 mL, 30 mg, 0.15 mmol, 1.5 equiv) and the solution color changed from orange to dark brown. The reaction mixture was stirred at 23° C. for 2 h and was then cooled to 0° C. Sodium bicarbonate (189 mg, 2.25 mmol, 22.5 equiv) and a 30% aqueous solution of hydrogen peroxide (0.31 mL, 3.0 mmol, 30 equiv) were added and the resulting suspension was warmed to 23° C. The reaction mixture was stirred at 23° C. for 2 h and was quenched with a saturated aqueous solution of ammonium chloride (20 mL). The mixture was extracted with dichloromethane (3×20 mL) and the combined organic layers were dried over sodium sulfate. The dried solution was concentrated in vacuo and the residue was purified by flash chromatography on silica gel (8%→10% ethyl acetate-hexanes) to provide the olefin (24.8 mg, 76%) as a pale yellow oil. ¹H NMR (1.5:1 rotamer ratio, “major” and “minor”, respectively, 600 MHz, CDCl₃) δ: 5.79 (ddd, J=6.8, 10.4, 17.2 Hz, 1H), 4.98 (dd, J=1.4, 17.2 Hz, 1H), 4.92 (dd, J=1.3, 10.4 Hz, 1H), 4.45 (d, J=8.1 Hz, 0.4H, minor), 4.36 (d, J=8.2 Hz, 0.6H, major), 4.18-4.08 (m, 1H), 3.95-3.70 (m, 6H), 2.89 (t, J=10.6 Hz, 0.4H, minor), 2.88 (t, J=10.6 Hz, 0.6H, major), 2.68-2.53 (m, 1H), 2.35-2.25 (m, 1H), 2.00 (d, J=13.6 Hz, 0.6H, major), 1.96 (d, J=14.3 Hz, 0.4H, minor), 1.87-1.69 (m, 2H), 1.43 (s, 3.6H, minor), 1.38 (s, 5.4H, major), 1.11 (app q, J=11.7 Hz, 1H).

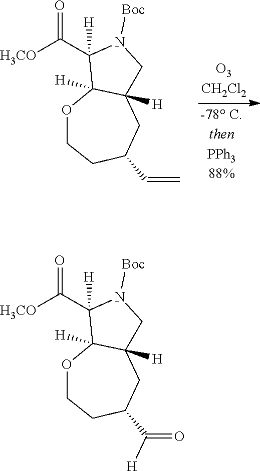 [SMILES: C=C[C@@H]1CCO[C@@H]2[C@@H](C1)CN(C(=O)OC(C)(C)C)[C@@H]2C(=O)OC ; COC(=O)[C@@H]1[C@@H]2OCC[C@@H](C=O)C[C@H]2CN1C(=O)OC(C)(C)C]

Olefin (46.1 mg, 0.136 mmol) was dissolved in dichloromethane (2.7 mL) and the resulting solution was cooled to −78° C. The solution was sparged with ozone until a blue color persisted (ca. 2 min), after which it was sparged with oxygen until the solution was colorless. Triphenylphosphine (107 mg, 0.407 mmol, 3 equiv) was added and the reaction mixture was warmed to 23° C. and was stirred at that temperature for 30 min. The volatiles were removed in vacuo and the residue was purified by flash chromatography on silica gel (8%→18% acetone-hexanes) to provide the aldehyde (39.1 mg, 88%) as a colorless oil. ¹H NMR (1.5:1 rotamer ratio, “major” and “minor”, respectively, 600 MHz, CDCl₃) δ: 9.65 (s, 1H), 4.44 (d, J=8.1 Hz, 0.4H, minor), 4.36 (d, J=8.3 Hz, 0.6H, minor), 4.04-3.79 (m, 4H), 3.72 (s, 1.2H, minor), 3.71 (s, 1.8H, minor), 2.94 (t, J=10.7 Hz, 0.4H, minor), 2.93 (t, J=10.7 Hz, 0.6H, minor), 2.64-2.51 (m, 2H), 2.33-2.18 (m, 2H), 1.92-1.83 (m, 1H), 1.42 (s, 3.6H, minor), 1.38 (s, 5.4H, major), 1.33-1.23 (m, 1H).

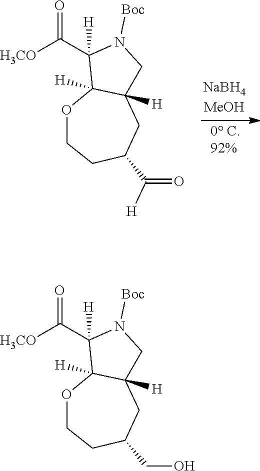 [SMILES: COC(=O)[C@@H]1[C@@H]2OCC[C@@H](C=O)C[C@H]2CN1C(=O)OC(C)(C)C ; COC(=O)[C@@H]1[C@@H]2OCC[C@@H](CO)C[C@H]2CN1C(=O)OC(C)(C)C]

Sodium borohydride (9.3 mg, 0.25 mmol, 3 equiv) was added to an ice-cooled solution of aldehyde (26.8 mg, 0.082 mmol) in methanol (1.6 mL). The resulting mixture was stirred at 0° C. for 30 min, after which water (10 mL) and a saturated aqueous solution of ammonium chloride (10 mL) were added. The resulting solution was stirred for 12 h at 23° C. and the mixture was extracted with dichloromethane (2×30 mL). The combined organic phases were dried over sodium sulfate and the dried solution was concentrated in vacuo. The residue was purified by flash chromatography on silica gel (40%→70% ethyl acetate-hexanes) to provide the alcohol (24.7 mg, 92%) as a colorless oil. ¹H NMR (1.5:1 rotamer ratio, “major” and “minor”, respectively, 600 MHz, CDCl₃) δ: 4.44 (d, J=8.2 Hz, 0.4H, minor), 4.36 (d, J=8.3 Hz, 0.6H, major), 4.12-4.04 (m, 1H), 3.97-3.69 (m, 6H), 3.55-3.41 (m, 2H), 2.88 (t, J=10.8 Hz, 0.4H, minor), 2.87 (t, J=10.8 Hz, 0.6H, minor), 2.64-2.46 (m, 1H), 2.06-1.58 (m, 5H), 1.43 (s, 3.6H, minor), 1.38 (s, 5.4H, major), 0.96 (app q, J=11.7 Hz, 1H).

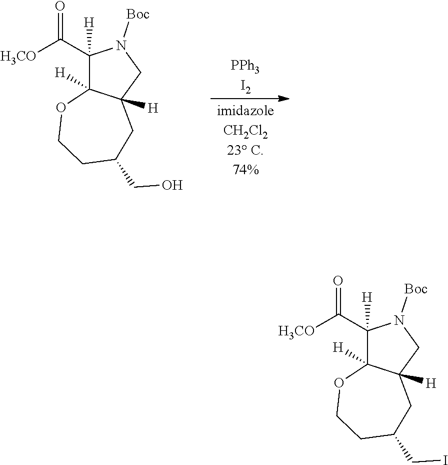 [SMILES: COC(=O)[C@@H]1[C@@H]2OCC[C@@H](CO)C[C@H]2CN1C(=O)OC(C)(C)C ; COC(=O)[C@@H]1[C@@H]2OCC[C@@H](CI)C[C@H]2CN1C(=O)OC(C)(C)C]

Triphenylphosphine (19.6 mg, 0.075 mmol, 1.2 equiv) was dissolved in dichloromethane (0.63 mL) and the solution was held at 23° C. in a water bath. Solid iodine (19.7 mg, 0.078 mmol, 1.25 equiv) was added to this solution and the resulting dark solution was stirred at 23° C. for 10 min. Solid imidazole (12.7 mg, 0.187 mmol, 3 equiv) was added and the resulting yellow suspension was stirred at 23° C. for a further 10 min. A solution of alcohol (20.5 mg, 0.062 mmol) in dichloromethane (0.63 mL) was added via cannula, and dichloromethane (0.20 mL) was used to quantitate the transfer. The solution was stirred at 23° C. for 1 h. The reaction mixture was quenched with a saturated aqueous solution of sodium thiosulfate (25 mL). The biphasic mixture was stirred at 23° C. for 30 min, after which water (20 mL) was added. The mixture was extracted with dichloromethane (3×30 mL) and the combined organic layers were dried over sodium sulfate. The dried solution was concentrated in vacuo and the residue was purified by flash chromatography on silica gel (12%→16% ethyl acetate-hexanes) to provide the iodide (20.1 mg, 74%) as a colorless oil. ¹H NMR (1.5:1 rotamer ratio, “major” and “minor”, respectively, 400 MHz, CDCl₃) δ: 4.45 (d, J=8.2 Hz, 0.4H, minor), 3.36 (d, J=8.2 Hz, 0.6H, major), 4.18-4.03 (m, 1H), 3.97-3.67 (m, 6H), 3.28-3.08 (m, 2H), 2.91 (t, J=10.6 Hz, 0.4H, minor), 2.89 (t, J=10.6 Hz, 0.6H, major), 2.70-2.48 (m, 1H), 2.14-1.70 (m, 4H), 1.43 (s, 3.6H, minor), 1.38 (s, 5.4H, major), 1.10 (app q, J=12.6 Hz, 1H).

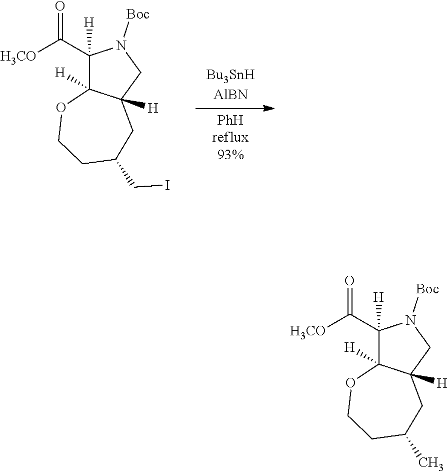 [SMILES: COC(=O)[C@@H]1[C@@H]2OCC[C@@H](CI)C[C@H]2CN1C(=O)OC(C)(C)C ; COC(=O)[C@@H]1[C@@H]2OCC[C@@H](C)C[C@H]2CN1C(=O)OC(C)(C)C]

Alkyl iodide (20.1 mg, 46 μmol) was dissolved in benzene (0.92 mL) and azo-bis-isobutyronitrile (1.5 mg, 9.2 μmol, 0.2 equiv) was added. Tributyltin hydride (0.03 mL, 29.3 mg, 0.10 mmol, 2.2 equiv) was added and the resulting solution was refluxed for 2 h. Additional tributyltin hydride (0.03 mL, 29.3 mg, 0.10 mmol, 2.2 equiv) was added and the mixture was refluxed for a further 1 h. The solution was cooled to 23° C. and potassium fluoride (50 mg) and water (0.10 mL) were added. The biphasic mixture was stirred at 23° C. for 1 h, after which sodium sulfate was added. The resulting suspension was filtered through a silica gel plug, eluting with ethyl acetate, and the filtrate was concentrated in vacuo. The residue was purified by flash chromatography on silica gel (0%→12% ethyl acetate-hexanes) to provide the desired product (13.4 mg, 93%) as a colorless oil. ¹H NMR (1.5:1 rotamer ratio, “major” and “minor”, respectively, 600 MHz, CDCl₃) δ: 4.44 (d, J=8.1 Hz, 0.4H, minor), 4.35 (d, J=8.3 Hz, 1H), 4.15-4.08 (m, 1H), 3.91-3.70 (m, 6H), 2.85 (t, J=10.6 Hz, 0.4H, minor), 2.84 (t, J=10.6 Hz, 0.6H, major), 2.63-2.48 (m, 1H), 1.92 (d, J=13.4 Hz, 0.6H, major), 1.87 (d, J=13.8 Hz, 0.4H, minor), 1.79-1.54 (m, 3H), 1.43 (s, 3.6H, minor), 1.39 (s, 5.4H, major), 0.97 (d, J=6.8 Hz, 3H), 0.94-0.88 (m, 1H).

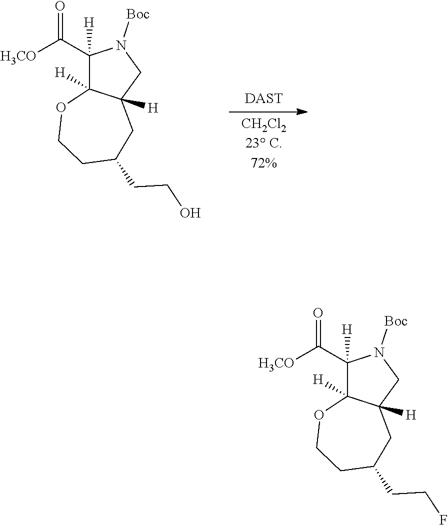 [SMILES: COC(=O)[C@@H]1[C@@H]2OCC[C@@H](CCO)C[C@H]2CN1C(=O)OC(C)(C)C ; "DAST" ; COC(=O)[C@@H]1[C@@H]2OCC[C@@H](CCF)C[C@H]2CN1C(=O)OC(C)(C)C]

Diethylaminosulfur trifluoride (0.02 mL, 19 mg, 0.12 mmol, 3 equiv) was added to a solution of alcohol (13.7 mg, 40 μmol) in dichloromethane (0.40 mL) at 23° C. The solution was stirred at 23° C. for 4 h and dichloromethane (20 mL) was added. The reaction mixture was quenched with a saturated aqueous solution of sodium carbonate (10 mL) and water (10 mL) and the mixture was vigorously stirred for 10 min. The layers were separated and the aqueous layer was further extracted with dichloromethane (20 mL). The combined organic layers were dried over sodium sulfate and the dried solution was concentrated in vacuo. The residue was purified by flash chromatography on silica gel (15%→25% ethyl acetate-hexanes) to provide the alkyl fluoride (9.9 mg, 72%) as a colorless oil. ¹H NMR (1.5:1 rotamer ratio, “major” and “minor”, respectively, 500 MHz, CDCl₃) δ: 4.57-4.33 (m, 3H), 4.16-4.08 (m, 1H), 3.95-3.71 (m, 6H), 2.89 (t, J=10.6 Hz, 0.4H, minor), 2.87 (t, J=10.6 Hz, 0.6H, major), 2.68-2.50 (m, 1H), 2.05-1.57 (m, 6H), 1.44 (s, 3.6H, minor), 1.39 (s, 5.4H, major), 1.00 (app q, J=12.4 Hz, 1H); ¹⁹F NMR (376.5 MHz, CDCl₃) δ: −218.50 (app ttd, J=21.4, 26.6, 47.3 Hz).

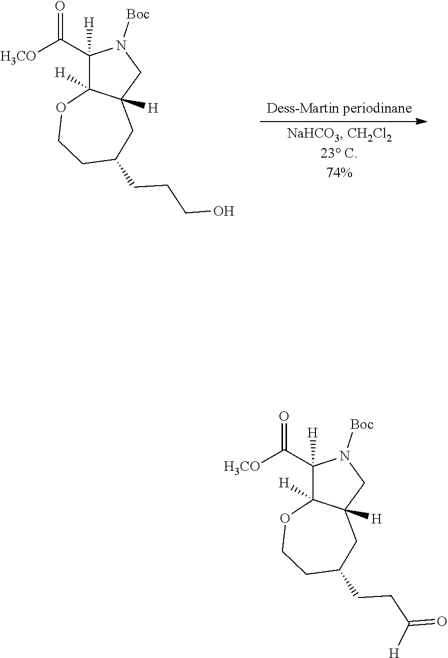 [SMILES: COC(=O)[C@@H]1[C@@H]2OCC[C@@H](CCCO)C[C@H]2CN1C(=O)OC(C)(C)C ; "Dess-Martin periodinane" ; COC(=O)[C@@H]1[C@@H]2OCC[C@@H](CCC=O)C[C@H]2CN1C(=O)OC(C)(C)C]

Sodium bicarbonate (81 mg, 0.97 mmol, 10 equiv) and Dess-Martin periodinane (103 mg, 0.242 mmol, 2.5 equiv) were added to a solution of alcohol (34.6 mg, 0.097 mmol) in dichloromethane (2.0 mL) at 23° C. and the resulting suspension was stirred at that temperature for 1 h. The reaction mixture was quenched with a saturated aqueous solution of sodium thiosulfate (10 mL) and was stirred at 23° C. for 45 min. A saturated aqueous solution of sodium bicarbonate (20 mL) was added and the mixture was extracted with dichloromethane (3×20 mL). The combined organic layers were dried over sodium sulfate and the dried solution was concentrated in vacuo. The residue was purified by flash chromatography on silica gel (25%→30% ethyl acetate-hexanes) to provide the aldehyde (25.5 mg, 74%) as a colorless oil. ¹H NMR (1.5:1 rotamer ratio, “major” and “minor”, respectively, 500 MHz, CDCl₃) δ: 9.76 (s, 1H), 4.44 (d, J=8.1 Hz, 0.4H, minor), 4.35 (d, J=8.2 Hz, 0.6H, major), 4.13-4.05 (m, 1H), 3.92-3.68 (m, 6H), 2.91-2.81 (m, 1H), 2.64-2.41 (m, 3H), 1.99-1.87 (m, 1H), 1.79-1.70 (m, 1H), 1.66-1.56 (m, 4H), 1.42 (s, 3.6H, minor), 1.38 (s, 5.4H, major), 1.00-0.89 (m, 1H).

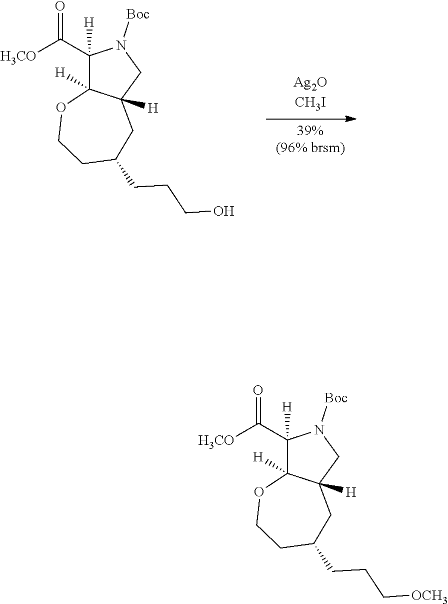 [SMILES: COC(=O)[C@@H]1[C@@H]2OCC[C@@H](CCCO)C[C@H]2CN1C(=O)OC(C)(C)C ; "Ag2OCH3I" ; COCCC[C@@H]1CCO[C@@H]2[C@@H](C1)CN(C(=O)OC(C)(C)C)[C@@H]2C(=O)OC]

Alcohol (18.1 mg, 51 μmol) was dissolved in iodomethane (0.95 mL, 2.2 g, 15.2 mmol, 300 equiv) and silver (I) oxide (70.4 mg, 0.304 mmol, 6 equiv). The resulting suspension was stirred at 23° C. for 18 h, after which it was diluted with ethyl acetate (15 mL) and filtered through a silica gel plug. The filtrate was concentrated in vacuo (in a fume hood) and the residue was purified by flash chromatography on silica gel (8%→13% acetone-hexanes) to provide separately the methyl ether (7.4 mg, 39%) and recovered alcohol (10.7 mg). ¹H NMR (1.5:1 rotamer ratio, “major” and “minor”, respectively, 600 MHz, CDCl₃) δ: 4.44 (d, J=8.2 Hz, 0.4H, minor), 4.35 (d, J=8.2 Hz, 0.6H, major), 4.14-4.06 (m, 1H), 3.93-3.70 (m, 6H), 3.34 (t, J=6.5 Hz, 2H), 3.31 (s, 3H), 2.87 (t, J=10.6 Hz, 0.4H, minor), 2.86 (t, J=10.6 Hz, 0.6H, major), 2.63-2.46 (m, 1H), 1.97 (d, J=12.9 Hz, 0.6H, major), 1.93 (d, J=13.6 Hz, 0.4H, minor), 1.79-1.72 (m, 1H), 1.64-1.53 (m, 4H), 1.43 (s, 3.6H, minor), 1.39 (s, 5.4H, major), 1.36-1.29 (m, 2H), 0.93 (app q, J=13.5 Hz, 1H).

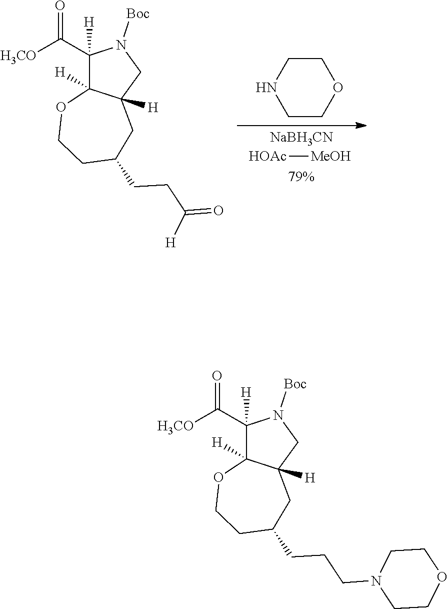

Morpholine (0.02 mL, 17 mg, 0.20 mmol, 4 equiv) was added to an ice-cold solution of aldehyde (17.7 mg, 0.050 mmol) in a mixture of methanol (0.45 mL) and acetic acid (0.05 mL) and the resulting solution was stirred at 0° C. for 10 min. Sodium cyanoborohydride (6.4 mg, 0.10 mmol, 2 equiv) was added and the reaction mixture was stirred at 0° C. for 2 h. The reaction mixture was quenched with a mixture of water (15 mL) and a saturated aqueous solution of sodium carbonate (10 mL) and the resulting mixture was extracted with dichloromethane (3×20 mL). The combined organic layers were dried over sodium sulfate and the dried solution was concentrated in vacuo. The residue was purified by flash chromatography on silica gel (25%→50% acetone-hexanes) to provide the amine (16.7 mg, 79%) as a colorless oil. ¹H NMR (1.5:1 rotamer ratio, “major” and “minor”, respectively, 500 MHz, CDCl₃) δ: 4.44 (d, J=8.2 Hz, 0.4H, minor), 4.35 (d, J=8.2 Hz, 0.6H, major), 4.14-4.04 (m, 1H), 3.94-3.65 (m, 10H), 2.90-2.81 (m, 1H), 2.62-2.36 (m, 5H), 2.29 (t, J=7.7 Hz, 2H), 2.01-1.88 (m, 1H), 1.78-1.71 (m, 1H), 1.64-1.55 (m, 2H), 1.51-1.45 (m, 2H), 1.42 (s, 3.6H, minor), 1.38 (s, 5.4H, major), 1.33-1.26 (m, 2H), 0.97-0.87 (m, 1H).

General procedure for Conversion of N-Boc Amino Methyl Esters to Lincosamides

### Ester Hydrolysis

The methyl ester was dissolved in a mixture of methanol (0.1 M) and tetrahydrofuran (0.1 M) at 23° C. and an aqueous solution of lithium hydroxide (1.0 M, 4-8 equiv) was added. The reaction mixture was stirred at ° C. for 12-18 h and was diluted with dichloromethane (ca. 100 mL per mmol of substrate) and water (ca. 100 mL per mmol of substrate). The aqueous layer was acidified to a pH of 2 using an aqueous solution of HCl (1.0 M) and the mixture was vigorously shaken. The layers were separated and the aqueous layer was further extracted with dichloromethane (2×100 mL per mmol of substrate). The combined organic layers were dried over sodium sulfate and the dried solution was concentrated in vacuo to provide the crude acid.

### Amide Coupling

The crude acid was dissolved in a solution of N,N-diisopropylethylamine in N,N-dimethylformamide (0.3 M, 3 equiv) and HATU (1.2 equiv) was added at 23° C. The resulting solution was stirred for 5 min at 23° C., at which point 7-Cl-MTL (1.3 equiv) was added. The reaction mixture was stirred at 23° C. for 12-18 h and was diluted with dichloromethane (ca. 100 mL per mmol of substrate). For amides not containing a basic nitrogen, the organic solution was washed sequentially with a 5% aqueous solution of citric acid (ca. 100 mL per mmol of substrate) and a saturated aqueous solution of sodium bicarbonate (ca. 100 mL per mmol of substrate). For amides containing a basic nitrogen, the organic solution was washed with a saturated aqueous solution of sodium carbonate (ca. 100 mL per mmol of substrate). The organic layer was dried over sodium sulfate and the dried solution was concentrated in vacuo. The crude amide was used in the deprotection step or, when noted, the residue was purified by flash chromatography on silica gel to provide the pure amide.

### N-Deprotection

The carbamate was dissolved in a freshly prepared solution of HCl in methanol (1 M, 100 equiv) and the resulting solution was heated to 50° C. for 10 min. The reaction mixture was cooled to 23° C. and the solvent was removed in vacuo. The residue was dissolved in a mixture of N,N-dimethylformamide-methanol (1:1) and passed through a 0.2 μm syringe filter. The filtered solution was purified by preparative high-performance liquid chromatography on C18 silica gel (eluent acetonitrile-water+0.1% formic acid) to provide the pure lincosamide as its formate salt.

### FSA-10-9-004

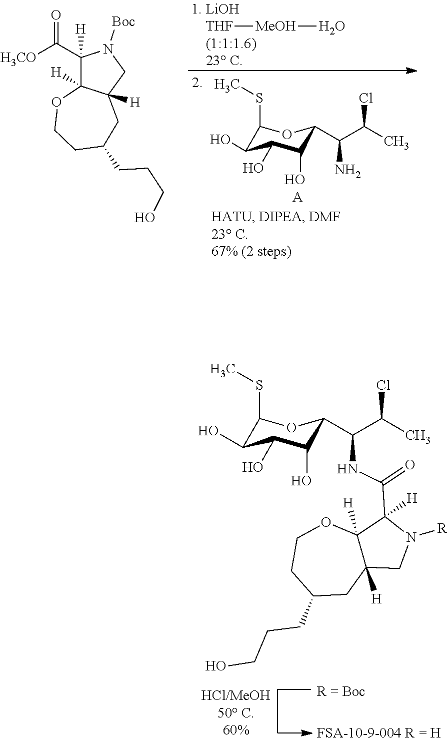

Ester (8.4 mg, 23 μmol) was hydrolyzed according to the general procedure using tetrahydrofuran (0.12 mL), methanol (0.12 mL) and 1 M aqueous lithium hydroxide solution (0.10 mL, 0.10 mmol, 4 equiv). The resulting crude acid was coupled according to the general procedure using a solution of N,N-diisopropylethylamine in N,N-dimethylformamide (0.3 M, 0.24 mL, 71 μmol, 3 equiv), HATU (10.7 mg, 28 μmol, 1.2 equiv), and 7-Cl-MTL (8.3 mg, 31 μmol, 1.3 equiv). The crude product was purified by flash chromatography on silica gel (6%→8% methanol-dichloromethane) to provide the amide (9.4 mg, 67%, 2 steps) as a white solid. ¹H NMR (1.5:1 rotamer ratio, “major” and “minor”, respectively, 600 MHz, D₃COD) δ: 5.30 (d, J=6.0 Hz, 0.4H, minor), 5.29 (d, J=6.0 Hz, 0.6H, major), 4.65-4.50 (m, 2H), 4.45-4.40 (m, 1H), 4.37 (d, J=9.8 Hz, 0.4H, minor), 4.27 (d, J=10.0 Hz, 0.6H, major), 4.20-4.01 (m, 3H), 3.94-3.89 (m, 1H), 3.80-3.69 (m, 2H), 3.61-3.56 (m, 1H), 3.53 (t, J=6.6 Hz, 2H), 2.91 (t, J=10.6 Hz, 1H), 2.53-2.40 (m, 1H), 2.15 (s, 1.2H, minor), 2.14 (s, 1.8H, major), 2.00 (d, J=13.6 Hz, 0.4H, minor), 1.95 (d, J=13.2 Hz, 0.6H, major), 1.82-1.74 (m, 1H), 1.73-1.65 (m, 1H), 1.63-1.50 (m, 6H), 1.45 (s, 9H), 1.41-1.32 (m, 2H), 1.05-0.95 (m, 1H).

Carbamate (7.6 mg, 13 μmol) was deprotected using the general procedure using 1 M HCl in methanol (1.3 mL, 1.3 mmol, 100 equiv). The crude product was purified by preparative high-performance liquid chromatography on C18 silica gel (5%→50% acetonitrile-water + 0.1% formic acid, gradient over 35 min) to provide FSA-10-9-004 as its formate salt (4.17 mg, 60%). ¹H NMR (600 MHz, D₃COD) δ:5.30 (d, J=5.6 Hz, 1H), 4.58 (q, J=7.2 Hz, 1H), 4.50 (d, J=9.9 Hz, 1H), 4.36 (d, J=8.9 Hz, 1H), 4.32 (t, J=9.1 Hz, 1H), 4.26 (d, J=10.0 Hz, 1H), 4.09 (dd, J=5.6, 10.2 Hz, 1H), 4.00 (dt, J=4.1, 11.9 Hz, 1H), 3.84 (s, 1H), 3.81 (dd, J=3.7, 11.3 Hz, 1H), 3.60-3.50 (m, 4H), 2.84 (t, J=11.5 Hz, 1H), 2.29-2.21 (m, 1H), 2.15 (s, 3H), 2.03 (d, J=13.4 Hz, 1H), 1.85-1.73 (m, 2H), 1.69-1.61 (m, 1H), 1.60-1.54 (m, 2H), 1.53 (d, J=6.9 Hz, 3H), 1.45-1.35 (m, 2H), 1.01 (app q, J=12.0 Hz, 1H).

### FSA-10-9-017

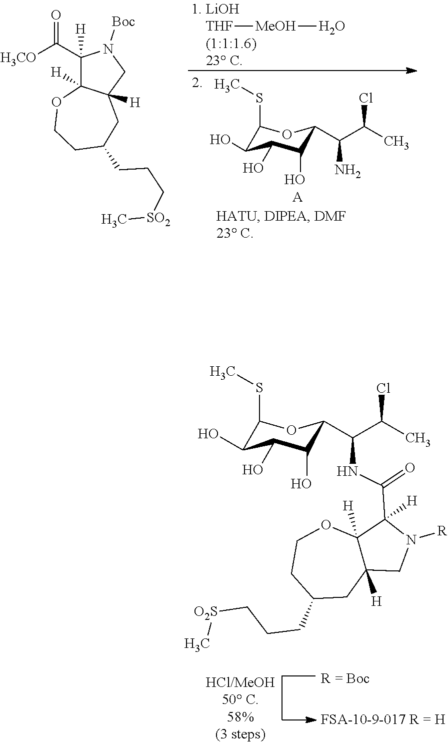

Ester (3.8 mg, 9.1 μmol) was hydrolyzed according to the general procedure using tetrahydrofuran (0.10 mL), methanol (0.10 mL) and 1 M aqueous lithium hydroxide solution (0.08 mL, 0.08 mmol, 8 equiv). The resulting crude acid was coupled according to the general procedure using a solution of N,N-diisopropylethylamine in N,N-dimethylformamide (0.3 M, 0.08 mL, 10.9 μmol, 2.5 equiv), HATU (4.2 mg, 10.9 μmol, 1.2 equiv), and 7-Cl-MTL (3.2 mg, 12 μmol, 1.3 equiv). The crude product was deprotected according to the general procedure using 1 M HCl in methanol (1.7 mL, 1.7 mmol, 189 equiv). The crude product was purified by preparative high-performance liquid chromatography on C18 silica gel (5%→70% acetonitrile-water +0.1% formic acid, gradient over 35 min) to provide FSA-10-9-017 as its formate salt (3.16 mg, 58%, 3 steps). ¹H NMR (600 MHz, D₃COD) δ: 5.30 (d, J=5.7 Hz, 1H), 4.57 (qd, J=1.6, 6.9 Hz, 1H), 4.50 (d, J=10.0 Hz, 1H), 4.40 (d, J=8.8 Hz, 1H), 4.32 (t, J=9.2 Hz, 1H), 4.27 (d, J=10.0 Hz, 1H), 4.08 (dd, J=5.6, 10.2 Hz, 1H), 4.00 (dt, J=4.1, 11.8 Hz, 1H), 3.86-3.79 (m, 2H), 3.60-3.52 (m, 2H), 3.13-3.08 (m, 2H), 2.95 (s, 3H), 2.90-2.83 (m, 1H), 2.31-2.23 (m, 1H), 2.14 (s, 3H), 2.06-2.01 (m, 1H), 1.88-1.77 (m, 4H), 1.73-1.66 (m, 1H), 1.53 (d, J=6.8 Hz, 3H), 1.51-1.45 (m, 2H), 1.03 (app q, J=12.1 Hz, 1H).

### FSA-10-9-018

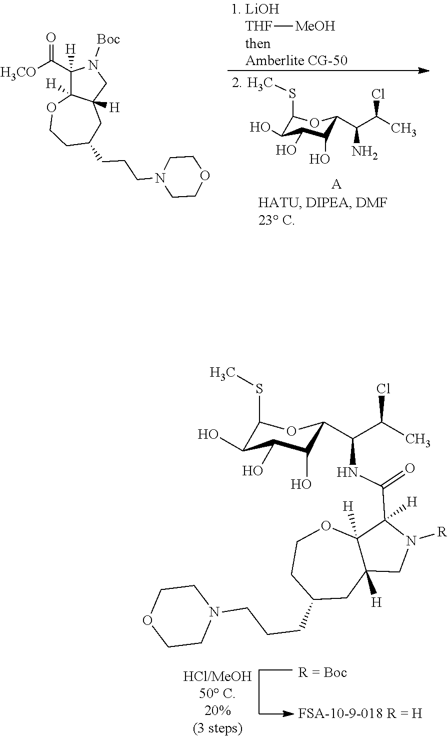

Methyl ester (16.7 mg, 0.039 mmol) was dissolved in a mixture of methanol (0.39 mL) and tetrahydrofuran (0.39 mL) at 23° C. and an aqueous solution of lithium hydroxide (1.0 M, 0.31 mL, 0.31 mmol, 8 equiv) was added. The resulting mixture was stirred at 23° C. for 40 h, after which it was diluted with methanol (3 mL) and water (4 mL) and the pH was adjusted to 7 using Amberlite CG-50 weakly acidic ion-exchange resin (ca. 1 g). The resulting suspension was filtered through a Celite pad and the filter cake was washed sequentially with methanol (35 mL) and water (35 mL). The filtrate was concentrated in vacuo to provide the crude amino acid, which was used in the next step without further purification.

The crude acid (assume 0.039 mmol) was coupled according to the general procedure using a solution of N,N-diisopropylethylamine in N,N-dimethylformamide (0.3 M, 0.33 mL, 98 μmol, 2.5 equiv), HATU (17.8 mg, 47 μmol, 1.2 equiv), and 7-Cl-MTL (14 mg, 51 μmol, 1.3 equiv). The crude product was deprotected according to the general procedure using 1 M HCl in methanol (4.0 mL, 4.0 mmol, 100 equiv). The crude product was purified by preparative high-performance liquid chromatography on C18 silica gel (5%-70% acetonitrile-water+0.1% formic acid, gradient over 35 min) to provide FSA-10-9-018 as its bis-formate salt (5.11 mg, 20%, 3 steps). ¹H NMR (600 MHz, D₃COD) δ: 8.38 (br s, 2H), 5.30 (d, J=5.6 Hz, 1H), 4.57 (qd, J=1.6, 6.8 Hz, 1H), 4.51 (d, J=9.9 Hz, 1H), 4.45 (d, J=8.6 Hz, 1H), 4.35 (t, J=9.2 Hz, 1H), 4.28 (d, J=10.0 Hz, 1H), 4.09 (dd, J=5.7, 10.2 Hz, 1H), 4.01 (dt, J=4.1, 11.8 Hz, 1H), 3.88-3.79 (m, 6H), 3.61-3.55 (m, 2H), 3.01-2.87 (m, 5H), 2.81 (t, J=8.1 Hz, 2H), 2.31-2.23 (m, 1H), 2.15 (s, 3H), 2.03 (d, J=13.2 Hz, 1H), 1.85-1.63 (m, 5H), 1.53 (d, J=6.8 Hz, 3H), 1.42-1.34 (m, 2H), 1.05 (app q, J=12.0 Hz, 1H).

### FSA-10-9-020

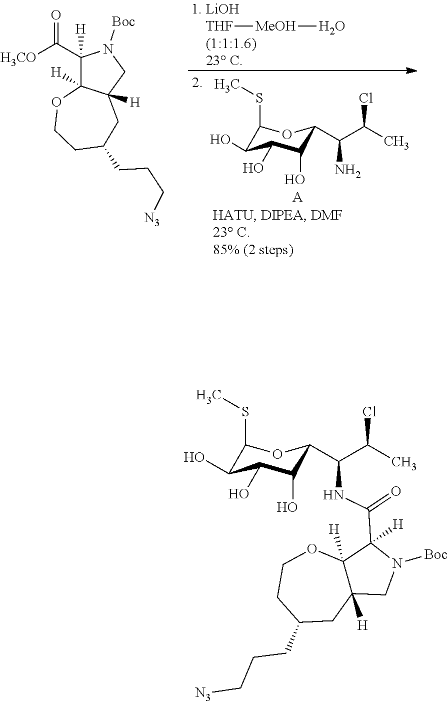

Ester (10.6 mg, 28 μmol) was hydrolyzed according to the general procedure using tetrahydrofuran (0.28 mL), methanol (0.28 mL) and 1 M aqueous lithium hydroxide solution (0.22 mL, 0.22 mmol, 8 equiv). The resulting crude acid was coupled according to the general procedure using a solution of N,N-diisopropylethylamine in N,N-dimethylformamide (0.3 M, 0.28 mL, 83 μmol, 3 equiv), HATU (12.6 mg, 33 μmol, 1.2 equiv), and 7-Cl-MTL (9.8 mg, 36 μmol, 1.3 equiv). The crude product was purified by flash chromatography on silica gel (30%→45% acetone-hexanes) to provide the amide (14.7 mg, 85%, 2 steps) as a white solid. ¹H NMR (1.5:1 rotamer ratio, “major” and “minor”, respectively, 600 MHz, D₃COD) δ: 5.30 (d, J=5.7 Hz, 0.4H, minor), 5.28 (d, J=5.7 Hz, 0.6H, major), 4.64-4.51 (m, 2H), 4.43 (t, J=8.5 Hz, 1H), 4.37 (d, J=10.0 Hz, 0.4H, minor), 4.27 (d, J=10.0 Hz, 0.6H, major), 4.22-4.00 (m, 3H), 3.96-3.87 (m, 1H), 3.82-3.68 (m, 2H), 3.62-3.52 (m, 1H), 3.28 (t, J=6.7 Hz, 2H), 2.91 (t, J=10.6 Hz, 1H), 2.55-2.41 (m, 1H), 2.15 (s, 1.2H, minor), 2.14 (s, 1.8H, major), 2.00-1.91 (m, 1H), 1.82-1.68 (m, 2H), 1.65-1.57 (m, 3H), 1.55 (d, J=6.8 Hz, 1.2H, minor), 1.52 (d, J=6.8 Hz, 1.8H, major), 1.46 (s, 9H), 1.41-1.34 (m, 2H), 1.07-0.97 (m, 1H).

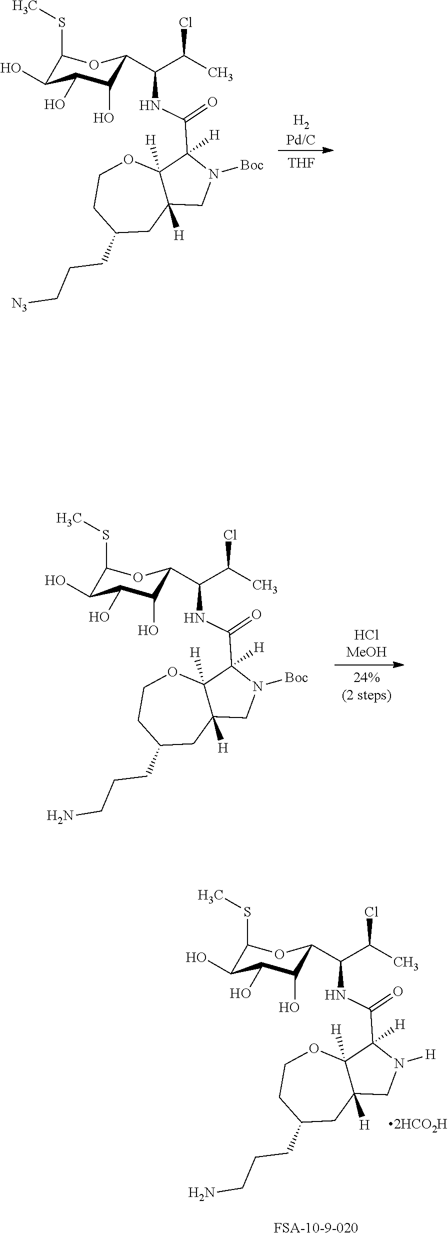

10% Palladium on carbon (14.4 mg, 1.44 mg of palladium, 14 μmol, 1.0 equiv) was added to a solution of the azide (8.4 mg, 14 μmol) in tetrahydrofuran (0.45 mL) under an argon atmosphere. The resulting suspension was sparged with hydrogen for 5 min and was then stirred under hydrogen atmosphere for 2 h. The reaction vessel was flushed with argon and the suspension was filtered through a Celite pad. The filter cake was washed with methanol (5 mL) and the filtrate was concentrated in vacuo. The residue was deprotected according to the general procedure using 1 M HCl in methanol (2.6 mL, 2.6 mmol, 189 equiv) and the crude product was purified by preparative high-performance liquid chromatography on C18 silica gel (3%→35% acetonitrile-water+0.1% formic acid, gradient over 35 min) to provide FSA-10-9-020 as its bis-formate salt (1.91 mg, 24%, 2 steps). ¹H NMR (600 MHz, D₃COD) δ: 5.30 (d, J=5.6 Hz, 1H), 4.57 (qd, J=1.6, 6.8 Hz, 1H), 4.48 (d, J=9.9 Hz, 1H), 4.31-4.28 (m, 2H), 4.25 (d, J=10.0 Hz, 1H), 4.08 (dd, J=5.7, 9.9 Hz, 1H), 3.99 (dt, J=4.1, 11.8 Hz, 1H), 3.85-3.78 (m, 2H), 3.57 (dd, J=3.5, 10.1 Hz, 1H), 3.49 (dd, J=7.4, 11.0 Hz, 1H), 2.90 (t, J=7.6 Hz, 2H), 2.80 (t, J=11.4 Hz, 1H), 2.29-2.21 (m, 1H), 2.14 (s, 3H), 2.01 (d, J=11.9 Hz, 1H), 1.83-1.76 (m, 2H), 1.71-1.63 (m, 3H), 1.52 (d, J=6.8 Hz, 3H), 1.45-1.35 (m, 2H), 1.03 (app q, J=12.1 Hz, 1H).

### FSA-10-9-044

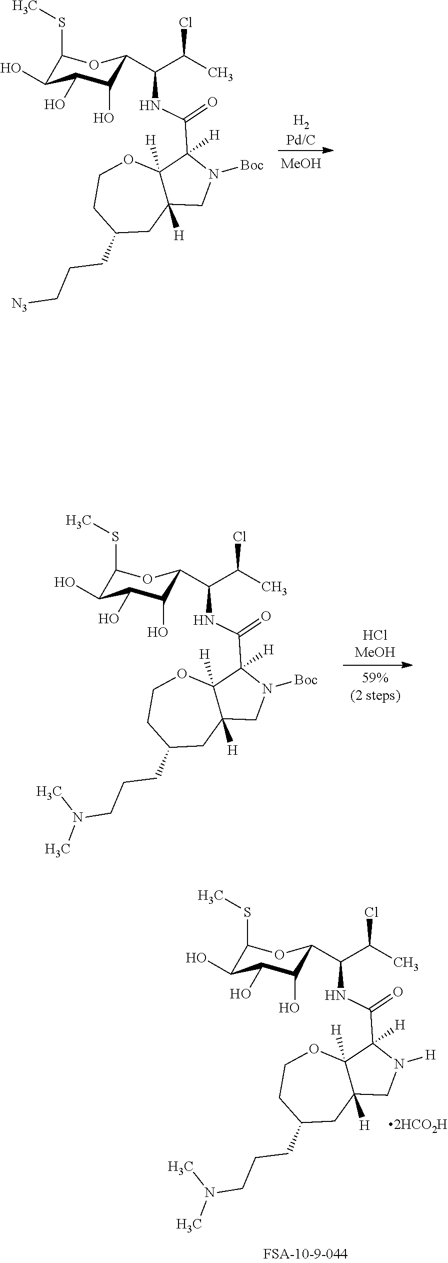

10% Palladium on carbon (7.0 mg, 0.70 mg of palladium, 6.6 μmol, 1.0 equiv) was added to a solution of the azide (4.1 mg, 6.6 μmol) in methanol (0.66 mL) under an argon atmosphere. The resulting suspension was sparged with hydrogen for 5 min and was then stirred under hydrogen atmosphere for 18 h. The reaction vessel was flushed with argon and additional 10% palladium on carbon (7.0 mg, 0.70 mg of palladium, 6.6 μmol, 1 equiv) was added and the suspension was stirred under hydrogen atmosphere for 36 h. The reaction vessel was flushed with argon and the suspension was filtered through a Celite pad. The filter cake was washed sequentially with methanol (10 mL) and a 9:1 mixture of methanol-saturated aqueous ammonium hydroxide (10 mL). The filtrate was concentrated in vacuo and the residue was deprotected according to the general procedure using 1 M HCl in methanol (1.2 mL, 1.2 mmol, 189 equiv). The crude product was purified by preparative high-performance liquid chromatography on C18 silica gel (5%→50% acetonitrile-water+0.1% formic acid, gradient over 35 min) to provide FSA-10-9-044 as its bis-formate salt (2.41 mg, 59%, 2 steps). ¹H NMR (600 MHz, D₃COD) δ: 5.30 (d, J=5.6 Hz, 1H), 4.58 (q, J=7.1 Hz, 1H), 4.49 (d, J=10.0 Hz, 1H), 4.33-4.29 (m, 2H), 4.26 (d, J=10.0 Hz, 1H), 4.08 (dd, J=5.6, 10.1 Hz, 1H), 4.00 (dt, J=4.0, 11.9 Hz, 1H), 3.86-3.80 (m, 2H), 3.60-3.46 (m, 2H), 3.07 (t, J=8.1 Hz, 2H), 2.87 (s, 6H), 2.84-2.78 (m, 1H), 2.31-2.22 (m, 1H), 2.15 (s, 3H), 2.02 (d, J=13.3 Hz, 1H), 1.84-1.65 (m, 5H), 1.52 (d, J=6.8 Hz, 3H), 1.43-1.35 (m, 2H), 1.03 (app q, J=12.2 Hz, 1H).

### FSA-10-9-045

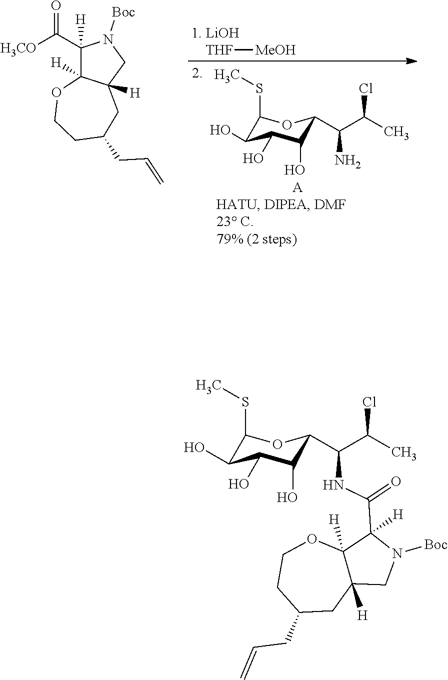

Ester (14.1 mg, 42 μmol) was hydrolyzed according to the general procedure using tetrahydrofuran (0.42 mL), methanol (0.42 mL) and 1 M aqueous lithium hydroxide solution (0.33 mL, 0.33 mmol, 8 equiv). The resulting crude acid was coupled according to the general procedure using a solution of N,N-diisopropylethylamine in N,N-dimethylformamide (0.3 M, 0.42 mL, 125 μmol, 3 equiv), HATU (19.2 mg, 50 μmol, 1.2 equiv), and 7-Cl-MTL (15 mg, 54 μmol, 1.3 equiv). The crude product was purified by flash chromatography on silica gel (25%→40% acetone-hexanes) to provide the amide (19.0 mg, 79%, 2 steps) as a white solid. ¹H NMR (1.5:1 rotamer ratio, “major” and “minor”, respectively, 500 MHz, D₃COD) δ: 5.84-5.74 (m, 1H), 5.30 (d, J=5.7 Hz, 0.4H, minor), 5.29 (d, J=5.7 Hz, 0.6H, major), 5.04-4.97 (m, 2H), 4.64-4.58 (m, 1H), 4.55 (qd, J=1.7, 6.9 Hz, 1H), 4.46-4.40 (m, 1H), 4.37 (d, J=9.9 Hz, 0.4H, minor), 4.26 (d, J=9.9 Hz, 0.6H, major), 4.21-4.02 (m, 3H), 3.95-3.87 (m, 1H), 3.81-3.67 (m, 2H), 3.62-3.52 (m, 1H), 2.90 (t, J=10.6 Hz, 1H), 2.54-2.40 (m, 1H), 2.16 (s, 1.2H, minor), 2.15 (s, 1.8H, major), 2.12-1.91 (m, 3H), 1.84-1.75 (m, 1H), 1.55 (d, J=6.8 Hz, 1.2H, minor), 1.52 (d, J=6.8 Hz, 1.8H, major), 1.46 (s, 9H), 1.04-0.97 (m, 1H).

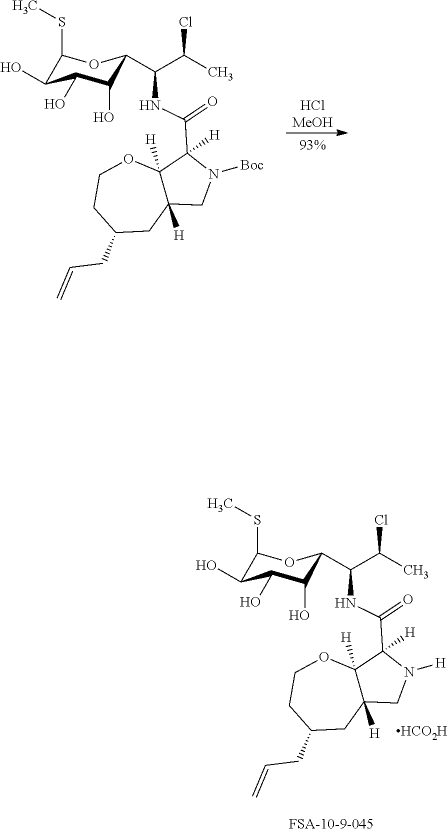

Carbamate (5.8 mg, 10.0 μmol) was deprotected according to the general procedure using 1 M HCl in methanol (1.9 mL, 1.9 mmol, 189 equiv). The crude product was purified by preparative high-performance liquid chromatography on C18 silica gel (10%→70% acetonitrile-water+0.1% formic acid, gradient over 25 min) to provide FSA-10-9-045 as its formate salt (4.91 mg, 93%). ¹H NMR (600 MHz, D₃COD) δ: 5.80 (ddt, J=7.1, 10.5, 17.4 Hz, 1H), 5.30 (d, J=5.6 Hz, 1H), 5.06-4.99 (m, 2H), 4.57 (qd, J=1.6, 6.9 Hz, 1H), 4.51 (d, J=10.0 Hz, 1H), 4.42 (d, J=8.9 Hz, 1H), 4.33 (t, J=9.2 Hz, 1H), 4.27 (d, J=10.0 Hz, 1H), 4.09 (dd, J=5.7, 10.2 Hz, 1H), 4.00 (dt, J=3.8, 11.9 Hz, 1H), 3.83 (d, J=3.3 Hz, 1H), 3.80 (td, J=3.8, 11.3 Hz, 1H), 3.61-3.53 (m, 2H), 2.88 (t, J=11.7 Hz, 1H), 2.31-2.20 (m, 1H), 2.15 (s, 3H), 2.14-2.05 (m, 2H), 2.02 (d, J=13.5 Hz, 1H), 1.86-1.69 (m, 3H), 1.53 (d, J=6.8 Hz, 3H), 1.02 (app q, J=11.9 Hz, 1H).

### FSA-10-9-051

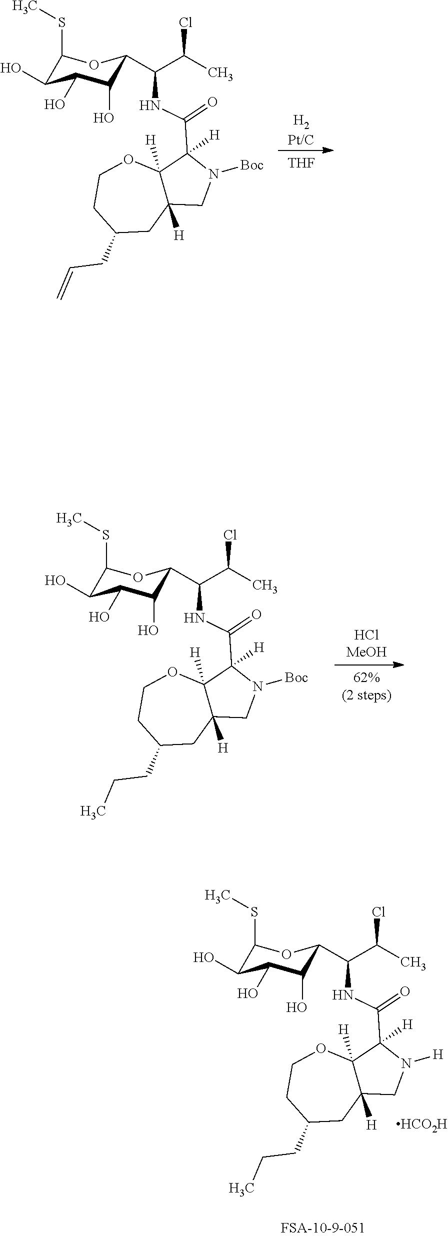

5% Platinum on carbon (71.2 mg, 3.56 mg of platinum, 18 μmol, 1.2 equiv) was added to a solution of the olefin (8.8 mg, 15 μmol) in tetrahydrofuran (0.50 mL) under an argon atmosphere. The resulting suspension was sparged with hydrogen for 5 min and was then stirred under hydrogen atmosphere for 30 min. The reaction vessel was flushed with argon and the suspension was filtered through a Celite pad. The filter cake was washed with tetrahydrofuran (20 mL) and the filtrate was concentrated in vacuo to provide the crude hydrogenation product ##. The crude carbamate was deprotected according to the general procedure using 1 M HCl in methanol (2.9 mL, 2.9 mmol, 189 equiv). The crude product was purified by preparative high-performance liquid chromatography on C18 silica gel (10%→70% acetonitrile-water+0.1% formic acid, gradient over 25 min) to provide FSA-10-9-051 as its formate salt (4.99 mg, 62%, 2 steps). ¹H NMR (600 MHz, D₃COD) δ: 8.41 (br s, 1H), 5.30 (d, J=5.7 Hz, 1H), 4.57 (qd, J=1.6, 6.8 Hz, 1H), 4.51 (dd, J=1.6, 9.9 Hz, 1H), 4.41 (d, J=8.9 Hz, 1H), 4.32 (t, J=9.2 Hz, 1H), 4.27 (d, J=10.0 Hz, 1H), 4.09 (dd, J=5.6, 10.2 Hz, 1H), 4.00 (dt, J=4.1, 11.8 Hz, 1H), 3.84 (br s, 1H), 3.80 (td, J=3.8, 11.3 Hz, 1H), 3.61-3.53 (m, 2H), 2.86 (t, J=11.7 Hz, 1H), 2.29-2.21 (m, 1H), 2.15 (s, 3H), 2.01 (d, J=13.5 Hz, 1H), 1.83-1.70 (m, 2H), 1.67-1.59 (m, 1H), 1.53 (d, J=6.8 Hz, 3H), 1.40-1.27 (m, 4H), 1.00 (app q, J=12.1 Hz, 1H), 0.92 (t, J=7.0 Hz, 3H).

### FSA-10-9-067

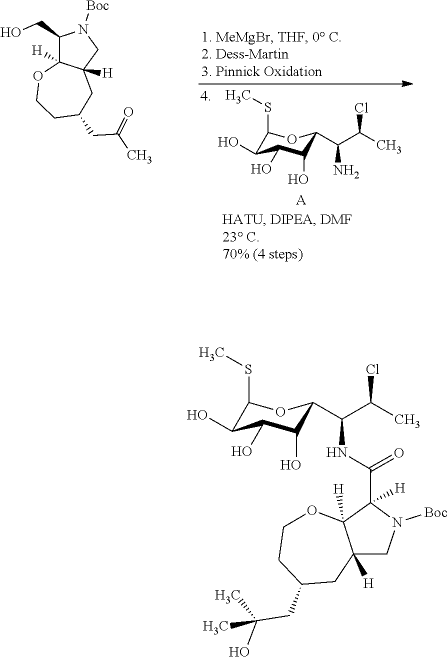

A 3.0 M solution of methylmagnesium bromide in ethyl ether (0.08 mL, 0.24 mmol, 8 equiv) was added dropwise to an ice-cold solution of ketone (9.4 mg, 29 μmol) in tetrahydrofuran (1.1 mL) and the resulting solution was stirred at 0° C. for 10 min. The reaction mixture was quenched with a saturated aqueous solution of ammonium chloride (20 mL) and the resulting solution was extracted with dichloromethane (3×20 mL). The combined organic layers were dried over sodium sulfate and the dried solution was concentrated in vacuo to provide the crude diol, which was used in the next step without further purification.

Sodium bicarbonate (29 mg, 0.345 mmol, 12 equiv) and Dess-Martin periodinane (37 mg, 0.086 mmol, 3 equiv) were added to a solution of crude diol (29 μmol) in dichloromethane (1.0 mL) at 23° C. and the resulting suspension was stirred at that temperature for 1 h. The reaction mixture was quenched with a saturated aqueous solution of sodium thiosulfate (10 mL) and was stirred at 23° C. for 45 min. A saturated aqueous solution of sodium bicarbonate (20 mL) was added and the mixture was extracted with dichloromethane (3×20 mL). The combined organic layers were dried over sodium sulfate and the dried solution was concentrated in vacuo to provide the crude aldehyde, which was used in the next step without further purification.

The crude aldehyde (29 μmol) was dissolved in a mixture of tetrahydrofuran (0.87 mL), tert-butanol (0.22 mL) and amylene (0.18 mL, 121 mg, 1.72 mmol, 60 equiv) at 23° C. Sodium dihydrogen phosphate (17 mg, 0.138 mmol, 4.8 equiv) was added, followed by an aqueous solution of sodium chlorite (1.0 M, 0.14 mL, 0.14 mmol, 4.8 equiv). The solution was stirred at 23° C. for 2 h, after which it was diluted with dichloromethane (20 mL). The organic solution was washed with a pH 2 aqueous 1 M sodium bisulfate—sodium sulfate buffer solution (20 mL) and the layers were separated. The aqueous layer was further extracted with dichloromethane (20 mL) and the combined organic layers were dried over sodium sulfate. The dried solution was concentrated in vacuo and the residue was passed through a plug of silica gel, eluting with 10% methanol-dichloromethane. The filtrate was concentrated in vacuo to provide the crude acid, which was used in the next step without further purification.

The crude acid (29 μmol) was coupled according to the general procedure using a solution of N,N-diisopropylethylamine in N,N-dimethylformamide (0.3 M, 0.29 mL, 86 μmol, 3 equiv), HATU (13.1 mg, 34 μmol, 1.2 equiv), and 7-Cl-MTL (10.1 mg, 37 μmol, 1.3 equiv). The crude product was purified by flash chromatography on silica gel (3%→6% methanol-dichloromethane) to provide the amide (12.2 mg, 70%, 4 steps) as a white solid. ¹H NMR (1.5:1 rotamer ratio, “major” and “minor”, respectively, 500 MHz, D₃COD) δ: 5.30 (d, J=6.2 Hz, 0.4H, minor), 5.29 (d, J=6.2 Hz, 0.6H, major), 4.63-4.57 (m, 1H), 4.55 (q, J=6.9 Hz, 1H), 4.46-4.40 (m, 1H), 4.37 (d, J=10.0 Hz, 0.4H, minor), 4.27 (d, J=9.9 Hz, 0.6H, major), 4.22-4.03 (m, 3H), 3.95-3.69 (m, 3H), 3.62-3.54 (m, 1H), 2.89 (t, J=10.6 Hz, 1H), 2.56-2.45 (m, 1H), 2.15 (s, 1.2H, minor), 2.14 (s, 1.8H, major), 2.08-1.99 (m, 1H), 1.96-1.72 (m, 3H), 1.56 (d, J=6.7 Hz, 1.2H, minor), 1.53 (d, J=6.8 Hz, 1.8H, major), 1.49-1.42 (m, 11H), 1.19 (s, 6H), 1.11-1.01 (m, 1H).

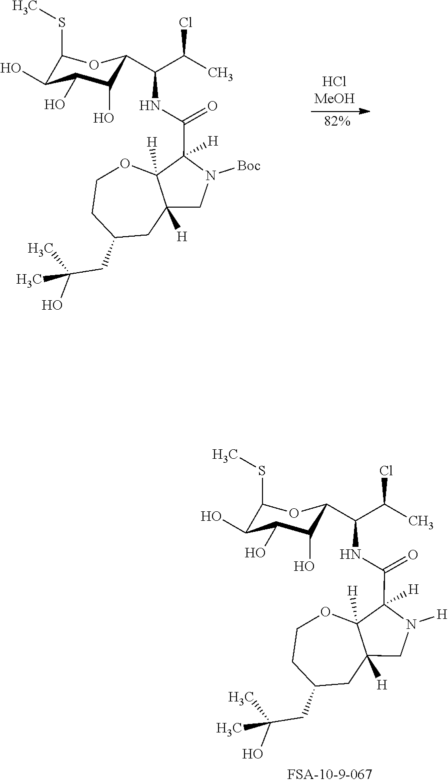

Carbamate (10.0 mg, 16.4 μmol) was deprotected according to the general procedure using 1 M HCl in methanol (3.0 mL, 3.0 mmol, 189 equiv). The crude product was purified by preparative high-performance liquid chromatography on C18 silica gel (5%→55% acetonitrile-water+0.1% formic acid, gradient over 25 min) to provide FSA-10-9-067 as its formate salt (7.50 mg, 82%). ¹H NMR (600 MHz, D₃COD) δ: 8.34 (br s, 1H), 5.31 (d, J=5.6 Hz, 1H), 4.57 (q, J=6.9 Hz, 1H), 4.52 (d, J=10.0 Hz, 1H), 4.46 (d, J=8.9 Hz, 1H), 4.36 (t, J=9.2 Hz, 1H), 4.28 (d, J=10.0 Hz, 1H), 4.09 (ddd, J=1.8, 5.7, 10.3 Hz, 1H), 4.01-3.96 (m, 1H), 3.87-3.81 (m, 2H), 3.62-3.54 (m, 2H), 2.89 (t, J=11.8 Hz, 1H), 2.34-2.24 (m, 1H), 2.15 (s, 3H), 2.11 (d, J=13.6 Hz, 1H), 1.95 (d, J=14.7 Hz, 1H), 1.91-1.79 (m, 2H), 1.54 (d, J=6.9 Hz, 3H), 1.52-1.44 (m, 2H), 1.21 (s, 6H), 1.09 (app q, J=11.9 Hz, 1H).

### FSA-10-09-093

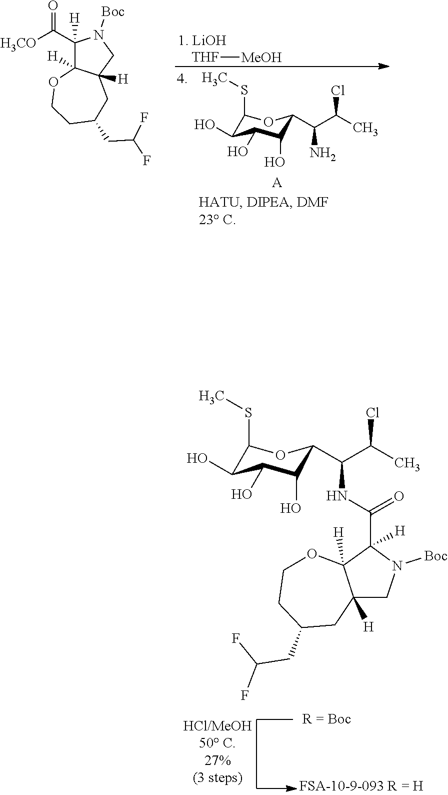

Ester (10.4 mg, 29 μmol) was hydrolyzed according to the general procedure using tetrahydrofuran (0.29 mL), methanol (0.29 mL) and 1 M aqueous lithium hydroxide solution (0.23 mL, 0.23 mmol, 8 equiv). The resulting crude acid was coupled according to the general procedure using a solution of N,N-diisopropylethylamine in N,N-dimethylformamide (0.3 M, 0.29 mL, 86 μmol, 3 equiv), HATU (13.1 mg, 34 μmol, 1.2 equiv), and 7-Cl-MTL (10.1 mg, 37 μmol, 1.3 equiv). The crude product was deprotected according to the general procedure using 1 M HCl in methanol (5.4 mL, 5.4 mmol, 189 equiv). The crude product was purified by preparative high-performance liquid chromatography on C18 silica gel (5%→40% acetonitrile-water+0.1% formic acid, gradient over 25 min) to provide FSA-10-9-093 as its formate salt (4.18 mg, 27%, 3 steps). ¹H NMR (600 MHz, D₃COD) δ: 5.98 (tt, J=4.6, 56.6 Hz, 1H), 5.30 (d, J=5.6 Hz, 1H), 4.58 (qd, J=1.6, 6.8 Hz, 1H), 4.50 (d, J=9.9 Hz, 1H), 4.40-4.30 (m, 2H), 4.26 (d, J=10.0 Hz, 1H), 4.09 (dd, J=5.6, 10.2 Hz, 1H), 3.99 (dt, J=4.0, 12.0 Hz, 1H), 3.87-3.78 (m, 2H), 3.58 (dd, J=3.2, 10.2 Hz, 1H), 3.53 (dd, J=7.4, 11.0 Hz, 1H), 2.84 (t, J=11.5 Hz, 1H), 2.34-2.22 (m, 1H), 2.15 (s, 3H), 2.06 (d, J=13.4 Hz, 1H), 1.99-1.81 (m, 5H), 1.53 (d, J=6.8 Hz, 3H), 1.13 (app q, J=12.0 Hz, 1H); ¹⁹F NMR (376.5 MHz, D₃COD) δ: −116.29 (m).

### FSA-10-09-098

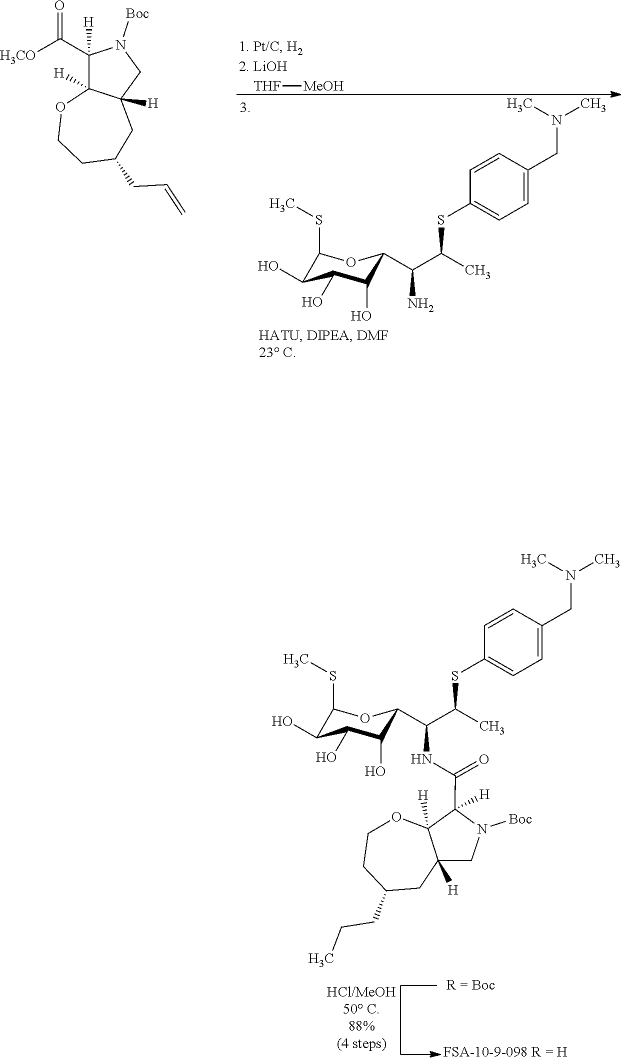

5% Platinum on carbon (23.8 mg, 1.19 mg of platinum, 6.1 μmol, 0.15 equiv) was added to a solution of olefin (13.8 mg, 41 μmol) in tetrahydrofuran (0.41 mL) under an argon atmosphere. The resulting suspension was sparged with hydrogen for 5 min, after which the suspension was vigorously stirred under hydrogen atmosphere for 1 h. The reaction vessel was flushed with argon and the reaction mixture was filtered through a Celite pad. The filter cake was washed with additional tetrahydrofuran (15 mL) and the combined filtrates were concentrated in vacuo to provide the crude hydrogenation product, which was used in the next step without further purification.

The crude ester (41 μmol, 3 equiv)) was hydrolyzed according to the general procedure using tetrahydrofuran (0.41 mL), methanol (0.41 mL) and 1 M aqueous lithium hydroxide solution (0.33 mL, 0.33 mmol, 8 equiv). The resulting crude acid was coupled according to the general procedure using a solution of N,N-diisopropylethylamine in N,N-dimethylformamide (0.3 M, 0.26 mL, 79 μmol, 6 equiv), HATU (15.5 mg, 41 μmol, 3 equiv), and amine (5.3 mg, 13 μmol, 1.0 equiv). The crude product was deprotected according to the general procedure using 1 M HCl in methanol (2.5 mL, 2.5 mmol, 189 equiv). The crude product was purified by preparative high-performance liquid chromatography on C18 silica gel (5%→30% acetonitrile-water+0.1% formic acid, gradient over 25 min) to provide FSA-10-9-098 as its bis-formate salt (8.21 mg, 88%, 4 steps). ¹H NMR (600 MHz, D₃COD) δ: 7.45 (d, J=8.1 Hz, 2H), 7.40 (d, J=8.2 Hz, 2H), 5.24 (d, J=5.6 Hz, 1H), 4.66 (dd, J=2.3, 9.8 Hz, 1H), 4.42 (d, J=10.0 Hz, 1H), 4.38 (d, J=8.7 Hz, 1H), 4.33 (t, J=9.1 Hz, 1H), 4.15 (s, 2H), 4.08 (dd, J=5.6, 10.2 Hz, 1H), 4.02 (dt, J=4.1, 11.8 Hz, 1H), 3.94-3.85 (m, 2H), 3.81 (td, J=3.9, 11.2 Hz, 1H), 3.59-3.53 (m, 2H), 2.87 (t, J=11.7 Hz, 1H), 2.76 (s, 6H), 2.30-2.21 (m, 1H), 2.01 (d, J=13.5 Hz, 1H), 1.83 (s, 3H), 1.81-1.70 (m, 2H), 1.66-1.58 (m, 1H), 1.43 (d, J=6.9 Hz, 3H), 1.39-1.25 (m, 4H), 1.00 (app q, J=12.1 Hz, 1H), 0.91 (t, J=7.0 Hz, 3H).

### FSA-10-09-099

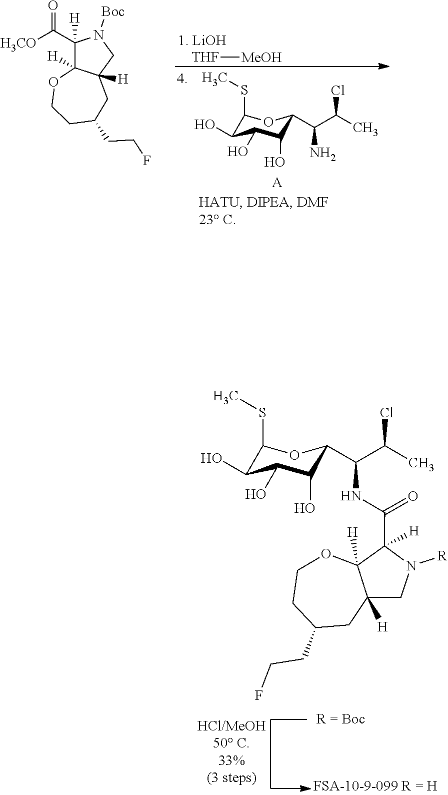

Ester (9.9 mg, 29 μmol) was hydrolyzed according to the general procedure using tetrahydrofuran (0.29 mL), methanol (0.29 mL) and 1 M aqueous lithium hydroxide solution (0.23 mL, 0.23 mmol, 8 equiv). The resulting crude acid was coupled according to the general procedure using a solution of N,N-diisopropylethylamine in N,N-dimethylformamide (0.3 M, 0.29 mL, 86 μmol, 3 equiv), HATU (13.1 mg, 34 μmol, 1.2 equiv), and 7-Cl-MTL (10.1 mg, 37 μmol, 1.3 equiv). The crude product was deprotected according to the general procedure using 1 M HCl in methanol (5.0 mL, 5.0 mmol, 189 equiv). The crude product was purified by preparative high-performance liquid chromatography on C18 silica gel (5%→40% acetonitrile-water+0.1% formic acid, gradient over 25 min) to provide FSA-10-9-099 as its formate salt (5.00 mg, 33%, 3 steps). ¹H NMR (600 MHz, D₃COD) δ: 8.34 (br s, 1H), 5.30 (d, J=5.6 Hz, 1H), 4.61-4.41 (m, 5H), 4.35 (t, J=9.2 Hz, 1H), 4.28 (d, J=10.0 Hz, 1H), 4.09 (dd, J=5.6, 10.2 Hz, 1H), 4.01 (dt, J=3.7, 11.7 Hz, 1H), 3.87-3.78 (m, 2H), 3.62-3.55 (m, 2H), 2.90 (t, J=11.7 Hz, 1H), 2.34-2.24 (m, 1H), 2.15 (s, 3H), 2.06 (d, J=13.7 Hz, 1H), 1.89-1.80 (m, 3H), 1.72 (dq, J=6.1, 26.5 Hz, 2H), 1.54 (d, J=6.8 Hz, 3H), 1.09 (app q, J=11.8 Hz, 1H); ¹⁹F NMR (376.5 MHz, D₃COD) δ: —220.29 (m).

### FSA-10-10-001

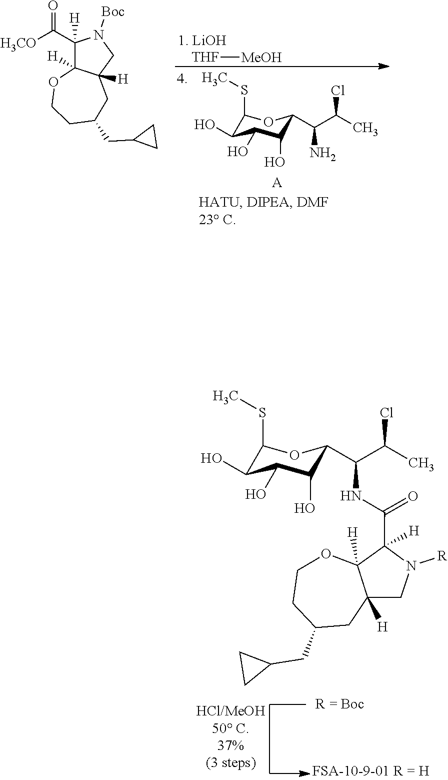

Ester (11.4 mg, 32 μmol) was hydrolyzed according to the general procedure using tetrahydrofuran (0.32 mL), methanol (0.32 mL) and 1 M aqueous lithium hydroxide solution (0.26 mL, 0.26 mmol, 8 equiv). The resulting crude acid was coupled according to the general procedure using a solution of N,N-diisopropylethylamine in N,N-dimethylformamide (0.3 M, 0.33 mL, 97 μmol, 3 equiv), HATU (14.7 mg, 39 μmol, 1.2 equiv), and 7-Cl-MTL (11.0 mg, 42 μmol, 1.3 equiv). The crude product was deprotected according to the general procedure using 1 M HCl in methanol (6.5 mL, 6.5 mmol, 189 equiv). The crude product was purified by preparative high-performance liquid chromatography on C18 silica gel (5%→40% acetonitrile-water+0.1% formic acid, gradient over 30 min) to provide FSA-10-10-001 as its formate salt (6.48 mg, 37%, 3 steps). ¹H NMR (600 MHz, D₃COD) δ: 5.31 (d, J=5.6 Hz, 1H), 4.57 (qd, J=1.6, 6.9 Hz, 1H), 4.51 (dd, J=1.6, 10.0 Hz, 1H), 4.43 (d, J=8.9 Hz, 1H), 4.34 (t, J=9.2 Hz, 1H), 4.28 (d, J=10.0 Hz, 1H), 4.09 (dd, J=5.6, 10.2 Hz, 1H), 4.02 (dt, J=3.8, 11.9 Hz, 1H), 3.86-3.77 (m, 2H), 3.61-3.54 (m, 2H), 2.89 (t, J=11.7 Hz, 1H), 2.33-2.22 (m, 1H), 2.15 (s, 3H), 2.11 (d, J=14.5 Hz, 1H), 1.92-1.73 (m, 3H), 1.54 (d, J=6.8 Hz, 3H), 1.30-1.22 (m, 2H), 1.05 (app q, J=11.9 Hz, 1H), 0.72 (dqd, J=4.9, 7.3, 14.5 Hz, 1H), 0.50-0.42 (m, 2H), 0.03 (dt, J=2.7, 5.5 Hz, 1H).

### FSA-10-10-008

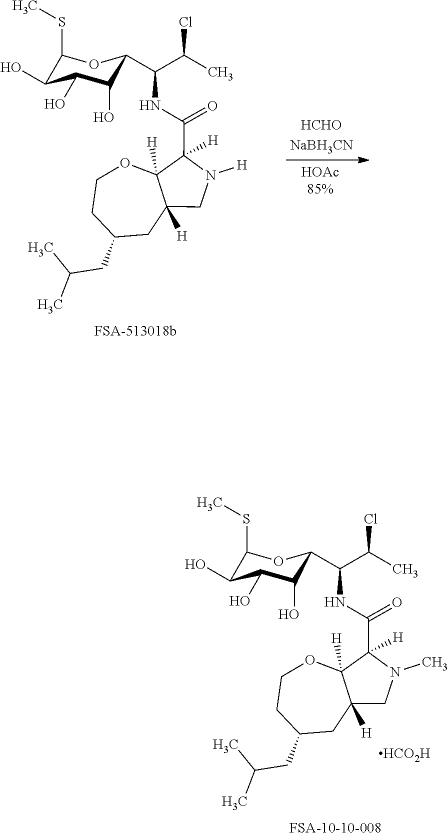

FSA-513018b (6.7 mg, 14 μmol) was dissolved in a mixture of acetic acid (0.31 mL) and a 37% aqueous solution of formaldehyde (0.05 mL, 20.3 mg of formaldehyde, 0.68 mmol, 50 equiv) at 23° C. Sodium cyanoborohydride (5.1 mg, 81 μmol, 6 equiv) was added and the solution was stirred at 23° C. for 45 min. The reaction mixture was quenched with a saturated aqueous solution of sodium carbonate (20 mL) and the mixture was extracted with dichloromethane (2×20 mL). The combined organic layers were dried over sodium sulfate and the dried solution was concentrated in vacuo. The residue was purified by preparative high-performance liquid chromatography on C18 silica gel (5%→45% acetonitrile-water+0.1% formic acid, gradient over 25 min) to provide FSA-10-10-008 as its formate salt (6.4 mg, 85%). ¹H NMR (600 MHz, D₃COD) δ: 5.30 (d, J=5.6 Hz, 1H), 4.63 (qd, J=1.5, 6.8 Hz, 1H), 4.36 (dd, J=1.6, 9.9 Hz, 1H), 4.24 (t, J=9.1 Hz, 1H), 4.16 (dd, J=1.2, 10.0 Hz, 1H), 4.07 (dd, J=5.6, 10.2 Hz, 1H), 4.03 (dd, J=1.2, 3.5 Hz, 1H), 3.97 (dt, J=3.3, 11.2 Hz, 1H), 3.83-3.76 (m, 1H), 3.58 (dd, J=3.5, 10.2 Hz, 1H), 3.20 (d, J=9.7 Hz, 1H), 3.14 (dd, J=6.1, 8.9 Hz, 1H), 2.36 (s, 3H), 2.32-2.25 (m, 1H), 2.19-2.15 (m, 1H), 2.13 (s, 3H), 1.94 (d, J=13.2, Hz, 1H), 1.71-1.60 (m, 3H), 1.48 (d, J=6.8 Hz, 3H), 1.24-1.10 (m, 2H), 0.92-0.87 (m, 1H), 0.89 (d, J=6.6 Hz, 3H), 0.88 (d, J=6.6 Hz, 3H).

### FSA-10-10-012

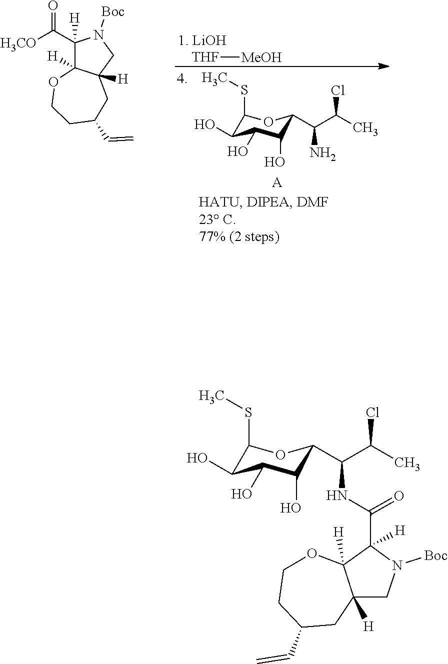

Ester (13.0 mg, 40 μmol) was hydrolyzed according to the general procedure using tetrahydrofuran (0.40 mL), methanol (0.40 mL) and 1 M aqueous lithium hydroxide solution (0.32 mL, 0.32 mmol, 8 equiv). The resulting crude acid was coupled according to the general procedure using a solution of N,N-diisopropylethylamine in N,N-dimethylformamide (0.3 M, 0.40 mL, 12 μmol, 3 equiv), HATU (18.3 mg, 48 μmol, 1.2 equiv), and 7-Cl-MTL (14 mg, 52 μmol, 1.3 equiv). The crude product was purified by flash chromatography on silica gel (30%→40% acetone-hexanes) to provide the amide (17.3 mg, 77%, 2 steps) as a white solid. ¹H NMR (1.5:1 rotamer ratio, “major” and “minor”, respectively, 600 MHz, D₃COD) δ: 5.86 (ddd, J=7.1, 10.5, 17.4 Hz, 1H), 5.30 (d, J=6.2 Hz, 0.4H, minor), 5.29 (d, J=6.2 Hz, 0.6H, major), 5.01 (d, J=17.3 Hz, 1H), 4.92 (d, J=10.4 Hz, 1H), 4.65-4.58 (m, 1H), 4.55 (q, J=7.0 Hz, 1H), 4.47-4.34 (m, 1.4H), 4.27 (d, J=10.0 Hz, 0.6H, major), 4.24-4.01 (m, 3H), 3.96-3.89 (m, 1H), 3.83-3.70 (m, 2H), 3.60 (dd, J=3.5, 10.3 Hz, 0.6H, major), 3.56 (dd, J=3.3, 10.2 Hz, 0.4H, minor), 2.93 (t, J=10.6 Hz, 1H), 2.58-2.46 (m, 1H), 2.36-2.28 (m, 1H), 2.15 (s, 1.2H, minor), 2.14 (s, 1.8H, major), 2.00 (d, J=13.5 Hz, 0.4H, minor), 1.96 (d, J=13.5 Hz, 0.6H, major), 1.88-1.78 (m, 2H), 1.56 (d, J=6.8 Hz, 1.2H, minor), 1.53 (d, J=6.8 Hz, 1.8H, major), 1.46 (s, 9H), 1.21-1.10 (m, 1H).

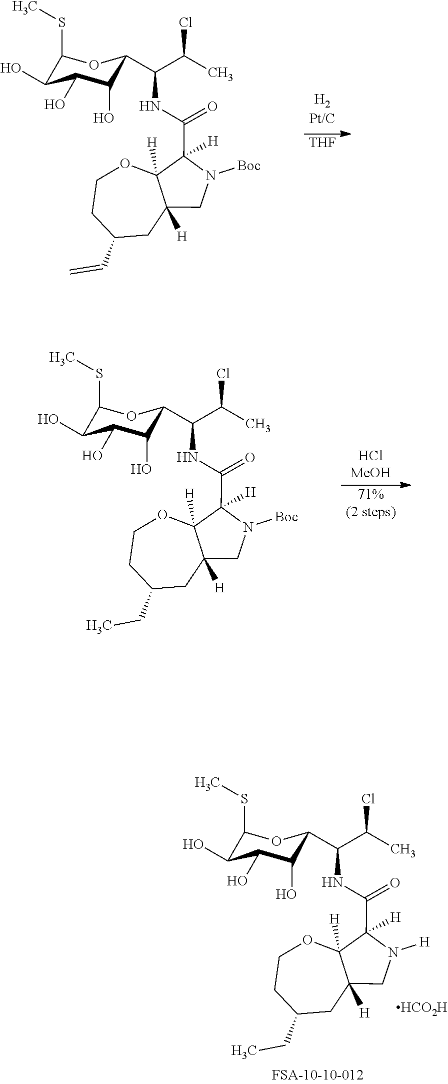

5% Platinum on carbon (43.5 mg, 4.35 mg of platinum, 11 μmol, 1.0 equiv) was added to a solution of olefin (6.3 mg, 11 μmol) in tetrahydrofuran (0.66 mL) under an argon atmosphere. The resulting suspension was sparged with hydrogen for 5 min and was then stirred under hydrogen atmosphere for 30 min. The reaction vessel was flushed with argon and the suspension was filtered through a Celite pad. The filter cake was washed with tetrahydrofuran (20 mL) and the filtrate was concentrated in vacuo to provide the crude hydrogenation product. The crude carbamate was deprotected according to the general procedure using 1 M HCl in methanol (2.1 mL, 2.1 mmol, 189 equiv). The crude product was purified by preparative high-performance liquid chromatography on C18 silica gel (5%→40% acetonitrile-water+0.1% formic acid, gradient over 25 min) to provide FSA-10-10-012 as its formate salt (4.06 mg, 71%, 2 steps). ¹H NMR (600 MHz, D₃COD) δ: 5.30 (d, J=5.6 Hz, 1H), 4.57 (qd, J=1.6, 6.8 Hz, 1H), 4.50 (d, J=10.0 Hz, 1H), 4.40 (d, J=8.8 Hz, 1H), 4.32 (t, J=9.2 Hz, 1H), 4.27 (d, J=10.0 Hz, 1H), 4.08 (dd, J=5.6, 10.2 Hz, 1H), 4.00 (dt, J=4.1, 11.7 Hz, 1H), 3.86-3.80 (m, 1H), 3.79 (td, J=3.7, 11.5 Hz, 1H), 3.61-3.52 (m, 2H), 2.86 (t, J=11.2 Hz, 1H), 2.28-2.20 (m, 1H), 2.14 (s, 3H), 2.02 (d, J=13.6 Hz, 1H), 1.85-1.68 (m, 2H), 1.53 (d, J=6.8 Hz, 3H), 1.42-1.31 (m, 2H), 0.99 (app q, J=12.1 Hz, 1H), 0.93 (t, J=7.4 Hz, 3H).

### FSA-10-10-013

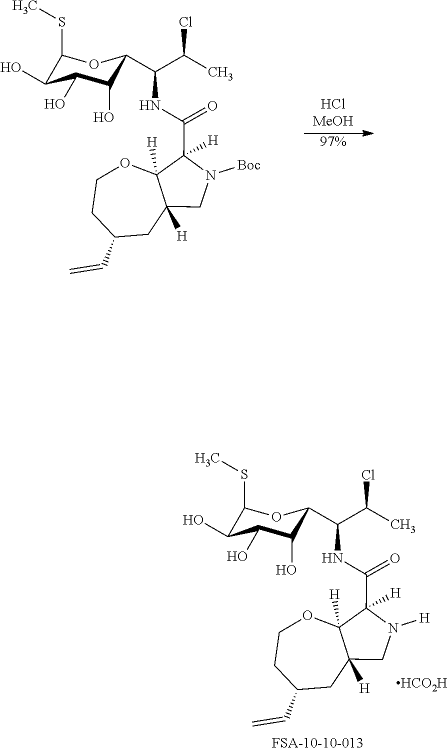

Carbamate (4.8 mg, 8.5 μmol) was deprotected according to the general procedure using 1 M HCl in methanol (0.85 mL, 0.85 mmol, 100 equiv). The crude product was purified by preparative high-performance liquid chromatography on C18 silica gel (5%→35% acetonitrile-water+0.1% formic acid, gradient over 25 min) to provide FSA-10-10-013 as its formate salt (4.20 mg, 97%). ¹H NMR (600 MHz, D₃COD) δ: 5.88 (ddd, J=7.0, 10.4, 17.3 Hz, 1H), 5.31 (d, J=5.6 Hz, 1H), 5.04 (dd, J=1.3, 17.3 Hz, 1H), 4.95 (dd, J=1.3, 10.4 Hz, 1H), 4.58 (qd, J=1.6, 6.8 Hz, 1H), 4.51 (d, J=10.0 Hz, 1H), 4.41 (d, J=8.9 Hz, 1H), 4.35 (t, J=9.2 Hz, 1H), 4.27 (d, J=10.0 Hz, 1H), 4.09 (dd, J=5.6, 10.2 Hz, 1H), 4.02 (dt, J=4.1, 11.9 Hz, 1H), 3.89-3.79 (m, 2H), 3.62-3.52 (m, 2H), 2.88 (t, J=11.7 Hz, 1H), 2.41-2.27 (m, 2H), 2.14 (s, 3H), 2.04 (d, J=13.4 Hz, 1H), 1.95-1.81 (m, 2H), 1.54 (d, J=6.8 Hz, 3H), 1.20 (app q, J=12.2 Hz, 1H).

### FSA-10-10-014

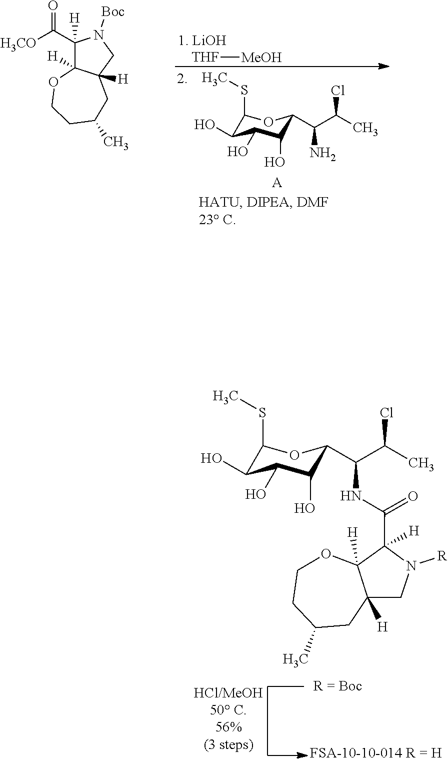

Ester (13.4 mg, 43 μmol) was hydrolyzed according to the general procedure using tetrahydrofuran (0.43 mL), methanol (0.43 mL) and 1 M aqueous lithium hydroxide solution (0.34 mL, 0.34 mmol, 8 equiv). The resulting crude acid was coupled according to the general procedure using a solution of N,N-diisopropylethylamine in N,N-dimethylformamide (0.3 M, 0.43 mL, 128 μmol, 3 equiv), HATU (19.4 mg, 51 μmol, 1.2 equiv), and 7-Cl-MTL (15.0 mg, 56 μmol, 1.3 equiv). The crude product was deprotected according to the general procedure using 1 M HCl in methanol (4.5 mL, 4.5 mmol, 100 equiv). The crude product was purified by preparative high-performance liquid chromatography on C18 silica gel (5%→35% acetonitrile-water+0.1% formic acid, gradient over 30 min) to provide FSA-10-10-014 as its formate salt (11.88 mg, 56%, 3 steps). ¹H NMR (600 MHz, D₃COD) δ: 8.39 (br s, 1H), 5.30 (d, J=5.6 Hz, 1H), 4.57 (qd, J=1.5, 6.9 Hz, 1H), 4.51 (d, J=10.0 Hz, 1H), 4.45 (d, J=8.8 Hz, 1H), 4.36 (t, J=9.2 Hz, 1H), 4.28 (d, J=10.0 Hz, 1H), 4.09 (dd, J=5.6, 10.2 Hz, 1H), 3.99 (dt, J=3.9, 12.1 Hz, 1H), 3.87-3.78 (m, 2H), 3.60-3.52 (m, 2H), 2.89 (t, J=11.8 Hz, 1H), 2.33-2.22 (m, 1H), 2.16 (s, 3H), 1.96 (d, J=13.2 Hz, 1H), 1.84-1.69 (m, 3H), 1.53 (d, J=6.9 Hz, 3H), 1.04 (d, J=5.8 Hz, 3H), 1.02-0.97 (m, 1H).

### FSA-10-10-022

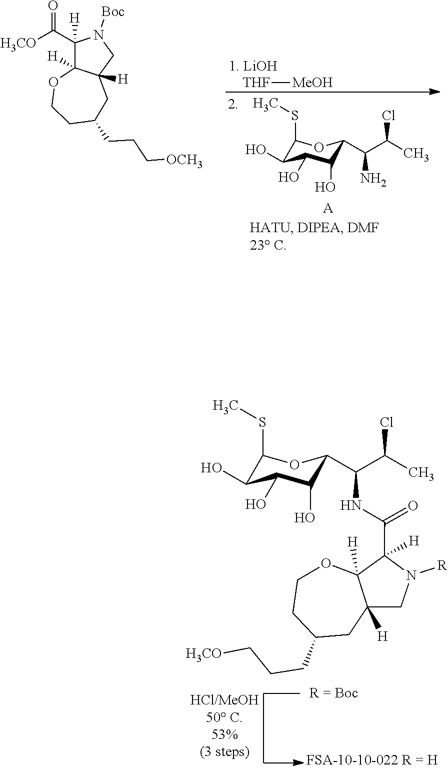

Ester (7.4 mg, 20 μmol) was hydrolyzed according to the general procedure using tetrahydrofuran (0.20 mL), methanol (0.20 mL) and 1 M aqueous lithium hydroxide solution (0.16 mL, 0.16 mmol, 8 equiv). The resulting crude acid was coupled according to the general procedure using a solution of N,N-diisopropylethylamine in N,N-dimethylformamide (0.3 M, 0.20 mL, 60 μmol, 3 equiv), HATU (9.1 mg, 24 μmol, 1.2 equiv), and 7-Cl-MTL (7.0 mg, 26 μmol, 1.3 equiv). The crude product was deprotected according to the general procedure using 1 M HCl in methanol (2.0 mL, 2.0 mmol, 100 equiv). The crude product was purified by preparative high-performance liquid chromatography on C18 silica gel (5%→35% acetonitrile-water+0.1% formic acid, gradient over 30 min) to provide FSA-10-10-022 as its formate salt (5.84 mg, 53%, 3 steps). ¹H NMR (600 MHz, D₃COD) δ: 5.30 (d, J=5.6 Hz, 1H), 4.57 (qd, J=1.6, 6.8 Hz, 1H), 4.48 (dd, J=1.6, 10.1 Hz, 1H), 4.34-4.27 (m, 2H), 4.25 (d, J=10.0 Hz, 1H), 4.08 (dd, J=5.6, 10.2 Hz, 1H), 3.99 (dt, J=4.1, 11.8 Hz, 1H), 3.84 (d, J=2.9 Hz, 1H), 3.80 (td, J=3.8, 11.5 Hz, 1H), 3.57 (dd, J=3.3, 10.2 Hz, 1H), 3.50 (dd, J=7.4, 11.0 Hz, 1H), 3.38 (t, J=6.5 Hz, 2H), 3.33 (s, 3H), 2.81 (t, J=11.5 Hz, 1H), 2.27-2.18 (m, 1H), 2.14 (s, 3H), 2.01 (d, J=13.6 Hz, 1H), 1.84-1.69 (m, 2H), 1.66-1.56 (m, 3H), 1.52 (d, J=6.8 Hz, 3H), 1.37 (tdd, J=6.8, 11.3, 13.9 Hz, 1H), 0.99 (app q, J=13.4 Hz, 1H).

### FSA-10-10-048

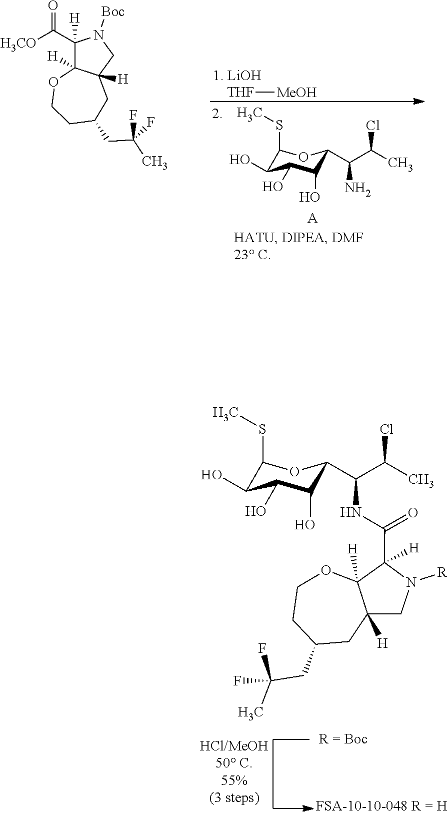

Ester (8.2 mg, 22 μmol) was hydrolyzed according to the general procedure using tetrahydrofuran (0.22 mL), methanol (0.22 mL) and 1 M aqueous lithium hydroxide solution (0.17 mL, 0.17 mmol, 8 equiv). The resulting crude acid was coupled according to the general procedure using a solution of N,N-diisopropylethylamine in N,N-dimethylformamide (0.3 M, 0.22 mL, 65 μmol, 3 equiv), HATU (9.9 mg, 26 μmol, 1.2 equiv), and 7-Cl-MTL (7.7 mg, 28 μmol, 1.3 equiv). The crude product was deprotected according to the general procedure using 1 M HCl in methanol (2.2 mL, 2.2 mmol, 100 equiv). The crude product was purified by preparative high-performance liquid chromatography on C18 silica gel (5%→40% acetonitrile-water+0.1% formic acid, gradient over 25 min) to provide FSA-10-10-048 as its formate salt (6.74 mg, 55%, 3 steps). ¹H NMR (600 MHz, D₃COD) δ: 5.28 (d, J=5.6 Hz, 1H), 4.55 (qd, J=1.6, 6.8 Hz, 1H), 4.47 (dd, J=1.6, 10.1 Hz, 1H), 4.37-4.28 (m, 2H), 4.24 (d, J=10.0 Hz, 1H), 4.06 (dd, J=5.6, 10.2 Hz, 1H), 3.96 (dt, J=4.1, 11.9 Hz, 1H), 3.83-3.77 (m, 2H), 3.55 (dd, J=3.3, 10.2 Hz, 1H), 3.51 (dd, J=7.4, 11.1 Hz, 1H), 2.32-2.22 (m, 1H), 2.12 (s, 3H), 2.08 (dt, J=3.6, 13.5 Hz, 1H), 2.03-1.94 (m, 1H), 1.93-1.79 (m, 4H), 1.57 (t, J=18.6 Hz, 3H), 1.51 (d, J=6.8 Hz, 3H), 1.09 (dt, J=11.5, 13.4 Hz, 1H).

The following compounds were prepared in an analogous manner:

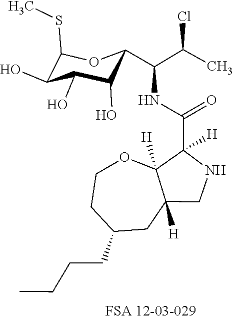

¹H NMR (600 MHz, D₃COD) δ 5.30 (d, J=5.7 Hz, 1H), 4.61-4.54 (m, 1H), 4.48 (d, J=10.0 Hz, 1H), 4.37-4.19 (m, 3H), 4.08 (dd, J=10.1, 5.6 Hz, 1H), 3.99 (dt, J=11.8, 4.0 Hz, 1H), 3.86-3.75 (m, 2H), 3.60-3.47 (m, 2H), 2.81 (t, J=11.3 Hz, 1H), 2.23 (t, J=16.1 Hz, 1H), 2.14 (s, 3H), 2.04-1.95 (m, 1H), 1.81-1.69 (m, 2H), 1.61 (s, 1H), 1.52 (d, J=6.8 Hz, 3H), 1.32 (s, 7H), 0.98 (q, J=12.1 Hz, 1H), 0.94-0.87 (m, 3H). LCMS (Low-res MS) (ES Pos) m/z: [M+H]+ Calcd. for C22H40ClN2O6S: 495.2290; Found 495.2.

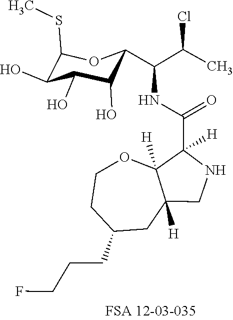

¹H NMR (500 MHz, D₃COD) δ 5.30 (d, J=5.6 Hz, 1H), 4.60-4.53 (m, 1H), 4.52-4.39 (m, 3H), 4.39-4.30 (m, 2H), 4.27 (d, J=10.0 Hz, 1H), 4.08 (dd, J=10.2, 5.6 Hz, 1H), 4.00 (dt, J=11.9, 4.1 Hz, 1H), 3.87-3.77 (m, 2H), 3.56 (dd, J=11.1, 7.6 Hz, 2H), 2.87 (t, J=11.7 Hz, 1H), 2.32-2.20 (m, 1H), 2.15 (s, 3H), 2.03 (d, J=13.3 Hz, 1H), 1.84-1.63 (m, 5H), 1.53 (d, J=6.8 Hz, 3H), 1.50-1.37 (m, J=6.7 Hz, 2H), 1.03 (q, J=12.0 Hz, 1H). ¹⁹F NMR (471 MHz, Methanol-d4) δ-220.01. LCMS (Low-res MS) (ES Pos) m/z: [M+H]+ Calcd. for C21H37ClFN2O6S: 499.2039; Found 499.3.

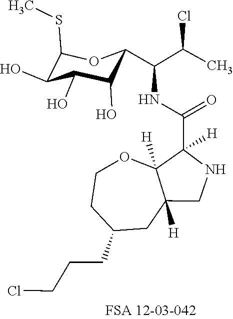

¹H NMR (500 MHz, D₃COD) δ 5.30 (d, J=5.7 Hz, 1H), 4.57 (q, J=6.7 Hz, 1H), 4.49 (d, J=10.0 Hz, 1H), 4.36-4.22 (m, 3H), 4.08 (dd, J=10.2, 5.6 Hz, 1H), 4.03-3.96 (m, 1H), 3.85-3.76 (m, 2H), 3.59-3.50 (m, 4H), 2.82 (t, J=11.5 Hz, 1H), 2.26 (d, J=10.4 Hz, 1H), 2.14 (s, 3H), 2.01 (d, J=16.3 Hz, 1H), 1.79 (h, J=9.0, 8.5 Hz, 4H), 1.66 (s, 1H), 1.50 (dd, J=21.8, 7.2 Hz, 5H), 1.02 (q, J=12.1 Hz, 1H). LCMS (Low-res MS) (ES Pos) m/z: [M+H]+ Calcd. for C21H37Cl2N2O6S: 515.1744; Found 515.2.

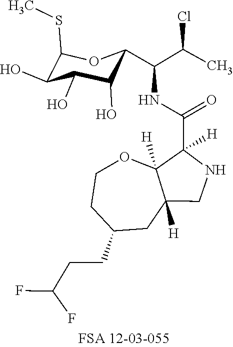

¹H NMR (500 MHz, D₃COD) δ 5.86 (tt, J=57.0, 4.4 Hz, 1H), 5.30 (d, J=5.6 Hz, 1H), 4.62-4.54 (m, 1H), 4.50 (dd, J=10.1, 1.6 Hz, 1H), 4.41 (d, J=8.9 Hz, 1H), 4.33 (t, J=9.2 Hz, 1H), 4.27 (d, J=10.0 Hz, 1H), 4.08 (dd, J=10.2, 5.6 Hz, 1H), 4.00 (dt, J=11.8, 4.1 Hz, 1H), 3.86-3.79 (m, 2H), 3.61-3.53 (m, 2H), 2.87 (t, J=11.7 Hz, 1H), 2.26 (p, J=11.5, 10.5 Hz, 1H), 2.14 (s, 3H), 2.05-1.99 (m, 1H), 1.93-1.75 (m, 4H), 1.72-1.62 (m, 1H), 1.52 (d, J=6.8 Hz, 3H), 1.47 (ddd, J=15.5, 9.2, 5.5 Hz, 2H), 1.03 (q, J=12.1 Hz, 1H). ¹⁹F NMR (471 MHz, Methanol-d4) δ-117.62. LCMS (Low-res MS) (ES Pos) m/z: [M+H]+ Calcd. for C₂₁H₃₆ClF₂N₂O₆S: 517.1945; Found 517.2.

Additional compounds were prepared as detailed below.

### Synthesis of Lincosamide Northern Halves

### 2R,3R,4S,5R,6R)-2-((1S,2S)-1-amino-2-((4-((dimethylamino)methyl)phenyl)thio)propyl)-6-(methylthio)tetrahydro-2H-pyran-3,4,5-triol

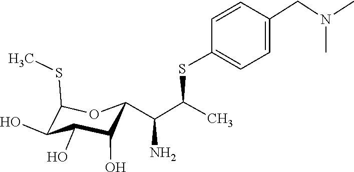

Prepared according to procedure described in The Journal of Antibiotics (2016) 69, 428-439, (2017) 70, 52-64, (2018) 71, 298-317. ¹H NMR (500 MHz, cd₃od) δ 7.37 (d, J=8.2 Hz, 2H), 7.25 (d, J=8.2 Hz, 2H), 5.22 (d, J=5.6 Hz, 1H), 4.24 (d, J=8.8 Hz, 1H), 4.07 (dd, J=9.9, 5.2 Hz, 2H), 3.69 (qd, J=6.9, 2.7 Hz, 1H), 3.59 (dd, J=10.2, 3.3 Hz, 1H), 3.46 (s, 2H), 3.23 (dd, J=8.8, 2.7 Hz, 1H), 2.24 (s, 6H), 1.88 (s, 3H), 1.44 (d, J=7.0 Hz, 3H). MS (QMS) m/z: [M+H]⁺ for C₁₈H₃₁N₂O₄S₂, found 403.2.

### (2R,3R,4S,5R,6R)-2-((1S,2S)-1-Amino-2-((4-(2-(dimethylamino)ethyl)phenyl)thio)propyl)-6-(methylthio)tetrahydro-2H-pyran-3,4,5-triol

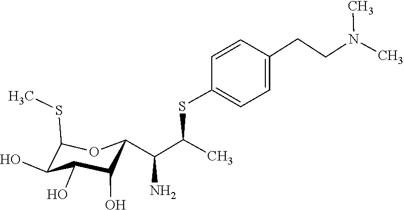

Prepared according to procedure described in The Journal of Antibiotics (2018) 71, 298-317. ¹H NMR (500 MHz, cd₃od) δ 7.37 (d, J=7.9 Hz, 2H), 7.21 (d, J=8.0 Hz, 2H), 5.25 (d, J=5.6 Hz, 1H), 4.26 (d, J=8.5 Hz, 1H), 4.11 (dd, J=9.6, 5.5 Hz, 2H), 3.68-3.58 (m, 2H), 3.31-3.20 (m, 2H), 2.84 (dd, J=10.3, 5.8 Hz, 2H), 2.73 (dd, J=10.2, 5.9 Hz, 6H), 1.94 (s, 3H), 1.43 (t, J=5.5 Hz, 3H). MS (QMS) m/z: [M+H]⁺ for C₁₉H₃₄N₂O₄S₂, found 417.2.

### Tert-butyl (5-(((1S,2S)-1-amino-1-((2R,3R,4S,5R,6R)-3,4,5-trihydroxy-6-(methylthio)tetrahydro-2H-pyran-2-yl)propan-2-yl)thio)-1,3,4-thiadiazol-2-yl)carbamate

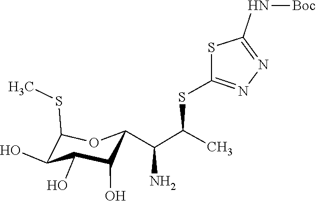

Prepared according to procedure described in The Journal of Antibiotics (2016) 69, 428-439, (2018) 71, 298-317. ¹H NMR (500 MHz, cd₃od) δ 5.26 (d, J=5.6 Hz, 1H), 4.22 (d, J=8.8 Hz, 1H), 4.15 (qd, J=6.8, 2.7 Hz, 1H), 4.10 (dd, J=8.9, 4.3 Hz, 2H), 3.61 (dd, J=10.2, 3.3 Hz, 1H), 3.39 (dd, J=8.8, 2.7 Hz, 1H), 3.28 (dd, J=14.6, 7.3 Hz, 1H), 1.99 (s, 3H), 1.59 (d, J=7.0 Hz, 3H), 1.55 (s, 9H). MS (QMS) m/z: [M+H]⁺ for C₁₆H₂₉N₄O₆S₃, found 469.1.

### (2R,3R,4S,5R,6R)-2-((1S,2S)-1-amino-2-((4-(pyridin-3-yl)phenyl)thio)propyl)-6-(methylthio)tetrahydro-2H-pyran-3,4,5-triol

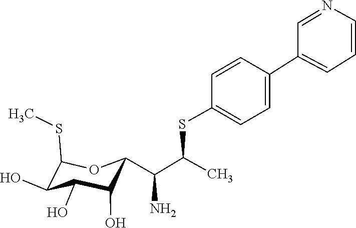

Prepared according to procedure described in The Journal of Antibiotics (2017) 70, 52-64. ¹H NMR (600 MHz, CD₃OD) δ 9.12 (s, 1H), 9.07 (s, 2H), 7.68 (d, J=8.4 Hz, 2H), 7.55 (d, J=8.4 Hz, 1H), 5.24 (d, J=5.5 Hz, 1H), 4.11 (dd, J=3.4, 1.2 Hz, 1H), 4.09 (dd, J=10.2, 5.5 Hz, 1H), 3.83 (qd, J=7.0, 2.8 Hz, 1H), 3.61 (dd, J=10.2, 3.4 Hz, 1H), 3.30 (dd, J=8.6,2.9 Hz, 1H), 1.89 (s, 3H), 1.51 (d, J=6.9 Hz, 3H). HRMS (ESI+, m/z): [M+H]+ calcd for C19H25N3O4S2, 424.1359; found 424.1369.

### 2,2,2-Trifluoro-N-((1S,2S)-2-mercapto-1-((2R,3R,4S,5R,6R)-3,4,5-trihydroxy-6-(methylthio)tetrahydro-2H-pyran-2-yl)propyl)acetamide

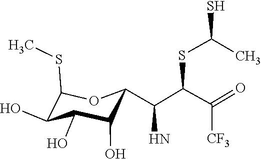

Prepared according to procedure described in The Journal of Antibiotics (2018) 71, 298-317. ¹H NMR (500 MHz, cd₃od) δ 5.27 (d, J=5.7 Hz, 1H), 4.55 (dd, J=10.0, 2.2 Hz, 1H), 4.38 (d, J=10.0 Hz, 1H), 4.08 (dd, J=10.2, 5.7 Hz, 1H), 3.84-3.78 (m, 1H), 3.54 (dd, J=10.2, 3.2 Hz, 1H), 3.45 (qd, J=7.0, 2.1 Hz, 1H), 2.16 (s, 3H), 1.29 (d, J=7.1 Hz, 3H). ¹⁹F NMR (471 MHz, cd₃od) δ −76.64. MS (QMS) m/z: [M+Na]⁺ for C₁₁H₁₈F₃NO₅S₂Na, found 388.1.

### Tert-butyl 4-(5-(((1S,2S)-1-(2,2,2-trifluoroacetamido)-1-((2R,3R,4S,5R,6R)-3,4,5-trihydroxy-6-(methylthio)tetrahydro-2H-pyran-2-yl)propan-2-yl)thio)-1,3,4-thiadiazol-2-yl)piperazine-1-carboxylate

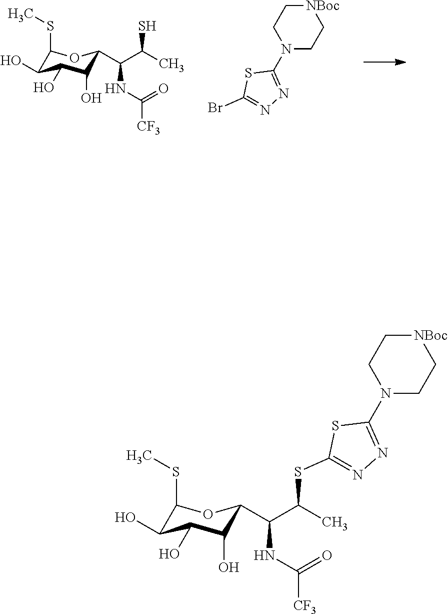

Tert-butyl 4-(5-bromo-1,3,4-thiadiazol-2-yl)piperazine-1-carboxylate (194 mg, 0.55 mmol, 1.5 eq.), 2,2,2-trifluoro-N-((1S,2S)-2-mercapto-1-((2R,3R,4S,5R,6R)-3,4,5-trihydroxy-6-(methylthio)tetrahydro-2H-pyran-2-yl)propyl)acetamide (135 mg, 0.37 mmol, 1.0 eq) and potassium carbonate (255 mg, 1.85 mmol, 5.0 eq.) were mixed in N,N-dimethylformamide (1.2 mL) and the reaction mixture was heated at 100° C. overnight. The reaction was diluted with ethyl acetate and brine, the organic phase was washed with brine again, dried over sodium sulfate and concentrated in vacuo. The residue was purified by flash column chromatography on silica gel with a gradient 2-5-10-20% methanol in dichloromethane to give desired material (55 mg, 23%). ¹H NMR (500 MHz, cd₃od) δ 5.30 (d, J=5.6 Hz, 1H), 4.69 (dd, J=9.8, 2.8 Hz, 1H), 4.53 (d, J=9.8 Hz, 1H), 4.15-4.04 (m, 2H), 3.86 (d, J=2.8 Hz, 1H), 3.58 (dd, J=10.0, 3.1 Hz, 5H), 3.54-3.47 (m, 4H), 2.12 (s, 3H), 1.49 (s, 9H), 1.43 (d, J=6.9 Hz, 3H). MS (QMS) m/z: [M+H]⁺ for C₂₂H₃₅F₃N₅O₇S₃, found 634.2.

### Tert-butyl 4-(5-(((1S,2S)-1-amino-1-((2R,3R,4S,5R,6R)-3,4,5-trihydroxy-6-(methylthio)tetrahydro-2H-pyran-2-yl)propan-2-yl)thio)-1,3,4-thiadiazol-2-yl)piperazine-1-carboxylate

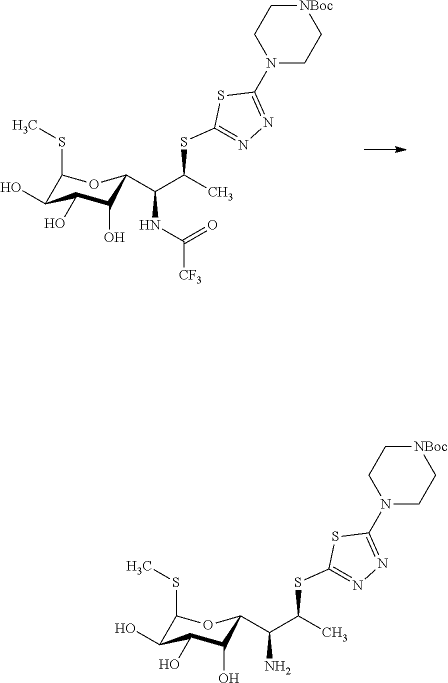

Tert-butyl 4-(5-(((1S,2S)-1-(2,2,2-trifluoroacetamido)-1-((2R,3R,4S,5R,6R)-3,4,5-trihydroxy-6-(methylthio)tetrahydro-2H-pyran-2-yl)propan-2-yl)thio)-1,3,4-thiadiazol-2-yl)piperazine-1-carboxylate (55 mg, 0.087 mmol, 1.0 eq.) and benzyltriethylammonium bromide (2.4 mg, 0.0087 mmol, 0.1 eq.) were dissolved in dichloromethane (0.9 mL), the solution was then cooled to 0° C. and 20% aqueous potassium hydroxide (90 μL) was added, the reaction was stirred at room temperature for 5.5 h, then LCMS and TLC showed full consumption of trifluoroacetamide. The pH of the reaction was adjusted to around 7 using phenolphtaleine as indicator. The solvent was then completely removed under reduced pressure to obtain a white residue that was treated with methanol causing precipitation of the inorganic salts that were filtered off on a pad of celite. The methanolic solution was evaporated and the residue obtained was purified by flash column chromatography on silica gel with a gradient 20-50-100% of eluent B (2% ammonium hydroxide—20% methanol—78% dichloromethane) in eluent A (dichloromethane) to obtain the desired aminotriol (15 mg, 32%). ¹H NMR (500 MHz, cd₃od) δ 5.24 (d, J=5.6 Hz, 1H), 4.17 (d, J=9.2 Hz, 1H), 4.11-4.04 (m, 3H), 3.60 (dd, J=10.2, 3.3 Hz, 5H), 3.50 (dd, J=6.2, 4.1 Hz, 4H), 2.03 (s, 3H), 1.55 (d, J=7.0 Hz, 3H), 1.49 (s, 9H). MS (QMS) m/z: [M+H]⁺ for C₂₀H₃₆N₅O₆S₃, found 538.2.

### 2,2,2-Trifluoro-N-((1S,2S)-2-((4-(hydroxymethyl)phenyl)thio)-1-((2R,3R,4S,5R,6R)-3,4,5-trihydroxy-6-(methylthio)tetrahydro-2H-pyran-2-yl)propyl)acetamide

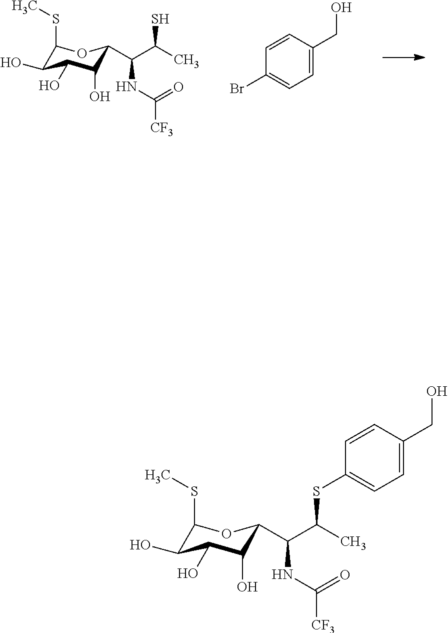

Tris(dibenzylideneacetone)dipalladium (0) (8.8 mg, 9.6 μmol, 0.05 eq.) and 9,9-Dimethyl-4,5-bis(diphenylphosphino)xanthene (11.1 mg, 19 μmol, 0.1 eq.) were dissolved in dioxane (0.5 mL), degassed prior to use, under an inert atmosphere. To this solution, 4-bromobenzyl alcohol (53.7 mg, 0.29 mmol, 1.5 eq.) in dioxane (0.5 mL) and 2,2,2-trifluoro-N-((1S,2S)-2-mercapto-1-((2R,3R,4S,5R,6R)-3,4,5-trihydroxy-6-(methylthio)tetrahydro-2H-pyran-2-yl)propyl)acetamide (70 mg, 0.19 mmol, 1.0 eq.) in dioxane (0.5 mL) were added followed by N,N-diisopropylethylamine (67 μL, 0.38 mmol, 2.0 eq.), the reaction was then heated to 115° C. for 3 hours. The cooled reaction was diluted with methanol and the mixture was filtered over a pad of celite, the filtrate was evaporated in vacuo and the residue thus obtained was purified by flash column chromatography on silica gel with a gradient 20-50-100% of eluent B (2% ammonium hydroxide—20% methanol—78% dichloromethane) in eluent A (dichloromethane) to obtain the desired aryl sulfide (70 mg, 77%). ¹H NMR (500 MHz, cd₃od) δ 7.41 (d, J=8.1 Hz, 2H), 7.31 (d, J=8.1 Hz, 2H), 5.31 (d, J=5.6 Hz, 1H), 4.69-4.59 (m, 2H), 4.59 (s, 2H), 4.12 (dd, J=10.2, 5.6 Hz, 1H), 3.91 (d, J=3.0 Hz, 1H), 3.81 (qd, J=6.8, 3.0 Hz, 1H), 3.61 (dd, J=10.2, 3.2 Hz, 1H), 2.02 (s, 3H), 1.29 (d, J=6.9 Hz, 3H). MS (QMS) m/z: [M+Na]⁺ for C₁₈H₂₄F₃NO₆S₂Na, found 494.1.

### (2R,3R,4S,5R,6R)-2-((1S,2S)-1-Amino-2-((4-(hydroxymethyl)phenyl)thio)propyl)-6-(methylthio)tetrahydro-2H-pyran-3,4,5-triol

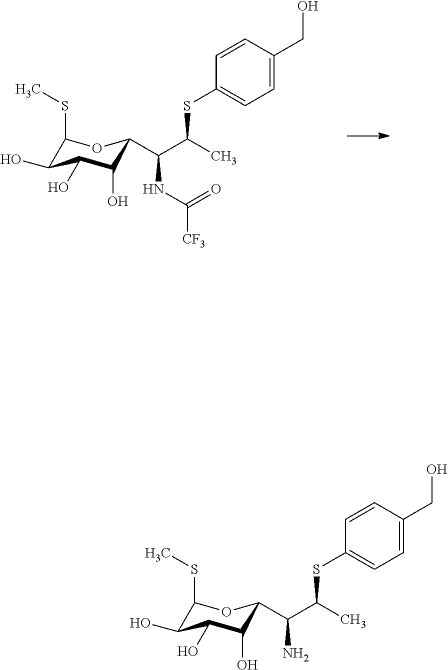

2,2,2-Trifluoro-N-((1S,2S)-2-((4-(hydroxymethyl)phenyl)thio)-1-((2R,3R,4S,5R,6R)-3,4,5-trihydroxy-6-(methylthio)tetrahydro-2H-pyran-2-yl)propyl)acetamide (440 mg, 0.93 mmol, 1.0 eq.) and benzyltriethylammonium bromide (25.4 mg, 0.093 mmol, 0.1 eq.) were suspended in dichloromethane (10 mL), the mixture was then cooled to 0° C. and 20% aqueous potassium hydroxide (1.0 mL) was added drop wise, the reaction was stirred at room temperature for 7 h, then LCMS showed full consumption of trifluoroacetamide. The pH of the reaction was adjusted to around 7 using phenolphtaleine as indicator. The solvent was then completely removed under reduced pressure to give a white residue, insoluble in the most common organic solvents, that was suspended in toluene and evaporated twice in vacuo prior being used in the amide coupling step as a crude residue.

### (2R,3S,4S,5R,6R)-2-((R)-1-(((R)-Tert-butylsulfinyl)amino)-2-(trifluoromethyl)allyl)-6-(methylthio)tetrahydro-2H-pyran-3,4,5-triyl tribenzoate

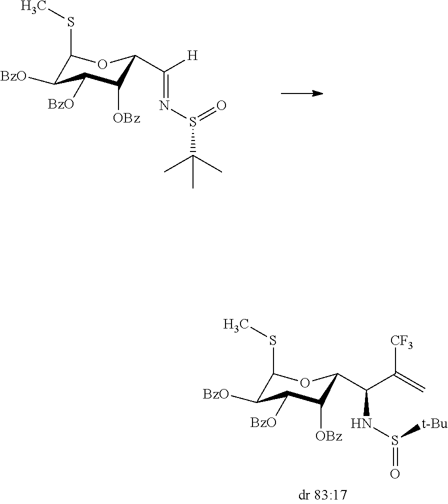

Organolithium reagent preparation according to Chem. Asian J. 2010, 5, 1875: A solution of 2-bromo-3,3,3-trifluoroprop-1-ene (300 mg, 1.72 mmol 1.0 eq) in diethyl ether (6.0 mL) was cooled below −105° C. using a diethyl ether/liquid nitrogen bath under an inert atmosphere. To this solution, a prechilled solution (−78° C.) of t-BuLi (1.7 M in pentane) (1.2 mL, 2.06 mmol, 1.2 eq) in diethyl ether (2.0 mL) was added via cannula (final con M=0.22). The reaction was stirred for 10 min keeping the temperature rigorously below −105° C. to ensure complete lithium-halogen exchange and avoid decomposition of the generated species.

Addition step: To the cooled solution of the lithium species (1.72 mmol) at −105° C., a solution of (2R,3S,4S,5R,6R)-2-((E)-(((R)-tert-butylsulfinyl)imino)methyl)-6-(methylthio)tetrahydro-2H-pyran-3,4,5-triyltribenzoate (200 mg, 0.32 mmol, 0.19 eq) in diethyl ether (2.0 mL) was added dropwise. The reaction was stirred for 25 minutes and check by TLC (40% ethyl acetate-hexane) showed complete conversion of the starting material to a more polar spot. The reaction was then quenched with a saturated solution of ammonium chloride and diluted with ethyl acetate, the watery phase was extracted again with ethyl acetate, the combined organic phases were washed with brine, dried over sodium sulfate, filtered and the volatiles were removed in vacuum. The residue obtained was purified by flash column chromatography on silica gel with a gradient 10-30-50% ethyl acetate in hexane to give the desired compound (191 mg, 83%) as an amorphous, foaming solid, mixture of diastereomers at the amine carbon (dr 83:17). ¹H NMR (500 MHz, CDCl₃) δ 8.12-8.07 (m, 2H), 8.04-7.93 (m, 2H), 7.73 (dt, J=6.9, 1.4 Hz, 2H), 7.68-7.60 (m, 1H), 7.52 (td, J=7.7, 1.9 Hz, 3H), 7.50-7.34 (m, 3H), 7.27-7.17 (m, 2H), 6.15 (d, J=1.6 Hz, 1H), 6.08-6.04 (m, 1H), 5.93 (d, J=5.8 Hz, 1H), 5.88 (d, J=6.3 Hz, 1H), 5.86 (t, J=5.3 Hz, 1H), 5.82-5.73 (m, 1H), 4.87 (d, J=6.3 Hz, 1H), 4.43 (dd, J=6.4, 2.6 Hz, 1H), 3.98 (d, J=2.7 Hz, 1H), 2.12 (s, 3H), 1.04 (s, 9H). ¹⁹F NMR (471 MHz, c₆d₆) δ-66.11. HRMS (ESI+, m/z): [M+H]⁺ calcd for C₃₅H₃₇F₃NO₈S₂ 720,1907; found 720.1897.

### General Experimental Procedures for Final Steps of Compound Synthesis

### Procedure A: Basic Cleavage of Benzoyl Esters and Acidic Cleavage of Sulfinyl Group of Synthetic Northern Halves

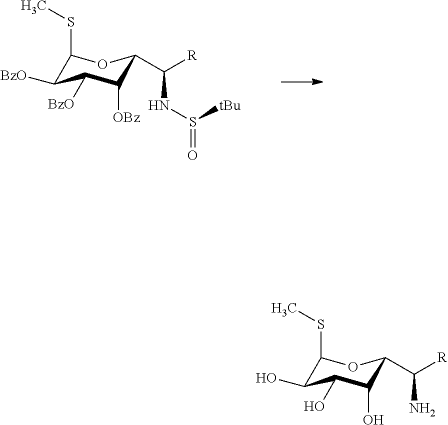

Benzoylated sulfinyl amine (0.1 mmol) was dissolved in anhydrous methanol (1.0 mL, M=0.1) and the solution was cooled to 0° C. followed by addition of a sodium methoxyde 0.5 M solution in methanol (0.1 mL), the reaction was stirred until complete consumption of starting material and formation of a single more polar spot as monitored by TLC (dichloromethane-methanol 98:2 and ethyl acetate-hexane 50:50), generally the reaction was completed within one hour.

The solution was cooled again to 0° C. and treated with freshly prepared 3 N methanolic HCl (1 mL, final reaction concentration ca M=0.05), stirring was prolonged for a time between one and sixteen hours until complete removal of the sulfinyl group as monitored by both TLC (50% “dichloromethane:methanol:ammonium hydroxide 78:20:2”-dichloromethane) and LCMS analysis. Once the reaction was completed, it was diluted with toluene (0.5 mL) and the solvent was removed under reduced pressure to obtain the crude aminotriol that could either be directly employed in the coupling step or purified by flash column chromatography with a gradient 20-50-100% eluent B (dichloromethane:methanol:aq. 30% ammonium hydroxide 78:20:2) in eluent A (dichloromethane).

### Procedure B: Amide Coupling of the Northern and Southern Halves

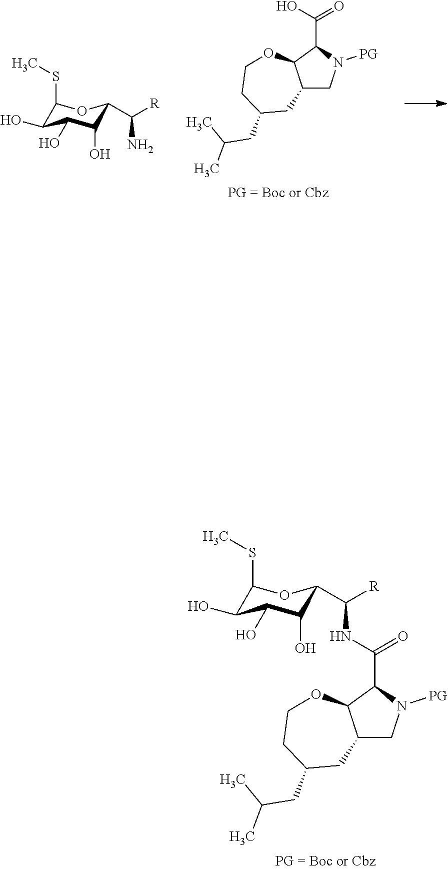

The carboxylic acid (1.2 eq) and HATU (1.2 eq) were dissolved in N,N-dimethylformamide (½ total solvent volume, final concentration M=0.1) cooled at 0° C. and then N,N-diisopropylethylamine (5.0 eq.) was added turning the solution an intense yellow that was stirred for 15 min at room temperature, at this point a solution of the amine coupling partner (1.0 eq.) in N,N-dimethylformamide (½ solvent volume) was added to the preformed activated ester. The reaction was stirred at room temperature for a time ranging between one and seven hours being monitored by means of LCMS. Upon complete consumption of the primary amine or once reached the seven hours, the reaction was diluted with ethyl acetate and a saturated solution of sodium bicarbonate, the organic phase was then washed with brine, dried over sodium sulfate, filtered and the organic solvent was evaporated in vacuo. The residue thus obtained could be either directly used in the final N-Boc cleavage step or purified by flash column chromatography on silica gel with a gradient 20-50-100% of eluent B (2% ammonium hydroxide—20% methanol—78% dichloromethane).

### Procedure C: Acidic N-Boc Cleavage

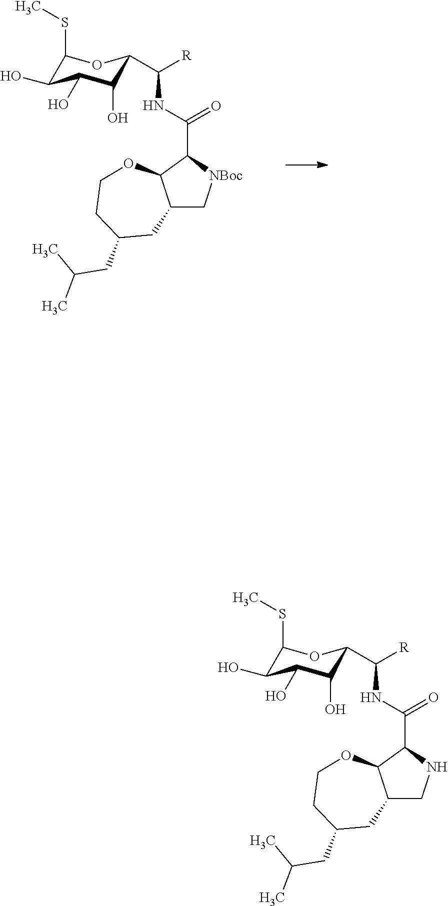

N-Boc protected lincosamide analog (1 eq.) was dissolved in methanol (½ volume, final reaction concentration M=0.02) and the reaction temperature was lowered to 0° C., at this point a freshly prepared 2N solution of HCl in methanol (½ volume) was added drop wise, the cooling bath was removed and the reaction was stirred at room temperature until complete consumption of starting material as monitored by LCMS and TLC, reaction time varied from one to five hours. Toluene (0.2 mL) was added and the volatiles were removed with a stream of nitrogen, the residue obtained was purified by preparative HPLC-MS with a gradient 5-50% in 25 minutes of acetonitrile +0.1% formic acid in water +0.1% formic acid, the fractions containing the desired compound were collected and the solvent was removed in vacuo to give a colorless material.

### Procedure D: N-Cbz Cleavage

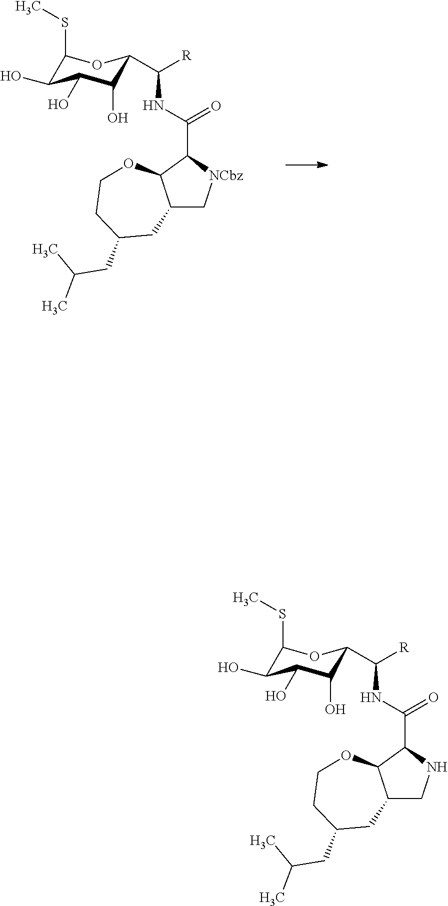

N-Cbz protected lincosamide analog was dissolved in methanol (M=0.1), the solution was purged with nitrogen for 5 minutes, after this, 10% Pd on charcoal (2.5×mass of N-Cbz lincosamide) was added and the reaction was bubbled with H₂ (balloon) for 5 minutes and then let stirring under an H₂ atmosphere for 2.5 hours, the mixture was filtered over a pad of celite and the solvent was evaporated in vacuo. The residue thus obtained was purified by preparative HPLC-MS with a gradient 5-50% of acetonitrile +0.1% formic acid in water +0.1% formic acid, the fractions containing the desired compound were collected and the solvent was removed in vacuo to give a colorless material.

### Benzyl (4S,5aS,8S,8aR)-8-(((1R,2R)-2-hydroxy-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

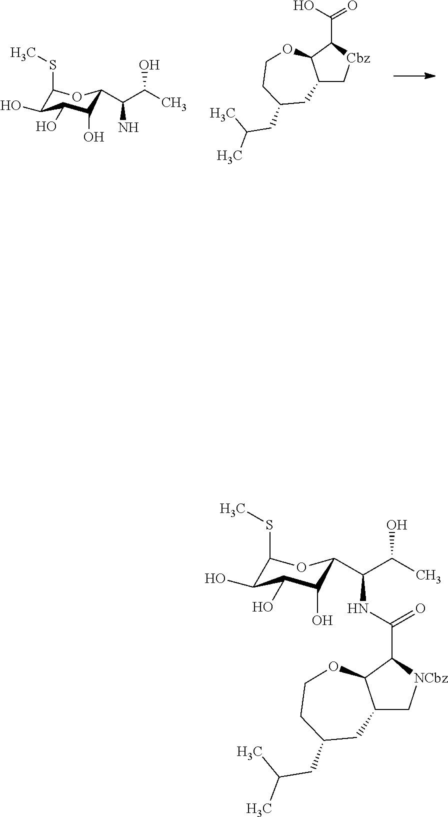

Procedure B (28 mg, 51%) using 7-methylthiolincosamine (Magerlein, B. J.; Birkenmeyer, R. D.; Kagan, F. J. Med. Chem. 1967, 10, 355-359).

### (4S,5aS,8S,8aR)-N-((1R,2R)-2-Hydroxy-1-((2R,3R,4S,5R,6R)-3,4,5-trihydroxy-6-(methylthio)tetrahydro-2H-pyran-2-yl)propyl)-4-isobutyloctahydro-2H-oxepino[2,3-c]pyrrole-8-carboxamide (FSA-09-04-072)

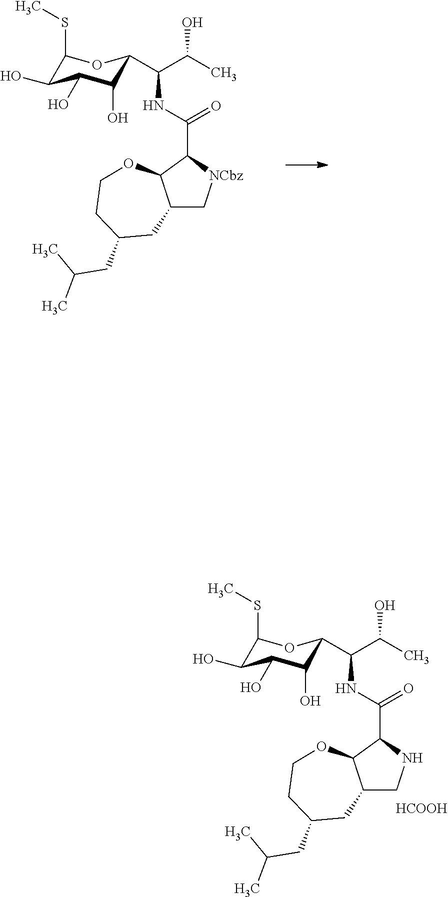

Procedure D (3 mg, 52%). Purification by preparative HPLC-MS with a gradient 5-70% in 25 minutes of acetonitrile +0.1% formic acid in water +0.1% formic acid. ¹H NMR (500 MHz, cd₃od) δ 5.27 (d, J=5.6 Hz, 1H), 4.38-4.25 (m, 4H), 4.10 (dd, J=10.2, 5.6 Hz, 1H), 4.04-3.94 (m, 3H), 3.83 (td, J=11.6, 11.2, 3.4 Hz, 1H), 3.60-3.51 (m, 2H), 2.84 (t, J=11.6 Hz, 1H), 2.28 (m, 1H), 2.11 (s, 3H), 2.00 (d, J=13.4 Hz, 1H), 1.80 (d, J=13.7 Hz, 1H), 1.69 (ddt, J=23.4, 13.4, 6.7 Hz, 3H), 1.24 (d, J=6.4 Hz, 3H), 1.19 (td, J=6.9, 5.2 Hz, 2H), 1.02-0.92 (m, 1H), 0.90 (d, J=6.5 Hz, 6H). HRMS (ESI+, m/z): [M+H]⁺ calcd for C22H41N₂O₇S 477.2629; found 477.2628.

### Benzyl (4S,5aS,8S,8aR)-4-isobutyl-8-(((R)-2-(trifluoromethyl)-1-((2R,3R,4S,5R,6R)-3,4,5-trihydroxy-6-(methylthio)tetrahydro-2H-pyran-2-yl)allyl)carbamoyl)octahydro-7H-oxepino[2,3-c]pyrrole-7-carboxylate

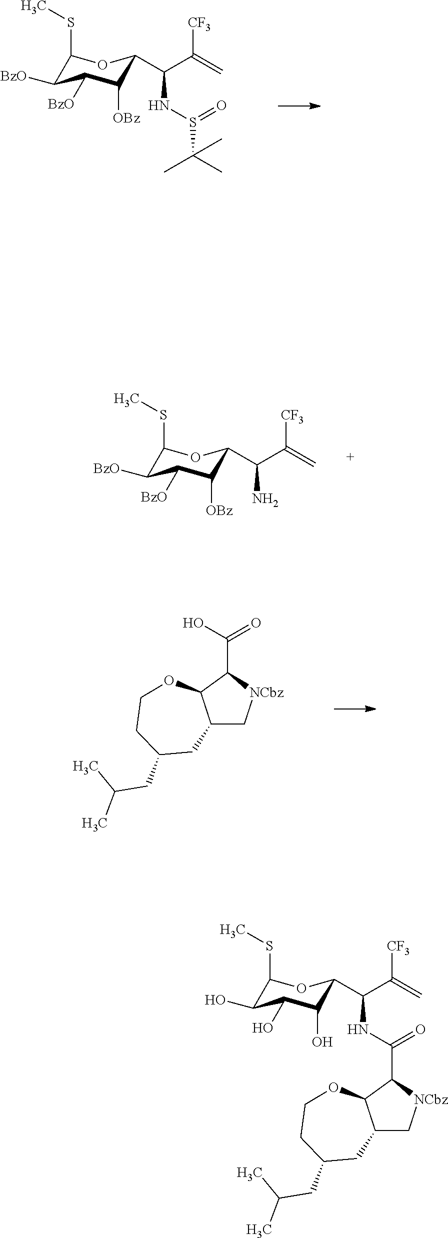

Procedure A followed by Procedure B afforded the desired Cbz-protected lincosamide analog (13 mg, 26%), rotameric mixture.¹H NMR (500 MHz, cd₃od) δ 7.38-7.27 (m, 5H), 5.98 (s, 1H), 5.84 (d, J=17.1 Hz, 1H), 5.22 (dd, J=9.1, 3.2 Hz, 1H), 5.14 (d, J=5.6 Hz, 0.5H), 5.11 (d, J=2.1 Hz, 1H), 5.00 (d, J=12.6 Hz, 0.5H), 4.90 (d, J=9.1 Hz, 0.5H), 4.80 (d, J=8.5 Hz, 0.5H), 4.43 (d, J=8.1 Hz, 0.5H), 4.34 (dd, J=17.4, 8.6 Hz, 1H), 4.17 (q, J=10.0 Hz, 1.5H), 4.08 (ddd, J=15.5, 10.0, 5.0 Hz, 1.6H), 3.86-3.76 (m, 2.5H), 3.73 (dd, J=12.7, 5.0 Hz, 1H), 3.57 (dd, J=10.1, 3.2 Hz, 0.5H), 3.34 (dd, J=10.1, 3.2 Hz, 0.5H), 2.96 (q, J=10.7 Hz, 1H), 2.54 (s, 1H), 2.01 (s, 1.5H), 1.98 (s, 1.5H), 1.95-1.87 (m, 1H), 1.74 (d, J=15.3 Hz, 1H), 1.69-1.51 (m, 3H), 1.19-1.08 (m, 2H), 0.94 (dd, J=23.9, 11.5 Hz, 1H), 0.87 (t, J=5.7 Hz, 6H). ¹⁹F NMR (471 MHz, cd₃od) δ −67.36 (s), −68.07 (s). MS (QMS) m/z: [M+H]⁺ for C₃₁H₄₄ F₃N₂O₈S, found 661.3.

### (4S,5aS,8S,8aR)-4-Isobutyl-N-((1R)-3,3,3-trifluoro-2-methyl-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-04-081)

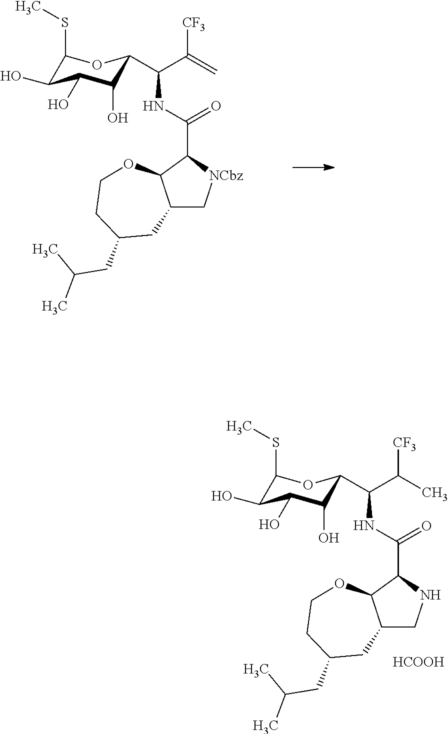

Procedure D (5 mg, d.r. 80:20, 52%). Purification by preparative HPLC-MS with a gradient 5-70% in 25 minutes of acetonitrile +0.1% formic acid in water +0.1% formic acid. Values are given for the major diastereoisomer, ¹H NMR (500 MHz, cd₃od) δ 8.42 (s, 1H), 5.26 (d, J=5.6 Hz, 1H), 4.59 (d, J=10.4 Hz, 2H), 4.31 (t, J=9.2 Hz, 1H), 4.29-4.24 (m, 1H), 4.11-4.05 (m, 1H), 3.95 (d, J=11.4 Hz, 1H), 3.83 (s, 1H), 3.77 (t, J=8.7 Hz, 1H), 3.54 (dt, J=17.2, 10.0 Hz, 2H), 2.92-2.80 (m, 2H), 2.29-2.22 (m, 1H), 2.10 (s, 3H), 1.99 (d, J=12.8 Hz, 1H), 1.80-1.63 (m, 4H), 1.23 (d, J=7.2 Hz, 3H), 1.18 (dd, J=11.2, 6.3 Hz, 2H), 0.98-0.91 (m, 1H), 0.89 (d, J=6.5 Hz, 6H). ¹⁹F NMR (471 MHz, cd₃od) δ −67.29 (s). HRMS (ESI+, m/z): [M+H]⁺ calcd for C23H40F₃N₂O₆S 529.2554; found 529.2552.

### Tert-butyl (4S,5aS,8S,8aR)-8-(((1S,2S)-2-((4-((dimethylamino)methyl)phenyl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

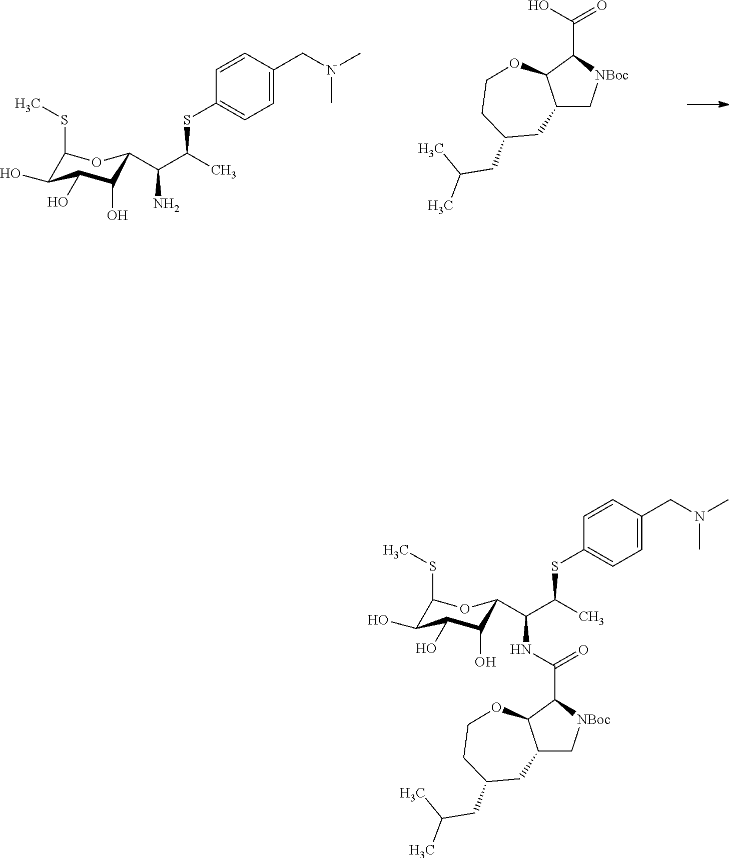

Procedure B (28 mg, 71%), rotameric mixture. ¹H NMR (500 MHz, cd₃od) δ 7.39 (dd, J=7.9, 5.5 Hz, 2H), 7.28 (t, J=7.8 Hz, 2H), 5.27 (dd, J=11.0, 5.6 Hz, 1H), 4.58-4.49 (m, 1H), 4.42 (t, J=9.5 Hz, 1H), 4.32 (d, J=9.9 Hz, 1H), 4.23-4.15 (m, 1H), 4.10 (ddd, J=13.1, 10.2, 4.8 Hz, 1H), 3.97 (d, J=2.9 Hz, 1H), 3.91 (dd, J=10.1, 6.3 Hz, 1H), 3.85-3.72 (m, 3H), 3.58 (td, J=10.2, 3.3 Hz, 1H), 3.47 (s, 2H), 2.91 (t, J=10.6 Hz, 1H), 2.50 (dt, J=20.0, 10.8 Hz, 1H), 2.25 (s, 6H), 1.99 (s, 1H), 1.94 (s, 2H), 1.75 (d, J=13.2 Hz, 1H), 1.65 (dt, J=13.4, 6.5 Hz, 3H), 1.47 (s, 9H), 1.37 (d, J=6.9 Hz, 3H), 1.22-1.10 (m, 2H), 0.96 (dd, J=24.0, 11.9 Hz, 1H), 0.89 (dd, J=6.6, 1.7 Hz, 6H). MS (QMS) m/z: [M+H]⁺ for C₃₆H₆₀N₃O₈S₂, found 726.3.

### (4S,5aS,8S,8aR)-N-((1S,2S)-2-((4-((Dimethylamino)methyl)phenyl)thio)-1-((2R,3R,4S,5R,6R)-3,4,5-trihydroxy-6-(methylthio)tetrahydro-2H-pyran-2-yl)propyl)-4-isobutyloctahydro-2H-oxepino[2,3-c]pyrrole-8-carboxamide formate salt (FSA-09-04-092)

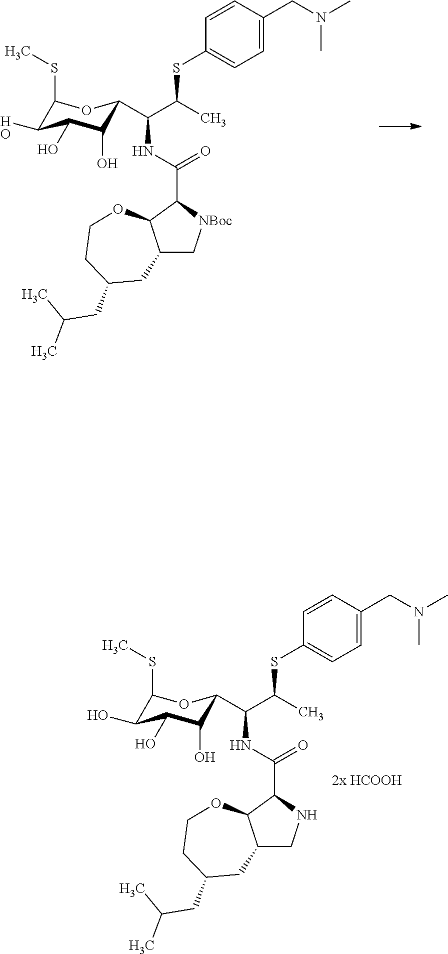

Procedure C (4.5 mg, 44%). ¹H NMR (500 MHz, cd₃od) δ 8.46 (s, 2H), 7.44 (d, J=8.0 Hz, 2H), 7.39 (d, J=8.0 Hz, 2H), 5.23 (d, J=5.5 Hz, 1H), 4.64 (d, J=10.1 Hz, 1H), 4.41 (d, J=10.0 Hz, 1H), 4.33 (dt, J=18.0, 8.8 Hz, 2H), 4.12 (s, 2H), 4.07 (dd, J=10.1, 5.6 Hz, 1H), 4.01 (d, J=11.9 Hz, 1H), 3.88 (dd, J=16.4, 4.9 Hz, 2H), 3.80 (dd, J=14.7, 6.9 Hz, 1H), 3.54 (dd, J=12.9, 5.3 Hz, 2H), 2.85 (t, J=11.6 Hz, 1H), 2.72 (s, 6H), 2.30-2.20 (m, 1H), 1.99 (d, J=12.8 Hz, 1H), 1.82 (s, 3H), 1.81-1.60 (m, 4H), 1.42 (d, J=6.8 Hz, 3H), 1.24-1.11 (m, 2H), 0.95 (dd, J=23.7, 12.0 Hz, 1H), 0.87 (d, J=6.5 Hz, 6H). HRMS (ESI+, m/z): [M+H]+ calcd for C31H52N₃O₆S₂ 626.3292; found 626.3290.

### Tert-butyl (4S,5aS,8S,8aR)-8-(((1S,2S)-2-((4-(2-(dimethylamino)ethyl)phenyl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

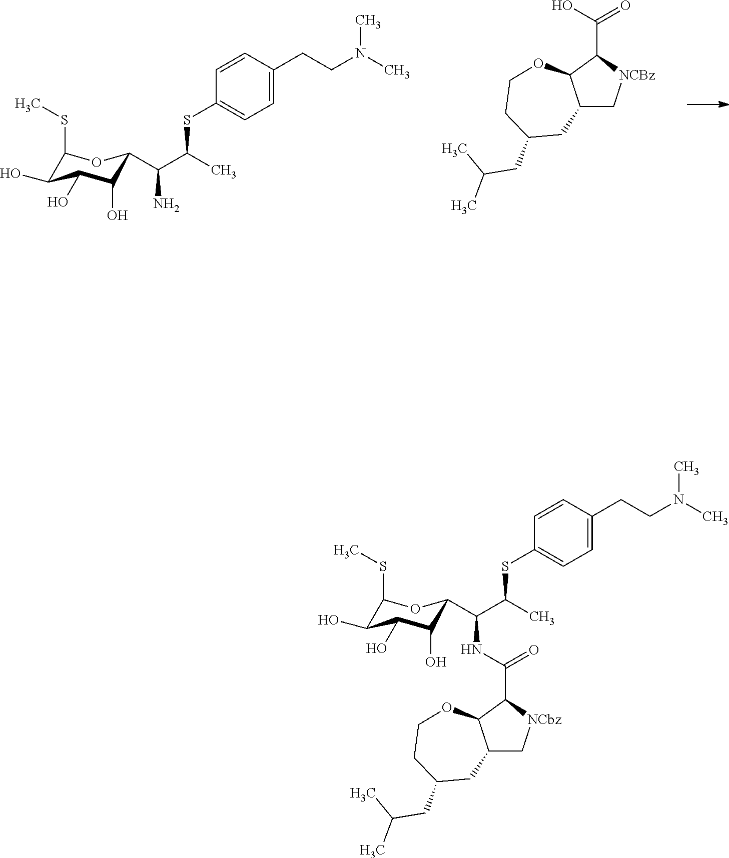

Procedure B (11 mg, 36%), rotameric mixture. ¹H NMR (500 MHz, cd₃od) δ 7.41-7.27 (m, 6H), 7.17 (dd, J=7.6, 3.9 Hz, 2H), 5.33 (d, J=12.6 Hz, 1H), 5.27-5.21 (m, 1H), 5.16-5.08 (m, 1H), 4.97 (d, J=12.6 Hz, 1H), 4.56 (dd, J=18.6, 8.1 Hz, 1H), 4.49-4.37 (m, 2H), 4.22-4.14 (m, 1H), 4.08 (ddt, J=18.1, 10.1, 5.0 Hz, 2H), 3.86 (dd, J=18.5, 10.5 Hz, 3H), 3.83-3.67 (m, 1H), 3.61-3.54 (m, 1H), 2.97 (dd, J=19.1, 10.4 Hz, 1H), 2.82-2.75 (m, 2H), 2.65-2.59 (m, 2H), 2.53 (dd, J=21.1, 10.8 Hz, 1H), 2.36 (s, 6H), 1.99 (d, J=3.9 Hz, 2H), 1.96 (s, 2H), 1.91 (d, J=16.1 Hz, 1H), 1.83 (dd, J=12.9, 6.4 Hz, 1H), 1.73 (d, J=13.8 Hz, 1H), 1.62 (dd, J=12.1, 7.8 Hz, 3H), 1.48-1.46 (m, 1H), 1.33 (dd, J=9.4, 6.9 Hz, 2H), 1.30-1.22 (m, 2H), 1.22-1.08 (m, 2H), 0.94 (dd, J=23.5, 11.5 Hz, 1H), 0.89-0.83 (m, 6H). MS (QMS) m/z: [M+H]⁺ for C₄₀H₆₀N₃O₈S₂, found 774.2.

### (4S,5aS,8S,8aR)-N-((1S,2S)-2-((4-(2-(Dimethylamino)ethyl)phenyl)thio)-1-((2R,3R,4S,5R,6R)-3,4,5-trihydroxy-6-(methylthio)tetrahydro-2H-pyran-2-yl)propyl)-4-isobutyloctahydro-2H-oxepino[2,3-c]pyrrole-8-carboxamide formate salt (FSA-09-04-087)

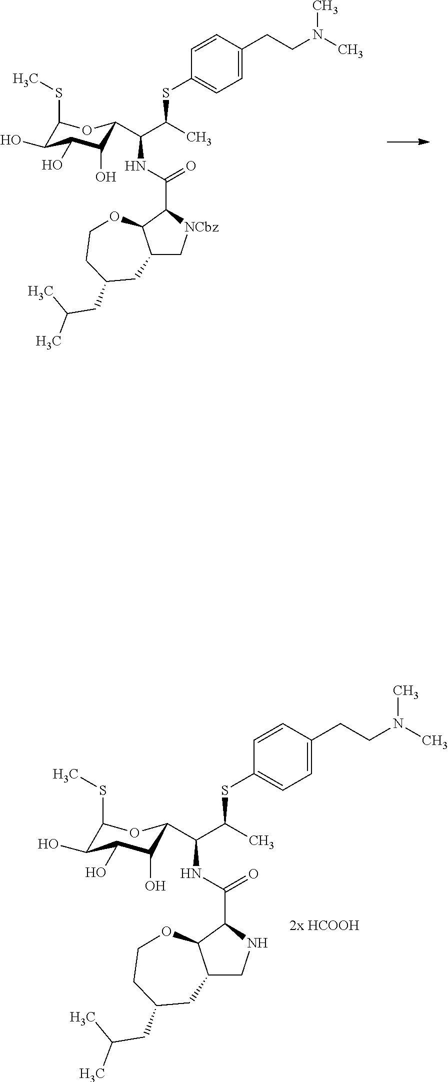

Procedure D (1.8 mg, 20%). ¹H NMR (500 MHz, CD₃OD) δ 8.46 (s, 2H), 7.39 (d, J=8.2 Hz, 2H), 7.26 (d, J=8.2 Hz, 2H), 5.26 (d, J=5.6 Hz, 1H), 4.60 (dd, J=10.0, 2.3 Hz, 1H), 4.42 (d, J=10.0 Hz, 1H), 4.32 (d, J=4.7 Hz, 2H), 4.09 (dd, J=10.3, 5.6 Hz, 1H), 4.02 (dt, J=12.2, 3.9 Hz, 1H), 3.88 (d, J=3.0 Hz, 1H), 3.87-3.79 (m, 2H), 3.55 (ddd, J=18.4, 10.6, 5.4 Hz, 2H), 3.31-3.24 (m, 2H), 3.01 (dd, J=9.9, 6.7 Hz, 2H), 2.88 (s, 6H), 2.83 (t, J=11.6 Hz, 1H), 2.26 (td, J=7.8, 4.3 Hz, 1H), 2.01 (d, J=12.6 Hz, 1H), 1.92 (s, 3H), 1.82-1.62 (m, 4H), 1.37 (d, J=6.9 Hz, 3H), 1.26-1.14 (m, 1H), 0.96 (dd, J=23.5, 11.9 Hz, 1H), 0.90 (dd, J=6.6, 1.3 Hz, 6H). HRMS (ESI+, m/z): [M+H]⁺ calcd for C32H54N₃O₆S₂ 640.3449; found 640.3447.

### Tert-butyl (4S,5aS,8S,8aR)-8-(((1S,2S)-2-((5-((tert-butoxycarbonyl)amino)-1,3,4-thiadiazol-2-yl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

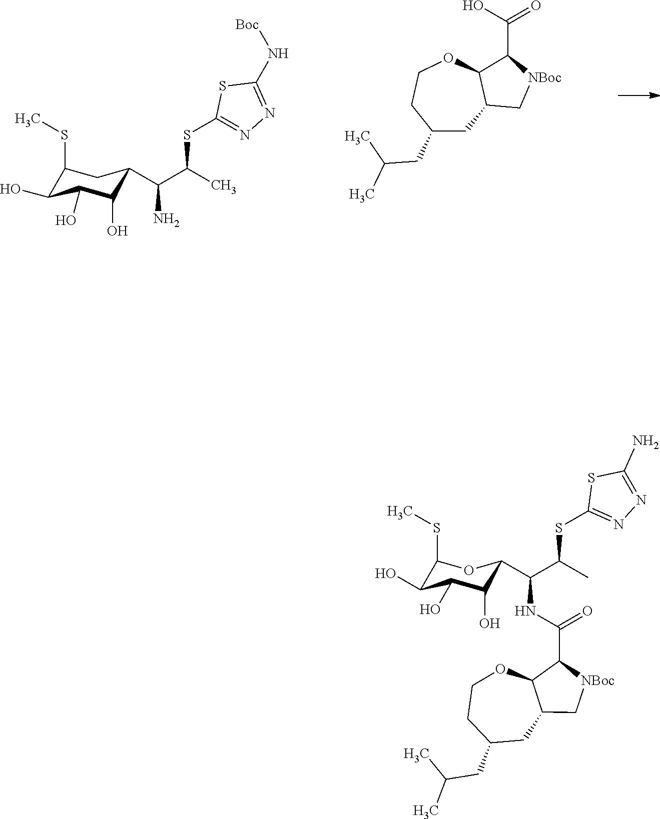

Procedure B (12 mg, 51%), rotameric mixture. ¹H NMR (500 MHz, cd₃od) δ 5.26 (dd, J=9.6, 5.6 Hz, 1H), 4.50 (dd, J=10.1, 2.3 Hz, 0.6H), 4.44 (dd, J=15.7, 5.4 Hz, 1H), 4.39-4.33 (m, 1H), 4.29 (d, J=10.1 Hz, 0.5H), 4.18-4.12 (m, 1H), 4.12-4.04 (m, 1.7H), 4.04-3.97 (m, 1.5H), 3.90-3.85 (m, 0.7H), 3.79 (ddd, J=23.8, 10.4, 4.0 Hz, 2H), 3.73-3.62 (m, 1H), 3.54 (td, J=10.0, 3.3 Hz, 1H), 2.91-2.84 (m, 1H), 2.48 (dd, J=19.2, 10.0 Hz, 1H), 2.12 (s, 1H), 2.07 (s, 2H), 1.93 (t, J=16.8 Hz, 1H), 1.75-1.57 (m, 4H), 1.54 (s, 9H), 1.44 (s, 4H), 1.42 (s, 5H), 1.19-1.07 (m, 2H), 0.96-0.89 (m, 1H), 0.87 (d, J=6.5 Hz, 6H).

### (4S,5aS,8S,8aR)-N-((1S,2S)-2-((5-Amino-1,3,4-thiadiazol-2-yl)thio)-1-((2R,3R,4S,5R,6R)-3,4,5-trihydroxy-6-(methylthio)tetrahydro-2H-pyran-2-yl)propyl)-4-isobutyloctahydro-2H-oxepino[2,3-c]pyrrole-8-carboxamide formate salt (FSA-09-04-094)

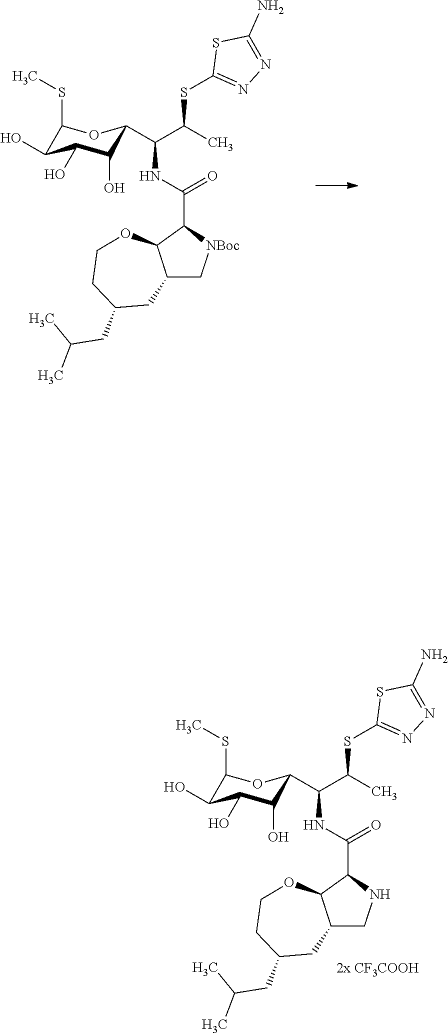

The bis N-Boc protected starting material (10 mg, 0.013 mmol) was dissolved in methanol (0.6 mL) and the reaction temperature was lowered to 0° C., at this point a freshly prepared 2N solution of HCl in methanol (0.6 mL) was added dropwise, the cooling bath was removed and the reaction was stirred at room temperature for 4 hours, LCMS showed mainly formation of single Boc cleaved product, therefore toluene (0.2 mL) was added and the volatiles were removed with a stream of nitrogen, the residue obtained dissolved in dichloromethane (0.4 mL) and trifluoroacetic acid (0.1 mL) was added, the reaction was stirred for 30 minutes then the solvent was removed under reduced pressure. The residue was purified by preparative HPLC-MS with a gradient 5-50% of acetonitrile +0.1% formic acid in water +0.1% formic acid, the fractions containing the desired compound were collected and the solvent was removed in vacuo to give a colorless material (2.8 mg, 38%). ¹H NMR (500 MHz, cd₃od) δ 8.32 (s, 2H), 5.26 (d, J=5.6 Hz, 1H), 4.51 (dd, J=10.1, 1.9 Hz, 1H), 4.42 (d, J=8.9 Hz, 1H), 4.36 (dd, J=9.2, 7.6 Hz, 2H), 4.08 (dd, J=10.2, 5.6 Hz, 1H), 3.97-3.90 (m, 1H), 3.87 (dd, J=8.0, 3.6 Hz, 2H), 3.75-3.67 (m, 1H), 3.61-3.53 (m, 2H), 2.89 (t, J=11.8 Hz, 1H), 2.32-2.21 (m, 1H), 2.09 (s, 3H), 1.99 (d, J=13.2 Hz, 1H), 1.76-1.60 (m, 4H), 1.44 (d, J=7.0 Hz, 3H), 1.23-1.12 (m, 2H), 0.94 (dd, J=23.7, 12.1 Hz, 1H), 0.88 (d, J=6.4 Hz, 6H). HRMS (ESI+, m/z): [M+H]⁺ calcd for C24H42N₅O₆S₃ 592.2292; found 592.2289.

### Tert-butyl (4S,5aS,8S,8aR)-8-(((1S,2S)-2-((5-(4-(tert-butoxycarbonyl)piperazin-1-yl)-1,3,4-thiadiazol-2-yl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

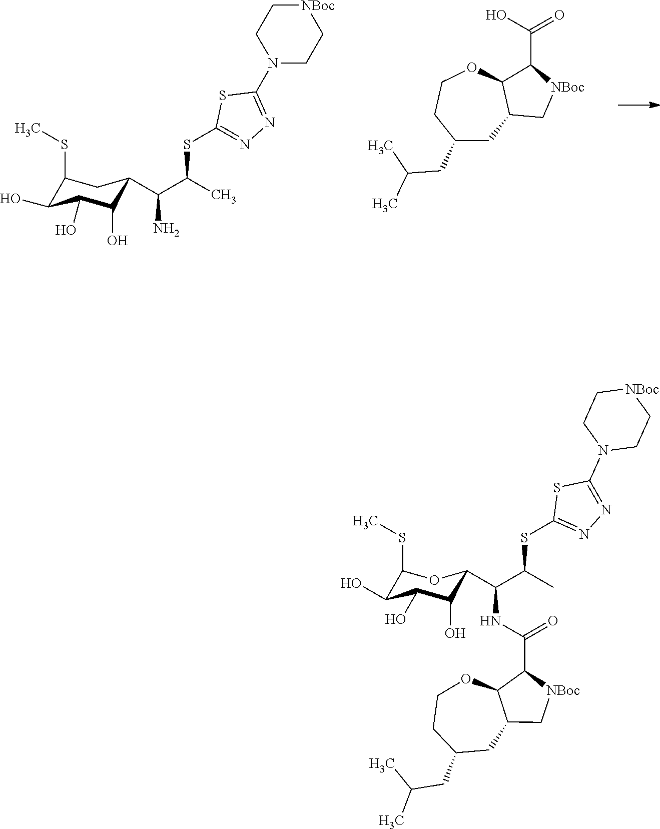

Procedure B (10 mg, 55%), rotameric mixture. ¹H NMR (500 MHz, cd₃od) δ 5.27 (dd, J=8.9, 5.7 Hz, 1H), 4.52-4.45 (m, 1H), 4.44-4.29 (m, 2H), 4.18 (t, J=9.3 Hz, 1H), 4.14-4.05 (m, 1.5H), 4.01 (d, J=3.1 Hz, 0.5H), 3.98-3.75 (m, 3H), 3.75-3.65 (m, 1H), 3.63-3.49 (m, 10H), 2.90 (t, J=10.6 Hz, 1H), 2.51 (s, 1H), 2.15 (s, 1H), 2.11 (s, 2H), 1.95 (dd, J=23.1, 13.6 Hz, 1H), 1.77-1.57 (m, 3H), 1.49 (s, 9H), 1.46 (d, J=2.6 Hz, 9H), 1.44-1.40 (m, 3H), 1.15 (dt, J=13.7, 6.9 Hz, 2H), 0.98-0.91 (m, 1H), 0.89 (d, J=6.5 Hz, 6H).

### (4S,5aS,8S,8aR)-4-Isobutyl-N-((1S,2S)-2-((5-(piperazin-1-yl)-1,3,4-thiadiazol-2-yl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide trifluoroacetate salt (FSA-09-05-011)

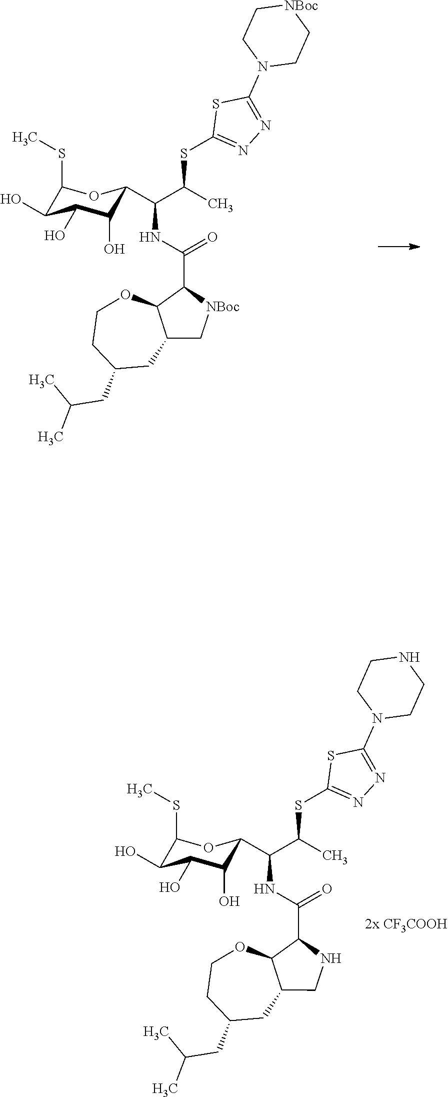

The bis N-Boc protected starting material (8 mg, 9.29 μmol) was dissolved in dichloromethane (0.8 mL) and trifluoroacetic acid (0.2 mL) was added at 0° C., the reaction was stirred for one hour at room temperature, the solvent was removed under reduced pressure and the residue obtained was purified by preparative HPLC-MS with a gradient 5-50% of acetonitrile +0.1% formic acid in water +0.1% formic acid, the fractions containing the desired compound were collected and the solvent was removed in vacuo to give a colorless material (4.5 mg, 73%). ¹H NMR (500 MHz, cd₃od) δ 8.40 (s, 2H), 5.28 (d, J=5.6 Hz, 1H), 4.62 (dd, J=10.1, 2.1 Hz, 1H), 4.44-4.34 (m, 3H), 4.11 (ddd, J=12.2, 8.7, 5.2 Hz, 2H), 3.95 (dt, J=11.9, 3.7 Hz, 1H), 3.88 (d, J=3.0 Hz, 1H), 3.82-3.75 (m, 1H), 3.74-3.69 (m, 4H), 3.58 (ddd, J=13.5, 10.8, 5.3 Hz, 2H), 3.31-3.27 (m, 4H), 2.91 (t, J=11.8 Hz, 1H), 2.35-2.24 (m, 1H), 2.08 (s, 3H), 2.01 (d, J=13.1 Hz, 1H), 1.81-1.61 (m, 4H), 1.50 (d, J=6.9 Hz, 3H), 1.26-1.14 (m, 2H), 0.97 (dd, J=23.6, 12.0 Hz, 1H), 0.90 (d, J=6.0 Hz, 6H). HRMS (ESI+, m/z): [M+H]⁺ calcd for C28H49N₆O₆S₃ 661.2870; found 661.2867.

### (4S,5aS,8S,8aR)-4-Isobutyl-N-((1S,2S)-2-((4-(pyridin-3-yl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-05-074)

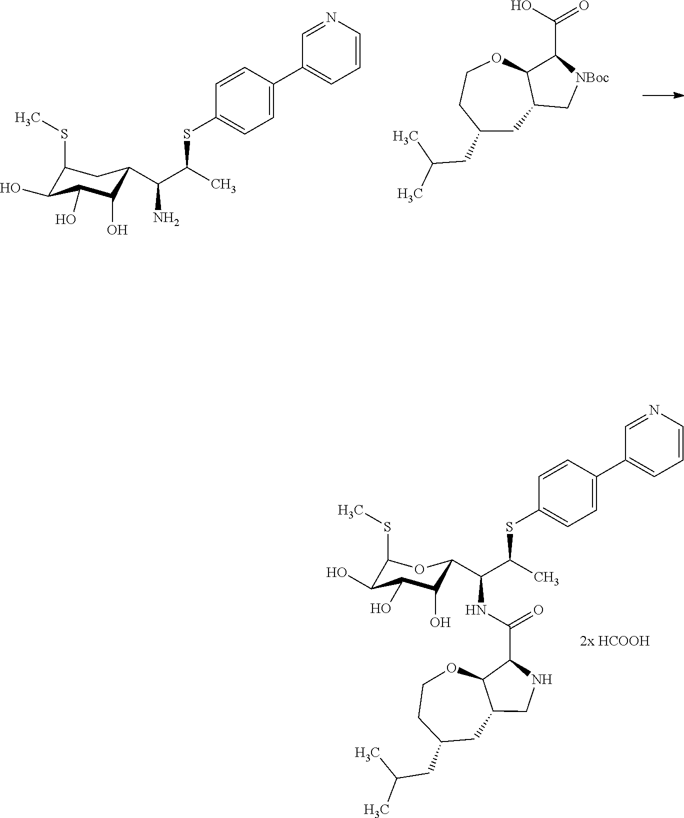

Procedure B followed directly by Procedure C (3.7 mg, 44% over two steps). ¹H NMR (500 MHz, cd₃od) δ 8.79 (d, J=1.8 Hz, 1H), 8.51 (dd, J=4.9, 1.5 Hz, 1H), 8.38 (s, 1H), 8.12-8.06 (m, 1H), 7.66-7.59 (m, 2H), 7.54-7.48 (m, 3H), 5.26 (d, J=5.6 Hz, 1H), 4.65 (dd, J=10.0, 2.3 Hz, 1H), 4.44 (dd, J=14.0, 9.5 Hz, 2H), 4.34 (t, J=9.2 Hz, 1H), 4.09 (dd, J=10.3, 5.6 Hz, 1H), 4.03 (dt, J=12.0, 3.8 Hz, 1H), 3.95-3.87 (m, 2H), 3.87-3.79 (m, 1H), 3.63-3.52 (m, 2H), 2.89 (t, J=11.8 Hz, 1H), 2.34-2.22 (m, 1H), 2.00 (d, J=12.9 Hz, 1H), 1.90 (s, 3H), 1.83-1.58 (m, 4H), 1.44 (d, J=6.9 Hz, 3H), 1.18 (tq, J=13.7, 6.9 Hz, 2H), 0.96 (dd, J=23.9, 11.8 Hz, 1H), 0.87 (dd, J=6.6, 2.1 Hz, 6H). HRMS (ESI+, m/z): [M+H]⁺ calcd for C28H49N₆O₆S₃ 646.2979; found 646.2979.

### Tert-butyl (4S,5aS,8S,8aR)-8-(((1S,2S)-2-((4-(hydroxymethyl)phenyl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate

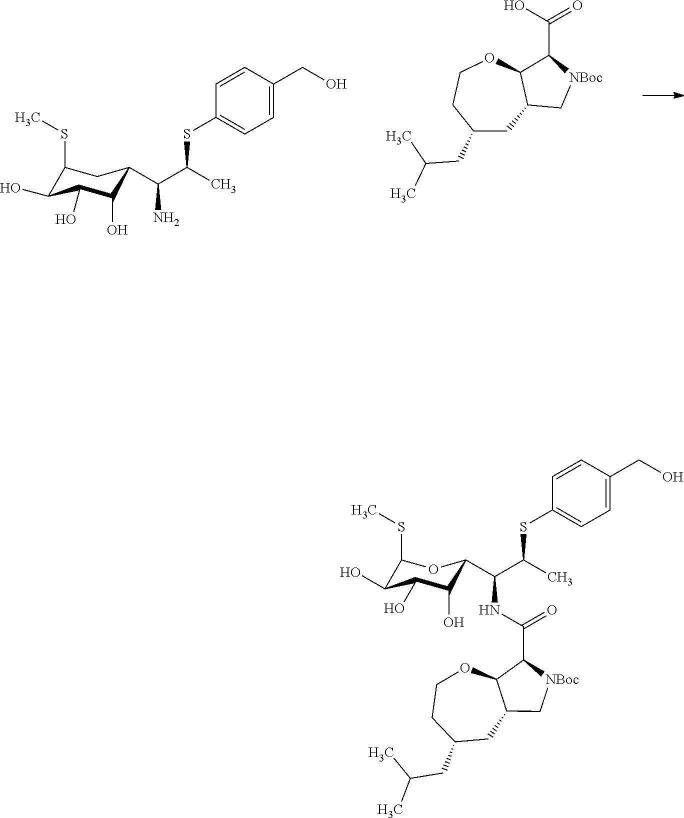

Procedure B, the desired material was a white powder (335 mg, 74% over two steps). ¹H NMR (500 MHz, cd₃od) δ 7.41-7.35 (m, 2H), 7.32-7.27 (m, 2H), 5.25 (dd, J=11.6, 5.6 Hz, 1H), 4.56 (s, 2H), 4.50 (dd, J=18.6, 10.5 Hz, 2H), 4.40 (dd, J=15.8, 9.3 Hz, 2H), 4.31 (d, J=9.8 Hz, 1H), 4.20-4.13 (m, 2H), 4.11-4.02 (m, 2H), 3.95 (s, 1H), 3.91-3.84 (m, 2H), 3.81-3.69 (m, 3H), 3.57 (dd, J=12.8, 6.1 Hz, 2H), 2.89 (t, J=10.5 Hz, 1H), 2.55-2.40 (m, 2H), 2.01 (s, 1H), 1.99-1.88 (m, 3H), 1.74 (d, J=12.9 Hz, 1H), 1.63 (dd, J=13.4, 6.7 Hz, 4H), 1.45 (s, 9H), 1.33 (d, J=6.8 Hz, 3H), 1.14 (dt, J=13.6, 6.8 Hz, 3H), 0.96-0.90 (m, 1H), 0.87 (dd, J=6.5, 1.5 Hz, 6H). HRMS (ESI+, m/z): [M+H]⁺ calcd for C₃₄H₅₅N₂O₉S₂ 699.3343; found 699.3347.

### (4S,5aS,8S,8aR)-N-((1S,2S)-2-((4-(Hydroxymethyl)phenyl)thio)-1-((2R,3R,4S,5R,6R)-3,4,5-trihydroxy-6-(methylthio)tetrahydro-2H-pyran-2-yl)propyl)-4-isobutyloctahydro-2H-oxepino[2,3-c]pyrrole-8-carboxamide formate salt (FSA-09-05-069)

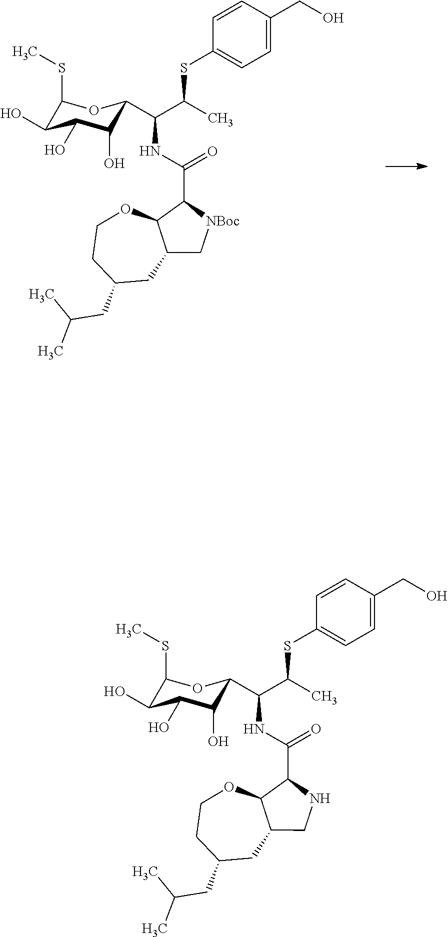

Procedure C (4.3 mg, 68%). ¹H NMR (500 MHz, cd₃od) δ 8.44 (s, 1H), 7.37 (d, J=7.9 Hz, 2H), 7.29 (d, J=8.1 Hz, 2H), 5.25 (d, J=5.6 Hz, 1H), 4.56 (s, 2H), 4.42 (d, J=9.2 Hz, 1H), 4.37-4.26 (m, 1H), 4.08 (dd, J=10.3, 5.6 Hz, 1H), 4.01 (d, J=11.5 Hz, 1H), 3.86 (d, J=2.9 Hz, 2H), 3.85-3.76 (m, 2H), 3.59-3.50 (m, 2H), 2.87-2.79 (m, 1H), 2.30-2.19 (m, 1H), 1.98 (d, J=13.1 Hz, 1H), 1.92 (s, 3H), 1.81-1.58 (m, 4H), 1.35 (d, J=6.9 Hz, 3H), 1.18 (dd, J=11.6, 5.9 Hz, 2H), 0.94 (dd, J=23.6, 12.0 Hz, 1H), 0.88 (d, J=5.9 Hz, 6H). HRMS (ESI+, m/z): [M+H]⁺ calcd for C29H47N₂O₇S₂ 599.2819; found 599.2818.

### Three Step Protocol for the Synthesis of Analogs Varied at the Benzylic Position

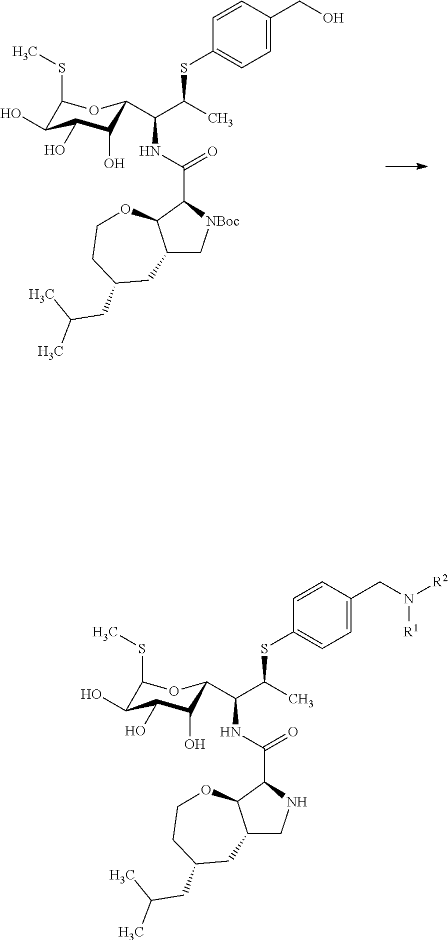

Tert-butyl (4S,5aS,8S,8aR)-8-(((1S,2S)-((4-(hydroxymethyl)phenyl)thio)-1-((2R,3R,4S,5R,6R)-3,4,5-trihydroxy-6-(methylthio)tetrahydro-2H-pyran-2-yl)propyl)carbamoyl)-4-isobutyloctahydro-7H-oxepino[2,3-c]pyrrole-7-carboxylate (1.0 eq) was dissolved in dichloromethane (M=0.02), the solution was cooled to 0° C. and Dess-Martin periodinane (1.1 eq.) was added portion wise, the reaction temperature slowly raised upon melting of the ice bath and as stirred for one hour. The reaction was cooled to 0° C. and diluted with ethyl acetate (1 volume), saturated solution of sodium bicarbonate (½ volume) and half saturated solution of sodium thiosulfate (½ volume). the organic phase was washed with brine, dried over sodium sulfate, filtered and the evaporated under reduced pressure.

The crude aldehyde thus obtained was directly used in the reductive amination step: The crude aldehyde (1.0 eq) was dissolved in dichloromethane (M=0.05), the amine (2.0 eq.) followed by acetic acid (2.0 eq.) were added and the solution was stirred at room temperature for five minutes before sodium triacetoxyborohydride (3.0 eq.) was added. The reaction was stirred at room temperature for one hour and then it was diluted with ethyl acetate and saturated solution of sodium bicarbonate, the organic phase was washed with brine, dried over sodium sulfate and evaporated in vacuo, the crude residue the obtained was directly submitted to the final acidic N-Boc cleavage.

### (4S,5aS,8S,8aR)-4-Isobutyl-N-((1S,2S)-2-((4-(morpholinomethyl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-05-068)

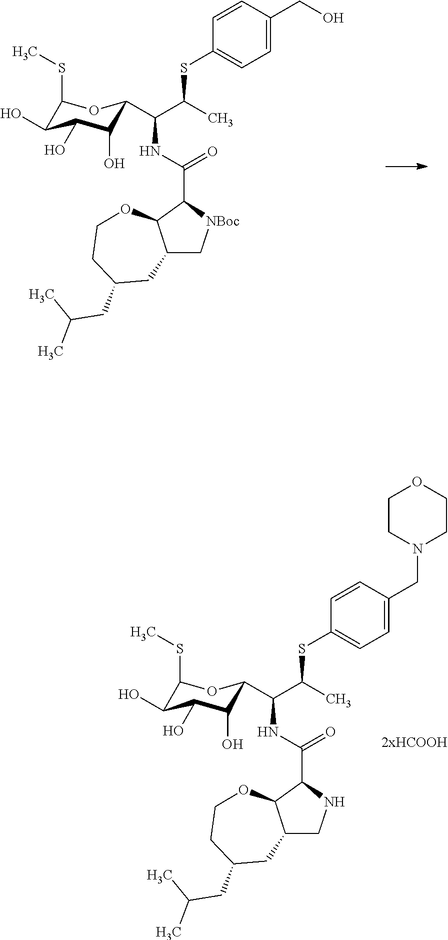

The three-step protocol gave the desired product (4.4 mg, 35%). ¹H NMR (500 MHz, cd₃od) δ 7.37 (d, J=8.2 Hz, 2H), 7.31 (d, J=8.2 Hz, 2H), 5.24 (d, J=5.6 Hz, 1H), 4.61 (dd, J=10.0, 2.2 Hz, 1H), 4.42 (d, J=10.0 Hz, 2H), 4.34 (t, J=9.2 Hz, 1H), 4.08 (dd, J=10.2, 5.6 Hz, 1H), 4.02 (dt, J=12.1, 3.7 Hz, 1H), 3.83 (ddd, J=16.0, 12.2, 3.3 Hz, 3H), 3.75-3.69 (m, 4H), 3.64 (s, 2H), 3.57 (ddd, J=13.5, 10.7, 5.3 Hz, 2H), 2.89 (t, J=11.8 Hz, 1H), 2.59 (s, 4H), 2.32-2.22 (m, 1H), 2.00 (d, J=12.6 Hz, 1H), 1.87 (s, 3H), 1.81-1.60 (m, 5H), 1.39 (d, J=6.9 Hz, 3H), 1.18 (tq, J=13.7, 6.8 Hz, 2H), 0.96 (dd, J=23.8, 11.6 Hz, 1H), 0.88 (dd, J=6.5, 1.4 Hz, 6H). HRMS (ESI+, m/z): [M+H]⁺ calcd for C33H55N₃O₇S₂ 668.3398; found 668.3394.

### (4S,5aS,8S,8aR)-4-Isobutyl-N-((1S,2S)-2-((4-(piperazin-1-ylmethyl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-05-076)

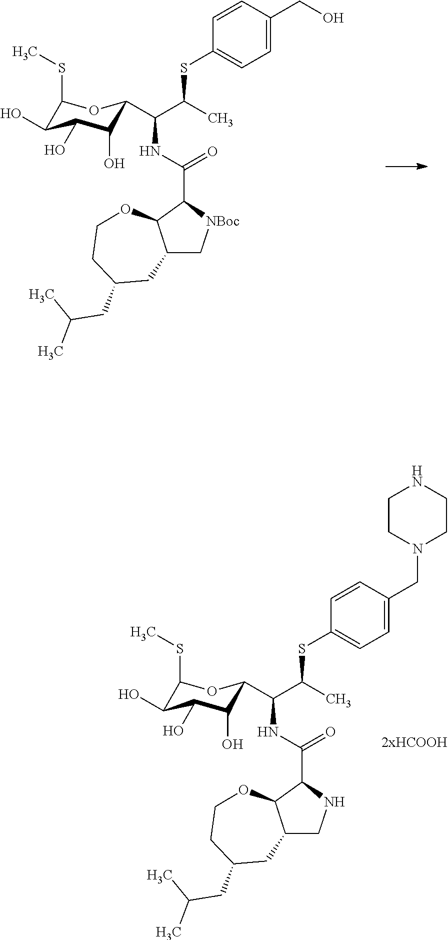

The three-step protocol gave the desired product (3.3 mg, 31%). ¹H NMR (500 MHz, cd₃od) δ 8.47 (s, 2H), 7.37 (d, J=8.3 Hz, 2H), 7.29 (d, J=8.3 Hz, 2H), 5.24 (d, J=5.6 Hz, 1H), 4.58 (dd, J=10.0, 2.2 Hz, 1H), 4.40 (d, J=10.1 Hz, 1H), 4.34-4.27 (m, 2H), 4.07 (dd, J=10.3, 5.6 Hz, 1H), 4.01 (dt, J=12.0, 3.8 Hz, 1H), 3.87 (d, J=2.9 Hz, 1H), 3.82 (ddd, J=16.1, 8.1, 3.3 Hz, 2H), 3.56-3.48 (m, 2H), 3.24-3.16 (m, 4H), 2.82 (t, J=11.5 Hz, 1H), 2.66 (s, 4H), 2.29-2.20 (m, 1H), 1.99 (d, J=15.2 Hz, 1H), 1.89 (s, 3H), 1.81-1.60 (m, 5H), 1.36 (d, J=6.9 Hz, 3H), 1.25-1.10 (m, 2H), 0.95 (dd, J=24.0, 11.4 Hz, 1H), 0.88 (dd, J=6.6, 1.3 Hz, 6H). HRMS (ESI+, m/z): [M+H]⁺ calcd for C33H55N₄O₆S₂ 667.3558; found 667.3553.

### (4S,5aS,8S,8aR)-4-Isobutyl-N-((1S,2S)-2-((4-(piperidin-1-ylmethyl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-05-077)

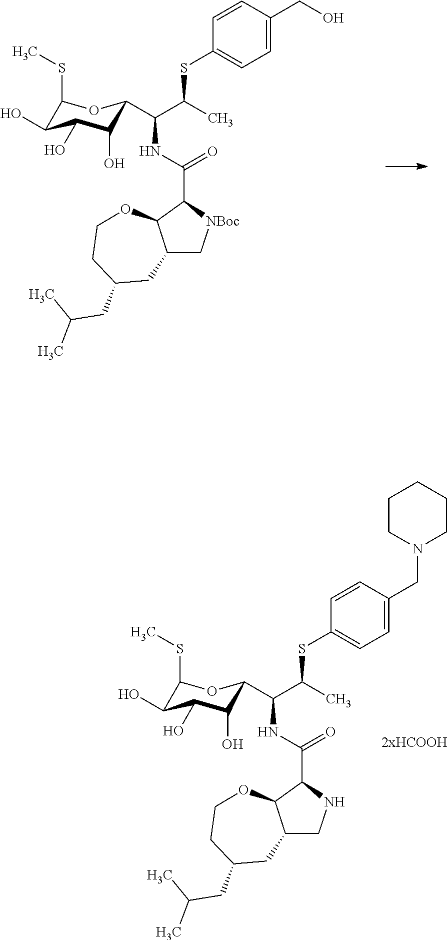

The three-step protocol gave the desired product (4.8 mg, 45%). ¹H NMR (500 MHz, cd₃od) δ 8.47 (s, 2H), 7.42 (q, J=8.5 Hz, 4H), 5.24 (d, J=5.6 Hz, 1H), 4.65 (dd, J=10.1, 2.1 Hz, 1H), 4.41 (d, J=10.1 Hz, 1H), 4.33 (dt, J=17.9, 8.9 Hz, 2H), 4.17 (s, 2H), 4.08 (dd, J=10.2, 5.6 Hz, 1H), 4.01 (dt, J=12.0, 3.8 Hz, 1H), 3.91 (dt, J=9.1, 5.6 Hz, 1H), 3.87 (d, J=2.7 Hz, 1H), 3.84-3.77 (m, 1H), 3.58-3.51 (m, 2H), 3.10 (s, 3H), 2.85 (t, J=11.6 Hz, 1H), 2.31-2.21 (m, 1H), 1.99 (d, J=12.9 Hz, 1H), 1.83 (s, 3H), 1.82-1.78 (m, 3H), 1.77-1.59 (m, 6H), 1.42 (d, J=6.9 Hz, 3H), 1.24-1.12 (m, 2H), 0.95 (dd, J=24.2, 11.6 Hz, 1H), 0.88 (dd, J=6.6, 1.8 Hz, 6H).
HRMS (ESI+, m/z): [M+H]⁺ calcd for C34H56N₃O₆S₂ 666.3605; found 666.3601.

### (4S,5aS,8S,8aR)-4-Isobutyl-N-((1S,2S)-2-((4-(pyrrolidin-1-ylmethyl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide formate salt (FSA-09-05-078)

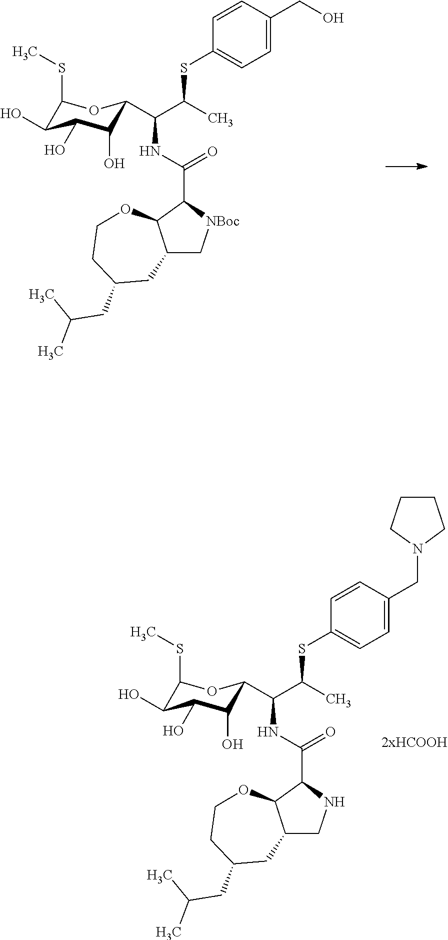

The three-step protocol gave the desired product (4.8 mg, 46%). ¹H NMR (500 MHz, cd₃od) δ 8.44 (s, 2H), 7.47-7.40 (m, 4H), 5.24 (d, J=5.6 Hz, 1H), 4.65 (dd, J=10.1, 2.2 Hz, 1H), 4.40 (dd, J=17.3, 9.5 Hz, 2H), 4.33 (t, J=9.1 Hz, 1H), 4.29 (s, 2H), 4.08 (dd, J=10.2, 5.6 Hz, 1H), 4.01 (dt, J=11.9, 3.8 Hz, 1H), 3.90 (qd, J=6.7, 2.0 Hz, 1H), 3.86 (d, J=2.8 Hz, 1H), 3.84-3.77 (m, 1H), 3.59-3.52 (m, 2H), 3.28 (t, J=5.9 Hz, 3H), 2.87 (t, J=11.7 Hz, 1H), 2.26 (ddd, J=15.2, 12.0, 6.1 Hz, 1H), 2.09-2.04 (m, 4H), 1.99 (dd, J=15.1, 2.2 Hz, 1H), 1.83 (s, 3H), 1.79-1.59 (m, 4H), 1.42 (d, J=6.9 Hz, 3H), 1.24-1.12 (m, 2H), 0.96 (dd, J=24.1, 11.7 Hz, 1H), 0.88 (dd, J=6.6, 1.8 Hz, 6H). HRMS (ESI+, m/z): [M+H]⁺ calcd for C33H54N₃O₆S₂ 652.3449; found 652.3447.

### (4S,5aS,8S,8aR)-4-Isobutyl-N-((1S,2S)-2-((4-((methylamino)methyl)phenyl)thio)-1-((2R,3R,4S,5R,6R)-3,4,5-trihydroxy-6-(methylthio)tetrahydro-2H-pyran-2-yl)propyl)octahydro-2H-oxepino[2,3-c]pyrrole-8-carboxamide (FSA-09-05-079)

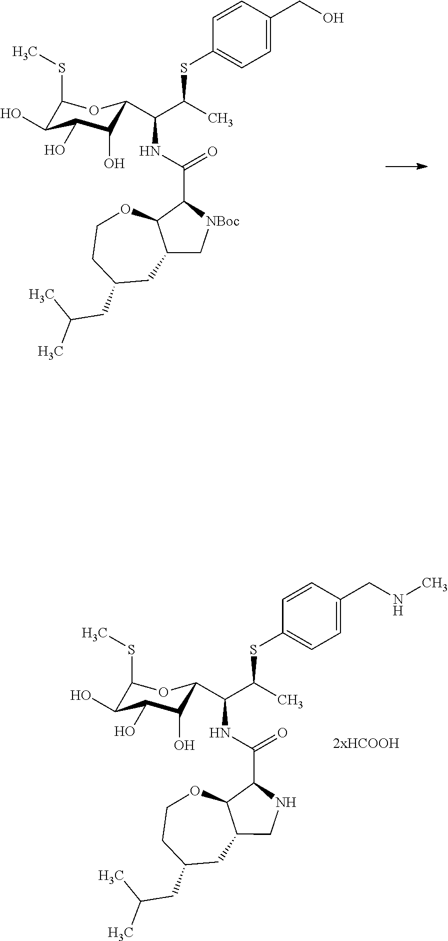

The three-step protocol gave the desired product (2.9 mg, 30%).¹H NMR (500 MHz, cd₃od) δ 8.44 (s, 2H), 7.45-7.42 (m, 2H), 7.41-7.38 (m, 2H), 5.24 (d, J=5.6 Hz, 1H), 4.64 (dd, J=10.0, 2.2 Hz, 1H), 4.41 (d, J=10.1 Hz, 1H), 4.32 (q, J=9.0 Hz, 2H), 4.08 (dd, J=10.2, 5.6 Hz, 2H), 4.01 (dt, J=11.9, 3.9 Hz, 1H), 3.94-3.87 (m, 1H), 3.86 (d, J=3.0 Hz, 1H), 3.83-3.76 (m, 1H), 3.54 (dt, J=11.1, 5.4 Hz, 2H), 2.84 (t, J=11.6 Hz, 1H), 2.69 (s, 3H), 2.30-2.21 (m, 2H), 1.99 (d, J=13.0 Hz, 2H), 1.84 (s, 3H), 1.80-1.59 (m, 5H), 1.41 (d, J=6.9 Hz, 3H), 1.23-1.12 (m, 2H), 0.95 (dd, J=24.4, 11.6 Hz, 1H), 0.88 (dd, J=6.6, 1.8 Hz, 6H). HRMS (ESI+, m/z): [M+H]⁺ calcd for C30H50N₃O₆S₂ 612.3136; found 612.3134.

In order to deliver material for advanced study, and to propel continued analog synthesis, a route to FSA-513018b providing full C7′ stereocontrol and access to >3-gram quantities was developed.

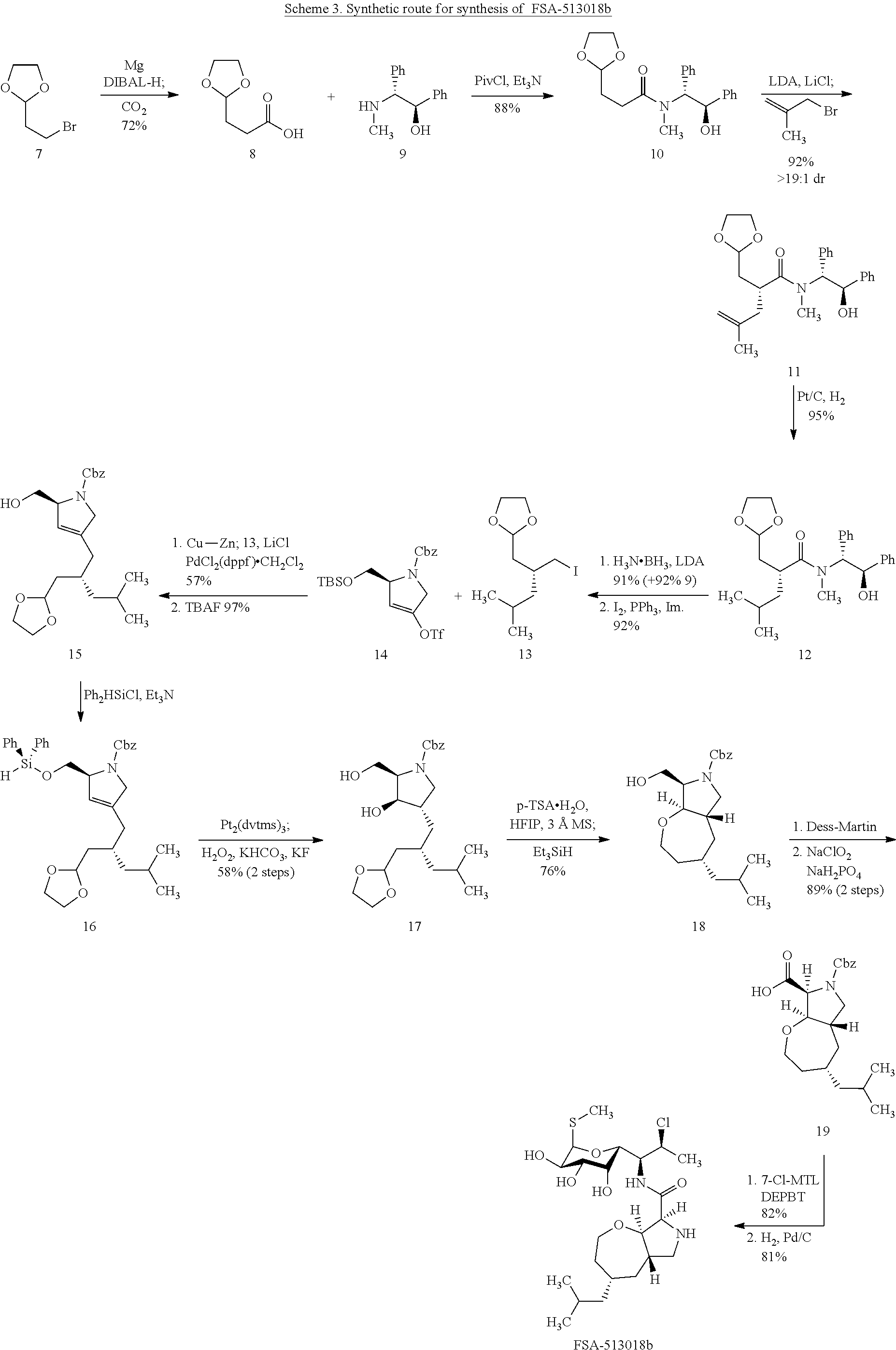

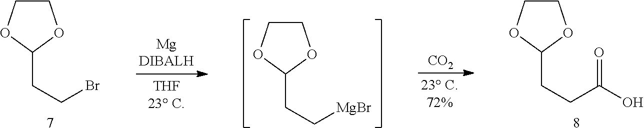

Acid 8: Freshly dried magnesium turnings (29.40 g, 1.21 mol, 3.55 equiv) were suspended in tetrahydrofuran (585 mL) at 23° C. and a pressure-equalizing addition funnel was charged with a solution of 2-(2-bromoethyl)-1,3-dioxolane (7) (40.0 mL, 61.68 g, 341 mmol) in tetrahydrofuran (175 mL). To the suspension were added sequentially approximately 10% of the alkyl bromide solution and a solution of diisobutylaluminum hydride (25% wt. in toluene, 2.3 mL, 485 mg of diisobutylaluminum hydride, 3.41 mmol, 1 mol %). A gentle exotherm indicated initiation of the reaction after ca. 5 min, and the remainder of the alkyl bromide solution was added to the magnesium turnings suspension dropwise at such a rate as to maintain a gentle exotherm (3.5 h). When the addition was complete, the mixture was stirred for an additional 30 min at 23° C. and was then sparged with carbon dioxide gas for 2 h, controlling the resulting exotherm with a room temperature water bath. The reaction mixture was then quenched with solid dry ice (ca. 20 g) and was stirred at 23° C. for 16 h. The suspension was filtered through a Celite pad, washing with ethyl ether (250 mL) and water (250 mL). The filtrate was concentrated in vacuo to remove the organic solvents. The aqueous solution was washed with ethyl ether (2×400 mL) and was then acidified to pH 3 using a 6 M aqueous solution of HCl. The solution was extracted with ethyl ether (12×500 mL) and the combined organic layers were dried over magnesium sulfate. The dried solution was filtered through a sintered glass funnel and was concentrated in vacuo to provide acid 8 (35.71 g, 72%) as a colorless oil that solidified to an amorphous white solid on standing. ¹H NMR (500 MHz, CDCl₃) δ: 10.84 (br s, 1H), 4.82 (t, J=4.2 Hz, 1H), 3.88-3.79 (m, 2H), 3.75-3.70 (m, 2H), 2.33 (t, J=7.6 Hz, 2H), 1.87 (td, J=4.2, 7.6 Hz, 2H); ¹³C NMR (125 MHz, CDCl₃) δ: 178.4, 102.8, 64.8, 28.3, 27.7; FTIR (neat), cm⁻¹: 3462, 2931, 1720, 1680, 1410, 1196, 905.

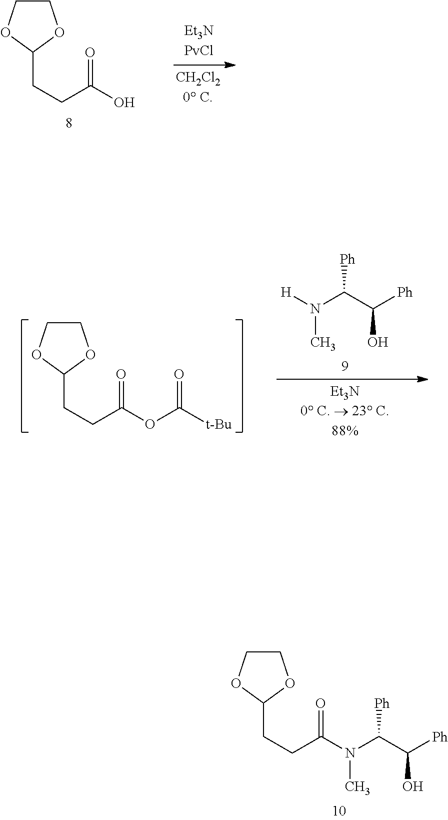

(R,R)-Pseudoephenamide 10: Triethylamine (35.5 mL, 25.8 g, 255 mmol, 1.2 equiv) was added to an ice-cooled solution of acid 12 (35.71 g, 244 mmol, 1.15 equiv) in dichloromethane (315 mL) and the resulting mixture was stirred at 0° C. for 30 min. Pivaloyl chloride (29.0 mL, 28.2 g, 234 mmol, 1.1 equiv) was then added and the resulting slush was stirred at 0° C. for 40 min. A second portion of triethylamine (35.5 mL, 25.8 g, 255 mmol, 1.2 equiv) was added, followed by powdered (R,R)-pseudoephenamine (9) (48.30 g, 212.5 mmol) in 3 portions over 10 min. The resulting yellow suspension was stirred at 0° C. for 30 min then was warmed to 23° C. and was stirred at that temperature for 1 h. The reaction mixture was then quenched with water (600 mL) and was extracted with dichloromethane (3×500 mL). The combined organic layers were washed with a saturated aqueous solution of sodium bicarbonate (300 mL) and dried over sodium sulfate. The dried solution was concentrated in vacuo. The resulting beige solid was recrystallized from 50% ethyl acetate-hexanes (ca. 800 mL) to provide amide 10 (55.98 g, 74%, mp=111-112° C.) as white needles. The supernatant was concentrated in vacuo and was subjected to another round of recrystallization from 50% ethyl acetate-hexanes (ca. 100 mL) to provide additional amide 10 (7.97 g, 10%). The supernatant was again concentrated in vacuo and the residue was purified by flash chromatography on silica gel (30%→35% acetone-hexanes) to provide amide 10 (3.35 g, 4%) as a white powder. ¹H NMR (5:1 rotamer ratio, “major” and “minor”, respectively, 500 MHz, CDCl₃) δ: 7.42-7.16 (m, 10H), 5.70 (d, J=8.2 Hz, 0.8H, major), 5.37 (d, J=7.8 Hz, 1H), 5.17 (d, J=7.8 Hz, 0.2H, minor), 4.95-4.89 (m, 1H), 4.01-3.82 (m, 5H), 2.97 (s, 0.5H, minor), 2.90 (s, 2.5H, major), 2.56-2.39 (m, 2H), 2.09-2.00 (m, 2H); ¹³C NMR (5:1 rotamer ratio, asterisks [*] denote those minor rotameric peaks which could be resolved, 125 MHz, CDCl₃) δ: 174.4, 173.5*, 141.8, 137.2, 128.63*, 128.59, 128.5, 128.4, 127.9, 127.7, 127.6, 127.2*, 127.0, 103.8*, 103.6, 73.7, 73.4*, 65.7*, 65.2*, 65.0, 33.8, 29.4*, 29.0, 28.2, 27.5*; FTIR (neat), cm⁻¹: 3383, 2884, 1619, 1401, 1032; HRMS (ESI-TOF) m/z: [M+H]⁺Calcd for C₂₁H₂₆NO₄: 356.1862; Found 356.1862.

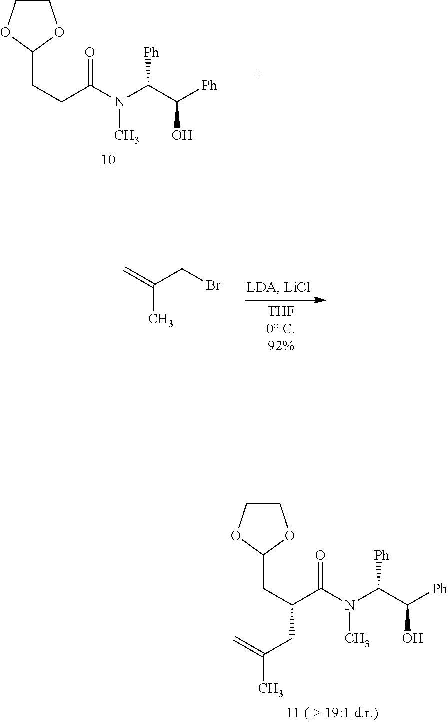

Alkylated amide 11: Lithium chloride (21.59 g, 509 mmol, 6 equiv) was dried by flame-heating under vacuum for 3 min and backfilling the headspace with argon. The dried lithium chloride was suspended in freshly distilled tetrahydrofuran (265 mL) at 23° C. and diisopropylamine (27.0 mL, 19.33 g, 191 mmol, 2.25 equiv) was added. The resulting suspension was cooled to −78° C. and a solution of n-butyllithium (2.17 M in hexanes, 82.0 mL, 178 mmol, 2.1 equiv) was added by syringe in 4 portions over 20 min. The resulting mixture was warmed to 0° C. for 10 min and was then cooled back to −78° C. A solution of amide 10 (30.17 g, 84.9 mmol) in tetrahydrofuran (250 mL) was added via cannula over 45 min, and tetrahydrofuran (10 mL) was used to quantitate the transfer. The reaction mixture was stirred at −78° C. for 30 min, then at 0° C. for 15 min, and at 23° C. for 5 min, after which it was cooled back to 0° C. Methallyl bromide (17.0 mL, 22.92 g, 170 mmol, 2 equiv) was then added and the suspension was stirred at 0° C. for 1.5 h. The mixture was quenched with a saturated aqueous solution of ammonium chloride (300 mL) and water (300 mL) and was warmed to 23° C. The mixture was extracted with ethyl acetate (3×600 mL) and the combined organic layers were washed with a saturated aqueous solution of sodium chloride (500 mL) and dried over sodium sulfate. The dried solution was concentrated in vacuo and the residue was purified by flash chromatography on silica gel (35%→50% ethyl acetate—hexanes) to provide alkylated amide 11 (31.95 g, 92%) as a thick colorless gum. ¹H NMR (500 MHz, CDCl₃) δ: 7.44 (d, J=7.7 Hz, 2H), 7.32-7.17 (m, 8H), 5.96 (d, J=9.3 Hz, 1H), 5.34 (dd, J=3.9, 9.3 Hz, 1H), 4.85 (t, J=4.6 Hz, 1H), 4.75 (s, 1H), 4.73 (s, 1H), 4.02-3.83 (m, 5H), 3.16-3.07 (m, 1H), 2.96 (s, 3H), 2.33-2.20 (m, 2H), 2.13 (dd, J=7.4, 14.0 Hz, 1H), 1.80 (dt, J=4.1, 14.0 Hz, 1H), 1.68 (s, 3H); ¹³C NMR (125 MHz, CDCl₃) δ: 177.3, 142.3, 141.2, 136.8, 128.8, 128.3, 128.2, 127.7, 127.5, 127.3, 112.9, 103.0, 73.0, 64.8, 64.7, 64.0, 41.1, 35.8, 35.3, 32.6, 22.5; FTIR (neat), cm⁻¹: 3409, 2965, 2882, 1624, 1450, 1065; HRMS (ESI-TOF) m/z: [M+H]⁺ Calcd for C₂₅H₃₂NO_{4:} 410.2331; Found 410.2323.

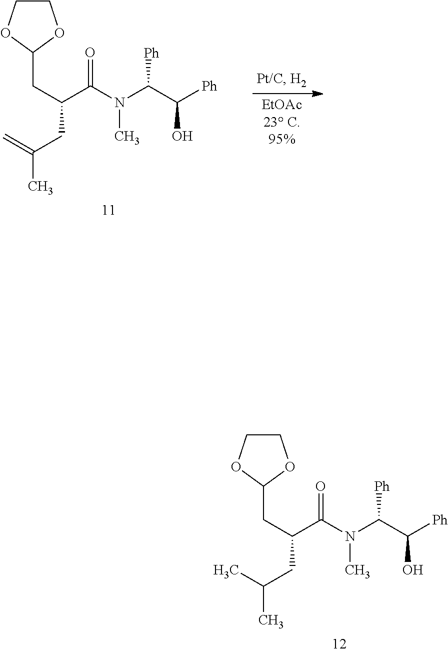 [SMILES: C=C(C)C[C@H](CC1OCCO1)C(=O)N(C)[C@H](c1ccccc1)[C@H](O)c1ccccc1 ; CC(C)C[C@H](CC1OCCO1)C(=O)N(C)[C@H](c1ccccc1)[C@H](O)c1ccccc1]

α-Isobutyl amide 12: Olefin 11 (31.95 g, 78.0 mmol) was dissolved in ethyl acetate (780 mL) under an argon atmosphere, and 5% platinum on carbon (1.52 g, 76 mg of platinum, 0.39 mmol, 0.005 equiv) was added. The reaction mixture was sparged with hydrogen for 3 h and then stirred under hydrogen (1 atm) for a further 16 h. The reaction vessel was flushed with argon and was filtered through a Celite pad, washing with ethyl acetate (500 mL) (note: the Celite pad containing platinum on carbon was moistened with water and disposed of in a designated waste container). The filtrate was concentrated in vacuo to provide amide 12 (30.49 g, 95%) as a thick colorless oil. ¹H NMR (13.5:1 rotamer ratio, “major” and “minor”, respectively, 500 MHz, CDCl₃) δ: 7.41 (d, J=7.3 Hz, 2H), 7.30-7.12 (m, 8H), 5.91 (d, J=8.6 Hz, 1H), 5.29 (d, J=8.6 Hz, 1H), 4.76 (t, J=4.7 Hz, 1H), 4.30 (br s, 1H), 3.08-2.85 (m, 4H), 2.14 (ddd, J=4.7, 9.7, 14.0 Hz, 1H), 1.70 (ddd, J=3.8, 4.7, 14.0 Hz, 1H), 1.50-1.41 (m, 2H), 1.28-1.21 (m, 1H), 0.97 and 0.95 (2 d, J=6.4 Hz, 0.4H, minor), 0.82 and 0.80 (2 d, J=6.0 Hz, 5.6H, major); ¹³C NMR (13.5:1 rotamer ratio, only the major rotamer is reported, 125 MHz, CDCl₃) δ: 177.8, 141.5, 137.0, 128.6, 128.3, 128.2, 127.5, 127.4, 127.1, 102.9, 72.9, 64.75, 64.68, 64.2, 42.5, 36.4, 35.0, 33.0, 25.5, 22.7, 22.6; FTIR (neat), cm⁻¹: 3391, 2968, 2887, 1619, 1447, 1064; HRMS (ESI-TOF) m/z: [M+H]⁺ Calcd for C₂₅H₃₄NO_{4:} 412.2488; Found 412.2479.

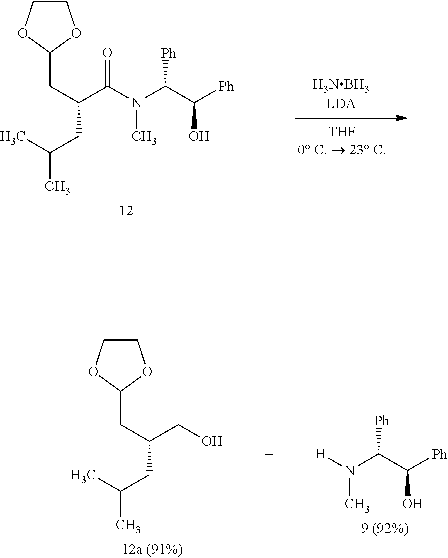 [SMILES: CC(C)C[C@H](CC1OCCO1)C(=O)N(C)[C@H](c1ccccc1)[C@H](O)c1ccccc1 ; CC(C)C[C@@H](CO)CC1OCCO1 ; CN[C@H](c1ccccc1)[C@H](O)c1ccccc1]

Alcohol 12a and recovered (R,R)-pseudoephenamine (9): n-Butyllithium (2.03 M in hexanes, 141 mL, 286 mmol, 3.9 equiv) was added in 4 portions by syringe over 20 min to an ice-cooled solution of diisopropylamine (43.5 mL, 31.2 g, 308 mmol, 4.2 equiv) in tetrahydrofuran (650 mL). The resulting mixture was stirred at 0° C. for 20 min and ammonia borane complex (90%, 10.06 g, 9.06 g of ammonia borane complex, 294 mmol, 4 equiv) was added in 5 portions over 20 min. The resulting white suspension was stirred at 0° C. for 15 min. A solution of amide 12 (30.20 g, 73.4 mmol) in tetrahydrofuran (235 mL) was added via cannula over 30 min, and tetrahydrofuran (15 mL) was used to quantitate the transfer. The resulting homogeneous solution was stirred at 0° C. for 30 min, then at 23° C. for 3 h. The reaction mixture was cooled to 0° C. and was carefully quenched with water (200 mL) (note: hydrogen evolution). The mixture was warmed to 23° C. for 12 h and was concentrated in vacuo to remove tetrahydrofuran. Dichloromethane (800 mL) was added and the aqueous layer was acidified to pH 1 using a 6 M aqueous solution of HCl (ca. 50 mL) (additional hydrogen evolution). The layers were separated and the aqueous phase was further extracted with dichloromethane (2×600 mL). The combined organic layers were washed with water (800 mL), dried over sodium sulfate, and the dried solution was concentrated in vacuo. The residue was purified by flash chromatography on silica gel (25%×40% ethyl acetate-hexanes) to provide alcohol 12a (12.52 g, 91%) as a colorless oil. ¹H NMR (500 MHz, CDCl₃) δ: 4.94 (dd, J=3.8, 5.3 Hz, 1H), 4.03-3.95 (m, 2H), 3.90-3.81 (m, 2H), 3.61 (dd, J=3.8, 11.2 Hz, 1H), 3.45 (dd, J=6.4, 11.2 Hz, 1H), 2.68 (br s, 1H), 1.86-1.75 (m, 2H), 1.71-1.58 (m, 2H), 1.23-1.16 (m, 1H), 1.15-1.07 (m, 1H), 0.88 (t, J=6.6 Hz, 6H); ¹³C NMR (125 MHz, CDCl₃) δ: 103.9, 66.5, 65.1, 64.9, 41.5, 36.6, 34.8, 25.3, 23.0, 22.7; FTIR (neat), cm⁻¹: 3423, 2953, 2870, 1467, 1124, 1031; HRMS (ESI-TOF) m/z: [M+Na]⁺ Calcd for C₁₀H₂₀O₃Na: 211.1310; Found 211.1303.

Recovery of (R,R)-pseudoephenamine (9): The combined aqueous layers from above were adjusted to pH 14 using a 2 M aqueous solution of sodium hydroxide (ca. 250 mL) and the basified solution was extracted with dichloromethane (3×600 mL). The combined organic layers were dried over sodium sulfate and the dried solution was concentrated in vacuo to provide (R,R)-pseudoephenamine (9) (15.41 g, 92%) as a white powder. ¹H NMR (500 MHz, CDCl₃) δ: 7.24-6.99 (m, 10), 4.58 (d, J=8.6 Hz, 1H), 3.51 (d, J=8.6 Hz, 1H), 3.13 (br s, 1H), 2.30 (s, 3H).

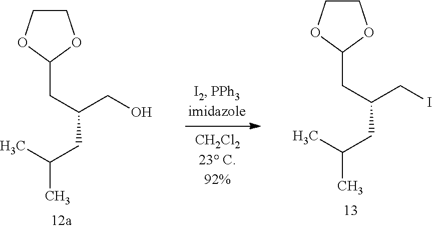

Alkyl iodide 13: Triphenylphosphine (18.87 g, 71.9 mmol, 1.1 equiv) was dissolved in dichloromethane (220 mL) and the solution was held at 23° C. in a water bath. Solid iodine (18.68 g, 73.6 mmol, 1.13 equiv) was added in 2 portions over 10 min to this solution and the resulting dark solution was stirred at 23° C. for 10 min. Solid imidazole (8.24 g, 121 mmol, 1.85 equiv) was added and the resulting yellow suspension was stirred at 23° C. for a further 10 min. A solution of alcohol 12a (12.314 g, 65.4 mmol) in dichloromethane (100 mL) was added via cannula over 10 min, and dichloromethane (10 mL) was used to quantitate the transfer. The solution was stirred at 23° C. for 1.5 h. The reaction mixture was quenched with a saturated aqueous solution of sodium thiosulfate (300 mL). The biphasic mixture was stirred at 23° C. for 30 min, after which water (200 mL) was added. The mixture was extracted with dichloromethane (3×500 mL) and the combined organic layers were dried over sodium sulfate. The dried solution was concentrated in vacuo to provide a crude mixture consisting of a white solid and a colorless oil. The mixture was suspended in 10% ethyl acetate-hexanes (200 mL) and was sonicated at 23° C. for 10 min, while pulverizing the clumps of solid with a metal spatula. The suspension was filtered through a sintered glass funnel, and the solid was washed with a second portion of 10% ethyl acetate-hexanes (200 mL). The filtrate was concentrated in vacuo and the residue was purified by flash chromatography on silica gel (1%→5% ethyl acetate-hexanes) to provide iodide 13 (18.017 g, 92%) as a colorless oil. ¹H NMR (500 MHz, CDCl₃) δ: 4.86 (t, J=5.1 Hz, 1H), 3.98-3.88 (m, 2H), 3.84-3.75 (m, 2H), 3.34 (dd, J=4.1, 9.9 Hz, 1H), 3.30 (dd, J=4.1, 9.9 Hz, 1H), 1.69-1.51 (m, 3H), 1.48-1.38 (m, 1H), 1.20 (dt, J=7.0, 13.9 Hz, 1H), 1.12 (dt, J=7.0, 13.9 Hz, 1H), 0.87 (d, J=6.1 Hz, 3H), 0.85 (d, J=6.1 Hz, 3H); ¹³C NMR (125 MHz, CDCl₃) δ: 103.1, 64.9, 64.8, 44.4, 38.5, 32.3, 24.8, 23.1, 22.2, 17.3; FTIR (neat), cm⁻¹: 2952, 2869, 1131, 1044; HRMS (ESI-TOF) m/z: [M+H]⁺ Calcd for C₁₀H₂₀IO₂: 299.0508; Found 299.0500.

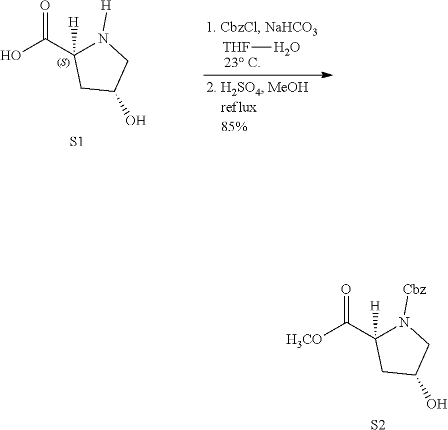

Cbz-Hyp-OCH₃ (S2): Benzyl chloroformate (70.0 mL, 84.0 g, 492 mmol, 1.3 equiv) was added dropwise over 1 h to a solution of trans-4-hydroxy-L-proline (S1) (50.0 g, 381 mmol) and sodium bicarbonate (41.5 g, 494 mmol, 1.3 equiv) in a mixture of tetrahydrofuran (325 mL) and water (100 mL) at 23° C. The reaction mixture was stirred at 23° C. for 6 h and was quenched by slow addition of a 20% aqueous solution of potassium hydroxide (250 mL). The resulting solution was diluted with ethyl ether (650 mL) and water (200 mL) and the mixture was vigorously shaken. The layers were allowed to separate and the organic layer was discarded. The aqueous layer was acidified to pH 2 using a 2 N aqueous solution of sulfuric acid (ca. 400 mL) and was extracted with ethyl acetate (3×700 mL). The combined organic layers were washed with a saturated aqueous solution of sodium chloride (500 mL) and dried over sodium sulfate. The dried solution was concentrated in vacuo to provide the crude carbamate (92.64 g) as a thick colorless oil which was used without further purification.

The crude carbamate was dissolved in methanol (500 mL) and concentrated sulfuric acid (10 mL) was added. The solution was refluxed for 12 h and was cooled to 23° C. The reaction mixture was quenched with a saturated aqueous solution of sodium bicarbonate (300 mL) and the mixture was concentrated in vacuo to remove methanol. The aqueous mixture was extracted with ethyl acetate (3×800 mL) and the combined organic layers were washed with a saturated aqueous solution of sodium chloride (500 mL). The organic solution was dried over sodium sulfate and the dried solution was concentrated in vacuo to provide ester S2 (90.04 g, 85% over 2 steps) as a thick colorless oil. ¹H NMR (1:1 rotamer ratio, 500 MHz, CDCl₃) δ: 7.46-7.14 (m, 5H), 5.22-4.95 (m, 2H), 4.58-4.37 (m, 2H), 3.84-3.46 (m, 5H), 2.47-2.22 (m, 2H), 2.16-2.02 (m, 1H); ¹³C NMR (1:1 rotamer ratio, 125 MHz, CDCl₃) δ: 173.3, 173.2, 155.2, 154.7, 136.4, 136.3, 128.54, 128.48, 128.11, 128.10, 127.93, 127.86, 70.0, 69.3, 67.4, 67.3, 58.0, 57.8, 55.3, 54.7, 52.4, 52.2, 39.2, 38.4; FTIR (neat), cm⁻¹: 3474, 2995, 1744, 1686, 1415, 1354, 1202, 1167, 733, 698.

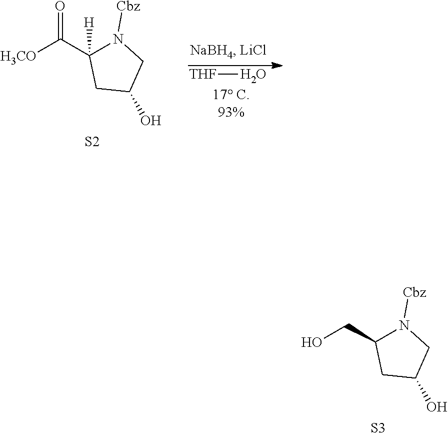

Diol S3: Lithium chloride (17.75 g, 419 mmol, 1.3 equiv) was added to a solution of ester S2 (89.94 g, 322 mmol) in a mixture of tetrahydrofuran (900 mL) and water (59 mL) at 17° C. To the resulting solution was added sodium borohydride (15.84 g, 419 mmol, 1.3 equiv) in 5 portions over 2 h. The reaction mixture was stirred at 17° C. for 1 h, after which additional lithium chloride (17.75 g, 419 mmol, 1.3 equiv) was added, followed by sodium borohydride (15.84 g, 419 mmol, 1.3 equiv) in 5 portions over 2 h. The solution was stirred at 17° C. for 5 h and a third portion of lithium chloride (7.10 g, 168 mmol, 0.5 equiv) and sodium borohydride (6.34 g, 168 mmol, 0.5 equiv) was added. The mixture was stirred at 23° C. for 12 h, at which point thin-layer chromatography indicated complete consumption of starting material. The solution was cooled to 5° C. and was carefully quenched using a 2 M aqueous solution of HCl (450 mL) over a period of 1 h. A saturated aqueous solution of sodium bicarbonate (200 mL) was added and tetrahydrofuran was removed in vacuo. The aqueous mixture was extracted with ethyl acetate (5×500 mL) and the combined organic layers were washed with a saturated aqueous solution of sodium chloride (500 mL). The organic phase was dried over sodium sulfate, and the dried solution was concentrated in vacuo to provide diol S3 (74.30 g, 93%) as a thick colorless oil. ¹H NMR (mixture of rotamers, 500 MHz, CDCl₃) δ: 7.56-7.18 (m, 5H), 5.22-5.01 (m, 2H), 4.86 (br s, 1H), 4.44-4.29 (m, 1H), 4.20-4.02 (m, 1H), 3.83-3.12 (m, 5H), 2.09-1.96 (m, 1H), 1.77-1.64 (m, 1H); ¹³C NMR (mixture of rotamers, only major is reported, 125 MHz, CDCl₃) δ: 157.3, 136.3, 128.6, 128.2, 128.0, 69.2, 67.5, 66.1, 59.3, 55.6, 37.4; FTIR (neat), cm⁻¹: 3384, 2945, 1669, 1419, 1357, 1117, 1049, 732, 697.

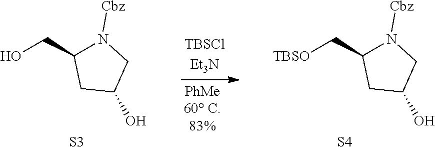 [SMILES: O=C(OCc1ccccc1)N1C[C@H](O)C[C@H]1CO ; CC(C)(C)[Si](C)(C)OC[C@@H]1C[C@@H](O)CN1C(=O)OCc1ccccc1 ; "S3" ; "S4"]

Silyl ether S4: tert-Butyldimethylsilyl chloride (53.5 g, 355 mmol, 1.2 equiv) was added to a solution of diol S3 (74.30 g, 296 mmol) and triethylamine (62.0 mL, 44.9 g, 444 mmol, 1.2 equiv) in toluene (430 mL) at 23° C. The resulting solution was stirred at 60° C. for 18 h as white solid precipitated. The resulting suspension was cooled to 23° C. and was quenched with water (600 mL) and the biphasic mixture was stirred for 1 h at 23° C. The layers were separated and the aqueous phase was further extracted with toluene (200 mL). The combined organic layers were washed with a saturated aqueous solution of sodium chloride (200 mL) and dried over sodium sulfate. The dried solution was concentrated in vacuo and the residue was purified by flash chromatography on silica gel (15%→40% ethyl acetate-hexanes) to provide silyl ether S4 (89.94 g, 83%) as a colorless oil. ¹H NMR (1:1 rotamer ratio, 500 MHz, CDCl₃) δ: 7.38-7.28 (m, 5H), 5.23-5.03 (m, 2H), 4.50-4.39 (m, 1H), 4.12-4.01 (m, 1H), 3.97 (dd, J=3.9, 10.2 Hz, 0.5H), 3.71 (dd, J=5.1, 10.2 Hz, 0.5H), 3.65-3.40 (m, 3H), 2.94 (br s, 0.5H), 2.79 (br s, 0.5H), 2.27-2.14 (m, 1H), 2.05-1.92 (m, 1H), 0.87 and 0.85 (2 s, 9H), 0.03, 0.00, −0.04, −0.05 (4 s, 6H); ¹³C NMR (1:1 rotamer ratio, 125 MHz, CDCl₃) δ: 155.4, 155.1, 136.9, 136.6, 128.6, 128.5, 128.1, 127.9, 127.8, 70.2, 69.6, 67.1, 66.7, 64.0, 62.8, 57.9, 57.5, 55.8, 55.3, 37.3, 36.5, 25.9, 18.2, −5.4, −5.5; FTIR (neat), cm⁻¹: 3433, 2952, 2856, 1702, 1677, 1414, 1099; HRMS (ESI-TOF) m/z: [M+H]⁺ Calcd for C₁₉H₃₂NO₄Si: 366.2095; Found 366.2097.

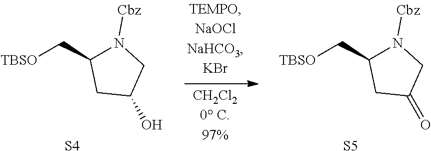 [SMILES: CC(C)(C)[Si](C)(C)OC[C@@H]1C[C@@H](O)CN1C(=O)OCc1ccccc1 ; CC(C)(C)[Si](C)(C)OC[C@@H]1CC(=O)CN1C(=O)OCc1ccccc1 ; "S4" ; "S5"]

Ketone S5: Potassium bromide (138 mg, 1.16 mmol, 0.015 equiv), sodium bicarbonate (12.30 g, 146 mmol, 1.9 equiv), and 2,2,6,6,-tetramethyl-1-piperidinyloxy, free radical (121 mg, 0.77 mmol, 0.01 equiv) were added to a solution of alcohol S4 (28.20 g, 77.0 mmol) in unstabilized dichloromethane (120 mL) at 0° C. To the resulting suspension was added an aqueous solution of sodium hypochlorite (5%, 285 mL, 193 mmol, 2.5 equiv) over a period of 1.5 h. Thin-layer chromatography indicated that some starting material remained, thus, additional sodium bicarbonate (2.46 g, 29.3 mmol, 0.38 equiv) was added, followed by additional sodium hypochlorite solution (5%, 60 mL, 38.5 mmol, 0.5 equiv) over a period of 30 min. The suspension was stirred at 0° C. for a further 30 min, at which point an orange color persisted and thin-layer chromatography indicated complete consumption of starting material. The reaction mixture was quenched with a saturated aqueous solution of sodium thiosulfate (100 mL) and was extracted with dichloromethane (3×200 mL). The combined organic layers were dried over magnesium sulfate and the dried solution was concentrated in vacuo to provide ketone S5 (27.40 g, 75.0 mmol, 97%) as a colorless oil that solidified to an off-white solid on standing. ¹H NMR (1.5:1 rotamer ratio, “major” and “minor”, respectively, 500 MHz, CDCl₃) δ: 7.42-7.26 (m, 5H), 5.29-5.11 (m, 2H), 4.46 (d, J=9.5 Hz, 0.6H, major), 4.40 (d, J=9.5 Hz, 0.4H, minor), 4.06 (d, J=10.0 Hz, 0.6H, major), 3.97-3.84 (m, 1.4H), 3.76 (s, 0.6H, major), 3.73 (s, 0.4H, minor), 3.56 (d, J=10.2 Hz, 1H), 2.73-2.61 (m, 1H), 2.46 (br s, 0.6H, major), 2.42 (br s, 0.4H, minor), 0.83 (s, 9H), 0.02 to −0.06 (m, 6H); ¹³C NMR (1.5:1 rotamer ratio, asterisks [*] denote those minor rotameric peaks which could be resolved, 125 MHz, CDCl₃) δ: 209.8*, 209.4, 154.4, 154.1*, 136.4, 128.6*, 128.5, 128.3*, 128.2, 128.1, 127.9, 67.3*, 67.1, 65.8*, 64.9, 55.8, 55.7*, 53.8*, 53.6, 40.7*, 40.1, 25.7, 18.0, −5.7, −5.8; FTIR (neat), cm⁻¹: 2952, 2928, 2856, 1763, 1703, 1413, 1098; HRMS (ESI-TOF) m/z: [M+H]⁺ Calcd for C₁₉H₃₀NO₄Si: 364.1939; Found 364.1938.

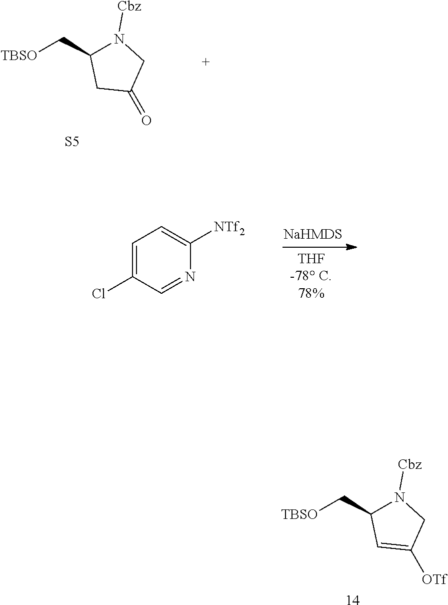

Vinyl triflate 14: A solution of sodium hexamethyldisilazide in tetrahydrofuran (1 M, 83.0 mL, 83.0 mmol, 1.5 equiv) was diluted with tetrahydrofuran (165 mL) and the resulting solution was cooled to −78° C. To this mixture was added a solution of ketone S8 (20.0 g, 55.0 mmol) in tetrahydrofuran (83 mL) over 10 min via cannula, and tetrahydrofuran (5 mL) was used to quantitate the transfer. The mixture was stirred at −78° C. for 30 min and a solution of Comins' reagent (27.0 g, 68.8 mmol, 1.25 equiv) in tetrahydrofuran (83 mL) was added over 10 min via cannula, and tetrahydrofuran (5 mL) was used to quantitate the transfer. The solution was stirred for 1 h at −78° C., at which point thin-layer chromatography indicated complete consumption of the starting material. The reaction mixture was quenched with water (300 mL) at −78° C. and was warmed to 23° C. The mixture was extracted with hexanes (1×500 mL, 2×250 mL) and the combined organic layers were dried over sodium sulfate. The dried solution was concentrated in vacuo and the residue was purified by flash chromatography on silica gel (2%→5%→10% ethyl acetate-hexanes) to provide separately pure enol triflate 7 (16.10 g, 59%) as a colorless oil and an impure fraction (ca. 10 g). The impure fraction was repurified by flash chromatography on silica gel (5% ethyl acetate-hexanes) to provide additional pure enol triflate 14 (5.10 g, 19%). ¹H NMR (1.5:1 rotamer ratio, “major” and “minor”, respectively, 500 MHz, CDCl₃) δ: 7.48-7.27 (m, 5H), 5.83-5.65 (m, 1H), 5.27-5.08 (m, 2H), 4.75-4.66 (m, 0.6H, major), 4.66-4.57 (m, 0.4H, minor),4.40 (d, J=14.6 Hz, 0.4H, minor), 4.34 (d, J=14.3 Hz, 0.6H, major), 4.27-4.17 (m, 1H), 3.93-3.78 (m, 1.6H), 3.68 (dd, J=6.1, 10.1 Hz, 0.4H, minor), 0.87 (s, 5.4H, major), 0.85 (s, 3.6H, minor), 0.05 to −0.06 (m, 6H); ¹³C NMR (1.5:1 rotamer ratio, asterisks [*] denote those minor rotameric peaks which could be resolved, 125 MHz, CDCl₃) δ: 154.2*, 154.0, 143.7*, 143.2, 136.3, 136.1*, 128.73, 128.68, 128.5, 128.42, 128.40, 128.2, 118.6 (q, J=321 Hz), 115.4, 115.3, 67.7*, 67.4, 64.1, 64.0*, 63.6*, 62.7, 51.3*, 51.0, 25.8, 18.2, −5.47, −5.54; FTIR (neat), cm⁻¹: 3035, 2858, 1711, 1414, 1211, 1101, 833; HRMS (ESI-TOF) m/z: [M+H]⁺ Calcd for C₂₀H₂₉F₃NO₆SSi: 496.1434; Found 496.1430.

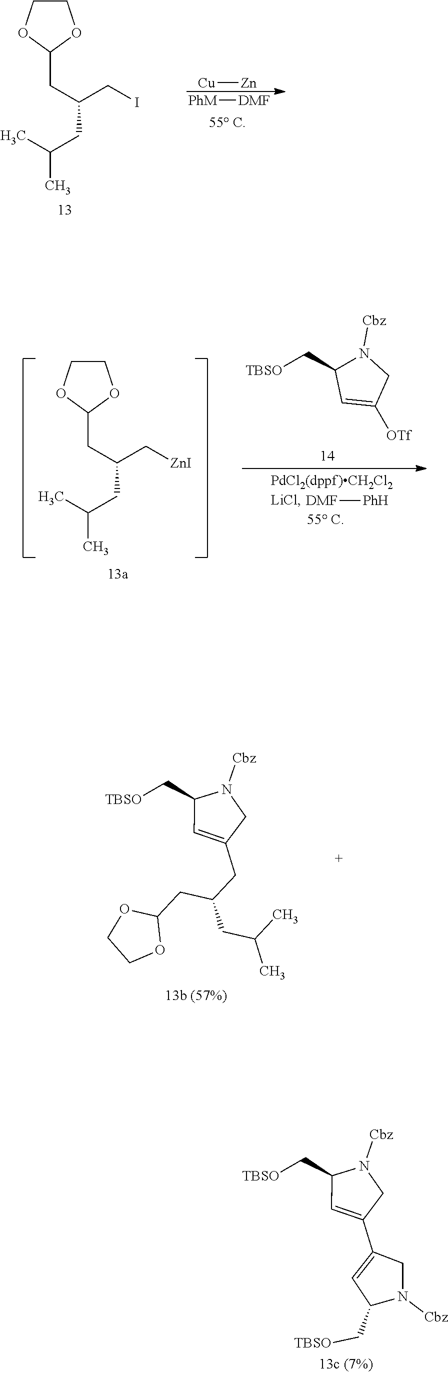

Coupled product 13b and diene 13c: Copper (II) acetate monohydrate (636 mg, 3.19 mmol, 0.1 equiv) was suspended in acetic acid (17 mL) and the mixture was heated to 100° C. Activated zinc dust (10.4 g, 159 mmol, 5.0 equiv) was added to the hot mixture and the resulting thick suspension was stirred/shaken by hand at 100° C. for an additional 2 min. The suspension was cooled to 23° C., and additional acetic acid (30 mL) was added. The liquid was removed via syringe and the solid was washed with a second portion of acetic acid (30 mL), followed by three portions of ethyl ether (40 mL), removing the solvent by syringe after each wash. The remaining solid was dried at ca. 1 mmHg at 55° C. for 2 h to give a free flowing, reddish copper-zinc couple.

To the freshly prepared copper-zinc couple under an argon atmosphere was added a solution of alkyl iodide 13 (10.0 g, 31.9 mmol, 1 equiv) in a mixture of degassed benzene (93 mL) and degassed N,N-dimethylformamide (8 mL) via cannula at 23° C. Degassed benzene (8 mL) was used to quantitate the transfer. The suspension was heated to 55° C. for 1 h. Completion of the zinc insertion was verified by ¹H NMR analysis of an aliquot that was quenched with water, extracted with ethyl ether, dried over magnesium sulfate, and briefly concentrated in vacuo. The suspension was left unagitated to allow the residual copper-zinc couple settle in order to make cannulation more facile.

Lithium chloride (2.03 g, 47.9 mmol, 1.5 equiv) was thoroughly flame dried at ca. 1 mmHg for 2 min and was cooled to room temperature at the same pressure. After backfilling the headspace with argon, [1,1′-bis(diphenylphosphino)ferrocene]dichloropalladium (II), complex with dichloromethane (521 mg, 0.683 mmol, 0.02 equiv) was added, followed by a solution of vinyl triflate 14 (17.4 g, 35.1 mmol, 1.1 equiv) in degassed N,N-dimethylformamide (76 mL) via cannula at 23° C. Degassed N,N-dimethylformamide (5 mL) was used to quantitate the transfer. The organozinc solution in benzene-N,N-dimethylformamide was then added via cannula, using N,N-dimethylformamide (5 mL) to quantitate the transfer. The resulting black solution was stirred at 55° C. for 20 h and then was cooled to 23° C. The reaction mixture was quenched with a saturated aqueous solution of sodium chloride (300 mL) and was extracted with ethyl ether (3×300 mL). The combined organic phases were washed with a 10% aqueous solution of lithium chloride (2×300 mL) and dried over sodium sulfate. The dried solution was concentrated in vacuo and the residue was purified by flash column chromatography on silica gel (5%→20% ethyl acetate-hexanes) to provide separately cross-coupled product 13b (9.48 g, 57%) as a colorless oil, recovered enol triflate 14 (5.7 g, 36% reisolated starting material), and dimeric diene 13c (802 mg, 7%) as a pale yellow oil. 13b: ¹H NMR (1:1 rotamer ratio, 500 MHz, CDCl₃) δ: 7.40-7.27 (m, 5H), 5.53-5.47 (m, 1H), 5.22-5.08 (m, 2H), 4.88 (t, J=4.9 Hz, 1H), 4.57 (br s, 0.5H), 4.49 (br s, 0.5H), 4.18-4.05 (m, 1H), 4.04-3.86 (m, 3.5H), 3.85-3.76 (m, 2.5H), 3.71 (dd, J=6.1, 9.6 Hz, 0.5H), 3.50 (dd, J=7.0, 9.6 Hz, 0.5H), 2.22-2.02 (m, 2H), 1.89-1.75 (m, 1H), 1.68-1.55 (m, 3H), 1.20-1.10 (m, 2H), 0.92-0.78 (m, 15H), 0.02, 0.01, and −0.06 (3 s, 6H); ¹³C NMR (1:1 rotamer ratio, 125 MHz, CDCl₃) δ: 154.7, 154.5, 138.6, 138.4, 137.1, 136.8, 128.6, 128.5, 128.2, 128.1, 128.0, 127.9, 123.3, 103.67, 103.65, 67.0, 66.6, 66.5, 65.8, 65.0, 64.8, 63.7, 56.6, 56.1, 44.1, 44.0, 38.3, 38.2, 34.2, 30.3, 25.97, 25.95, 25.3, 23.1, 23.0, 22.70, 22.67, 18.3, −5.3, −5.4; FTIR (neat), cm⁻¹: 2952, 2927, 2855, 1706, 1411, 1102; HRMS (ESI-TOF) m/z: [M+H]⁺ Calcd for C₂₉H₄₈NO₅Si: 518.3302; Found 518.3295. 13c: ¹H NMR (1:1 rotamer ratio, 500 MHz, CDCl₃) δ: 7.47-7.25 (m, 10H), 5.74-5.62 (4 s, 2H), 5.27-5.10 (m, 4H), 4.69 (d, J=11.7 Hz, 1H), 4.61 (d, J=14.7 Hz, 1H), 4.46 (d, J=14.2 Hz, 1H), 4.38 (d, J=14.0 Hz, 1H), 4.30-4.20 (m, 2H), 3.94 (td, J=3.1, 10.2 Hz, 1H), 3.87 (ddd, J=3.3, 6.5, 9.8 Hz, 1H), 3.75 (dt, J=5.2, 10.2 Hz, 1H), 3.54 (ddd, J=2.0, 6.9, 9.3 Hz, 1H), 0.87-0.82 (2 s, 18H), 0.04 to −0.07 (3 s, 6H); ¹³C NMR (1:1 rotamer ratio, 125 MHz, CDCl₃) δ: 154.6, 154.5, 136.9, 136.6, 132.52, 132.47, 128.7, 128.6, 128.3, 128.24, 128.16, 128.1, 125.9, 125.8, 125.5, 125.3, 76.9, 67.3, 67.2, 67.0, 66.9, 66.3, 64.7, 64.6, 63.4, 54.11, 54.07, 53.64, 53.61, 26.0, 25.9, 18.3, −5.3, −5.4; FTIR (neat), cm⁻¹: 2928, 1703, 1254, 1214, 1104, 836, 734, 697; HRMS (ESI-TOF) m/z: [M+H]⁺ Calcd for C₃₈H₅₇N₂O₆Si₂: 693.3750; Found 693.3739.

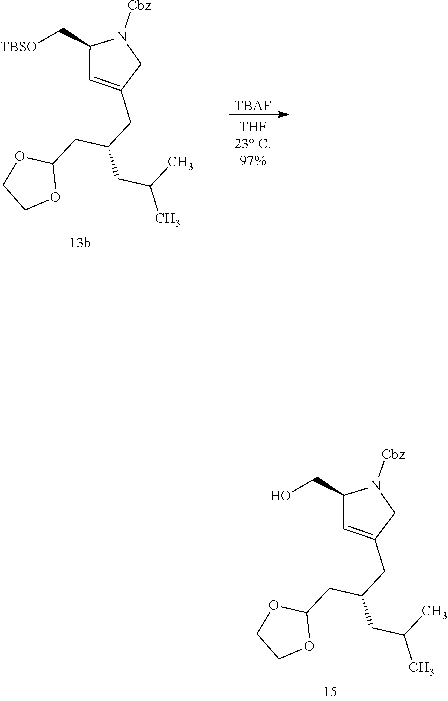 [SMILES: CC(C)C[C@@H](CC1=C[C@@H](CO[Si](C)(C)C(C)(C)C)N(C(=O)OCc2ccccc2)C1)CC1OCCO1 ; "TBAF" ; CC(C)C[C@@H](CC1=C[C@@H](CO)N(C(=O)OCc2ccccc2)C1)CC1OCCO1]

Homoallylic alcohol 15: A solution of tetra(n-butyl)ammonium fluoride in tetrahydrofuran (1.0 M, 29 mL, 29 mmol, 1.6 equiv) was added to a solution of silyl ether 13b (9.48 g, 18.31 mmol) in tetrahydrofuran (185 mL) at 23° C. The solution was stirred at 23° C. for 1.5 h and was quenched with a saturated aqueous solution of ammonium chloride (200 mL). The mixture was extracted with ethyl acetate (3×300 mL) and the combined organic phases were washed with a saturated aqueous solution of sodium chloride (300 mL). The organic layer was dried over sodium sulfate and the dried solution was concentrated in vacuo. The residue was purified by flash chromatography on silica gel (30%→60% ethyl acetate-hexanes) to provide homoallylic alcohol 15 (7.20 g, 97%) as a light yellow oil. ¹H NMR (3:1 rotamer ratio, “major” and “minor”, respectively, 500 MHz, CDCl₃) δ: 7.41-7.28 (m, 5H), 5.35 (br s, 0.25H, minor), 5.30 (br s, 0.75H, major), 5.18 (d, J=12.4 Hz, 1H), 5.14 (d, J=12.4 Hz, 1H), 4.86 (t, J=4.9 Hz, 1H), 4.75 (br s, 0.75H, major), 4.61 (br s, 0.25H, minor), 4.24 (br s, 1H), 4.16-4.05 (m, 2H), 3.96-3.88 (m, 2H), 3.83-3.74 (m, 3H), 3.58 (dd, J=6.8, 11.5 Hz, 1H), 2.16-2.05 (m, 2H), 1.82-1.71 (m, 1H), 1.68-1.51 (m, 3H), 1.23-1.05 (m, 2H), 0.86 and 0.85 (2 d, J=6.7 Hz, 6H); ¹³C NMR (3:1 rotamer ratio, asterisks [*] denote those minor rotameric peaks which could be resolved, 125 MHz, CDCl₃) δ: 156.6, 154.7*, 140.5*, 139.8, 136.5, 128.6, 128.2, 128.1, 128.0, 121.8*, 121.2, 103.5*, 103.4, 68.3, 67.4, 67.2*, 66.8, 66.4*, 64.8, 64.4*, 56.5*, 55.9, 44.6*, 44.3, 38.2, 38.1*, 34.5*, 34.3, 30.2, 30.1*, 25.2, 22.9, 22.80*, 22.78*, 22.7; FTIR (neat), cm⁻¹: 3400, 2952, 1685, 1413, 1113, 1029, 698; HRMS (ESI-TOF) m/z: [M+H]⁺ Calcd for C₂₃H₃₄NO₅: 404.2437; Found 404.2429.

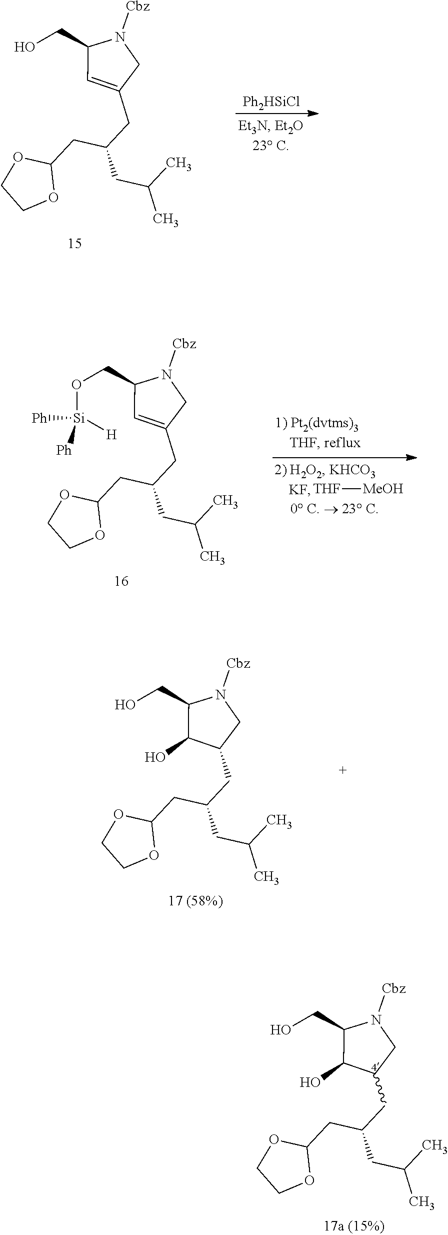

Diol 17: Chlorodiphenylsilane (3.6 mL, 4.02 g, 18.4 mmol, 1.05 equiv) was added to a solution of homoallylic alcohol 15 (7.07 g, 17.5 mmol) and triethylamine (3.7 mL, 2.69 g, 26.3 mmol, 1.5 equiv) in ethyl ether (350 mL) at 23° C. The resulting suspension was stirred at 23° C. for 1 h then was filtered through a Celite pad, washing with additional ethyl ether (100 mL). The filtrate was concentrated in vacuo to provide crude silyl ether 16, which was used without further purification and had characterization data identical to those described above.

The crude silyl ether 16 was dissolved in freshly distilled tetrahydrofuran (700 mL) and the solution was heated to reflux. A solution of Karstedt's catalyst (˜2% in xylenes, 2.0 mL, 34.2 mg of platinum, 0.175 mmol, 0.01 equiv) was added and the solution was heated at reflux for 3.5 h, then was cooled to 0° C. Methanol (700 mL) was added, followed by sequential addition of a 30% aqueous solution of hydrogen peroxide (36 mL, 350 mmol, 20 equiv), potassium bicarbonate (5.26 g, 52.6 mmol, 3 equiv), and potassium fluoride (10.2 g, 175 mmol, 10 equiv). The resulting suspension was warmed to 23° C. and was stirred at that temperature for 18 h. The reaction mixture was filtered through a sintered glass funnel, rinsing the solid with methanol (ca. 100 mL). The filtrate was diluted with water (200 mL) and was concentrated in vacuo to a volume of ca. 500 mL. Additional water (300 mL) was added and the mixture was extracted with dichloromethane (700 mL, then 2×500 mL). The combined organic layers were dried over magnesium sulfate and the dried solution was concentrated in vacuo. The residue was purified by flash chromatography on silica gel (40%→100% ethyl acetate-hexanes) to provide separately diol 17 (4.31 g, 58%) as a colorless oil that solidified on standing and an inseparable mixture of homoallylic alcohol 6 and hydrogenation product 17a (2.42 g, 1.3:1 mixture of 15-17a by ¹H NMR spectroscopy, 20% of 15, 15% of 17a) as a colorless oil. Hydrogenation/Protodesilylation Product S18: ¹H NMR (600 MHz, CDCl₃) δ: 7.49-7.29 (m, 5H), 5.20-5.06 (m, 2H), 4.85 (t, J=4.4 Hz, 1H), 4.12-4.06 (m, 1H), 3.98-3.90 (m, 2H), 3.86-3.77 (m, 2H), 3.68-3.54 (m, 3H), 3.06 (dd, J=8.1, 10.7 Hz, 1H), 2.30 (p, J=7.8 Hz, 1H), 1.80-1.72 (m, 1H), 1.72-1.65 (m, 1H), 1.64-1.52 (m, 4H), 1.39-1.32 (m, 1H), 1.33-1.23 (m, 1H), 1.21-1.12 (m, 1H), 1.13-1.05 (m, 1H), 0.92-0.80 (m, 6H);¹³C NMR (125 MHz, CDCl₃) δ: 157.4, 136.6, 128.6, 128.2, 128.0, 103.6, 67.6, 67.4, 64.8, 60.2, 53.0, 44.5, 38.5, 34.9, 34.7, 30.6, 29.8, 25.3, 23.0, 22.9; FTIR (neat), cm⁻¹: 3451, 2952, 2869, 1681, 1414, 1356, 1118, 1042, 732; HRMS (ESI-TOF) m/z: [M+H]⁺ Calcd for C₂₃H₃₆NO₅: 406.2588; Found 406.2582.

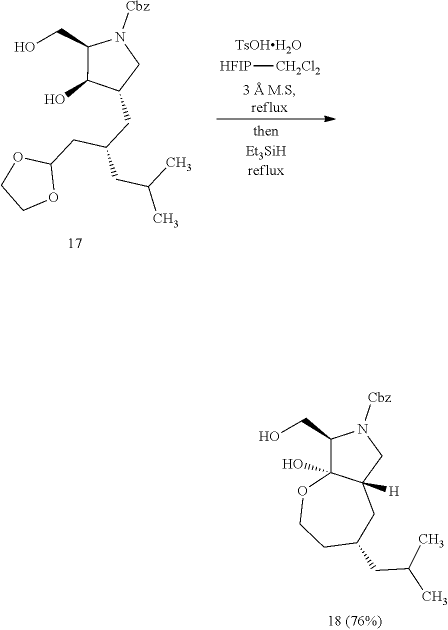

Oxepane 18: Freshly activated molecular sieves (3 Å, 8-12 mesh beads, 26.5 g) were suspended in a mixture of dichloromethane (265 mL) and 1,1,1,3,3,3-hexafluoroisopropanol (93 mL, 148 g, 882 mmol, 100 equiv) and p-toluenesulfonic acid monohydrate (2.85 g, 15 mmol, 1.7 equiv) was added at 23° C. The mixture was stirred at 23° C. for 30 min, after which solid dioxolane 17 (3.720 g, 8.82 mmol) was added. The mixture was refluxed for 3 h, after which thin-layer chromatography indicated complete consumption of dioxolane 17. Triethylsilane (11.5 mL, 8.21 g, 70.6 mmol, 8 equiv) was added to the refluxing solution, followed by a second portion of p-toluenesulfonic acid monohydrate (2.85 g, 15 mmol, 1.7 equiv), and the reaction mixture was refluxed for a further 30 min. The suspension was cooled to 23° C. and was filtered through a sintered glass funnel. The filtrate was quenched with a saturated aqueous solution of sodium bicarbonate (300 mL). The mixture was extracted with dichloromethane (3×300 mL) and the combined organic layers were dried over sodium sulfate. The dried solution was concentrated in vacuo and the residue was purified by flash chromatography on silica gel (20%→30% ethyl acetate-hexanes→100% acetone) to provide oxepane 18 (2.431 g, 76%) as a colorless oil that solidified to an amorphous white solid on standing. ¹H NMR (1:1 rotamer ratio, 500 MHz, CDCl₃) δ: 7.39-7.28 (m, 5H), 5.17-5.06 (m, 2H), 4.17-4.00 (m, 2H), 3.97-3.57 (m, 5H), 3.21 (br s, 0.5H), 2.97 (br s, 0.5H), 2.93-2.85 (m, 1H), 2.41-2.29 (m, 1H), 1.96 and 1.89 (2 d, J=13.5 Hz, 1H), 1.80-1.56 (m, 4H), 1.18-1.09 (m, 2H), 0.94-0.80 (m, 7H); ¹³C NMR (1:1 rotamer ratio, 125 MHz, CDCl₃) δ: 155.7, 155.0, 136.7, 128.60, 128.58, 128.13, 128.10, 128.0, 82.1, 81.0, 68.3, 68.1, 67.1, 62.5, 62.3, 60.6, 59.2, 50.2, 49.9, 47.1, 42.0, 40.7, 38.5, 35.6, 35.5, 35.0, 34.8, 25.3, 22.9, 22.7; FTIR (neat), cm⁻¹: 3451, 2951, 2910, 2867, 1699, 1413, 1356, 1100; HRMS (ESI-TOF) m/z: [M+H]⁺ Calcd for C₂₁H₃₂NO₄: 362.2331; Found 362.2324.

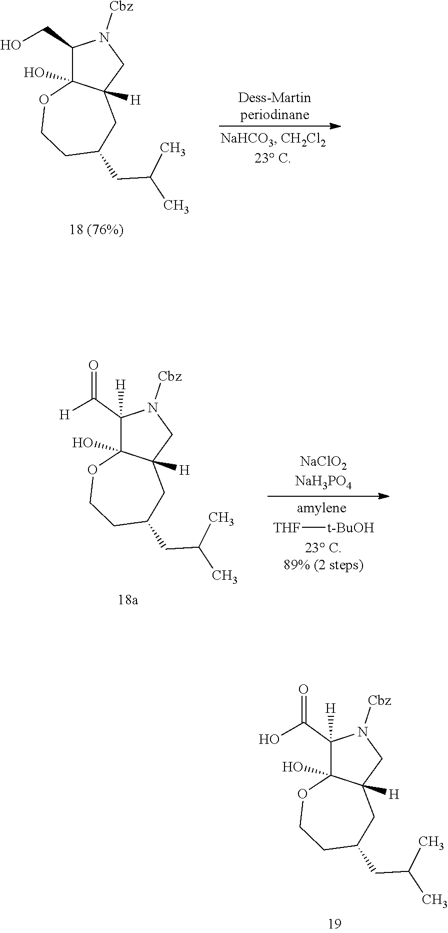

Acid 19: Sodium bicarbonate (6.69 g, 80 mmol, 12 equiv) and Dess-Martin periodinane (8.44 g, 19.9 mmol, 3 equiv) were added to a solution of alcohol 18 (2.398 g, 6.63 mmol) in dichloromethane (135 mL) at 23° C. and the resulting suspension was stirred at that temperature for 1 h. The reaction mixture was quenched with a saturated aqueous solution of sodium thiosulfate (200 mL) and was stirred at 23° C. for 45 min. A saturated aqueous solution of sodium bicarbonate (200 mL) was added and the mixture was extracted with dichloromethane (3×200 mL). The combined organic layers were dried over sodium sulfate and the dried solution was concentrated in vacuo to provide the crude aldehyde 18a, which was used without further purification. For characterization purposes, a sample was purified by flash chromatography on silica gel (15%→20% ethyl acetate-hexanes) to provide aldehyde 18a as a colorless oil. ¹H NMR (1:1 rotamer ratio, 500 MHz, CDCl₃) δ: 9.70 (s, 0.5H), 9.62 (s, 0.5H), 7.40-7.25 (m, 5H), 5.19-5.00 (m, 2H), 4.66 (d, J=8.4 Hz, 0.5H), 4.54 (d, J=8.4 Hz, 0.5H), 4.28 (ddd, J=3.2, 8.4, 10.0 Hz, 1H), 3.96-3.82 (m, 2H), 3.76 (td, J=3.2, 11.5 Hz, 1H), 2.98 (q, J=11.0 Hz, 1H), 2.33-3.18 (m, 1H), 1.96 (dt, J=3.7, 13.8 Hz, 0.5H), 1.90 (dt, J=3.7, 13.4 Hz, 0.5H), 1.78-1.70 (m, 1H), 1.68-1.54 (m, 2H), 1.21-1.07 (m, 2H), 0.94-0.83 (m, 7H); ¹³C NMR (1:1 rotamer ratio, 125 MHz, CDCl₃) δ: 199.9, 199.5, 154.6, 154.3, 136.6, 136.4, 128.6, 128.5, 128.2, 128.1, 128.0, 127.9, 82.4, 81.9, 68.34, 68.31, 67.3, 67.2, 66.5, 66.4, 50.2, 50.0, 47.00, 46.96, 42.6, 42.0, 38.03, 38.00, 35.33, 35.31, 35.21, 35.19, 25.2, 22.9, 22.7, 22.6; FTIR (neat), cm⁻¹: 2952, 2912, 1736, 1703, 1414, 1357, 1099, 734, 697; HRMS (ESI-TOF) m/z: [M+H]⁺ Calcd for C₂₁H₃₀NO₄: 360.2175; Found 360.2166.

The crude aldehyde 18a (6.63 mmol) was dissolved in a mixture of tetrahydrofuran (200 mL), tert-butanol (51 mL) and amylene (42 mL, 27.9 g, 398 mmol, 60 equiv) at 23° C. Sodium dihydrogen phosphate (3.82 g, 32 mmol, 4.8 equiv) was added, followed by an aqueous solution of sodium chlorite (1.0 M, 32 mL, 32 mmol, 4.8 equiv). The solution was stirred at 23° C. for 2 h, after which it was diluted with dichloromethane (300 mL) and water (300 mL). The aqueous layer was acidified to pH 3 using a 1 M aqueous solution of HCl (ca. 10 mL) and the layers were separated. The aqueous layer was further extracted with dichloromethane (2×200 mL) and the combined organic layers were dried over sodium sulfate. The dried solution was concentrated in vacuo and the residue was purified by flash chromatography on silica gel (0%→4% methanol-dichloromethane) to provide acid 19 (2.218 g, 89% over 2 steps) as a white powder. ¹H NMR (1:1 rotamer ratio, 600 MHz, CDCl₃) δ: 10.61 (br s, 1H), 7.63-7.19 (m, 5H), 5.20-5.03 (m, 2H), 4.55 (d, J=8.2 Hz, 0.5H), 4.49 (d, J=8.2 Hz, 0.5H), 4.15 (dt, J=5.2, 8.4 Hz, 1H), 3.99-3.82 (m, 2H), 3.80-3.72 (m, 1H), 2.98 (td, J=3.0, 10.6 Hz, 1H), 2.66-2.53 (m, 1H), 1.96 (d, J=14.1 Hz, 0.5H), 1.90 (d, J=14.1 Hz, 0.5H), 1.79-1.49 (m, 4H), 1.19-1.04 (m, 2H), 0.94-0.75 (m, 7H); ¹³C NMR (1:1 rotamer ratio, 150 MHz, CDCl₃) δ: 175.4, 174.9, 154.7, 154.3, 136.4, 136.3, 128.50, 128.46, 128.1, 128.0, 127.9, 127.6, 81.0, 80.4, 68.2, 68.1, 67.4, 67.3, 62.4, 62.2, 50.4, 50.3, 46.9, 41.2, 40.3, 38.0, 35.5, 35.3, 35.2, 25.2, 22.8, 22.63, 22.59; FTIR (neat), cm⁻¹: 3549, 3034, 1744, 1707, 1418, 1359, 1061; HRMS (ESI-TOF) m/z: [M+H]⁺ Calcd for C₂₁H₃₀NO₅: 376.2124; Found 376.2116.

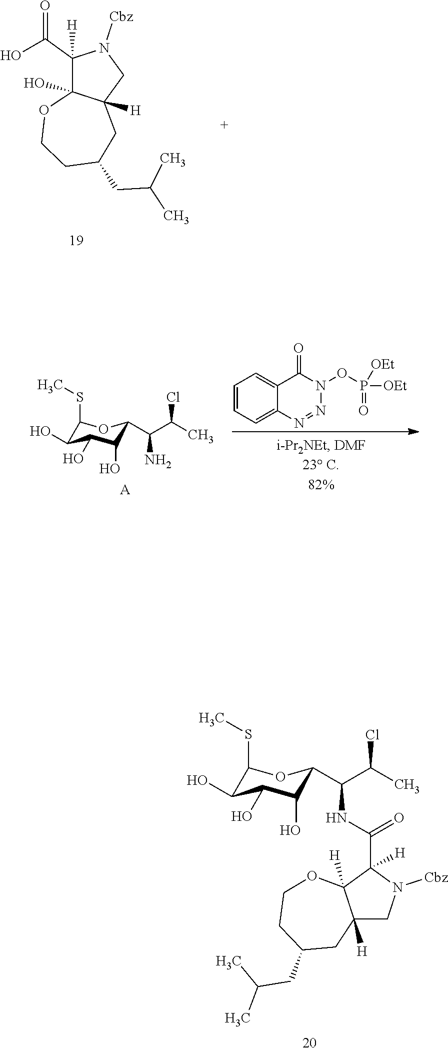

Amide 20: Acid 19 (2.016 g, 5.37 mmol) and amine A (2.043 g, 7.52 mmol, 1.4 equiv) were dissolved in a mixture of N,N-dimethylformamide (54 mL) and N,N-diisopropylethylamine (3.8 mL, 2.78 g, 21.5 mmol, 4 equiv) at 23° C. 3-(Diethoxyphosphoryloxy)-1,2,3-benzotriazin-4(3H)-one (DEPBT) (2.089 g, 6.98 mmol, 1.3 equiv) was added and the resulting bright yellow solution was stirred at 23° C. for 20 h. The reaction mixture was diluted with dichloromethane (200 mL), water (100 mL), and a saturated aqueous solution of sodium carbonate (100 mL). The layers were separated and the aqueous layer was further extracted with dichloromethane (2×150 mL). The combined organic layers were washed with a 5% aqueous solution of citric acid (200 mL) and the aqueous layer was further extracted with dichloromethane (150 mL). The combined organic layers were dried over sodium sulfate and the dried solution was concentrated in vacuo at 50° C. The residue was purified by flash chromatography on silica gel (25%→35% acetone-hexanes) to provide amide 20 (2.774 g, 82%) as a white powder. ¹H NMR (1:1 rotamer ratio, 500 MHz, D₃COD) δ: 7.42-7.23 (m, 5H), 5.34 (d, J=12.7 Hz, 0.5H), 5.28 (d, J=5.6 Hz, 0.5H), 5.24 (d, J=5.6 Hz, 0.5H), 5.13 (d, J=12.4 Hz, 0.5H), 5.10 (d, J=12.4 Hz, 0.5Hz), 4.95 (d, J=12.6 Hz, 0.5H), 4.68 (d, J=8.2 Hz, 0.5H), 4.59-4.49 (m, 1.5H), 4.45 (dd, J=1.7, 10.0 Hz, 0.5H), 4.34 (dd, J=1.8, 10.1 Hz, 0.5H), 4.27 (d, J=10.0 Hz, 0.5H), 4.20 (ddd, J=3.1, 8.2, 10.4 Hz, 1H), 4.08 (dd, J=5.6, 10.2 Hz, 0.5H), 4.05-3.98 (m, 1.5H), 3.94-3.69 (m, 3H), 3.67 (d, J=3.4 Hz, 0.5H), 3.60 (dd, J=3.4, 10.1 Hz, 0.5H), 3.25 (dd, J=3.4, 10.1 Hz, 0.5H), 2.97 (td, J=6.8, 10.6 Hz, 1H), 2.52 (pentet, 10.6 Hz, 1H), 2.14 and 2.13 (2s, 3H), 1.96 (d, J=13.6 Hz, 0.5H), 1.91 (d, J=13.4 Hz, 0.5H), 1.74 (d, J=13.7 Hz, 1H), 1.70-1.57 (m, 3H), 1.52 (2d, J=6.8 Hz, 3H), 1.21-1.09 (m, 2H), 1.00-0.92 (m, 1H), 0.96-0.84 (m, 6H); ¹³C NMR (1:1 rotamer ratio, 125 MHz, CDCl₃) δ: 172.1, 171.8, 155.0, 154.2, 136.3, 136.0, 128.6, 128.5, 128.3, 128.0, 127.9, 127.8, 87.8, 80.8, 80.2, 71.0, 70.8, 70.0, 68.9, 67.9, 67.8, 67.5, 63.6, 63.5, 57.8, 57.5, 53.6, 53.4, 50.6, 50.5, 46.9, 41.0, 40.3, 38.0, 35.6, 35.3, 25.1, 22.8, 22.5, 21.8, 13.4; FTIR (neat), cm⁻¹: 3413, 2921, 1688, 1665, 1385, 1094, 727; HRMS (ESI-TOF) m/z: [M+H]⁺ Calcd for C₃₀H₄₆ClN₂O₈S: 629.2663; Found 629.2655.

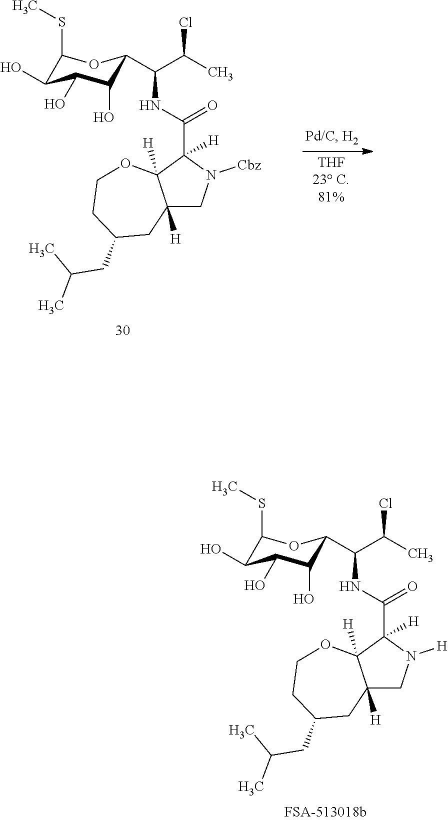

FSA-513018b: 20 (2.357 g, 3.75 mmol) was dissolved in tetrahydrofuran (120 mL) under an argon atmosphere. 10% Palladium on carbon (3.99 g, 399 mg of palladium, 3.75 mmol, 1.0 equiv) was added and the suspension was sparged with hydrogen for 5 min. The reaction mixture was then stirred under hydrogen (1 atm) for 5 h, after which the headspace was flushed with argon. The suspension was filtered through a Celite pad. The filter cake was sequentially washed with dichloromethane (150 mL), methanol (150 mL), and a 1:9 (v/v) mixture of a saturated aqueous solution of ammonium hydroxide:methanol (450 mL) (note: the Celite pad containing palladium on carbon was moistened with water and disposed of in a designated waste container). The filtrate was concentrated in vacuo and the residue was purified by flash chromatography on silica gel (4%→10% methanol-dichloromethane+0.1% saturated aqueous ammonium hydroxide throughout) to provide FSA-513018b (1.496 g, 81%) as a white powder. ¹H NMR (500 MHz, CDCl₃) δ: 5.29 (d, J=5.6 Hz, 1H), 4.59 (q, J=6.5 Hz, 1H), 4.40 (dd, J=1.6, 10.0 Hz, 1H), 4.22-4.13 (m, 2H), 4.08 (dd, J=5.6, 10.2 Hz, 1H), 3.94 (ddd, J=2.7, 5.0, 11.8 Hz, 1H), 3.91-3.87 (m, 2H), 3.79 (td, J=2.7, 10.2 Hz, 1H), 3.57 (dd, J=3.4, 10.2 Hz, 1H), 3.22 (dd, J=7.2, 10.1 Hz, 1H), 2.52 (t, J=10.6 Hz, 1H), 2.22-2.15 (m, 1H), 2.14 (s, 3H), 1.94 (dd, J=3.5, 13.5 Hz, 1H), 1.77-1.61 (m, 4H), 1.51 (d, J=6.7 Hz, 3H), 1.22-1.10 (m, 2H), 0.93-0.81 (m, 7H); ¹³C NMR (125 MHz, D₃COD) δ: 174.7, 89.6, 83.5, 71.9, 71.2, 69.7, 69.6, 68.9, 64.0, 59.2, 54.4, 51.2, 48.3, 44.6, 38.3, 37.3, 36.0, 26.3, 23.2, 23.0, 22.6, 13.3; FTIR (neat), cm⁻¹: 3317, 2954, 2919, 1663, 1531, 1256, 1083, 1055, 731; HRMS (ESI-TOF) m/z: [M+H]⁺ Calcd for C₂₂H₄₀ClN₂O₆S: 495.2290; Found 495.2289.

A sample (1.312 g) was recrystallized from ethanol to provide fine white needles (1.027 g, 78% recovery, mp=205-206° C.). The mother liquor was concentrated to a white solid, which was recrystallized from ethanol to provide an additional 155 mg (12% recovery) of white needles. Single crystals suitable for X-ray diffraction analysis were obtained by subjecting a sample of the above material to a second recrystallization from ethanol. The recrystallized material had characterization data identical to those described above. Anal. Calcd for C₂₂H₃₉ClN₂O₆S: C, 53.37; H, 7.94; N, 5.66. Found: C, 53.12; H, 7.59; N, 5.50.

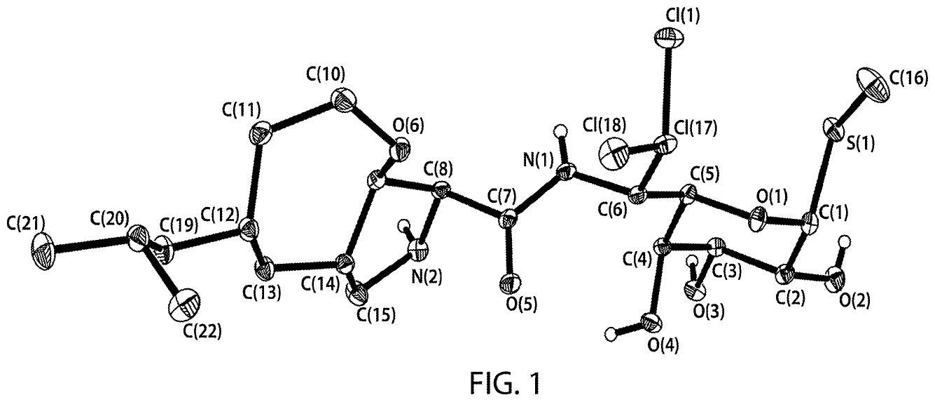

X-ray Crystallography: A crystal mounted on a diffractometer was collected data at 100 K. The intensities of the reflections were collected by means of a Bruker APEX II CCD diffractometer (Mo_{Kα} radiation, X=0.71073 Å), and equipped with an Oxford Cryosystems nitrogen flow apparatus. The collection method involved 0.5° scans in ω at 28° in 2θ. Data integration down to 0.78 Å resolution was carried out using SAINT V8.37A (Bruker diffractometer, 2016) with reflection spot size optimization. Absorption corrections were made with the program SADABS (Bruker diffractometer, 2016). The structure was solved by the Intrinsic Phasing methods and refined by least-squares methods again F² using SHELXT-2014 (Sheldrick, 2015) and SHELXL-2014 (Sheldrick, 2015) with OLEX 2 interface. Non-hydrogen atoms were refined anisotropically, and hydrogen atoms were allowed to ride on the respective atoms. Crystal data as well as details of data collection and refinement are summarized in Table A, geometric parameters are shown in Table B, and hydrogen-bond parameters are listed in Table C. The Ortep plots produced with SHELXL-2014 program, and the other drawings were produced with Accelrys DS Visualizer 2.0 (Accelrys, 2007). FIG. 1 shows a depiction of the crystal structure.

TABLE A
Experimental details

FSA-513018b

Crystal data

| | |
|---|---|
| Chemical formula | C₂₂H₃₉ClN₂O₆S |
| Mᵣ | 495.06 |
| Crystal system, space group | Monoclinic, C2 |
| Temperature (K) | 100 |
| a, b, c (Å) | 30.4694 (14), 5.1579 (2), 16.4988 (8) |
| b (°) | 105.6838 (7) |
| V (Å³) | 2496.38 (19) |
| Z | 4 |
| Radiation type | Mo Ka |
| m (mm⁻¹) | 0.28 |
| Crystal size (mm) | 0.26 × 0.12 × 0.08 |

Data collection

| | |
|---|---|
| Diffractometer | Bruker D8 goniometer with CCD area detector |
| Absorption correction | Multi-scan |
| | SADABS |
| Tₘᵢₙ, Tₘₐₓ | 0.827, 0.862 |
| No. of measured, independent | 13009, 5434, 5196 |
| and observed [I > 2 s(I)] | |
| reflections | |
| Rᵢₙₜ | 0.016 |
| (sin q/l)ₘₐₓ (Å⁻¹) | 0.641 |

Refinement

| | |
|---|---|
| R[F² > 2 s(F²)], wR(F²), S | 0.025, 0.062, 1.02 |
| No. of reflections | 5434 |
| No. of parameters | 313 |
| No. of restraints | 1 |
| H-atom treatment | H atoms treated by a mixture of |
| | independent and constrained refinement |
| Dρₘₐₓ, Dρₘᵢₙ (e Å⁻³) | 0.27, −0.16 |
| Absolute structure | Flack × determined using 2176 |
| | quotients [(I+) − (I−)]/[(I+) + (I−)] |
| | (Parsons, Flack and Wagner, Acta |
| | Cryst. B69 (2013) 249-259). |
| Absolute structure parameter | −0.001 (17) |

Computer programs: SAINT 8.37A (Bruker-AXS, 2015), SHELXT2014 (Sheldrick, 2015), SHELXL2014 (Sheldrick, 2015), Bruker SHELXTL (Sheldrick, 2015).

TABLE B
Geometric parameters (Å, °)

| | | | |
|---|---|---|---|
| C11—C17 | 1.813 (2) | C10—C11 | 1.534 (3) |
| S1—C16 | 1.800 (2) | C10—H10A | 0.9900 |
| S1—C1 | 1.838 (2) | C10—H10B | 0.9900 |
| O1—C1 | 1.408 (2) | C11—C12 | 1.539 (3) |
| O1—C5 | 1.438 (2) | C11—H11A | 0.9900 |
| O2—C2 | 1.424 (2) | C11—H11B | 0.9900 |
| O2—H2 | 0.73 (4) | C12—C13 | 1.535 (2) |
| O3—C3 | 1.422 (2) | C12—C19 | 1.539 (2) |
| O3—H3 | 0.89 (3) | C12—H12 | 1.0000 |
| O4—C4 | 1.426 (2) | C13—C14 | 1.521 (2) |
| O4—H4 | 0.85 (3) | C13—H13A | 0.9900 |
| O5—C7 | 1.222 (2) | C13—H13B | 0.9900 |
| O6—C9 | 1.414 (2) | C14—C15 | 1.531 (3) |
| O6—C10 | 1.430 (2) | C14—H14 | 1.0000 |
| N1—C7 | 1.350 (2) | C15—H15A | 0.9900 |
| N1—C6 | 1.451 (2) | C15—H15B | 0.9900 |
| N1—H1 | 0.82 (3) | C16—H16A | 0.9800 |
| N2—C8 | 1.483 (2) | C16—H16B | 0.9800 |
| N2—C15 | 1.493 (2) | C16—H16C | 0.9800 |
| N2—H2A | 0.88 (2) | C17—C18 | 1.517 (3) |
| C1—C2 | 1.530 (2) | C17—H17 | 1.0000 |
| C1—H1A | 1.0000 | C18—H18A | 0.9800 |
| C2—C3 | 1.524 (3) | C18—H18B | 0.9800 |
| C2—H2B | 1.0000 | C18—H18C | 0.9800 |
| C3—C4 | 1.530 (2) | C19—C20 | 1.540 (2) |
| C3—H3A | 1.0000 | C19—H19A | 0.9900 |
| C4—C5 | 1.529 (2) | C19—H19B | 0.9900 |
| C4—H4A | 1.0000 | C20—C22 | 1.524 (3) |
| C5—C6 | 1.527 (2) | C20—C21 | 1.527 (3) |
| C5—H5 | 1.0000 | C20—H20 | 1.0000 |
| C6—C17 | 1.527 (2) | C21—H21A | 0.9800 |
| C6—H6 | 1.0000 | C21—H21B | 0.9800 |
| C7—C8 | 1.518 (3) | C21—H21C | 0.9800 |
| C8—C9 | 1.536 (2) | C22—H22A | 0.9800 |
| C8—H8 | 1.0000 | C22—H22B | 0.9800 |
| C9—C14 | 1.526 (2) | C22—H22C | 0.9800 |
| C9—H9 | 1.0000 | | |
| C16—S1— C1 | 97.90 (10) | C12—C11— H11A | 107.8 |
| C1—O1—C5 | 114.85 (13) | C10—C11—H11B | 107.8 |
| C2—O2—H2 | 105 (3) | C12—C11—H11B | 107.8 |
| C3—O3—H3 | 106.8 (17) | H11A—C11—H11B | 107.2 |
| C4—O4—H4 | 105.2 (19) | C13—C12—C19 | 108.88 (14) |
| C9—O6—C10 | 114.52 (14) | C13—C12—C11 | 111.41 (15) |
| C7—N1—C6 | 121.05 (16) | C19—C12—C11 | 110.14 (16) |
| C7—N1—H1 | 117.3 (16) | C13—C12—H12 | 108.8 |
| C6—N1—H1 | 121.4 (16) | C19—C12—H12 | 108.8 |
| C8—N2—C15 | 108.66 (13) | C11—C12—H12 | 108.8 |
| C8—N2—H2A | 106.6 (14) | C14—C13—C12 | 114.03 (15) |
| C15—N2—H2A | 108.3 (14) | C14—C13—H13A | 108.7 |
| O1—C1—C2 | 111.43 (14) | C12—C13—H13A | 108.7 |
| O1—C1—S1 | 113.08 (13) | C14—C13—H13B | 108.7 |
| C2—C1—S1 | 109.90 (13) | C12—C13—H13B | 108.7 |
| O1—C1—H1A | 107.4 | H13A—C13—H13B | 107.6 |
| C2—C1—H1A | 107.4 | C13—C14—C9 | 114.60 (16) |
| S1—C1—H1A | 107.4 | C13—C14—C15 | 114.97 (15) |
| O2—C2—C3 | 112.93 (15) | C9—C14—C15 | 103.04 (15) |
| O2—C2—C1 | 112.22 (15) | C13—C14—H14 | 108.0 |
| C3—C2—C1 | 109.68 (15) | C9—C14—H14 | 108.0 |
| C2—C2—H2B | 107.2 | C15—C14—H14 | 108.0 |
| C3—C2—H2B | 107.2 | N2—C15—C14 | 106.46 (14) |
| C1—C2—H2B | 107.2 | N2—C15—H15A | 110.4 |
| O3—C3—C2 | 111.18 (15) | C14—C15—H15A | 110.4 |
| O3—C3—C4 | 111.40 (14) | N2—C15—H15B | 110.4 |
| C2—C3—C4 | 108.78 (14) | C14—C15—H15B | 110.4 |
| O3—C3—H3A | 108.5 | H15A—C15—H15B | 108.6 |
| C2—C3—H3A | 108.5 | S1—C16—H16A | 109.5 |
| C4—C3—H3A | 108.5 | S1—C16—H16B | 109.5 |
| O4—C4—C5 | 110.28 (14) | H16A—C16—H16B | 109.5 |
| O4—C4—C3 | 110.23 (15) | S1—C16—H16C | 109.5 |
| C5—C4—C3 | 108.61 (14) | H16A—C16—H16C | 109.5 |
| O4—C4—H4A | 109.2 | H16B—C16—H16C | 109.5 |
| C5—C4—H4A | 109.2 | C18—C17—C6 | 112.63 (15) |
| C3—C4—H4A | 109.2 | C18—C17—C11 | 109.59 (14) |
| O1—C5—C6 | 105.37 (13) | C6—C17—C11 | 109.28 (13) |
| O1—C5—C4 | 110.87 (14) | C18—C17—H17 | 108.4 |
| C6—C5—C4 | 112.48 (14) | C6—C17—H17 | 108.4 |
| O1—C5—H5 | 109.3 | C11—C17—H17 | 108.4 |
| O6—C5—H5 | 109.3 | C17—C18—H18A | 109.5 |
| C4—C5—H5 | 109.3 | C17—C18—H18B | 109.5 |
| N1—C6—C17 | 112.21 (15) | H18A—C18—H18B | 109.5 |
| N1—C6—C5 | 109.13 (14) | C17—C18—H18C | 109.5 |
| C17—C6—C5 | 114.73 (14) | H18A—C18—H18C | 109.5 |
| N1—C6—H6 | 106.8 | H18B—C18—H18C | 109.5 |
| C17—C6—H6 | 106.8 | C12—C19—C20 | 116.47 (15) |
| C5—C6—H6 | 106.8 | C12—C19—H19A | 108.2 |
| O5—C7—N1 | 122.68 (17) | C20—C19—H19A | 108.2 |
| O5—C7—C8 | 121.90 (16) | C12—C19—H19B | 108.2 |
| N1— C7—C8 | 115.37 (16) | C20—C19—H19B | 108.2 |
| N2—C8—C7 | 108.67 (15) | H19A—C19—H19B | 107.3 |
| N2—C8—C9 | 104.78 (13) | C22—C20—C21 | 110.20 (17) |
| C7—C8—C9 | 110.74 (14) | C22—C20—C19 | 110.79 (16) |
| N2—C8—H8 | 110.8 | C21—C20—C19 | 110.29 (15) |
| C7—C8—H8 | 110.8 | C22—C20—H20 | 108.5 |
| C9—C8—H8 | 110.8 | C21—C20—H20 | 108.5 |
| O6—C9—C14 | 112.14 (15) | C19—C20—H20 | 108.5 |
| O6—C9—C8 | 111.04 (14) | C20—C21—H21A | 109.5 |
| C14—C9—C8 | 103.61 (14) | C20—C21—H21B | 109.5 |
| O6—C9—H9 | 110.0 | H21A—C21—H21B | 109.5 |
| C14—C9—H9 | 110.0 | C20—C21—H21C | 109.5 |
| C8—C9—H9 | 110.0 | H21A—C21—H21C | 109.5 |
| O6—C10—C11 | 115.97 (15) | H21B—C21—H21C | 109.5 |
| O6—C10—H10A | 108.3 | C20—C22—H22A | 109.5 |
| C11— C10—H10A | 108.3 | C20—C22—H22B | 109.5 |
| O6—C10—H10B | 108.3 | H22A—C22—H22B | 109.5 |
| C11—C10—H10B | 108.3 | C20—C22—H22C | 109.5 |
| H10A—C10—H10B | 107.4 | H22A—C22—H22C | 109.5 |
| C10—C11—C12 | 117.93 (16) | H22B—C22—H22C | 109.5 |
| C10—C11—H1 1A | 107.8 | | |
| C5—O1—C1—C2 | −56.0 (2) | N1—C7—C8—N2 | 124.05 (16) |
| C5—O1—C1—S1 | 68.41 (16) | O5—C7—C8—C9 | 61.0 (2) |
| C16—S1—C1—O1 | 62.26 (15) | N1—C7—C8—C9 | −121.37 (17) |
| C16—S1—C1—C2 | −172.52 (14) | C10—O6—C9—C14 | −93.32 (18) |
| O1—C1—C2—O2 | −178.26 (16) | C10—O6—C9—C8 | 151.30 (15) |
| S1—C1—C2—O2 | 55.59 (18) | N2—C8—C9—O6 | 153.80 (15) |
| O1—C1—C2—C3 | 55.4 (2) | C7—C8—C9—O6 | 36.8 (2) |
| S1—C1—C2—C3 | −70.79 (16) | N2—C8—C9—C14 | 33.23 (18) |
| O2—C2—C3—O3 | 53.8 (2) | C7—C8—C9—C14 | −83.76 (17) |
| C1—C2—C3—O3 | 179.80 (14) | C9—O6—C10—C11 | 41.6 (2) |
| O2—C2—C3—C4 | 176.85 (15) | O6—C10—C11—C12 | 38.5 (3) |
| C1—C2—C3—C4 | −57.17 (18) | C10—C11— C12— | −82.6 (2) |
| | | C13 | |
| O3—C3—C4—O4 | 60.05 (19) | C10—C11— C12— | 156.46 (17) |
| | | C19 | |
| C2—C3—C4—O4 | −62.84 (18) | C19—C12—C13— | −171.28 (17) |
| | | C14 | |
| O3—C3—C4—C5 | −179.02 (15) | C11— C12—C13— | 67.0 (2) |
| | | C14 | |
| C2—C3—C4—C5 | 58.09 (19) | C12—C13—C14—C9 | −55.7 (2) |
| C1—O1—C5—C6 | 179.19 (14) | C12—C13—C14— | −174.85 (16) |
| | | C15 | |
| C1—O1—C5—C4 | 57.23 (19) | O6—C9—C14—C13 | 78.32 (19) |
| O4—C4—C5—O1 | 63.88 (18) | C8—C9—C14—C13 | −161.86 (15) |
| C3—C4—C5—O1 | −57.01 (19) | O6—C9—C14—C15 | −156.04 (15) |
| O4—C4—C5—C6 | −53.82 (19) | C8—C9—C14—C15 | −36.23 (18) |
| C3—C4—C5—C6 | −174.71 (15) | C8—N2—C15—C14 | −5.7 (2) |
| C7— N1— C6—C17 | −108.98 (19) | C13—C14—C15—N2 | 151.62 (16) |
| C7—N1— C6—C5 | 122.71 (18) | C9—C14—C15—N2 | 26.2 (2) |
| O1— C5—C6—N1 | −179.20 (14) | N1—C6—C17—C18 | 61.4 (2) |
| C4—C5—C6—N1 | −58.29 (19) | C5—C6—C17—C18 | −173.29 (17) |
| O1—C5—C6—C17 | 53.91 (19) | N1—C6—C17—C11 | −60.63 (17) |
| C4—C5—C6—C17 | 174.82 (16) | C5—C6—C17—C11 | 64.66 (18) |
| C6—N1—C7—O5 | 2.5 (3) | C13—C12—C19— | 162.41 (17) |
| | | C20 | |
| C6—N1—C7—C8 | −175.11 (15) | C11—C12—C19— | −75.2 (2) |
| | | C20 | |
| C15—N2—C8—C7 | 101.36 (17) | C12—C19—C20— | −64.7 (2) |
| | | C22 | |
| C15—N2—C8—C9 | −17.0 (2) | C12—C19—C20— | 173.00 (18) |
| | | C21 | |
| O5—C7—C8—N2 | −53.6 (2) | | |

TABLE C
Hydrogen-bond parameters

| D—H^{...}A | D—H (Å) | H^{...}A (Å) | D^{...}A (Å) | D—H^{...}A(°) |
|---|---|---|---|---|
| O2—H2^{...}S1 | 0.73 (4) | 2.59 (3) | 3.0681 (17) | 125 (3) |
| O3—H3^{...}O5ⁱ | 0.89 (3) | 2.67 (3) | 3.1934 (19) | 118 (2) |
| O3—H3^{...}N2ⁱ | 0.89 (3) | 1.88 (3) | 2.748 (2) | 165 (2) |
| O4—H4^{...}O3ⁱ | 0.85 (3) | 1.95 (3) | 2.8018 (18) | 173 (2) |
| N2—H2A^{...}O2ⁱⁱ | 0.88 (2) | 2.38 (2) | 3.149 (2) | 146.5 (18) |

Symmetry code(s): ⁱ−x + 1, y, −z + 2; ⁱⁱ−x + 1, y − 1, −z + 2.

### Biological Assays

Minimum inhibitory concentrations (MICs) for compounds described herein have been determined for strains of several Gram positive and Gram negative strains. Data for exemplary compounds described herein is shown in Tables 2-11.

TABLE 2
MIC (μg/mL) of compounds against Gram positive and Gram negative strains

| | | | FSA- | FSA- | FSA- | FSA- | FSA- | FSA- |
|---|---|---|---|---|---|---|---|---|
| Species | Genotype | Clindamycin | 513003a | 513004a | 513001a | 513001b | 513002a | 513002b |
| S. aureus | ATCC 29213 | 0.12 | 0.12 | 1 | 0.25 | 0.12 | 1 | 0.5 |
| | BAA 977 iErmA | 0.12 | 0.12 | — | — | 0.12 | — | — |
| | MMX 3035 | >64 | 16 | — | — | 32 | — | — |
| | cErmA | | | | | | | |
| | MP-549 msrA | 0.12 | 0.12 | — | — | 0.12 | — | — |
| | MMX USA 300; | 0.25 | — | — | — | — | — | — |
| | MRSA | | | | | | | |
| S. | ATCC 49619 | ≤0.06 | 0.06 | 0.5 | 0.12 | 0.06 | 0.5 | 0.25 |
| pneumoniae | MMX 3028 | >64 | 8 | — | — | 16 | — | — |
| | cErmB | | | | | | | |
| | MMX 3031 | 0.06 | ≤0.06 | — | — | ≤0.06 | — | — |
| | cMefA | | | | | | | |
| S. pyogenes | ATCC 19615 | ≤0.06 | 0.06 | 0.25 | 0.12 | 0.06 | 1 | 0.25 |
| | MMX 946; | >64 | 8 | — | — | 16 | — | — |
| | cErmB | | | | | | | |
| E. faecalis | ATCC 29212 | 16 | 0.12 | 1 | 0.25 | 0.12 | 2 | 0.5 |
| | MMX 847; | >64 | 8 | — | — | 16 | — | — |
| | cErmB | | | | | | | |
| E. coli | ATCC 25922 | >64 | 32 | >128 | >64 | 64 | >64 | 128 |
| | MP-9 ΔTolC | 8 | 1 | >128 | 8 | 2 | 64 | 8 |
| | MP-74 LptD | 4 | 2 | 128 | 32 | 4 | 64 | 16 |
| | mutant | | | | | | | |
| K. | ATCC 10031 | 16 | — | — | — | — | — | — |
| pneumoniae | | | | | | | | |
| H. | ATCC 49247 | 8 | 2 | 128 | 16 | 4 | >64 | 8 |
| influenzae | | | | | | | | |

TABLE 3
MIC (μg/mL) of compounds against Gram positive and Gram negative strains

| | | | FSA- | FSA- | FSA- | FSA- | FSA- | FSA- |
|---|---|---|---|---|---|---|---|---|
| Species | Genotype | Clindamycin | 512078a | 512078b | 513018a | 513018b | 513013a | 513013b |
| S. aureus | ATCC 29213 | 0.12 | 0.06 | 0.5 | ≤0.06 | ≤0.06 | ≤0.06 | ≤0.06 |
| | BAA 977 iErmA | 0.12 | 0.06 | — | ≤0.06 | ≤0.06 | ≤0.06 | ≤0.06 |
| | MMX 3035 | >64 | 32 | — | 8 | 4 | 64 | 32 |
| | cErmA | | | | | | | |
| | MP-549 msrA | 0.12 | 0.06 | 0.25 | ≤0.06 | ≤0.06 | ≤0.06 | ≤0.06 |
| | MMX USA 300; | 0.25 | — | — | — | — | — | — |
| | MRSA | | | | | | | |
| S. | ATCC 49619 | ≤0.06 | 0.03 | — | ≤0.06 | ≤0.06 | ≤0.06 | ≤0.06 |
| pneumoniae | MMX 3028 | >64 | 8 | — | 0.5 | 0.25 | 8 | 4 |
| | cErmB | | | | | | | |
| | MMX 3031 | 0.06 | 0.03 | — | ≤0.06 | ≤0.06 | ≤0.06 | ≤0.06 |
| | cMefA | | | | | | | |
| S. pyogenes | ATCC 19615 | ≤0.06 | 0.02 | ≤0.12 | ≤0.06 | ≤0.06 | ≤0.06 | ≤0.06 |
| | MMX 946; | >64 | 8 | — | 0.5 | 0.25 | 8 | 4 |
| | cErmB | | | | | | | |
| E. faecalis | ATCC 29212 | 16 | 0.25 | 0.5 | ≤0.06 | ≤0.06 | ≤0.06 | ≤0.06 |
| | MMX 847; | >64 | 32 | — | 2 | 1 | 32 | 16 |
| | cErmB | | | | | | | |
| E. coli | ATCC 25922 | >64 | 32 | 128 | 16 | 8 | 32 | 32 |
| | MP-9 ΔTolC | 8 | 2 | 4 | 0.5 | 0.5 | 1 | 1 |
| | MP-74 LptD | 4 | 4 | 16 | 2 | 1 | 4 | 4 |
| | mutant | | | | | | | |
| K. | ATCC 10031 | 16 | 1 | 4 | — | — | — | — |
| pneumoniae | | | | | | | | |
| H. | ATCC 49247 | 8 | 8 | 32 | 2 | 1 | 8 | 4 |
| influenzae | | | | | | | | |

TABLE 4
MIC (μg/mL) of compounds against Gram positive and Gram negative strains

| | | | FSA- | FSA-10- | FSA-10- | FSA-09- |
|---|---|---|---|---|---|---|
| Species | Genotype | Clindamycin | 513018b | 09-045 | 09-051 | 04-092 |
| S. aureus | ATCC BAA- | 0.125 | 0.06 | 0.06 | 0.06 | 0.25 |
| | 1707 (MRSA) | | | | | |
| | MRSA | 0.25 | 0.06 | 0.06 | 0.06 | 0.25 |
| | ATCC 700699 | >128 | 2 | 8 | 4 | 8 |
| | hVISA | >128 | 1 | 8 | 8 | 16 |
| S. | MMX 3028 | >128 | 0.25 | 0.5 | 0.25 | 0.125 |
| pneumoniae | cErmB | | | | | |
| | MMX 3031 | 0.06 | 0.03 | 0.06 | 0.06 | 0.06 |
| | cMefA | | | | | |
| | ATCC 6303 | 0.06 | 0.03 | 0.06 | 0.06 | 0.06 |
| S. | MMX 946; | >128 | 0.25 | 1 | 0.5 | 0.25 |
| pyogenes | cErmB | | | | | |
| E. faecalis | MMX 847; | >128 | 1 | 8 | 4 | 1 |
| | cErmB | | | | | |
| | VRE | >128 | — | 16 | 8 | 4 |
| | VRE | >128 | 1 | 32 | 8 | 8 |
| | VRE | >128 | 1 | 8 | 2 | 4 |
| E. faecium | VRE | >128 | 1 | 8 | 4 | 4 |
| | Line R & VRE | >128 | 0.06 | 16 | 8 | 2 |
| E. coli | armA | >128 | 8 | 8 | 8 | 4 |
| P. | ATCC 27853 | >128 | 128 | >128 | >128 | 64 |
| aeruginosa | ΔAmex OprM | 32 | 4 | 2 | 2 | — |
| A. | ATCC 19606 | >128 | 4 | 16 | 8 | 16 |
| baumannii | | | | | | |

TABLE 5
MIC (μg/mL) of compounds against Gram positive and Gram negative strains

| | | | FSA- | FSA-10- | FSA-10- | FSA-10- |
|---|---|---|---|---|---|---|
| Species | Genotype | Clindamycin | 513018b | 09-093 | 09-098 | 09-099 |
| S. aureus | ATCC 29213 | 0.125 | 0.06 | 0.25 | 0.5 | 0.25 |
| | cErmA | >128 | 4 | 32 | 16 | 32 |
| S. pneumoniae | ATCC 49619 | 0.06 | 0.015 | 0.5 | 1 | 0.25 |
| S. pyogenes | ATCC 19615 | 0.06 | 0.03 | 0.5 | 1 | 0.5 |
| E. faecalis | ATCC 29212 | 16 | 0.06 | 0.25 | 0.25 | 0.5 |
| E. faecium | Clinical VRE | >128 | 1 | 8 | 4 | 16 |
| E. coli | ATCC 25922 | >128 | 8 | 16 | 8 | 32 |
| | ΔTolC | 4 | 0.5 | 1 | 1 | 1 |
| | LptD | 2 | 1 | 4 | 1 | 4 |
| P. aeruginosa | ATCC 27853 | >128 | 128 | >128 | >128 | >128 |
| A. baumannii | ATCC 19606 | >128 | 4 | 128 | 64 | >128 |
| K. oxytoca | Clinical | >128 | 8 | 16 | 16 | 32 |

TABLE 6
MIC (μg/mL) of compounds against Gram positive and Gram negative strains

| | | FSA-10- | FSA-10- | FSA-10- | FSA-10- | FSA-10- |
|---|---|---|---|---|---|---|
| Species | Genotype | 10-001 | 10-012 | 10-013 | 10-014 | 10-022 |
| S. aureus | ATCC 29213 | 0.125 | 0.125 | 0.25 | 1 | 0.125 |
| | cErmA | 4 | 8 | 64 | 64 | 16 |
| S. pneumoniae | ATCC 49619 | 0.125 | 0.125 | 0.25 | 2 | 0.25 |
| S. pyogenes | ATCC 19615 | 0.125 | 0.125 | 0.25 | 1 | 0.25 |
| E. faecalis | ATCC 29212 | 0.06 | 0.25 | 2 | 16 | 2 |
| E. faecium | Clinical VRE | 2 | 8 | 32 | 64 | 8 |
| E. coli | ATCC 25922 | 4 | 16 | 64 | 64 | 32 |
| | ΔTolC | 0.25 | 0.5 | 2 | 8 | 0.5 |
| | LptD | 0.5 | 2 | 4 | 8 | 4 |
| P. aeruginosa | ATCC 27853 | >128 | >128 | >128 | >128 | >128 |
| A. baumannii | ATCC 19606 | 16 | 32 | >128 | >128 | >128 |
| K. oxytoca | Clinical | 8 | 16 | 128 | 128 | 32 |

TABLE 7
MIC (μg/mL) of compounds against Gram positive and Gram negative strains

| | | | FSA-12- | FSA-12- | FSA-12- | FSA-12- |
|---|---|---|---|---|---|---|
| Species | Genotype | Clindamycin | 03-029 | 03-035 | 03-042 | 03-055 |
| S. aureus | ATCC 29213 | 0.125 | 0.25 | 0.125 | 0.125 | 0.125 |
| | cErmA | >128 | 8 | 8 | 16 | 16 |
| S. pneumoniae | ATCC 49619 | 0.06 | 0.06 | 0.06 | 0.06 | 0.06 |
| S. pyogenes | ATCC 19615 | 0.06 | 0.06 | 0.06 | 0.06 | 0.06 |
| E. faecalis | ATCC 29212 | 16 | 0.25 | 0.25 | 0.125 | 0.25 |
| E. faecium | Clinical VRE | >128 | 8 | 4 | 4 | 8 |
| E. coli | ATCC 25922 | >128 | 16 | 8 | 8 | 16 |
| | ΔTolC | 4 | 4 | 4 | 0.5 | 1 |
| | LptD | 2 | 4 | 4 | 2 | 4 |
| K. oxytoca | Clinical | >128 | 32 | 4 | 8 | 16 |

TABLE 8
MIC (μg/mL) of compounds against Gram positive and Gram negative strains

| | | FSA-09- | FSA-09- | FSA-09- | FSA-09- | FSA-09- | FSA-09- |
|---|---|---|---|---|---|---|---|
| Species | Genotype | 04-072 | 04-081 | 04-092 | 05-069 | 05-078 | 05-077 |
| S. aureus | ATCC 29213 | 1 | 0.125 | 0.5 | 1 | 2 | 2 |
| | cErmA | 32 | 8 | 4 | 64 | 4 | 4 |
| S. pneumoniae | ATCC 49619 | 0.5 | 0.125 | 0.25 | 0.125 | 0.25 | 0.25 |
| S. pyogenes | ATCC 19615 | 0.5 | 0.125 | 0.25 | 0.25 | 0.125 | 0.125 |
| E. faecalis | ATCC 29212 | 0.25 | 0.125 | 0.25 | 1 | 1 | 1 |
| E. faecium | Clinical VRE | 0.5 | 0.25 | 1 | 16 | 1 | 1 |
| E. coli | ATCC 25922 | 16 | 32 | 4 | 128 | 16 | 16 |
| | ΔTolC | 1 | 2 | 0.25 | 4 | 4 | 4 |
| | LptD | 2 | 2 | 0.5 | 16 | 2 | 1 |
| P. aeruginosa | ATCC 27853 | nd | nd | 64 | nd | nd | nd |
| A. baumannii | ATCC 19606 | nd | nd | 16 | nd | nd | nd |
| K. oxytoca | Clinical | 16 | 32 | 8 | 128 | 2 | 1 |

TABLE 9
MIC (μg/mL) of compounds against Gram positive and Gram negative strains

| | | FSA-09- | FSA-09- | FSA-09- | FSA-09- | FSA-09- | FSA-09- | FSA-09- |
|---|---|---|---|---|---|---|---|---|
| Species | Genotype | 05-68 | 05-076 | 04-087 | 04-094 | 05-011 | 05-074 | 05-079 |
| S. aureus | ATCC 29213 | 2 | 8 | 1 | 4 | 4 | 1 | 1 |
| | cErmA | 8 | 128 | 8 | 128 | 64 | 2 | 8 |
| S. pneumoniae | ATCC 49619 | 0.125 | 1 | 0.5 | 0.5 | 1 | 0.125 | 0.5 |
| S. pyogenes | ATCC 19615 | 0.25 | 0.25 | 0.5 | 1 | 1 | 0.25 | 0.5 |
| E. faecalis | ATCC 29212 | 1 | 4 | 0.5 | 2 | 2 | 0.5 | 0.5 |
| E. faecium | Clinical VRE | 4 | 16 | 4 | 128 | 32 | 2 | 4 |
| E. coli | ATCC 25922 | 32 | 128 | 16 | >128 | 32 | 32 | 16 |
| | ΔTolC | 2 | 8 | 1 | 32 | 2 | 4 | 1 |
| | LptD | 2 | 16 | 1 | 64 | 8 | 4 | 1 |
| P. aeruginosa | ATCC 27853 | nd | nd | nd | nd | nd | nd | nd |
| A. baumannii | ATCC 19606 | nd | nd | nd | nd | nd | nd | nd |
| K. oxytoca | Clinical | 32 | 16 | 16 | >128 | 64 | 32 | 16 |

TABLE 10
MIC (μg/mL) of compounds against Gram positive and Gram negative strains

| Species | Genotype | Clindamycin | FSA- 513018b | FSA- 512078a |
|---|---|---|---|---|
| C. difficile | ATCC 700057 | 8 | 0.25 | 1 |
| C. scindens | ATCC 35704 | 2 | 0.125 | 0.25 |
| B. fragilis | ATCC 25285 | 1 | 0.5 | 1 |

TABLE 11
MIC (μg/mL) of FSA-513018b in comparison to known antibiotics

| | | FSA- | | | | | |
|---|---|---|---|---|---|---|---|
| Species | Description | 513018b | Clindamycin | Lincomycin | Amikacin | Gentamicin | Ceftriaxone |
| C. difficile | HAV#011, ATCC | 0.25 | 8 | | | | |
| | 700057 | | | | | | |
| C. scindens | HAV#012, ATCC | 0.125 | 2 | | | | |
| | 35704 | | | | | | |
| E. faecalis | HAV#005, ATCC | 0.06 | 16 | 32 | | 16 | |
| | 29212 | | | | | | |
| E. faecalis | HAV#036, MMX | 1 | >256 | | | >64 | |
| | 847; mac-resistant, | | | | | | |
| | cErmB | | | | | | |
| E. faecalis | HAV#607; Clinical | 2 | >256 | | | >256 | >256 |
| E. faecalis | HAV#610; Clinical | 1 | >256 | | | >256 | >256 |
| E. faecalis | HAV#606; Clinical, | 1 | >256 | | | >256 | >256 |
| | VRE | | | | | | |
| E. faecalis | HAV#609; Clinical | 2 | >256 | | | >256 | >256 |
| E. faecalis | HAV#608; Clinical | 1 | >256 | | | >256 | >256 |
| E. faecalis | HAV#612; Line R | ≤0.06 | 8 | | | >128 | |
| E. faecalis | HAV#611; Line R & | 0.125 | 128 | | | >128 | |
| | Vanco R | | | | | | |
| E. faecalis | HAV#251; Clinical | 1 | >128 | | | 16 | |
| | VRE | | | | | | |
| E. faecalis | HAV#602, Clincal | ≤0.06 | 16 | | | | |
| E. faecium | HAV#327; Clinical | 0.06 | 8 | | | 8 | 0.5 |
| E. faecium | HAV#329; Clinical | 1 | >256 | | | 4 | >256 |
| E. faecium | HAV#324; Clinical | 1 | >256 | | | >256 | |
| E. faecium | HAV#328; Clinical | 1 | >256 | | | >256 | >128 |
| E. faecium | HAV#319; Line R & | ≤0.06 | >128 | | | 64 | >256 |
| | Vanco R, VRE | | | | | | |
| E. faecium | HAV#320; Clinical, | 0.25 | >128 | | | | |
| | VRE | | | | | | |
| E. faecium | HAV#318; VanA, | 1 | >128 | | | 8 | |
| | VRE | | | | | | |
| E. faecium | HAV#317; Clinical | 0.25 | 8 | | | 16 | |
| E. faecium | HAV#326; Clinical, | 0.25 | 8 | 16 | | | |
| | VRE | | | | | | |
| E. faecium | HAV#604; Clinical | 0.06 | 8 | | | | |
| S. aureus | HAV#001, ATCC | 0.06 | 0.125 | 0.5 | | 1 | 2 |
| | 29213, MP-549 | | | | | | |
| S. aureus | HAV#016, ATCC | 0.06 | 0.125 | 0.25 | | 1 | 64 |
| | BAA-1707; MRSA | | | | | | |
| S. aureus | HAV#031, MMX | 4 | >256 | | | 1 | |
| | 3035; cErmA | | | | | | |
| S. aureus | #HAV002, ATCC | 0.06 | 0.125 | | | 2 | 2 |
| | BAA 977; iErmA, | | | | | | |
| | MRSA | | | | | | |
| S. aureus | HAV#032, MP-549; | 0.06 | 0.125 | | | 1 | >128 |
| | MSR(A) | | | | | | |
| S. aureus | HAV#017; MRSA | 0.06 | 0.25 | | | 0.5 | >128 |
| | Col(Colindale, | | | | | | |
| | England) | | | | | | |
| S. aureus | HAV#216; Clinical, | 2 | 128 | | | 8 | 128 |
| | cfr | | | | | | |
| S. aureus | HAV#217; cfr | 4 | >128 | | | 4 | 64 |
| S. aureus | HAV#203; Clinical | 1 | >64 | | | >64 | |
| | (VISA | | | | | | |
| S. aureus | HAV#204, hVISA | 1 | >128 | | | >64 | |
| S. aureus | HAV#052, mecA, | ≤0.03 | 0.06 | | 4 | 1 | 64 |
| | norA, fosB, blaZ | | | | | | |
| S. aureus | HAV#053 mecA, | ≤0.03 | 0.06 | | 4 | 1 | 128 |
| | norA, fosB | | | | | | |
| S. aureus | HAV#054, mecA, | 1 | >256 | | 64 | 128 | >256 |
| | norA, spcl, aad1, | | | | | | |
| | fosB, ermA, aac6′- | | | | | | |
| | aph2″, blaZ | | | | | | |
| S. aureus | HAV#051; mecA, | ≤0.03 | 0.06 | | 4 | 2 | |
| | norA, spcl, ermA, | | | | | | |
| | aad1, aac6′-aph2″ | | | | | | |
| S. aureus | HAV#202, ATCC | 2 | >128 | | | >64 | >128 |
| | 700699 (VISA) | | | | | | |
| S. aureus | #HAV002, ATCC | 2 | >128 | | | | |
| | BAA 977; iErmA, | | | | | | |
| | MRSA, induced with | | | | | | |
| | 32 ug/mL | | | | | | |
| | Erythromycin | | | | | | |
| S. aureus | HAV#218; Line R, | 0.125 | 0.25 | | | | 32 |
| | MRSA | | | | | | |
| S. aureus | HAV#219; Line R, | >128 | >128 | | | | 2 |
| | MSSA | | | | | | |
| S. aureus | HAV#220; Line R, | >128 | >128 | | | | >128 |
| | MRSA | | | | | | |
| S. epidermidis | HAV# 275 Clinical; | 8 | >128 | | | >64 | >128 |
| | cfr | | | | | | |
| S. epidermidis | HAV# 276; cfr | >16 | >128 | | | >64 | >128 |
| S. epidermidis | HAV#277; Line R | 16 | >128 | | | | >128 |
| S. epidermidis | HAV#278 Line R | 32 | >128 | | | | >128 |
| S. epidermidis | HAV#279; Line R | 0.125 | 0.25 | | | | 4 |
| S. faecalis | 5060, Line R & | 0.125 | 128 | | | | >256 |
| | Vanco R | | | | | | |
| S. faecalis | 5074, Line R | ≤0.06 | 8 | | | | 256 |
| S. haemolyticus | 5052, Line R & | 0.06 | 2 | | | | >256 |
| | Methicillin R | | | | | | |
| S. haemolyticus | HAV#252; Line R & | 0.125 | 2 | | | >128 | |
| | Methicillin R | | | | | | |
| S. pneumoniae | HAV#003, ATCC | 0.015 | 0.06 | 1 | | 16 | 0.06 |
| | 49619 | | | | | | |
| S. pneumoniae | HAV#351, ATCC | 0.03 | 0.06 | | | 32 | 0.06 |
| | 6303 | | | | | | |
| S. pneumoniae | HAV#033; MMX | 0.25 | 256 | | | 4 | |
| | 3028, cErmB | | | | | | |
| S. pneumoniae | HAV#034; MMX | 0.03 | <0.06 | | | 8 | |
| | 3031,cMefA | | | | | | |
| S. pneumoniae | HAV#352, ATCC | 0.5 | >64 | | | | 1 |
| | 700673; MDR | | | | | | |
| S. pneumoniae | HAV#353, ATCC- | 0.125 | 0.25 | | | | 1 |
| | BAA-334 | | | | | | |
| S. pneumoniae | HAV#354, ATCC- | 0.125 | 0.25 | | | | 1 |
| | BAA-255 | | | | | | |
| S. pneumoniae | HAV#356; Clinical | 0.25 | >64 | | | | 1 |
| S. pneumoniae | HAV#358; Clinical | 0.125 | 0.25 | | | | 1 |
| S. aureus | HAV#211; Clinical | 2 | >64 | | | | >64 |
| S. aureus | HAV#212; Clinical | 0.06 | 0.125 | | | | 32 |
| S. aureus | HAV#213; Clinical | 0.06 | 0.125 | | | | 1 |
| S. aureus | HAV#214; Clinical | 0.03 | 0.06 | | | | 4 |
| S. aureus | HAV#215; Clinical | 0.06 | 0.125 | | | | 1 |
| S. pyogenes | HAV#004, ATCC | 0.03 | 0.06 | 0.5 | | 8 | 0.06 |
| | 19615 | | | | | | |
| S. pyogenes | HAV#035, MMX | 0.25 | >256 | | | 16 | ≤0.03 |
| | 946; cErmB, mac- | | | | | | |
| | resistant | | | | | | |
| A. baumanii | HAV#010, ATCC | 4 | >128 | | | >64 | |
| | 19606 | | | | | | |
| B. fragilis | HAV#013, ATCC | 0.5 | 0.5 | | | | |
| | 25285 | | | | | | |
| E. coli | HAV#006, ATCC | 8 | >128 | >256 | | 2 | 0.125 |
| | 25922 | | | | | | |
| E. coli | HAV#038; ΔTolC | 0.5 | 8 | 128 | | 1 | ≤0.06 |
| E. coli | HAV#040; LptD | 1 | 4 | 64 | | 1 | ≤0.06 |
| E. coli | HAV#056; Clinical, | 8 | >256 | | | >256 | ≤0.06 |
| | armA | | | | | | |
| E. coli | HAV#501; Clinical | 8 | >128 | | | 2 | |
| E. coli | HAV#504; Clinical, | 8 | >128 | | | | |
| | cErmB | | | | | | |
| E. coli | HAV#503; Clinical | 8 | 64 | | | 4 | >128 |
| E. coli | HAV#511; Clinical | 4 | 128 | | | 16 | 16 |
| E. coli | HAV#512; Clinical | 8 | >128 | | | 2 | <0.06 |
| E. coli | HAV#513; Clinical | 16 | >128 | | | 2 | 0.125 |
| E. coli | HAV#514; Clinical | 8 | 128 | | | 1 | 0.125 |
| E. coli | HAV#516; ESBL- | 8 | 128 | | | 2 | 64 |
| | producing | | | | | | |
| H. influenzae | HAV#376, ATCC- | 0.5 | 8 | | | 4 | |
| | 9007 | | | | | | |
| H. influenzae | HAV#007, ATCC | 1 | 8 | | | | |
| | 49247 | | | | | | |
| K. oxytoca | HAV#450; Clinical | 8 | >128 | >256 | | 4 | 64 |
| K. pneumoniae | HAV#008, ATCC | 0.25 | 8 | | | 0.25 | |
| | 10031 | | | | | | |
| K. pneumoniae | HAV#402; FQ-R | 16 | >128 | | | | |
| K. pneumoniae | HAV#041; Clinical, | 32 | 256 | | | 8 | |
| | KPC | | | | | | |
| K. pneumoniae | HAV#404; Clinical | 32 | 128 | | | 0.5 | 64 |
| K. pneumoniae | HAV#405; Clinical | 16 | >128 | | | 0.5 | 64 |
| K. pneumoniae | HAV#406; Clinical | 64 | >128 | | | 1 | <0.06 |
| K. pneumoniae | HAV#407; Clinical | 128 | >128 | | | 0.5 | 1 |
| K. pneumoniae | HAV#408; Clinical | 128 | >128 | | | 2 | 2 |
| K. pneumoniae | HAV#409; ESBL- | 16 | >128 | | | 0.5 | 128 |
| | producing | | | | | | |
| P. aeruginosa | HAV#009, ATCC | 128 | >128 | | | >64 | >64 |
| | 27853 | | | | | | |
| P. aeruginosa | HAV#039; ΔAmex | 4 | 32 | | | >64 | 16 |
| | OprM | | | | | | |
| N. gonorrhoeae | HAV#701, Clinical | 0.125 | 2 | | | | 0.125 |
| N. gonorrhoeae | HAV#702, Clinical | 0.125 | 0.5 | | | | 0.25 |
| A. baumannii | HAV#651; Clinical | 4 | 128 | | | 2 | 4 |
| A. baumannii | HAV#652 Clinical | 8 | 128 | | | 16 | 16 |
| A. baumannii | HAV#653; Clinical | 4 | 64 | | | 0.5 | 2 |
| A. baumannii | HAV#654; Clinical | 8 | 64 | | | 2 | 4 |
| A. baumannii | HAV#655; Clinical | 8 | 128 | | | 2 | 64 |
| A. baumannii | HAV#656; Clinical | 16 | 128 | | | 4 | 64 |
| A. baumannii | HAV#657; Clinical | 4 | 128 | | | 4 | 64 |
| A. baumannii | HAV#658; Clinical | 4 | 64 | | | 1 | 16 |
| A. baumannii | HAV#659; Clinical | 8 | 128 | | | 32 | 32 |
| A. baumannii | HAV#660; Clinical | 16 | >128 | | | 32 | 32 |
| A. baumannii | HAV#661; Clinical | 8 | 64 | | | 1 | 2 |
| A. baumannii | HAV#662; Clinical | 8 | 64 | | | 4 | 32 |
| A. baumannii | HAV#663; Clinical | 4 | 32 | | | 0.5 | 16 |
| A. baumannii | HAV#664; | 16 | 128 | | | >128 | 64 |
| | Carbapenem R | | | | | | |

| | | FSA- | | | | | | |
|---|---|---|---|---|---|---|---|---|
| Species | Description | 513018b | Chloramphenicol | Thiamphenicol | Azithromycin | Erythromycin | Ciprofloxacin | Enrofloxacin |
| C. difficile | HAV#011, | 0.25 | | | | | | 32 |
| | ATCC 700057 | | | | | | | |
| C. scindens | HAV#012, | 0.125 | | | | | | |
| | ATCC 35704 | | | | | | | |
| E. faecalis | HAV#005, | 0.06 | 8 | 16 | 8 | 2 | 1 | 0.5 |
| | ATCC 29212 | | | | | | | |
| E. faecalis | HAV#036, | 1 | | | >64 | >64 | 16 | |
| | MMX 847; | | | | | | | |
| | mac-resistant, | | | | | | | |
| | cErmB | | | | | | | |
| E. faecalis | HAV#607; | 2 | | | >128 | | 64 | |
| | Clinical | | | | | | | |
| E. faecalis | HAV#610; | 1 | | | >128 | | 0.25 | |
| | Clinical | | | | | | | |
| E. faecalis | HAV#606; | 1 | | | >128 | | >256 | |
| | Clinical, VRE | | | | | | | |
| E. faecalis | HAV#609; | 2 | | | >128 | | 128 | |
| | Clinical | | | | | | | |
| E. faecalis | HAV#608; | 1 | | | >128 | | 64 | |
| | Clinical | | | | | | | |
| E. faecalis | HAV#612; | ≤0.06 | | | | | | |
| | Line R | | | | | | | |
| E. faecalis | HAV#611; | 0.125 | | | | | | |
| | Line R & | | | | | | | |
| | Vanco R | | | | | | | |
| E. faecalis | HAV#251; | 1 | 4 | 8 | >64 | >64 | 4 | |
| | Clinical VRE | | | | | | | |
| E. faecalis | HAV#602, | ≤0.06 | | | 4 | | | |
| | Clincal | | | | | | | |
| E. faecium | HAV#327; | 0.06 | | | >128 | | 0.5 | |
| | Clinical | | | | | | | |
| E. faecium | HAV#329; | 1 | | | >128 | | 128 | |
| | Clinical | | | | | | | |
| E. faecium | HAV#324; | 1 | | | >128 | | 256 | |
| | Clinical | | | | | | | |
| E. faecium | HAV#328; | 1 | | | >128 | | 128 | |
| | Clinical | | | | | | | |
| E. faecium | HAV#319; | ≤0.06 | | | 8 | | | |
| | Line R & | | | | | | | |
| | Vanco R, VRE | | | | | | | |
| E. faecium | HAV#320; | 0.25 | | | >128 | | | |
| | Clinical, VRE | | | | | | | |
| E. faecium | HAV#318; | 1 | 8 | | >64 | | | |
| | VanA, VRE | | | | | | | |
| E. faecium | HAV#317; | 0.25 | | | 8 | | | |
| | Clinical | | | | | | | |
| E. faecium | HAV#326; | 0.25 | | | | | | |
| | Clinical, VRE | | | | | | | |
| E. faecium | HAV#604; | 0.06 | | | | | | |
| | Clinical | | | | | | | |
| S. aureus | HAV#001, | 0.06 | 8 | 32 | 1 | 0.5 | 0.5 | 0.125 |
| | ATCC 29213, | | | | | | | |
| | MP-549 | | | | | | | |
| S. aureus | HAV#016, | 0.06 | | | 1 | 0.5 | 0.5 | |
| | ATCC BAA- | | | | | | | |
| | 1707; MRSA | | | | | | | |
| S. aureus | HAV#031, | 4 | 16 | 32 | >64 | >64 | 32 | |
| | MMX 3035; | | | | | | | |
| | cErmA | | | | | | | |
| S. aureus | #HAV002, | 0.06 | | | >64 | >64 | 0.5 | |
| | ATCC BAA | | | | | | | |
| | 977; iErmA, | | | | | | | |
| | MRSA | | | | | | | |
| S. aureus | HAV#032, | 0.06 | | | >64 | 64 | 4 | 0.5 |
| | MP-549; | | | | | | | |
| | MSR(A) | | | | | | | |
| S. aureus | HAV#0107; | 0.06 | | | 1 | 0.5 | 0.5 | |
| | MRSA | | | | | | | |
| | Col(Colindale, | | | | | | | |
| | England) | | | | | | | |
| S. aureus | HAV#216; | 2 | >128 | | >128 | 32 | 16 | |
| | Clinical, cfr | | | | | | | |
| S. aureus | HAV#217; cfr | 4 | >128 | | >128 | 64 | >64 | |
| S. aureus | HAV#203; | 1 | | | >64 | >64 | 64 | |
| | Clinical (VISA | | | | | | | |
| S. aureus | HAV#204, | 1 | | | >64 | >64 | 64 | |
| | hVISA | | | | | | | |
| S. aureus | HAV#052, | ≤0.03 | | | 2 | | 2 | |
| | mecA, norA, | | | | | | | |
| | fosB, blaZ | | | | | | | |
| S. aureus | HAV#053 | ≤0.03 | | | 2 | | 1 | |
| | mecA, norA, | | | | | | | |
| | fosB | | | | | | | |
| S. aureus | HAV#054, | 1 | | | >256 | | 128 | |
| | mecA, norA, | | | | | | | |
| | spcl, aad1, | | | | | | | |
| | fosB, ermA, | | | | | | | |
| | aac6′-aph2″, | | | | | | | |
| | blaZ | | | | | | | |
| S. aureus | HAV#051; | ≤0.03 | | | 2 | | 1 | |
| | mecA, norA, | | | | | | | |
| | spcl, ermA, | | | | | | | |
| | aad1, aac6′- | | | | | | | |
| | aph2″ | | | | | | | |
| S. aureus | HAV#202, | 2 | | | >64 | | 32 | |
| | ATCC 700699 | | | | | | | |
| | (VISA) | | | | | | | |
| S. aureus | #HAV002, | 2 | | | | | | |
| | ATCC BAA | | | | | | | |
| | 977; iErmA, | | | | | | | |
| | MRSA, | | | | | | | |
| | induced with | | | | | | | |
| | 32 ug/mL | | | | | | | |
| | Erythromycin | | | | | | | |
| S. aureus | HAV#218; | 0.125 | | | >64 | | | |
| | Line R, MRSA | | | | | | | |
| S. aureus | HAV#219; | >128 | | | >64 | | | |
| | Line R, MSSA | | | | | | | |
| S. aureus | HAV#220; | >128 | | | >64 | | | |
| | Line R, MRSA | | | | | | | |
| S. epidermidis | HAV# 275 | 8 | >128 | | 32 | 8 | 64 | |
| | Clinical; cfr | | | | | | | |
| S. epidermidis | HAV# 276; cfr | >16 | >128 | | >64 | >128 | 64 | |
| S. epidermidis | HAV#277; | 16 | | | >64 | | | |
| | Line R | | | | | | | |
| S. epidermidis | HAV#278 Line | 32 | | | >64 | | | |
| | R | | | | | | | |
| S. epidermidis | HAV#279; | 0.125 | | | >64 | | | |
| | Line R | | | | | | | |
| S. faecalis | 5060, Line R | 0.125 | | | >256 | | | |
| | & Vanco R | | | | | | | |
| S. faecalis | 5074, Line R | ≤0.06 | | | 4 | | | |
| S. haemolyticus | 5052, Line R | 0.06 | | | >256 | | | |
| | & Methicillin | | | | | | | |
| | R | | | | | | | |
| S. haemolyticus | HAV#252; | 0.125 | | | | | 32 | |
| | Line R & | | | | | | | |
| | Methicillin R | | | | | | | |
| S. pneumoniae | HAV#003, | 0.015 | 4 | 2 | 0.125 | ≤0.06 | 0.25 | 0.25 |
| | ATCC 49619 | | | | | | | |
| S. pneumoniae | HAV#351, | 0.03 | | | 2 | 0.5 | 1 | |
| | ATCC 6303 | | | | | | | |
| S. pneumoniae | HAV#033; | 0.25 | | | >64 | >64 | 1 | |
| | MMX 3028, | | | | | | | |
| | cErmB | | | | | | | |
| S. pneumoniae | HAV#034; | 0.03 | | | 16 | 8 | 0.5 | |
| | MMX 3031, | | | | | | | |
| | cMefA | | | | | | | |
| S. pneumoniae | HAV#352, | 0.5 | | | >64 | | | |
| | ATCC 700673; | | | | | | | |
| | MDR | | | | | | | |
| S. pneumoniae | HAV#353, | 0.125 | | | 1 | | | |
| | ATCC-BAA- | | | | | | | |
| | 334 | | | | | | | |
| S. pneumoniae | HAV#354, | 0.125 | | | 2 | | | |
| | ATCC-BAA- | | | | | | | |
| | 255 | | | | | | | |
| S. pneumoniae | HAV#356; | 0.25 | | | >64 | | | |
| | Clinical | | | | | | | |
| S. pneumoniae | HAV#358; | 0.125 | | | 2 | | | |
| | Clinical | | | | | | | |
| S. aureus | HAV#211; | 2 | | | >64 | | | |
| | Clinical | | | | | | | |
| S. aureus | HAV#212; | 0.06 | | | 1 | | | |
| | Clinical | | | | | | | |
| S. aureus | HAV#213; | 0.06 | | | 0.25 | | | |
| | Clinical | | | | | | | |
| S. aureus | HAV#214; | 0.03 | | | 0.25 | | | |
| | Clinical | | | | | | | |
| S. aureus | HAV#215; | 0.06 | | | 0.25 | | | |
| | Clinical | | | | | | | |
| S. pyogenes | HAV#004, | 0.03 | 4 | 4 | 0.06 | <0.06 | 0.25 | 0.25 |
| | ATCC 19615 | | | | | | | |
| S. pyogenes | HAV#035, | 0.25 | | | >64 | >64 | 0.25 | |
| | MMX 946; | | | | | | | |
| | cErmB, mac- | | | | | | | |
| | resistant | | | | | | | |
| A. baumanii | HAV#010, | 4 | | | 64 | 64 | 2 | |
| | ATCC 19606 | | | | | | | |
| B. fragilis | HAV#013, | 0.5 | | | | | | 2 |
| | ATCC 25285 | | | | | | | |
| E. coli | HAV#006, | 8 | 8 | 64 | 4 | 64 | ≤0.03 | ≤0.06 |
| | ATCC 25922 | | | | | | | |
| E. coli | HAV#038; | 0.5 | 8 | 16 | 1 | 4 | ≤0.03 | ≤0.06 |
| | ΔTolC | | | | | | | |
| E. coli | HAV#040; | 1 | 4 | 32 | ≤0.03 | 1 | ≤0.03 | ≤0.06 |
| | LptD | | | | | | | |
| E. coli | HAV#056; | 8 | | | 4 | 32 | ≤0.03 | |
| | Clinical, armA | | | | | | | |
| E. coli | HAV#501; | 8 | | | 64 | >64 | 64 | |
| | Clinical | | | | | | | |
| E. coli | HAV#504; | 8 | | | | | | |
| | Clinical, | | | | | | | |
| | cErmB | | | | | | | |
| E. coli | HAV#503; | 8 | | | 8 | | >128 | |
| | Clinical | | | | | | | |
| E. coli | HAV#511; | 4 | | | 4 | | | |
| | Clinical | | | | | | | |
| E. coli | HAV#512; | 8 | | | 8 | | | |
| | Clinical | | | | | | | |
| E. coli | HAV#513; | 16 | | | 8 | | | |
| | Clinical | | | | | | | |
| E. coli | HAV#514; | 8 | | | 8 | | | |
| | Clinical | | | | | | | |
| E. coli | HAV#516; | 8 | | | 4 | | | |
| | ESBL- | | | | | | | |
| | producing | | | | | | | |
| H. influenzae | HAV#376, | 0.5 | | | 2 | | | |
| | ATCC-9007 | | | | | | | |
| H. influenzae | HAV#007, | 1 | | | | 8 | | ≤0.06 |
| | ATCC 49247 | | | | | | | |
| K. oxytoca | HAV#450; | 8 | 8 | 64 | 16 | >64 | ≤0.06 | |
| | Clinical | | | | | | | |
| K. pneumnoniae | HAV#008, | 0.25 | | | 4 | 8 | ≤0.06 | ≤0.06 |
| | ATCC 10031 | | | | | | | |
| K. pneumnoniae | HAV#402; | 16 | | | | | | |
| | FQ-R | | | | | | | |
| K. pneumoniae | HAV#041; | 32 | >128 | >64 | >64 | >64 | >64 | |
| | Clinical, KPC | | | | | | | |
| K. pneumoniae | HAV#404; | 32 | | | 8 | | | |
| | Clinical | | | | | | | |
| K. pneumoniae | HAV#405; | 16 | | | 8 | | | |
| | Clinical | | | | | | | |
| K. pneumoniae | HAV#406; | 64 | | | 8 | | | |
| | Clinical | | | | | | | |
| K. pneumoniae | HAV#407; | 128 | | | 8 | | | |
| | Clinical | | | | | | | |
| K. pneumoniae | HAV#408; | 128 | | | 8 | | | |
| | Clinical | | | | | | | |
| K. pneumoniae | HAV#409; | 16 | | | 4 | | 0.5 | |
| | ESBL- | | | | | | | |
| | producing | | | | | | | |
| P. aeruginosa | HAV#009, | 128 | | | >64 | >64 | 0.5 | |
| | ATCC 27853 | | | | | | | |
| P. aeruginosa | HAV#039; | 4 | | | 8 | 16 | 0.06 | |
| | ΔAmex OprM | | | | | | | |
| N. gonorrhoeae | HAV#701, | 0.125 | | | | | 0.03 | |
| | Clinical | | | | | | | |
| N. gonorrhoeae | HAV#702, | 0.125 | | | | | 4 | |
| | Clinical | | | | | | | |
| A. baumannii | HAV#651; | 4 | | | 4 | | | |
| | Clinical | | | | | | | |
| A. baumannii | HAV#652 | 8 | | | 4 | | | |
| | Clinical | | | | | | | |
| A. baumannii | HAV#653; | 4 | | | 4 | | | |
| | Clinical | | | | | | | |
| A. baumannii | HAV#654; | 8 | | | 4 | | | |
| | Clinical | | | | | | | |
| A. baumannii | HAV#655; | 8 | | | 4 | | | |
| | Clinical | | | | | | | |
| A. baumannii | HAV#656; | 16 | | | 4 | | | |
| | Clinical | | | | | | | |
| A. baumannii | HAV#657; | 4 | | | 4 | | | |
| | Clinical | | | | | | | |
| A. baumannii | HAV#658; | 4 | | | 4 | | | |
| | Clinical | | | | | | | |
| A. baumannii | HAV#659; | 8 | | | 4 | | | |
| | Clinical | | | | | | | |
| A. baumannii | HAV#660; | 16 | | | 4 | | | |
| | Clinical | | | | | | | |
| A. baumannii | HAV#661; | 8 | | | 4 | | | |
| | Clinical | | | | | | | |
| A. baumannii | HAV#662; | 8 | | | 8 | | | |
| | Clinical | | | | | | | |
| A. baumannii | HAV#663; | 4 | | | 4 | | | |
| | Clinical | | | | | | | |
| A. baumannii | HAV#664; | 16 | | | 4 | | | |
| | Carbapenem R | | | | | | | |

| | | FSA- | | | | | | |
|---|---|---|---|---|---|---|---|---|
| Species | Description | 513018b | Levofloxacin | Vancomycin | Linezolid | Doxycycline | Minocycline | Oxacillin |
| C. difficile | HAV#011, | 0.25 | | | | | | |
| | ATCC 700057 | | | | | | | |
| C. scindens | HAV#012, | 0.125 | | | | | | |
| | ATCC 35704 | | | | | | | |
| E. faecalis | HAV#005, | 0.06 | 2 | 4 | 2 | 4 | 4 | 32 |
| | ATCC 29212 | | | | | | | |
| E. faecalis | HAV#036, | 1 | >64 | >64 | 2 | 16 | | 64 |
| | MMX 847; | | | | | | | |
| | mac-resistant, | | | | | | | |
| | cErmB | | | | | | | |
| E. faecalis | HAV#607; | 2 | 64 | >128 | 16 | 1 | | |
| | Clinical | | | | | | | |
| E. faecalis | HAV#610; | 1 | 8 | 32 | 2 | 0.5 | | |
| | Clinical | | | | | | | |
| E. faecalis | HAV#606; | 1 | 128 | >128 | 2 | 16 | | |
| | Clinical, VRE | | | | | | | |
| E. faecalis | HAV#609; | 2 | 32 | 32 | 4 | 8 | | |
| | Clinical | | | | | | | |
| E. faecalis | HAV#608; | 1 | 64 | 16 | 2 | 0.5 | | |
| | Clinical | | | | | | | |
| E. faecalis | HAV#612; | ≤0.06 | | | | | | |
| | Line R | | | | | | | |
| E. faecalis | HAV#611; | 0.125 | | | | | | |
| | Line R & | | | | | | | |
| | Vanco R | | | | | | | |
| E. faecalis | HAV#251; | 1 | 2 | 8 | 2 | 0.125 | | >64 |
| | Clinical VRE | | | | | | | |
| E. faecalis | HAV#602, | ≤0.06 | 1 | 4 | 4 | 0.25 | | |
| | Clincal | | | | | | | |
| E. faecium | HAV#327; | 0.06 | 4 | 4 | 4 | 0.25 | | |
| | Clinical | | | | | | | |
| E. faecium | HAV#329; | 1 | 128 | >128 | 4 | 0.25 | | |
| | Clinical | | | | | | | |
| E. faecium | HAV#324; | 1 | >128 | >128 | 4 | 0.25 | | |
| | Clinical | | | | | | | |
| E. faecium | HAV#328; | 1 | 128 | >128 | | 0.25 | | |
| | Clinical | | | | | | | |
| E. faecium | HAV#319; | ≤0.06 | 128 | >256 | 64 | 16 | | |
| | Line R & | | | | | | | |
| | Vanco R, VRE | | | | | | | |
| E. faecium | HAV#320; | 0.25 | 4 | >128 | 2 | >128 | | |
| | Clinical, VRE | | | | | | | |
| E. faecium | HAV#318; | 1 | | >64 | 4 | 4 | | |
| | VanA, VRE | | | | | | | |
| E. faecium | HAV#317; | 0.25 | 2 | 2 | 2 | 0.25 | | |
| | Clinical | | | | | | | |
| E. faecium | HAV#326; | 0.25 | | | | | 0.125 | |
| | Clinical, VRE | | | | | | | |
| E. faecium | HAV#604; | 0.06 | | | | | | |
| | Clinical | | | | | | | |
| S. aureus | HAV#001, | 0.06 | 0.25 | 1 | 2 | 0.25 | 0.25 | 0.5 |
| | ATCC 29213, | | | | | | | |
| | MP-549 | | | | | | | |
| S. aureus | HAV#016, | 0.06 | >128 | 1 | 2 | 0.25 | 0.25 | >64 |
| | ATCC BAA- | | | | | | | |
| | 1707; MRSA | | | | | | | |
| S. aureus | HAV#031, | 4 | 4 | 2 | 2 | 0.25 | | >64 |
| | MMX 3035; | | | | | | | |
| | cErmA | | | | | | | |
| S. aureus | #HAV002, | 0.06 | 0.5 | 1 | 2 | 0.25 | 0.25 | 0.5 |
| | ATCC BAA | | | | | | | |
| | 977; iErmA, | | | | | | | |
| | MRSA | | | | | | | |
| S. aureus | HAV#032, | 0.06 | 1 | 1 | 4 | 8 | | 64 |
| | MP-549; | | | | | | | |
| | MSR(A) | | | | | | | |
| S. aureus | HAV#0107; | 0.06 | 1 | 2 | 2 | 64 | | >64 |
| | MRSA | | | | | | | |
| | Col(Colindale, | | | | | | | |
| | England) | | | | | | | |
| S. aureus | HAV#216; | 2 | 8 | >64 | 16 | 0.25 | | |
| | Clinical, cfr | | | | | | | |
| S. aureus | HAV#217; cfr | 4 | 2 | >64 | 64 | 4 | | |
| S. aureus | HAV#203; | 1 | 8 | 8 | 2 | 8 | | 64 |
| | Clinical (VISA | | | | | | | |
| S. aureus | HAV#204, | 1 | 8 | 2 | 2 | 16 | | 64 |
| | hVISA | | | | | | | |
| S. aureus | HAV#052, | ≤0.03 | 1 | 2 | 4 | 0.5 | | |
| | mecA, norA, | | | | | | | |
| | fosB, blaZ | | | | | | | |
| S. aureus | HAV#053 | ≤0.03 | 0.5 | 2 | 4 | 0.5 | | |
| | mecA, norA, | | | | | | | |
| | fosB | | | | | | | |
| S. aureus | HAV#054, | 1 | 32 | 1 | 1 | 2 | | |
| | mecA, norA, | | | | | | | |
| | spcl, aad1, | | | | | | | |
| | fosB, ermA, | | | | | | | |
| | aac6′-aph2″, | | | | | | | |
| | blaZ | | | | | | | |
| S. aureus | HAV#051; | ≤0.03 | 0.5 | 2 | 4 | 0.5 | | |
| | mecA, norA, | | | | | | | |
| | spcl, ermA, | | | | | | | |
| | aad1, aac6′- | | | | | | | |
| | aph2″ | | | | | | | |
| S. aureus | HAV#202, | 2 | 32 | 8 | 2 | 16 | | |
| | ATCC 700699 | | | | | | | |
| | (VISA) | | | | | | | |
| S. aureus | #HAV002, | 2 | | | 4 | | | |
| | ATCC BAA | | | | | | | |
| | 977; iErmA, | | | | | | | |
| | MRSA, | | | | | | | |
| | induced with | | | | | | | |
| | 32 ug/mL | | | | | | | |
| | Erythromycin | | | | | | | |
| S. aureus | HAV#218; | 0.125 | 0.25 | 1 | 8 | 16 | | |
| | Line R, MRSA | | | | | | | |
| S. aureus | HAV#219; | >128 | 0.25 | 1 | 32 | 1 | | |
| | Line R, MSSA | | | | | | | |
| S. aureus | HAV#220; | >128 | 64 | 1 | 32 | 8 | | |
| | Line R, MRSA | | | | | | | |
| S. epidermidis | HAV# 275 | 8 | 16 | >64 | >64 | 1 | | |
| | Clinical; cfr | | | | | | | |
| S. epidermidis | HAV# 276; cfr | >16 | 128 | >64 | >64 | 2 | | |
| S. epidermidis | HAV#277; | 16 | >128 | 2 | >128 | 8 | | |
| | Line R | | | | | | | |
| S. epidermidis | HAV#278 Line | 32 | 16 | 2 | 64 | 4 | | |
| | R | | | | | | | |
| S. epidermidis | HAV#279; | 0.125 | 8 | 2 | 4 | 32 | | |
| | Line R | | | | | | | |
| S. faecalis | 5060, Line R | 0.125 | 64 | >256 | 8 | 16 | | |
| | & Vanco R | | | | | | | |
| S. faecalis | 5074, Line R | ≤0.06 | 8 | 2 | 8 | 64 | | |
| S. haemolyticus | 5052, Line R & | 0.06 | 8 | 1 | 64 | 4 | | |
| | Methicillin R | | | | | | | |
| S. haemolyticus | HAV#252; | 0.125 | | | | | | |
| | Line R & | | | | | | | |
| | Methicillin R | | | | | | | |
| S. pneumoniae | HAV#003, | 0.015 | 1 | 0.5 | 1 | 0.125 | | 2 |
| | ATCC 49619 | | | | | | | |
| S. pneumoniae | HAV#351, | 0.03 | 0.5 | 0.5 | 1 | 0.125 | | 0.5 |
| | ATCC 6303 | | | | | | | |
| S. pneumoniae | HAV#033; | 0.25 | 0.5 | 0.25 | 0.5 | 1 | | 2 |
| | MMX 3028, | | | | | | | |
| | cErmB | | | | | | | |
| S. pneumoniae | HAV#034; | 0.03 | 0.5 | 0.5 | 1 | 0.125 | | 16 |
| | MMX 3031, | | | | | | | |
| | cMefA | | | | | | | |
| S. pneumoniae | HAV#352, | 0.5 | 0.5 | 0.5 | 4 | 4 | | |
| | ATCC 700673; | | | | | | | |
| | MDR | | | | | | | |
| S. pneumoniae | HAV#353, | 0.125 | 0.5 | 0.5 | 4 | 0.25 | | |
| | ATCC-BAA- | | | | | | | |
| | 334 | | | | | | | |
| S. pneumoniae | HAV#354, | 0.125 | 0.25 | 1 | 4 | 0.25 | | |
| | ATCC-BAA- | | | | | | | |
| | 255 | | | | | | | |
| S. pneumoniae | HAV#356; | 0.25 | 0.25 | 2 | 4 | 2 | | |
| | Clinical | | | | | | | |
| S. pneumoniae | HAV#358; | 0.125 | 0.25 | 1 | 4 | 0.25 | | |
| | Clinical | | | | | | | |
| S. aureus | HAV#211; | 2 | 8 | 0.5 | 4 | 4 | | |
| | Clinical | | | | | | | |
| S. aureus | HAV#212; | 0.06 | 0.25 | 0.5 | 4 | 0.25 | | |
| | Clinical | | | | | | | |
| S. aureus | HAV#213; | 0.06 | 2 | 0.5 | 2 | 0.125 | | |
| | Clinical | | | | | | | |
| S. aureus | HAV#214; | 0.03 | 0.25 | 0.5 | 4 | 0.25 | | |
| | Clinical | | | | | | | |
| S. aureus | HAV#215; | 0.06 | 0.25 | 1 | 2 | 0.25 | | |
| | Clinical | | | | | | | |
| S. pyogenes | HAV#004, | 0.03 | 0.5 | 0.5 | 2 | 0.125 | | 0.125 |
| | ATCC 19615 | | | | | | | |
| S. pyogenes | HAV#035, | 0.25 | 0.5 | 0.5 | 1 | 0.125 | | 0.25 |
| | MMX 946; | | | | | | | |
| | cErmB, mac- | | | | | | | |
| | resistant | | | | | | | |
| A. baumanii | HAV#010, | 4 | 1 | | | | | |
| | ATCC 19606 | | | | | | | |
| B. fragilis | HAV#013, | 0.5 | | | | | | |
| | ATCC 25285 | | | | | | | |
| E. coli | HAV#006, | 8 | ≤0.06 | >128 | >128 | | 0.5 | >64 |
| | ATCC 25922 | | | | | | | |
| E. coli | HAV#038; | 0.5 | ≤0.06 | >128 | 8 | | ≤.0125 | 16 |
| | ΔTolC | | | | | | | |
| E. coli | HAV#040; | 1 | ≤0.06 | 1 | 32 | | 0.25 | 4 |
| | LptD | | | | | | | |
| E. coli | HAV#056; | 8 | ≤0.06 | | | | | |
| | Clinical, armA | | | | | | | |
| E. coli | HAV#501; | 8 | 32 | | | | | |
| | Clinical | | | | | | | |
| E. coli | HAV#504; | 8 | | | | | | |
| | Clinical, | | | | | | | |
| | cErmB | | | | | | | |
| E. coli | HAV#503; | 8 | 64 | 128 | 128 | 32 | | |
| | Clinical | | | | | | | |
| E. coli | HAV#511; | 4 | 4 | >128 | 32 | 2 | | |
| | Clinical | | | | | | | |
| E. coli | HAV#512; | 8 | <0.06 | >128 | 32 | 2 | | |
| | Clinical | | | | | | | |
| E. coli | HAV#513; | 16 | 2 | >128 | 32 | 32 | | |
| | Clinical | | | | | | | |
| E. coli | HAV#514; | 8 | <0.06 | 128 | 32 | 2 | | |
| | Clinical | | | | | | | |
| E. coli | HAV#516; | 8 | 16 | 128 | 64 | 8 | | |
| | ESBL- | | | | | | | |
| | producing | | | | | | | |
| H. influenzae | HAV#376, | 0.5 | 0.03 | >64 | 4 | 0.25 | | |
| | ATCC-9007 | | | | | | | |
| H. influenzae | HAV#007, | 1 | | >64 | | | | |
| | ATCC 49247 | | | | | | | |
| K. oxytoca | HAV#450; | 8 | 0.5 | | | 8 | | >64 |
| | Clinical | | | | | | | |
| K. pneumnoniae | HAV#008, | 0.25 | ≤0.03 | >64 | | | | |
| | ATCC 10031 | | | | | | | |
| K. pneumnoniae | HAV#402; | 16 | | | | | | |
| | FQ-R | | | | | | | |
| K. pneumoniae | HAV#041; | 32 | 64 | | | | | |
| | Clinical, KPC | | | | | | | |
| K. pneumoniae | HAV#404; | 32 | 1 | >128 | 32 | 4 | | |
| | Clinical | | | | | | | |
| K. pneumoniae | HAV#405; | 16 | 1 | >128 | 32 | 2 | | |
| | Clinical | | | | | | | |
| K. pneumoniae | HAV#406; | 64 | 0.125 | >128 | 32 | 2 | | |
| | Clinical | | | | | | | |
| K. pneumoniae | HAV#407; | 128 | >128 | >128 | 32 | 32 | | |
| | Clinical | | | | | | | |
| K. pneumoniae | HAV#408; | 128 | 1 | >128 | 32 | 32 | | |
| | Clinical | | | | | | | |
| K. pneumoniae | HAV#409; | 16 | 0.5 | >128 | 64 | 16 | | |
| | ESBL- | | | | | | | |
| | producing | | | | | | | |
| P. aeruginosa | HAV#009, | 128 | 2 | | | | | |
| | ATCC 27853 | | | | | | | |
| P. aeruginosa | HAV#039; | 4 | 0.125 | | | | | |
| | ΔAmex OprM | | | | | | | |
| N. gonorrhoeae | HAV#701, | 0.125 | | | | | | |
| | Clinical | | | | | | | |
| N. gonorrhoeae | HAV#702, | 0.125 | | | | | | |
| | Clinical | | | | | | | |
| A. baumannii | HAV#651; | 4 | 0.125 | 128 | 16 | 0.125 | | |
| | Clinical | | | | | | | |
| A. baumannii | HAV#652 | 8 | 1 | 128 | 32 | 0.5 | | |
| | Clinical | | | | | | | |
| A. baumannii | HAV#653; | 4 | 0.25 | 128 | 32 | 0.125 | | |
| | Clinical | | | | | | | |
| A. baumannii | HAV#654; | 8 | 0.125 | 128 | 32 | <0.06 | | |
| | Clinical | | | | | | | |
| A. baumannii | HAV#655; | 8 | 0.5 | 128 | 128 | 0.5 | | |
| | Clinical | | | | | | | |
| A. baumannii | HAV#656; | 16 | 32 | >128 | 64 | 32 | | |
| | Clinical | | | | | | | |
| A. baumannii | HAV#657; | 4 | 0.5 | >128 | 64 | 0.5 | | |
| | Clinical | | | | | | | |
| A. baumannii | HAV#658; | 4 | 0.25 | >128 | 64 | 0.06 | | |
| | Clinical | | | | | | | |
| A. baumannii | HAV#659; | 8 | 16 | >128 | 64 | 0.5 | | |
| | Clinical | | | | | | | |
| A. baumannii | HAV#660; | 16 | 64 | >128 | 64 | 1 | | |
| | Clinical | | | | | | | |
| A. baumannii | HAV#661; | 8 | 0.125 | 128 | 32 | 0.125 | | |
| | Clinical | | | | | | | |
| A. baumannii | HAV#662; | 8 | 8 | >128 | 64 | 0.25 | | |
| | Clinical | | | | | | | |
| A. baumannii | HAV#663; | 4 | 0.25 | 128 | 32 | 0.125 | | |
| | Clinical | | | | | | | |
| A. baumannii | HAV#664; | 16 | 64 | >128 | 64 | 2 | | |
| | Carbapenem R | | | | | | | |

Abbreviations: iErmA/B, inducible erythromycin ribosome methyltransferase A; c-ermA/B, constitutively expressed erythromycin methyltransferase A/B gene; MRSA, methicillin-resistant Staphylococcus aureus; cfr, chloramphenicol-florfenicol resistance gene; PIRSP, penicillin-intermediate resistant Streptococcus pneumoniae; armA, aminoglycoside resistance methylase gene; KPC, Klebsiella pneumoniae carbapenemase; FQ-R, fluoroquinolone-resistant; msr(A), erythromycin resistance ATP-binding protein MsrA gene; VISA, vancomycin-intermediate resistant Staphylococcus aureus; hVISA, vancomycin-heteroresistant Staphylococcus aureus; MDR, multidrug-resistant; c-mefA, constitutively expressed macrolide efflux protein A gene; VRE, vancomycin-resistant Enterococcus; Line-R, linezolid-resistant.

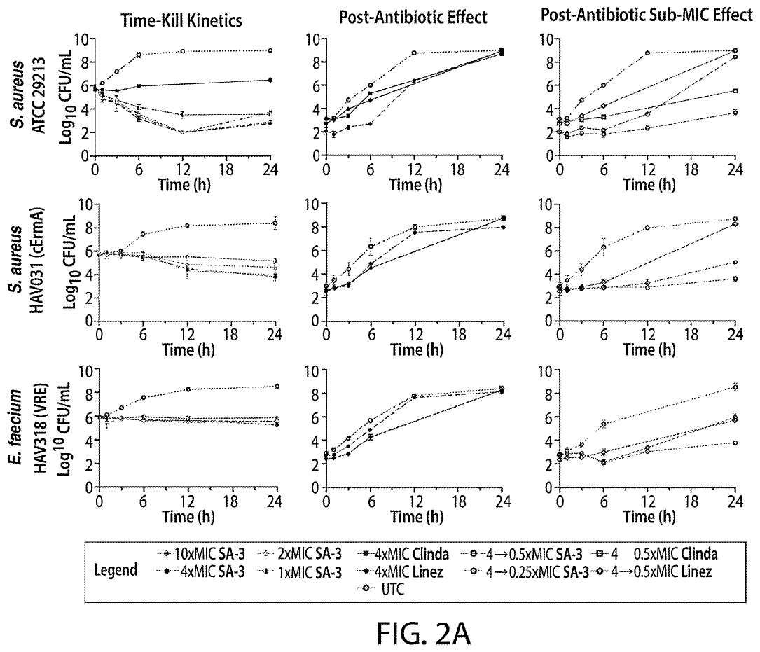

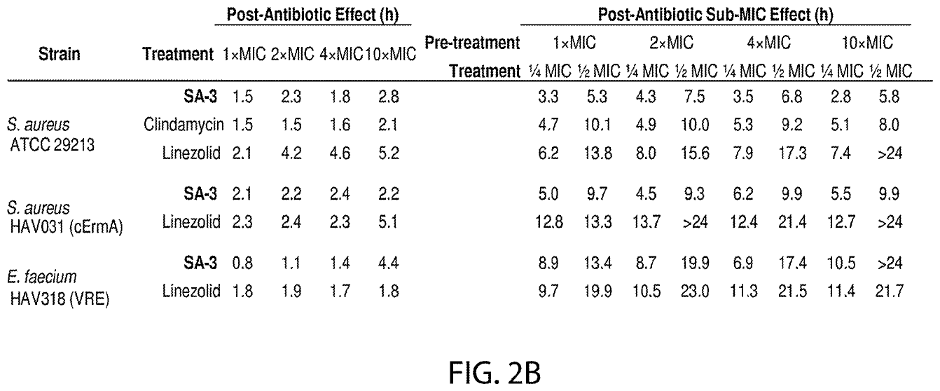

FSA-513018b exhibits markedly improved antibacterial activity when compared to clindamycin and in side-by-side comparisons with azithromycin, vancomycin, doxycycline, levofloxacin, and linezolid against a broad panel of different clinical isolates (e.g., Tables 11). Broth culture susceptibility analysis revealed that FSA-513018b displayed an extended spectrum of activity against common bacterial pathogens, including those constitutively expressing erm genes that confer resistance to MLS_{B} antibiotics. For instance, in streptococci, FSA-513018b overcame c-ermB-mediated resistance, an increasingly widespread phenotype, displaying minimum inhibitory concentrations (MICs) some ≥1,000× lower than that of clindamycin against S. pneumoniae and S. pyogenes strains. FSA-513018b displayed uniformly high activity against a panel of clinically derived strains of methicillin-resistant Staphylococcus aureus (MRSA). Enterococcal pathogens, responsible for one of the largest shares of healthcare-associated infections and historically invulnerable to lincosamides, appear broadly susceptible as well: FSA-513018b displayed≤2μg/mL MICs against a panel of Enterococcus faecium and Enterococcus faecalis strains resistant to vancomycin, linezolid, azithromycin, ciprofloxacin, levofloxacin, gentamicin, and doxycycline. On the basis of its potent activity against C. difficile (a CDC-listed Urgent Threat), FSA-513018b may also carry reduced liability to promote C. difficile colitis. FSA-513018b showed considerable activity in Gram-negative species as well—a further departure from approved lincosamides—even inhibiting the growth of a clinically derived strain of Escherichia coli with acquired resistance (c-ermB, MIC 8 μg/mL). Notably, this expanded spectrum of activity relative to clindamycin is not readily explained by traditional physicochemical predictors of Gram-negative activity such as rotatable-bond count, molecular weight, relative polar surface area, or lipophilicity. Broth culture profiling revealed that, like clindamycin, FSA-513018b is bacteriostatic but exhibits prolonged effects on bacterial growth following even brief exposure; concentration-dependent cidality was observed for certain highly susceptible strains (FIG. 2A-B).

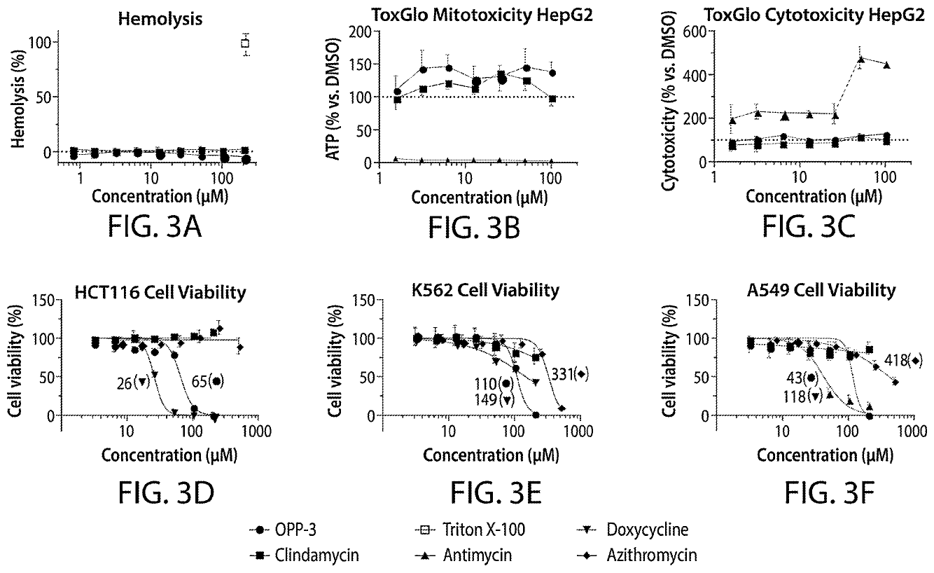

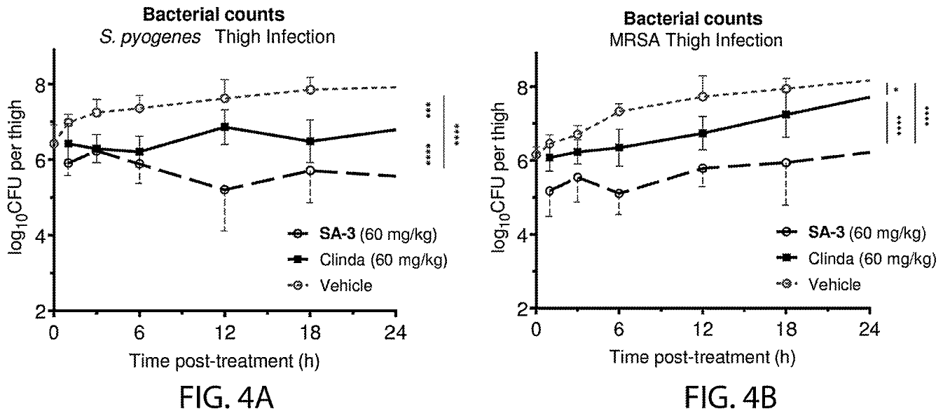

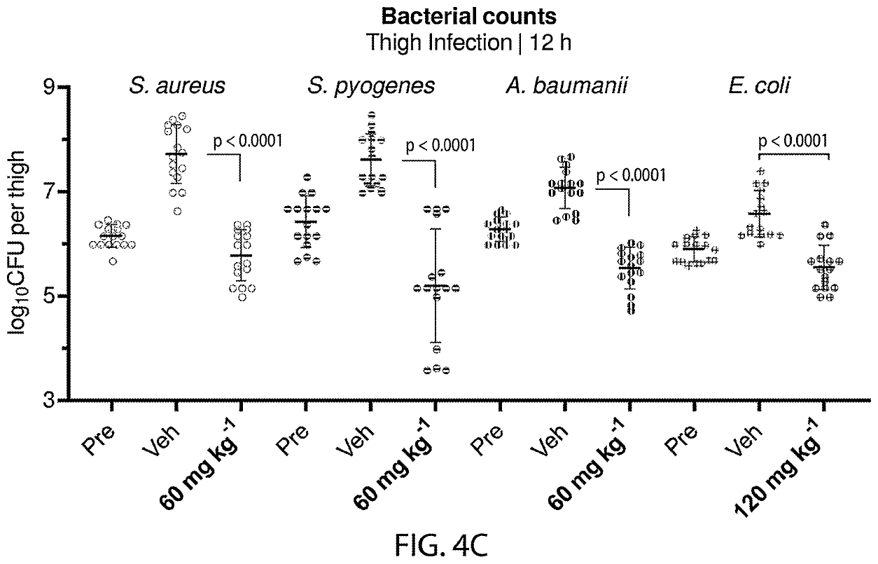

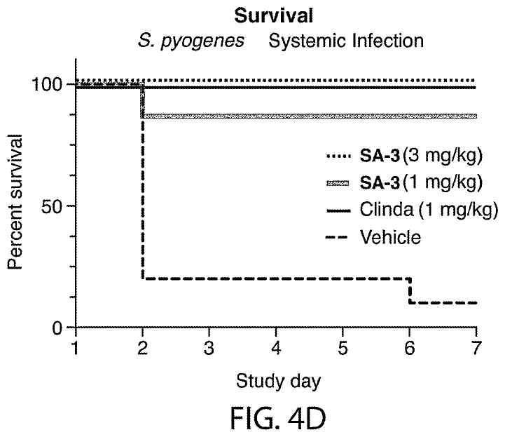

The efficacy of FSA-513018b in animal infection models was next evaluated. In cell-culture safety profiling, FSA-513018b was non-hemolytic and non-toxic toward mammalian cells (GI₅₀>50 μM, FIGS. 3A, D-F), and had no effect on membrane integrity or mitochondrial function (FIG. 3B-C). In mice, FSA-513018b achieved greater exposure than clindamycin, exhibiting an intravenous mean residence time (MRT) of 1.2 h and 24% oral bioavailability (Table 12). With these data in hand, the pharmacodynamic effect of FSA-513018b in a murine neutropenic thigh-infection model using S. pyogenes ATCC 19615 and S. aureus (MRSA) as test organisms was evaluated. In both experiments, FSA-513018b achieved statistically significant reductions in bacterial burden 24 h following an intraperitoneal dose, when compared to clindamycin- and vehicle-treated controls (FIG. 4A-B). In addition, FSA-513018b is also effective against Gram-negative bacteria such as A. bamanii and E. coli (FIG. 4C). The in vivo efficacy of FSA-513018b was also studied in a murine model of systemic infection, in which mice received an intraperitoneal dose of S. pyogenes ATCC 19615, followed by intravenous administrations of FSA-513018b (1, 3, or 10 mg/kg), clindamycin phosphate (1, 3, or 10 mg/kg), or vehicle twice daily for two days. Treatment with FSA-513018b was well-tolerated at all dose levels in this experiment, and resulted in complete rescue of infected animals, with all mice treated at the 3 and 10 mg/kg dose levels surviving (FIG. 4D, Table 13, SA-3=FSA-513018b).

TABLE 12

| | Spe- | Clindamycin | | FSA-513018b | |
|---|---|---|---|---|---|
| | cies | i.v. | p.o. | i.v. | p.o. |
| Dose (mg/kg) | m | 60 | 60 | 60 | 60 |
| AUC (μg · h/mL) | | 2.1 ± 0.5 | 0.51 ± 0.09 | 6.8 ± 1.3 | 1.6 ± 0.4 |
| Cₘₐₓ (μg/mL) | | 2.9 ± 0.7 | 0.52 ± 0.10 | 7.9 ± 3.0 | 1.6 ± 0.7 |
| MRT (h) | | 0.9 ± 0.3 | — | 1.2 ± 0.2 | — |
| CL (L/kg/h) | | 29 + 7 | — | 8.9 ± 1.8 | — |
| Vₛₛ (L/kg) | | 25 ± 1 | — | 12.5 ± 0.4 | — |
| Bioavailability | | — | 24% + 7% | — | 24% ± 7% |

| | | | |
|---|---|---|---|
| Cl_{int,mic} | m | ND | 21 |
| (μg/min/mg) | h | 55 | 34 |
| Plasma protein | m | 72% | 95% |
| binding | h | 87% | 92% |

TABLE 13
Mouse survival in S. pyogenes systemic infection model

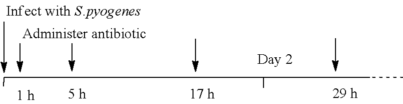

| Mice | | | | | | | | | |
|---|---|---|---|---|---|---|---|---|---|
| surviving | | Day 1 | Day 2 | Day 3 | Day 4 | Day 5 | Day 6 | Day 7 | n |
| | Vehicle | 10 | 3 | 2 | 2 | 2 | 1 | 1 | 10 |
| | (10% Captisol) | | | | | | | | |
| Clindamycin | 1 mg/kg | 8 | 8 | 8 | 8 | 8 | 8 | 8 | 8 |
| | 3 mg/kg | 8 | 8 | 8 | 8 | 8 | 8 | 8 | 8 |
| | 10 mg/kg | 8 | 7 | 7 | 7 | 7 | 7 | 7 | 8 |
| SA-3 | 1 mg/kg | 8 | 7 | 7 | 7 | 7 | 7 | 7 | 8 |
| | 3 mg/kg | 8 | 8 | 8 | 8 | 8 | 8 | 8 | 8 |
| | 10 mg/kg | 8 | 8 | 8 | 8 | 8 | 8 | 8 | 8 |

The encouraging profile of FSA-513018b prompted an investigation into its on-target activity and the structural basis for its improved antibacterial properties. While spontaneous resistance was not observed in standard strains of S. aureus or E. faecalis over the course of three-day experiments, FSA-513018b-resistant mutants were successfully selected using an E. coli strain SQ110DTC specifically designed for selecting mutations in rRNA that render cells resistant to ribosome-targeting inhibitors. This strain lacks six out of seven rRNA alleles and is devoid of the major multidrug efflux pump TolC. Upon plating SQ110DTC cells on an agar plate containing FSA-513018b, resistant clones with MICs in the range of 2-4 μg/mL appeared with a frequency of ˜10⁻⁸. All of the 14 randomly selected clones carried the single-nucleotide mutations A2058G or A2059G within the 23S rRNA (Table 14), corresponding to changes in the canonical lincosamide binding site. This result demonstrated that, analogously to other lincosamides, FSA-513018b targets the bacterial ribosome.

TABLE 14
Mutations selected in E. coli strain SQ110DTC conferring
resistance to FSA-513018b

| | FSA-513018b | | MIC (μg/mL) | |
|---|---|---|---|---|
| | concentration on | | FSA- | |
| Clone | selection plate (μg/mL) | Mutation | 513018b | Clindamycin |
| SQ110DTC | | WT | 0.03 | 16 |
| SA-3R1 | 1 | 2058G | 4 | >128 |
| SA-3R13 | 1 | 2059G | 2 | >128 |
| SA-3R14 | 1 | 2058G | ND | ND |
| SA-3R2 | 0.25 | 2059G | 2 | >128 |
| SA-3R3 | 0.25 | 2059G | ND | ND |
| SA-3R4 | 0.25 | 2058G | ND | ND |
| SA-3R5 | 0.25 | 2059G | ND | ND |
| SA-3R6 | 0.25 | 2059G | ND | ND |
| SA-3R7 | 0.25 | 2059G | ND | ND |
| SA-3R8 | 0.25 | 2059G | ND | ND |
| SA-3R9 | 0.25 | 2058G | 4 | >128 |
| SA-3R10 | 0.25 | 2058G | ND | ND |
| SA-3R11 | 0.25 | 2058G | ND | ND |
| SA-3R12 | 0.25 | 2059G | ND | ND |

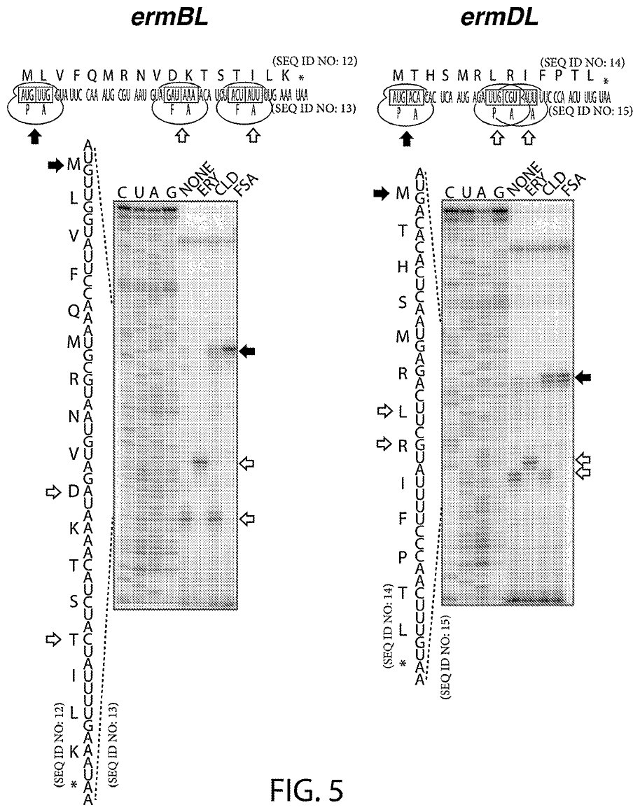

In order to understand the effect of FSA-513018b on ribosomal function, primer extension inhibition analysis (“toeprinting”) was performed. This is an in vitro technique that detects the position of the drug-arrested ribosome on mRNA in cell-free translation. Because the ribosome is exposed to the antibiotic prior to the initiation of translation in this assay, a strong inhibitor of peptide-bond formation would be expected to arrest the ribosome at the start codons of open reading frame (ORFs), whereas a more weakly binding inhibitor of the catalytic center allow a degree of escape from start codons. In the toeprinting experiment, consistent with prior reports, clindamycin arrested translation at the start codons of the model ORFs, yet allowed a fraction of ribosomes to translate ORFs up to the trap codon, at which point ribosomes are arrested due to the lack of isoleucyl-tRNA in the reaction (FIG. 5). In contrast, present at the same concentration (50 μM), FSA-513018b firmly locked the ribosome at the start codons, allowing virtually no escape, suggesting that FSA-513018b associates much more strongly with the ribosome than does clindamycin.

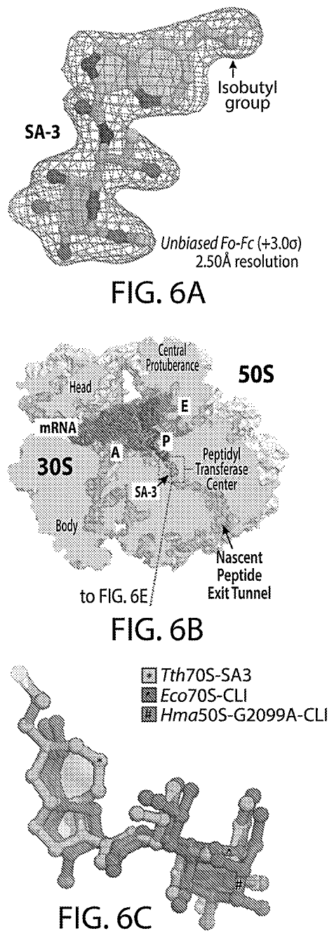

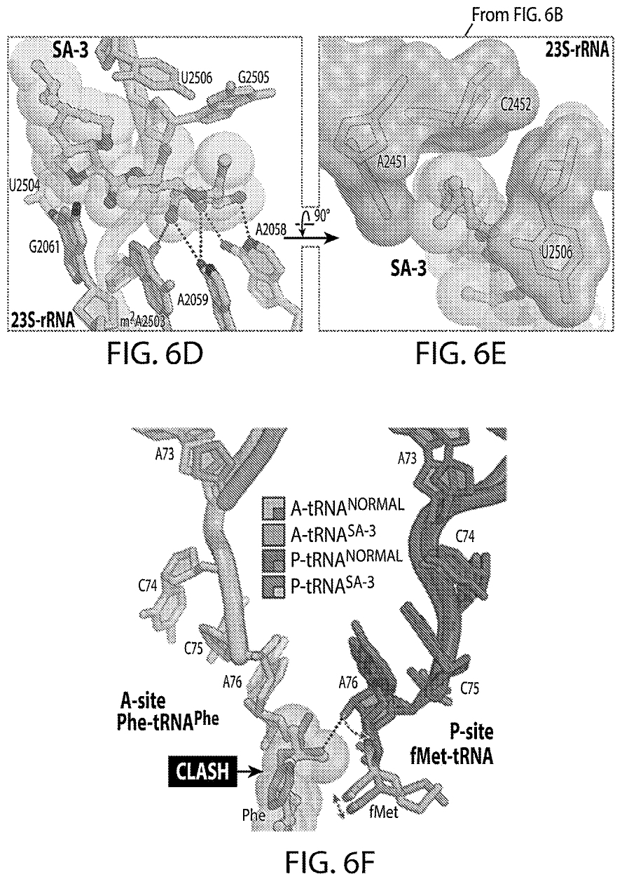

To illuminate the structural basis for this improved activity, the crystal structure of FSA-513018b bound to the bacterial ribosome was determined. 70S ribosomes from the Gram negative bacterium Thermus thermophilus (Tth) were co-crystallized in the presence of FSA-513018b, mRNA, non-hydrolyzable aminoacyl-tRNA analog fMet-NH-tRNAᵢ^{Met} (located within the P site), and deacylated tRNA^{Phe} (within the A and E sites). The crystals we obtained diffracted to 2.50 Å resolution. The unbiased Fₒ-F_{c} difference Fourier map revealed positive electron density peaks resembling characteristic chemical features of FSA-513018b (FIG. 6), confirming that the antibiotic binds in the canonical binding pocket within the large ribosomal subunit (FIG. 6B), spanning the peptidyl transferase center (PTC) and extending into the nascent peptide exit tunnel (NPET).

The binding of FSA-513018b to the functional Tth 70S ribosome complex containing mRNA and tRNAs corresponds closely to the binding of clindamycin to the tRNA-free 70S ribosome of E. coli or the large ribosomal subunit of the archaeon Haloarcula marismortui (FIG. 6C). As with historical lincosamides, a network of hydrogen bonds anchors the aminooctose moiety of FSA-513018b to the NPET nucleotides A2058, A2059, and A2503. Likewise, the cationic prolyl residue of FSA-513018b occupies a hydrophilic pocket formed by the PTC residues G2061 and U2504 (FIG. 6D), displacing a divalent magnesium ion typically observed in this location in the drug-free ribosome. The oxepane ring of ribosome-bound FSA-513018b overlays closely with the C6′ and C7′ n-propyl-group atoms of clindamycin, stabilizes the Cγ-endo proline ring pucker, and presents the 7′-isobutyl group for interaction with A-site cleft formed by 23S rRNA residues A2451 and C2452 (FIG. 6E). This cleft, which normally accommodates the side-chains of incoming amino acids, plays a key role in the positioning of aminoacylated 3′ ends of A-site tRNAs within the PTC during translation. The electron density corresponding to the CCA terminus of the A-site tRNA is poorly resolved in the structure, suggesting that FSA-513018b blocks proper coordination of the 3′-terminal nucleotides and aminoacyl residue of A-site tRNA. The 7′-isobutyl substituent of FSA-513018b extends deep enough within the A-site cleft to overlap not only with incoming amino acids (as the n-propyl group of clindamycin does, albeit to a lesser extent) but also with the P-site amino acid (FIG. 6F). This observation, together with the toeprinting results described above, points to FSA-513018b functioning as a specific inhibitor of translation initiation, similar to the pleuromutulin antibiotics.

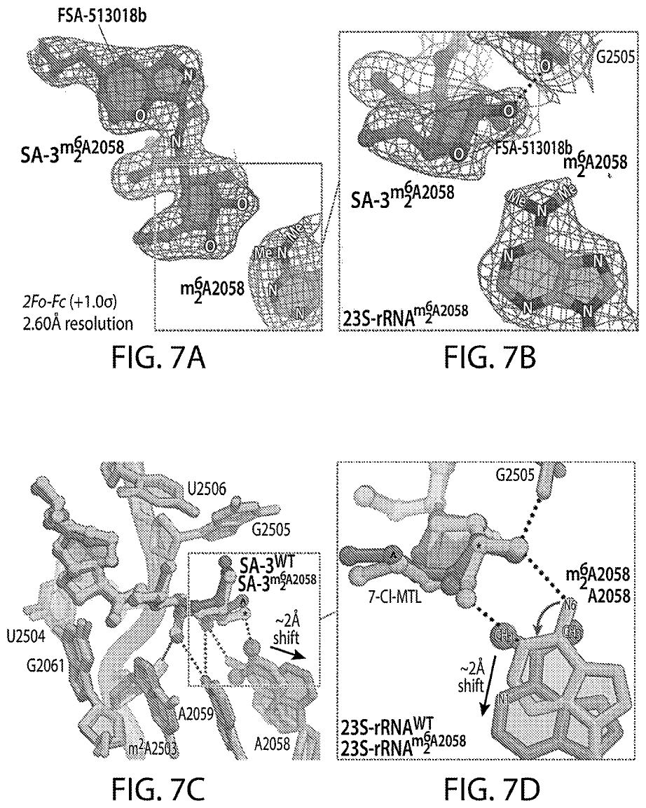

The extraordinary activity of FSA-513018b against bacteria harboring erm and cfr resistance genes prompted a determination of the crystal structure of the compound bound to the m₂⁶A2058 ribosome as well. Erm-mediated methylation of A2058 confers resistance to clindamycin by blocking the association of its 7-Cl-MTL residue with the NPET, and because FSA-513018b bears the same aminooctose residue as clindamycin, its ability to overcome MLS_{B} resistance was unexpected. Erm-modified ribosomes with A2058 dimethylation levels reaching ˜60% were isolated from Tth cells expressing the erm gene from Bifidobacterium thermophilum. Co-crystallization of these ribosomes with FSA-513018b provided crystals diffracting to 2.60-Å resolution. Remarkably, the corresponding electron-density map revealed that FSA-513018b binds to the methylated ribosome in a manner almost identical to its positioning within the wild-type ribosome (FIG. 7A-B), while m₂⁶A2058 undergoes a movement of ˜2 Å relative to its wild-type position in order to accommodate the antibiotic (FIG. 7C-D). N⁶-dimethylation, together with this previously unknown displacement of A2058, disrupts the two hydrogen bonds typically formed between this residue and the aminooctose residue of lincosamides. Loss of these bonds is sufficient to destabilize binding of clindamycin to the Erm-modified ribosome; however, new hydrophobic interactions of FSA-513018b with the A-site cleft appear to compensate for the loss of these interactions, resulting in sufficiently high affinity for Erm-modified ribosomes for FSA-513018b to overcome resistance

### Methods

Minimum inhibitory concentrations (MICs) were determined by broth microdilution method or agar dilution method following the Clinical and Laboratory Standards Institute guidelines. Standard bacterial strains were obtained from American Type Culture Collection (ATCC). Other bacterial strains were obtained from hospitals and commercial sources, so noted in Table 11. Before the start of the MIC experiment, standard and test compound stock solutions were prepared in dimethyl sulfoxide (DMSO, Aldrich D2650) at a stock concentration of 5,120 μg/mL. The compound concentration range typically employed for each experiment was 128-0.06 μg/mL (2.5% final DMSO concentration). Clindamycin was used as a comparator in all experiments and each experiment was performed in triplicate. Specialized procedures were employed for streptococcal and anaerobic strains. All other bacterial strains were sub-cultured on blood agar plates (tryptic soy agar with 5% sheep blood, Hardy Diagnostics) and incubated overnight at 35° C. Organisms were suspended in cation-adjusted Mueller-Hinton broth (CaMHB, BD 212322) and optical density was adjusted to 0.5 McFarland standard. Suspensions were further diluted to obtain a final inoculum of 5×10⁵ CFU/mL for broth microdilution experiments. The minimum concentration of compound required to inhibit visible bacterial growth after 24 h of incubation was recorded as the MIC.

Time-kill studies: Time kill studies were performed using four different concentrations of standard and test compounds (1, 2, 4, and 10×MIC). Experiments were performed in duplicate following CLSI guidelines. An inoculum was prepared in Mueller-Hinton broth (MHB) containing 0.5-5×10⁶ CFU/mL of test organism. Cultures were incubated at 37° C. in a shaker incubator at 110 rpm; a flask containing bacteria left unexposed to antibiotic was used as untreated control. At time points 0, 1, 3, 6, and 24 h following administration of antibiotic, bacterial counts were determined from each flask by serial dilution and plating on brain heart infusion agar (BD, Sparks, M D). Plates were incubated at 37° C. in incubators for 18-24 hours to determine bacterial counts. For streptococcal strains, blood agar plates (Tryptic Soy Agar with 5% Sheep Blood) were used for culturing the organisms (plates were incubated in the presence of 5% CO₂), and time-kill studies were performed in media supplemented with 5% laked horse blood. Compounds exhibiting 3 log₁₀ CFU/mL reduction compared to initial counts were considered to exhibit bactericidal activity. Compounds exhibiting ≤2 log₁₀ CFU reduction or maintaining the counts similar to initial bacterial counts are considered to be bacteriostatic

Post-antibiotic and post-antibiotic sub-MIC effect: Measurements of PAE and PA-SME durations were performed according to the methods described by Odenholt-Tornqvist and co-workers. Bacterial strains were cultured overnight on blood agar plates. Optical density was adjusted to 0.5 McFarland standard in CaMHB and suspensions were diluted further into 50-mL sterile Erlenmeyer flasks containing 20 mL CaMHB to achieve a bacterial load of 0.5-1×10⁶ CFU/mL. Cultures were then exposed to test and standard antibiotic at 1, 2, 4, and 10×MIC concentrations for 1 hour at 37° C. in a shaker incubator at 110 rpm (0.5% final DMSO concentration). A flask containing the unexposed bacterial strain was left as untreated control. Post-exposure, 100 μL of bacterial culture was diluted in 900 μL of sterile CaMHB; 100 μL of this diluted sample was immediately diluted further in 19.9 mL of sterile CaMHB (1:2,000 final dilution). For PA-SME studies, post exposure and dilution, each flask (except those in the control group) was supplemented with 0.25 and 0.5×MIC per mL of specific antibiotic (0.5% final DMSO concentration). Flasks were incubated at 37° C. in a shaker incubator (110 rpm). Samples (100 μL from each flask per time point) were collected at 0, 1, 3, 6, and 24 h from each flask and were serially diluted. From each dilution, 100 μL and 10 μL from each dilution were plated in duplicate on TSA plates. Plates were incubated overnight at 35° C. to determine the bacterial counts (limit of detection=20 CFU/plate or 2×10² CFU/mL). These counts were plotted against time with linear interpolation between time points to obtain the growth-kinetic curves. PAE and PA-SME were determined from these curves using the formulae:

PAE=T−C

where T is the time required for bacteria previously exposed to antibiotic to multiply 1 log₁₀ above counts immediately following dilution, and C is the corresponding time required for untreated culture to do the same; and

PA-SME=Tₚₐ−C

where Tₚₐ is the time taken for bacteria previously exposed to antibiotic and then re-exposed to sub-MIC concentrations to increase 1 log₁₀ above counts immediately following dilution.

Mammalian cell experiments: All compounds were dissolved in sterile DMSO to a stock concentration of 20 μM and aliquoted prior to freezing at −20° C. Aliquots were limited to a maximum of 3 freeze-thaw cycles. A549 (human pulmonary carcinoma; male) cells were cultured in Dulbecco's Modified Eagle Medium (DMEM) supplemented with 10% fetal bovine serum. K562 (human chronic myelogenous leukemia; female) cells were cultured in Iscove's Modification of DMEM (IMDM) media supplemented with 10% fetal bovine serum. HCT116 (human colorectal carcinoma; male) cells were cultured in McCoy's 5a (Iwakata & Grace Modification) media supplemented with 10% fetal bovine serum. HepG2 (human hepatocellular carcinoma; male) cells were cultured in Eagle's Minimum Essential Medium (EMEM) supplemented with 10% fetal bovine serum. All cell cultures were maintained in a 5% CO₂ incubator at 37° C.

Growth inhibition was studied using the Promega CellTiter-Blue cell viability assay pursuant to the manufacturer's protocol. Briefly, cells were harvested, diluted, and mixed with the desired concentration of test compound. This suspension was then added to a 96-well plate (5×10⁴ cells/mL, 100 μL/well, 5 replicates per test compound), and the plates were incubated for 72 h at 37° C. in a 5% CO₂ incubator. Next, 20 μL of CellTiter-Blue reagent was added to each well and the plates were incubated for 4 h. Plates were read on a SpectraMax i3 plate reader. GI₅₀ values were determined by nonlinear regression using GraphPad Prism and represent the mean of at least three independent experiments.

Mitochondrial toxicity assays were conducted using the Promega Mitochondrial ToxGlo assay, pursuant to the manufacturer's protocol.

Animal studies: Animal experiments were performed at the Biology Research Infrastructure Laboratory of Harvard University following IACUC-approved protocols. Animals were maintained in accordance with National Research Council guidelines. Pathogen-free, 5-6-week-old, female CD-1 mice weighing 22-26 grams were obtained from Charles River Laboratories, Inc. (Kingston, N.Y.). Animals were acclimated for a minimum of three days prior to start of the studies. Mice were caged as a group of four per cage, were housed at 21-23° C. with humidity ranging from 30-70%, were exposed to 12-hour light and dark cycles, and were supplied with food and water ad libitum.

Mouse thigh infection studies: Mice were rendered neutropenic by administering cyclophosphamide intraperitoneally, 150 mg/kg four days prior, and 100 mg/kg one day prior to infection. Bacterial strains were cultured overnight on blood agar plates at 35° C. in 5% CO₂. Bacterial inoculum was prepared in sterile brain heart infusion broth (BHIB) and OD was adjusted to 0.1 at 600 nm; the inoculum was further diluted to achieve a bacterial load of 0.5-1×10⁷ CFU/mL. Animals were infected by intramuscular injection of 0.1 mL of inoculum into each thigh (0.2 mL of inoculum was used per animal). Mice were divided into 4 groups: FSA-513018b (n=12 animals), clindamycin (n=12), vehicle-treated control (n=12), and untreated baseline (n=2) At 2 h post-infection (t=0), the untreated animals were euthanized to obtain baseline bacterial counts; the others received a single dose of FSA-513018b (6 mg/mL in 10% Captisol, 250 μL, 60 mg/kg), clindamycin phosphate (6 mg/mL in sterile saline, 250 μL, 60 mg/kg), or sterile vehicle (10% Captisol, 250 μL) by intraperitoneal injection. Intramuscular bacterial counts in treated animals were determined 1, 3, 6, 12, 18, and 24 h after treatment. At each time point animals from each group (n=2) were euthanized by CO₂ inhalation, and thighs (4 per time point) were aseptically removed and homogenized in sterile saline. Homogenates were serially diluted and plated on blood agar plates (each sample was plated in duplicate), and bacterial counts (log₁₀ CFU/thigh) were determined after incubating the plates overnight at 35° C. in 5% CO₂. Each experiment was repeated twice, and mean values were calculated (n=4 mice per time point).

Mouse systemic infection study: S. pyogenes ATCC 19615 was cultured overnight on blood agar plates in 5% CO₂ at 35° C. before being suspended in BHIB. The optical density of the bacterial suspension was adjusted to 0.1 at 600 nm using sterile BHIB and was then further diluted 1:1 in 10% hog gastric mucin (HGM, type III) to prepare the infecting inoculum in 5% HGM. Mice were infected by intraperitoneal injection of 250 μL, infecting inoculum, representing 1-2×10⁶ CFU per animal. At time points 1, 5, 17, and 29 h post-infection, animals were administered intravenous infusions of FSA-513018b (1, 3, or 10 mg/kg, 250 μL in 10% Captisol; 8 mice per dose level), clindamycin phosphate (1, 3, or 10 mg/kg, 250 μL in normal saline; 8 mice per dose level), or vehicle as negative control (10% Captisol, 250 μL; 10 mice). Animals were monitored for survival for seven days following infection.

Spontaneous resistance in standard bacterial strains: Cation-adjusted Mueller-Hinton agar plates containing FSA-513018b or clindamycin at concentrations corresponding to 4×MIC, 8×MIC, and 16×MIC were prepared. Bacterial strains (S. aureus ATCC 29213, S. aureus MRSA, and E. faecalis ATCC 29212) were suspended in normal saline and optical density was adjusted to 1.50 at 600 nm. Bacterial counts were measured by serial dilution and plating. Bacterial inoculum (250 μL) was transferred to the media plate and was spread evenly. Plates were incubated at 37° C. for 3 days, and were observed daily for resistant colonies to emerge. No colonies were noted on any plate during the course of this experiment.

Selection of FSA-513018b resistant mutants in SQ110DTC: The MICs of FSA-513018b and clindamycin against E. coli strain SQ110DTC were determined in lysogeny broth (LB) medium by placing 100 μL of exponentially growing cells in the wells of a 96-well plate, adding 2-fold dilutions of antibiotics and incubating plates for 18 h at 37° C. (Table 16). For selection of the resistant mutants, ˜10⁹ colony forming units (1.2 ml of the exponentially growing cell culture with opticxal density of A₆₀₀=1.2), were plated on LB/agar plates containing either 0.25 μg/mL or 1 μg/mL of FSA-513018b (-10× or ˜30×MIC, respectively). Approximately 20 colonies appeared on both plates after incubation at 37° C. for 24 h. The segment of the 23S rRNA gene corresponding to domains V and VI of the 23S rRNA was PCR-amplified from 14 randomly selected colonies using the primers 2020R (CCC GAG ACT CAG TGA AAT TGA ACT C) and L2904 (AAG GTT AAG CCT CAC GG). PCR products were sequenced using the primer L2667 (GGT CCT CTC GTA CTA GGA GCA G).

Toeprinting analysis: The ermBL DNA template for toeprinting was generated by a 4-primer cross-over PCR using primers T7, NV1, ermB3-F and ermB3-R. The ermDL template was generated in the same way using primers RLR-fwd, RLR-rev, T7-SD-fwd and T7-SD-rev. In vitro translation in the PURExpress system (New England Biolabs) and toeprinting analysis were carried out as described previously. The antibiotics (mupirocin, clindamycin, erythromycin, FSA-513018b) were present in the reactions at a final concentration of 50 μg/mL.

Primer sequences used for toeprinting are as follows: T7 (TAA TAC GAC TCA CTA TAG GG); NV1 (GGT TAT AAT GAA TTT TGC TTA TTA AC); ermB3-F (TAA TAC GAC TCA CTA TAG GGC TTA AGT ATA AGG AGG AAA AAA TAT GTT GGT ATT CCA AAT GCG TAA TGT AGA TAA AAC ATC TAC); ermB3-R (GGT TAT AAT GAA TTT TGC TTA TTA ACG ATA GAA TTC TAT CAC TTA TTT CAA AAT AGT AGA TGT TTT ATC TAC ATT ACG); RLR-fwd (GGA GGA AAA AAT ATG ACA CAC TCA ATG AGA CTT CGT ATT TTC CC); RLR-rev (CTA TCA CTT ACA AAG TTG GGA AAA TAC GAA GTC TCA TTG AG); T7-SD-fwd (TAA TAC GAC TCA CTA TAG GGC TTA AGT ATA AGG AGG AAA AAA TAT GAC ACA CTC AAT G); T7-SD-rev (GGT TAT AAT GAA TTT TGC TTA TTA ACG ATA GAA TTC TAT CAC TTA CAA AGT TGG GAA AAT).

Crystallization of FSA-513018b with WT Tth ribosomes: Ribosome complexes comprising 70S ribosomes from Thermus thermophilus (strain HB8), mRNA, and tRNAs were prepared as described previously. FSA-513018b was added to the pre-formed ribosome complexes to a final concentration of 250 μM prior to crystallization. All Tth 70S ribosome complexes were formed in a buffer containing 5 mM HEPES-KOH (pH 7.6), 50 mM KCl, 10 mM NH₄Cl, and 10 mM Mg(OAc)₂, and were then crystallized in a buffer containing 100 mM Tris-HCl (7.6), 2.9% (w/v) PEG-20K, 9-10% (w/v) MPD, 175 mM arginine, and 0.5 mM β-mercaptoethanol. Crystals were grown by the vapor diffusion method in sitting drops at 19° C. and stabilized as described previously, with FSA-513018b added to the stabilization buffers (100 μM).

Diffraction data were collected at the beamlines 24ID-C and 24ID-E at the Advanced Photon Source (Argonne National Laboratory, Argonne, Ill.). A complete dataset for each ribosome complex was collected using 0.979-Å wavelength at 100K from multiple regions of the same crystal using 0.3-degree oscillations. The raw data were integrated and scaled using the XDS software package. All crystals belonged to the primitive orthorhombic space group P2₁2₁2₁ with approximate unit cell dimensions of 210×450×620 Å and contained two copies of the 70S ribosome per asymmetric unit. Each structure was solved by molecular replacement using PHASER from the CCP4 program suite. The initial search model was generated from the previously published structure of T. thermophilus 70S ribosome with bound mRNA and tRNAs (PDB entry 6XHW). The initial molecular replacement solutions were refined by rigid-body refinement with the ribosome split into multiple domains, followed by positional and individual B-factor refinement using PHENIX. Non-crystallographic symmetry restraints were applied to four parts of the 30S ribosomal subunit (head, body, spur, helix 44), and four parts of the 505 subunit (body, L1-stalk, L10-stalk, C-terminus of the L9 protein).

Atomic models of FSA-513018b were generated from its known chemical structure using PRODRG online software, which was also used to generate restraints for energy minimization and refinement based on idealized 3D geometry. Atomic models and restraints were used to fit and refine FSA-513018b into the obtained electron density map (FIG. 6A). The final models of the Tth 70S ribosome in complex with mRNA, tRNAs, and FSA-513018b were generated by multiple rounds of model building in COOT, followed by refinement in PHENIX. The statistics of data collection and refinement are compiled in Tables A-C. All figures showing atomic models were generated using PyMol software.

Statistical analyses: Statistical analysis was performed using GraphPad Prism. Mouse thigh-infection study data (24-h time points) were compared using a one-way ANOVA followed by Tukey's multiple comparisons test.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 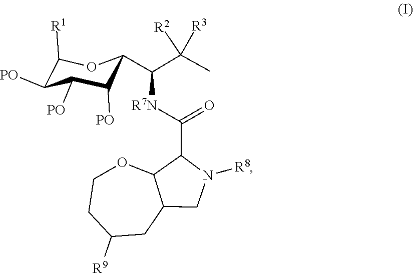
or a pharmaceutically acceptable salt thereof, wherein:
each P is independently hydrogen or an oxygen protecting group;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, or —SR^{A};
R² is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};
R³ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroaliphatic;
R⁷ is hydrogen or unsubstituted alkyl;
R⁸ is hydrogen, substituted or unsubstituted alkyl, substituted or unsubstituted carbocyclyl, substituted or unsubstituted heterocyclyl, substituted or unsubstituted heteroaliphatic, —C(═O)R^{A}, —C(═O)OR^{A}, —C(═O)N(R^{A})₂, —S(O)₂R^{A}, or a nitrogen protecting group;
R⁹ is substituted or unsubstituted alkyl, substituted or unsubstituted alkenyl, substituted or unsubstituted alkynyl, substituted or unsubstituted carbocyclyl, or substituted or unsubstituted heteroaliphatic, provided that R⁹ is not aralkyl; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

2. The compound claim 1, or a pharmaceutically acceptable salt thereof, wherein:
R¹ is substituted or unsubstituted alkyl, substituted or unsubstituted alkenyl, substituted or unsubstituted carbocyclyl, substituted or unsubstituted heterocyclyl, substituted or unsubstituted heteroaryl, substituted or unsubstituted heteroaralkyl, substituted or unsubstituted heteroalkyl, —OR^{A}, —N(R^{A})₂, or —SR^{A}.

3. The compound of claim 1 or 2, or a pharmaceutically acceptable salt thereof, wherein:
R¹ is substituted or unsubstituted alkyl, substituted or unsubstituted carbocyclyl, or —SR^{A}.

4. The compound of any of claims 1-3, or a pharmaceutically acceptable salt thereof, wherein:
R¹ is —SR^{A}.

5. The compound of any of claims 1-4, or a pharmaceutically acceptable salt thereof, wherein:
R¹ is —SR^{A}, and R^{A} is unsubstituted alkyl.

6. The compound of any of claims 1-5, or a pharmaceutically acceptable salt thereof, wherein:
R¹ is —SR^{A}, and R^{A} is unsubstituted C₁₋₄ alkyl.

7. The compound of any of claims 1-6, or a pharmaceutically acceptable salt thereof, wherein:
R¹ is —SCH₃.

8. The compound of any of claims 1-7, or a pharmaceutically acceptable salt thereof, wherein:
R² is halogen, substituted or unsubstituted alkyl, —OR^{A}, —N₃, —N(R^{A})₂, or —SR^{A}.

9. The compound of any of claims 1-8, or a pharmaceutically acceptable salt thereof, wherein:
R² is halogen or —SR^{A}.

10. The compound of any of claims 1-9, or a pharmaceutically acceptable salt thereof, wherein:
R² is halogen.

11. The compound of any of claims 1-10, or a pharmaceutically acceptable salt thereof, wherein:
R² is —Cl.

12. The compound of any of claims 1-11, or a pharmaceutically acceptable salt thereof, wherein:
R² is —SR^{A}.

13. The compound of any of claims 1-12, or a pharmaceutically acceptable salt thereof, wherein:
R² is —SR^{A}; and R^{A} is substituted or unsubstituted aryl.

14. The compound of any of claims 1-13, or a pharmaceutically acceptable salt thereof, wherein:
R² is —SR^{A}; and R^{A} is substituted aryl.

15. The compound of any of claims 1-14, or a pharmaceutically acceptable salt thereof, wherein:
R² is —SR^{A}; and R^{A} is substituted phenyl.

16. The compound of any of claims 1-15, or a pharmaceutically acceptable salt thereof, wherein:
R² is

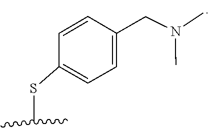 [SMILES: *Sc1ccc(CN(C)C)cc1]

17. The compound of any of claims 1-16, or a pharmaceutically acceptable salt thereof, wherein:
R³ is hydrogen, halogen, substituted or unsubstituted alkyl, or substituted or unsubstituted alkenyl.

18. The compound of any of claims 1-17, or a pharmaceutically acceptable salt thereof, wherein:
R³ is hydrogen or substituted or unsubstituted alkyl.

19. The compound of any of claims 1-18, or a pharmaceutically acceptable salt thereof, wherein:
R³ is hydrogen.

20. The compound of any of claims 1-19, or a pharmaceutically acceptable salt thereof, wherein:
each P is hydrogen.

21. The compound of any of claims 1-20, or a pharmaceutically acceptable salt thereof, wherein:
R⁷ is hydrogen.

22. The compound of any of claims 1-21, or a pharmaceutically acceptable salt thereof, wherein:
R⁸ is hydrogen, substituted or unsubstituted alkyl, substituted or unsubstituted carbocyclyl, or —C(═O)R^{A}.

23. The compound of any of claims 1-22, or a pharmaceutically acceptable salt thereof, wherein:
R⁸ is hydrogen or substituted or unsubstituted alkyl.

24. The compound of any of claims 1-23, or a pharmaceutically acceptable salt thereof, wherein:
R⁸ is hydrogen or methyl.

25. The compound of any of claims 1-24, or a pharmaceutically acceptable salt thereof, wherein:
R⁸ is hydrogen.

26. The compound of any of claims 1-24, or a pharmaceutically acceptable salt thereof, wherein:
R⁸ is methyl.

27. The compound of any of claims 1-26, or a pharmaceutically acceptable salt thereof, wherein:
R⁹ is substituted or unsubstituted alkyl, substituted or unsubstituted alkenyl, substituted or unsubstituted carbocyclyl, or unsubstituted heteroalkyl.

28. The compound of any of claims 1-27, or a pharmaceutically acceptable salt thereof, wherein:
R⁹ is substituted or unsubstituted alkyl, substituted or unsubstituted alkenyl, or substituted or unsubstituted carbocyclyl.

29. The compound of any of claims 1-28, or a pharmaceutically acceptable salt thereof, wherein:
R⁹ is substituted or unsubstituted alkenyl.

30. The compound of any of claims 1-29, or a pharmaceutically acceptable salt thereof, wherein:
R⁹ is unsubstituted alkenyl.

31. The compound of any of claims 1-30, or a pharmaceutically acceptable salt thereof, wherein:
R⁹ is substituted or unsubstituted carbocyclyl.

32. The compound of any of claims 1-31, or a pharmaceutically acceptable salt thereof, wherein:
R⁹ is unsubstituted carbocyclyl.

33. The compound of any of claims 1-32, or a pharmaceutically acceptable salt thereof, wherein:
R⁹ is unsubstituted C₃₋₆ cycloalkyl.

34. The compound of any of claims 1-33, or a pharmaceutically acceptable salt thereof, wherein:
R⁹ is unsubstituted C₅₋₆ cycloalkyl.

35. The compound of any of claims 1-34, or a pharmaceutically acceptable salt thereof, wherein:
R⁹ is unsubstituted cyclopentyl.

36. The compound of any of claims 1-35, or a pharmaceutically acceptable salt thereof, wherein:
R⁹ is unsubstituted cyclohexyl.

37. The compound of any of claims 1-36, or a pharmaceutically acceptable salt thereof, wherein:
R⁹ is substituted or unsubstituted alkyl.

38. The compound of any of claims 1-37, or a pharmaceutically acceptable salt thereof, wherein:
R⁹ is substituted or unsubstituted C₁₋₆ alkyl.

39. The compound of any of claims 1-38, or a pharmaceutically acceptable salt thereof, wherein:
R⁹ is C₁₋₆ alkyl substituted with halogen, alkenyl, C₃₋₆ cycloalkyl, heterocyclyl, —N(R^{A})₂, SO₂R^{A}, or —OR^{A}.

40. The compound of any of claims 1-39, or a pharmaceutically acceptable salt thereof, wherein:
R⁹ is —CH═CH₂, —CH₂CH═CH₂, —CH₃, —CH₂CH₃, —CH₂CH₂CH₃, —CH₂CH₂CH₂CH₃, —CH(CH₃)₂, —CH₂CH(CH₃)₂, —CH₂C(CH₃)₃, —CH₂CH₂F, —CH₂CHF₂, —CH₂CF₂CH₃, —CH₂CH₂CH₂F, —CH₂CH₂CF₂H, —CH₂CH₂CH₂Cl, 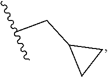 [SMILES: *CC1CC1]
—CH₂CH₂CH₂OCH₃, —CH₂CH₂CH₂OH, —CH₂C(CH₃)₂OH, —CH₂CH₂CH₂SO₂CH₃, CH₂CH₂CH₂NH₂, —CH₂CH₂CH₂N(CH₃)₂,

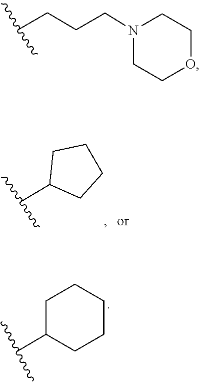 [SMILES: *CCCN1CCOCC1 ; *C1CCCC1 ; *C1CCCCC1]

41. The compound of any of claims 1-40, or a pharmaceutically acceptable salt thereof, wherein:
R⁹ is —CH₂CH═CH₂, —CH₂CH₂F, —CH₂CHF₂, —CH₂CF₂CH₃, —CH₂CH₂CH₂F, —CH₂CH₂CF₂H, —CH₂CH₂CH₂Cl, 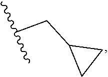 [SMILES: *CC1CC1]
—CH₂CH₂CH₂OCH₃, —CH₂CH₂CH₂OH, —CH₂C(CH₃)₂OH, —CH₂CH₂CH₂SO₂CH₃, CH₂CH₂CH₂NH₂, —CH₂CH₂CH₂N(CH₃)₂, or

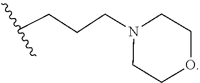 [SMILES: *CCCN1CCOCC1]

42. The compound of any of claims 1-41, or a pharmaceutically acceptable salt thereof, wherein:
R⁹ is unsubstituted C₁₋₆ alkyl.

43. The compound of any of claims 1-42, or a pharmaceutically acceptable salt thereof, wherein:
R⁹ is —CH₃, —CH₂CH₃, —CH₂CH₂CH₃, —CH₂CH₂CH₂CH₃, —CH(CH₃)₂, —CH₂CH(CH₃)₂, or —CH(CH₃)₃.

44. The compound of any of claims 1-43, or a pharmaceutically acceptable salt thereof, wherein:
R⁹ is —CH₂CH(CH₃)₂.

45. The compound of any of claims 1-44, wherein the compound is of Formula (I-a): 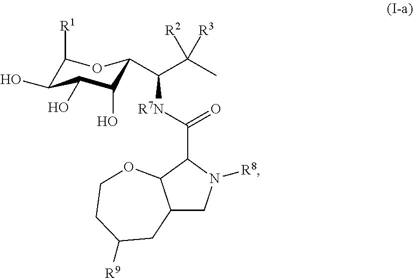
or a pharmaceutically acceptable salt thereof.

46. The compound of any of claims 1-45, wherein the compound is of Formula (I-b): 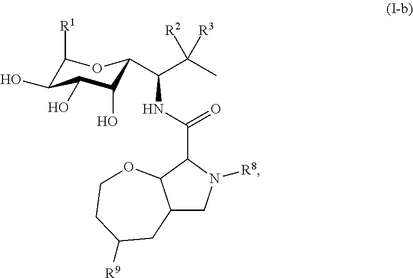
or a pharmaceutically acceptable salt thereof.

47. The compound of any of claims 1-47, wherein the compound is of Formula (I-c): 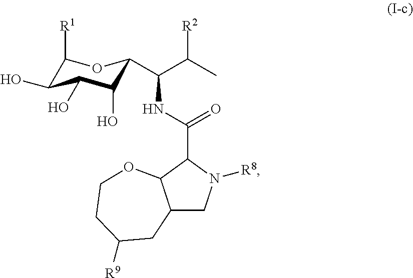
or a pharmaceutically acceptable salt thereof.

48. The compound of any of claims 1-47, wherein the compound is of Formula (I-d): 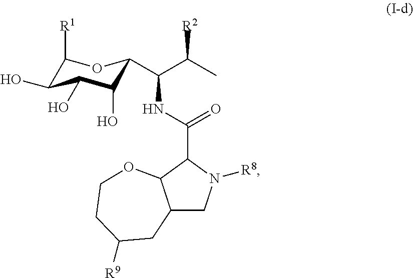
or a pharmaceutically acceptable salt thereof.

49. The compound of any of claims 1-48, wherein the compound is of Formula (I-e): 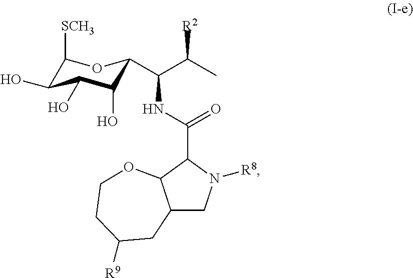
or a pharmaceutically acceptable salt thereof.

50. The compound of any of claims 1-49, wherein the compound is of Formula (I-f): 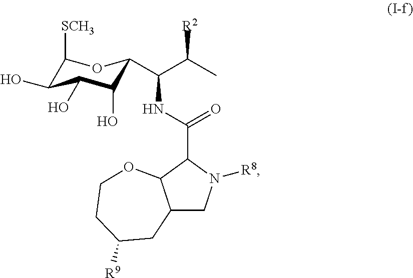
or a pharmaceutically acceptable salt thereof.

51. The compound of any of claims 1-50, wherein the compound is of Formula (I-g): 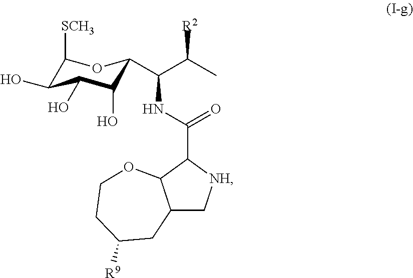
or a pharmaceutically acceptable salt thereof.

52. The compound of any of claims 1-51, wherein the compound is of Formula (I-h): 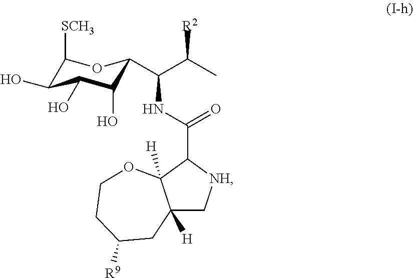
or a pharmaceutically acceptable salt thereof.

53. The compound of any of claims 1-49, wherein the compound is of Formula (I-i): 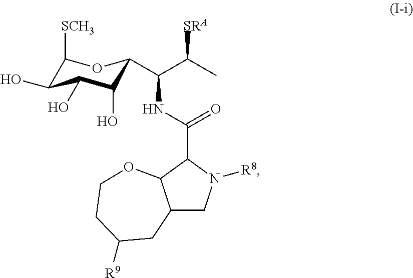
or a pharmaceutically acceptable salt thereof.

54. The compound of any of claim 1-49, or 53, wherein the compound is of Formula (I-j): 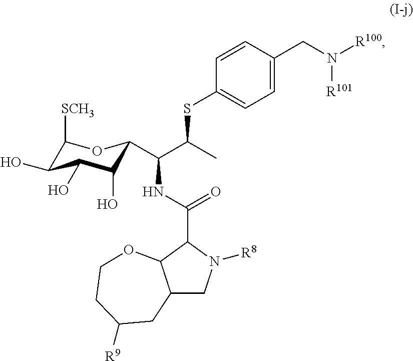
or a pharmaceutically acceptable salt thereof,
wherein:
R¹⁰⁰ and R¹⁰¹ are each independently hydrogen or alkyl, or together with the atoms to which they are attached form a substituted or unsubstituted heterocyclyl ring.

55. The compound of any of claim 1-49, 53, or 54, wherein the compound is of Formula (I-k): 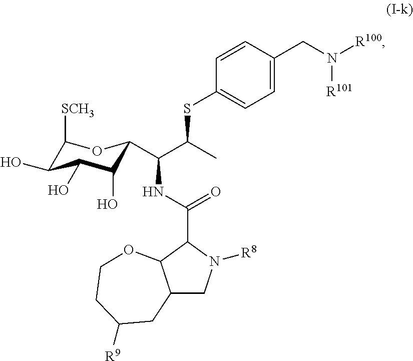
or a pharmaceutically acceptable salt thereof,
wherein:
R¹⁰⁰ and R¹⁰¹ are each independently hydrogen or alkyl, or together with the atoms to which they are attached form a substituted or unsubstituted heterocyclyl ring.

56. The compound of any of claim 1-49 or 53-55, wherein the compound is of Formula (I-l): 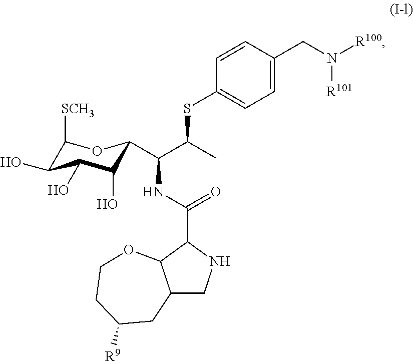
or a pharmaceutically acceptable salt thereof,
wherein:
R¹⁰⁰ and R¹⁰¹ are each independently hydrogen or alkyl, or together with the atoms to which they are attached form a substituted or unsubstituted heterocyclyl ring.

57. The compound of any of claim 1-49 or 53-56, wherein the compound is of Formula (I-m): 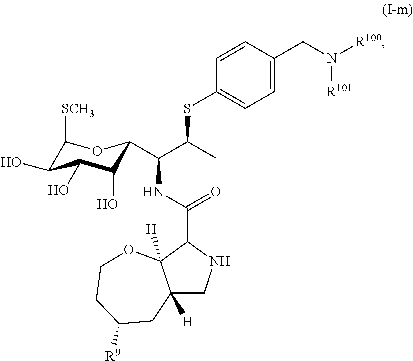
or a pharmaceutically acceptable salt thereof,
wherein:
R¹⁰⁰ and R¹⁰¹ are each independently hydrogen or alkyl, or together with the atoms to which they are attached form a substituted or unsubstituted heterocyclyl ring.

58. The compound of any of claims 1-49, wherein the compound is of Formula (I-n): 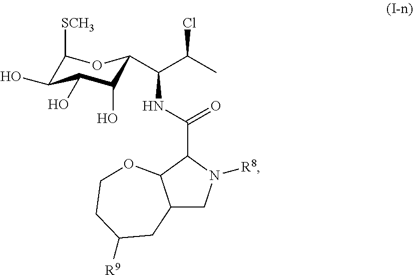
or a pharmaceutically acceptable salt thereof.

59. The compound of any of claim 1-49 or 58, wherein the compound is of Formula (I-o): 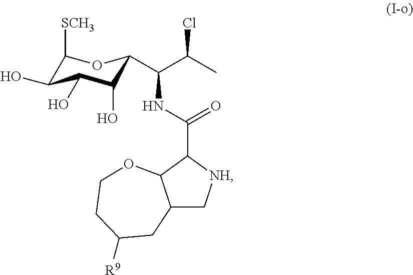
or a pharmaceutically acceptable salt thereof.

60. The compound of any of claim 1-49, 58, or 59, wherein the compound is of Formula (I-p): 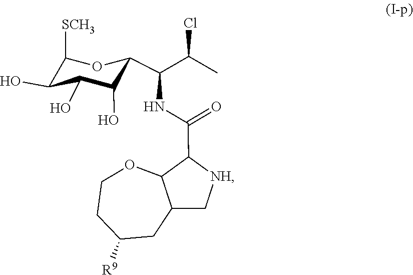
or a pharmaceutically acceptable salt thereof.

61. The compound of any of claim 1-49 or 58-60, wherein the compound is of Formula (I-q): 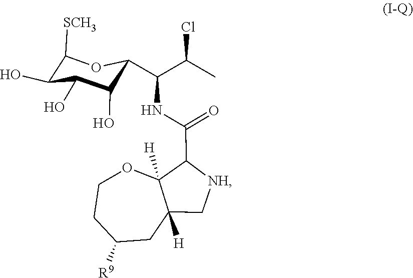
or a pharmaceutically acceptable salt thereof.

62. The compound of any of claims 1-61, wherein the compound is of Formula (I-r): 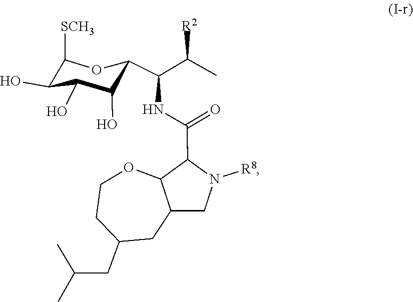
or a pharmaceutically acceptable salt thereof.

63. The compound of any of claims 1-62, wherein the compound is of Formula (I-s): 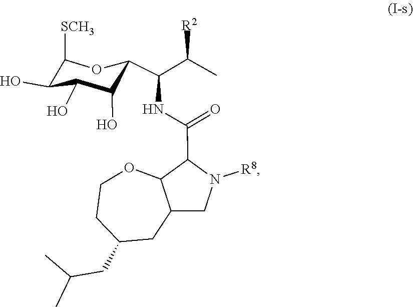
or a pharmaceutically acceptable salt thereof.

64. The compound of any of claims 1-63, wherein the compound is of Formula (I-t): 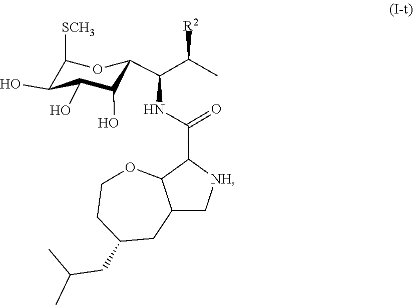
or a pharmaceutically acceptable salt thereof.

65. The compound of any of claims 1-64, wherein the compound is of Formula (I-u): 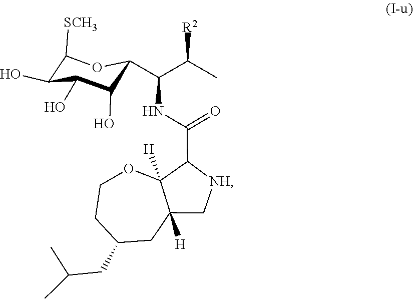
or a pharmaceutically acceptable salt thereof.

66. The compound of claim 1, wherein the compound is: 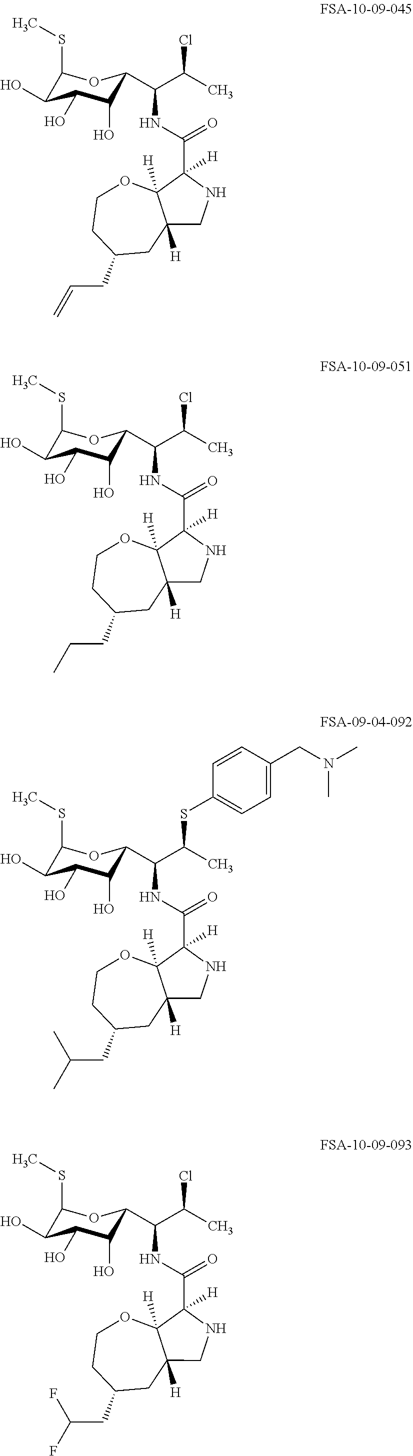 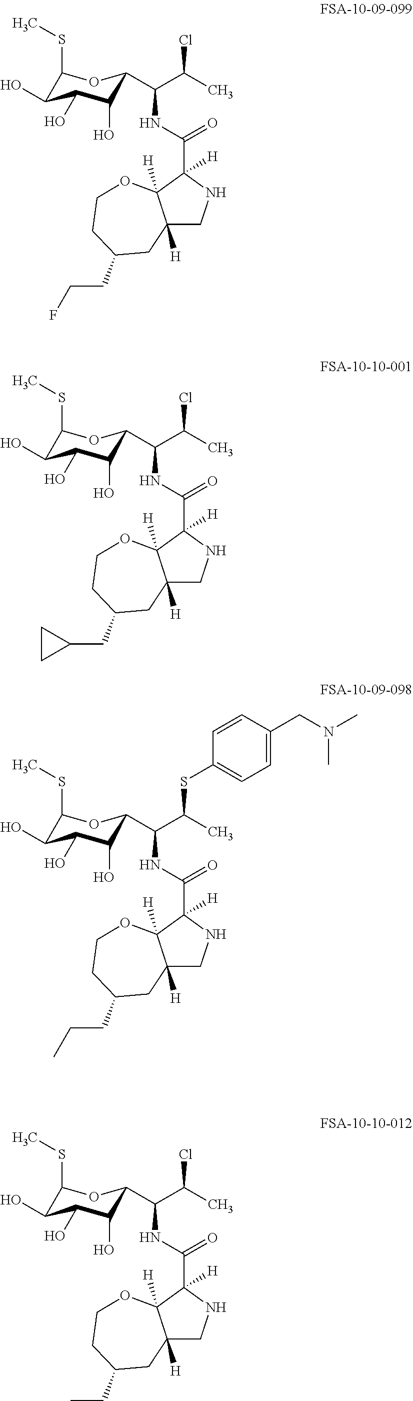 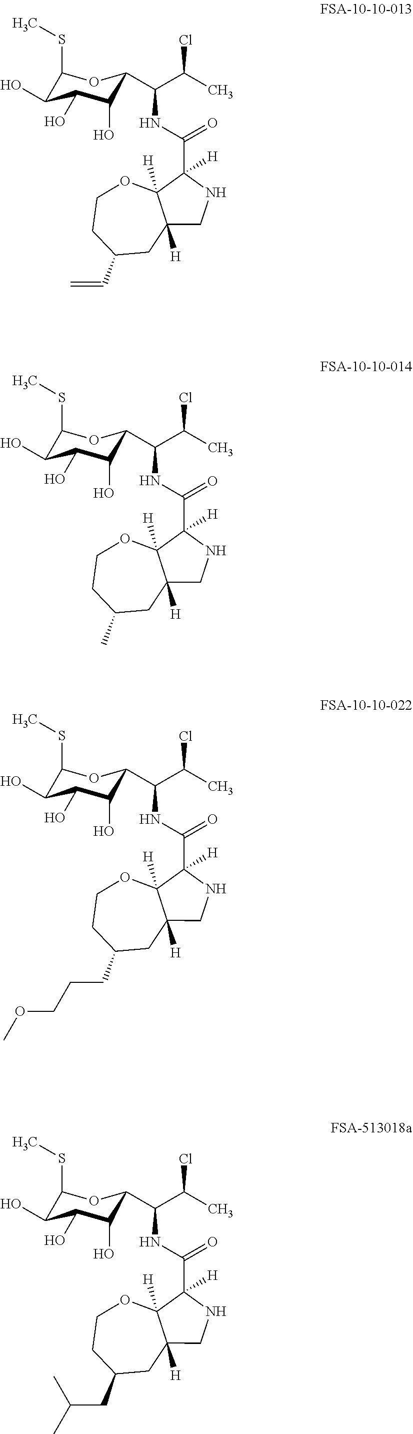 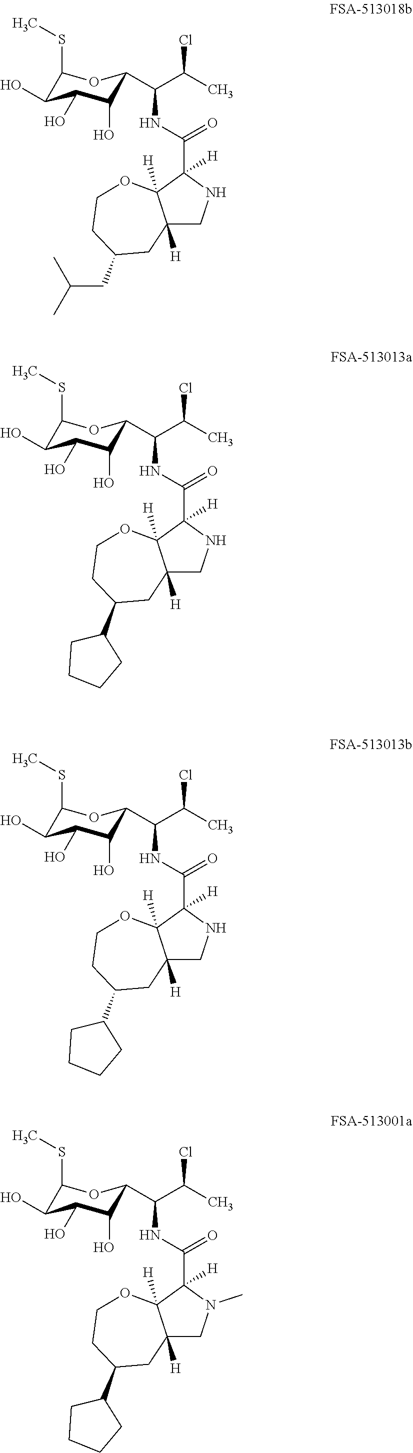 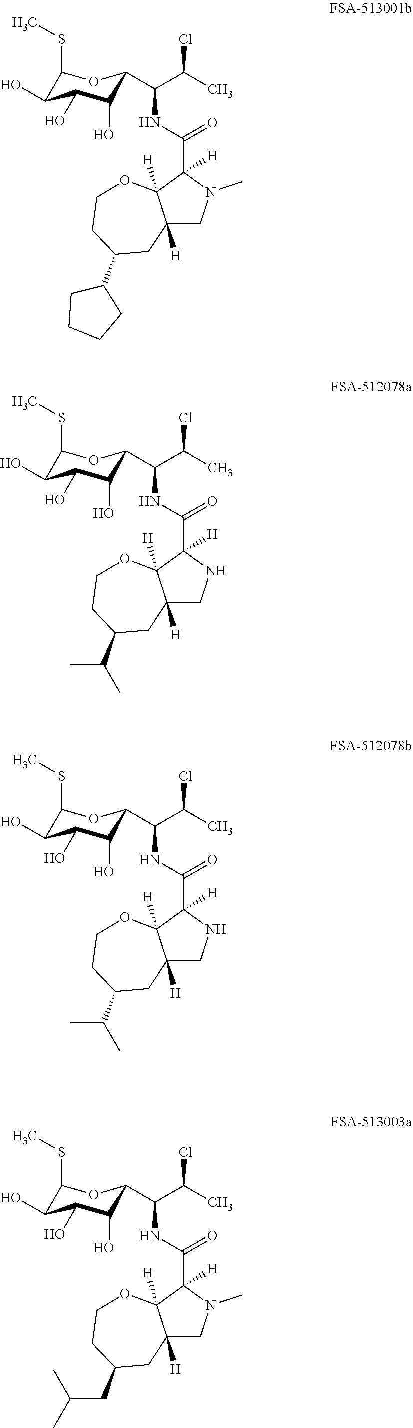 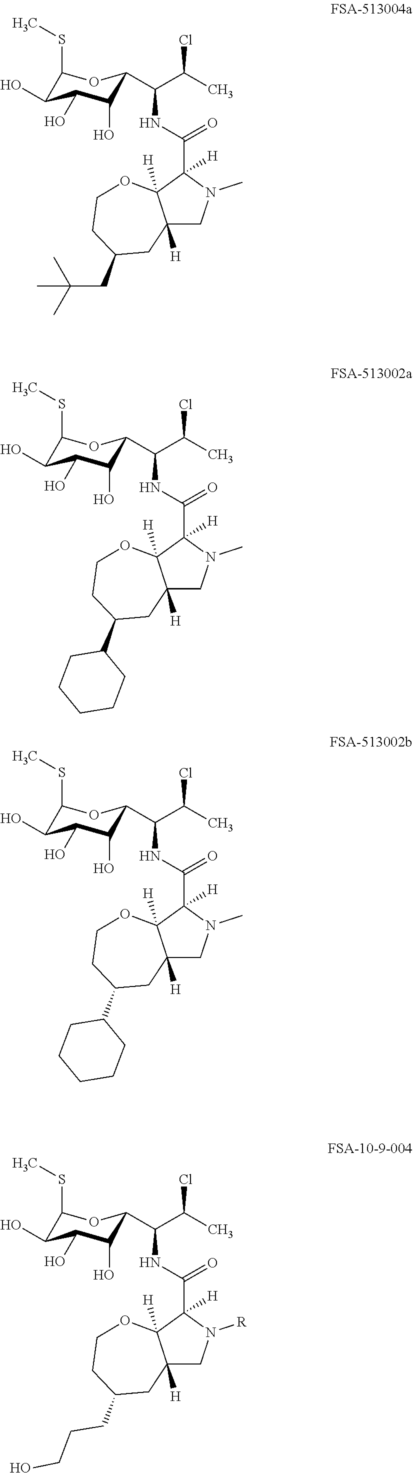 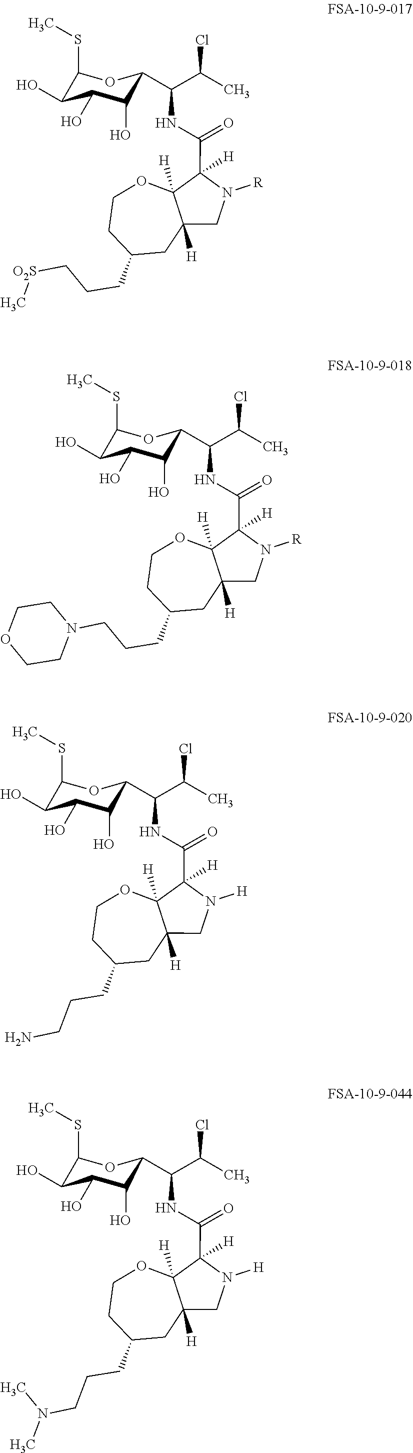 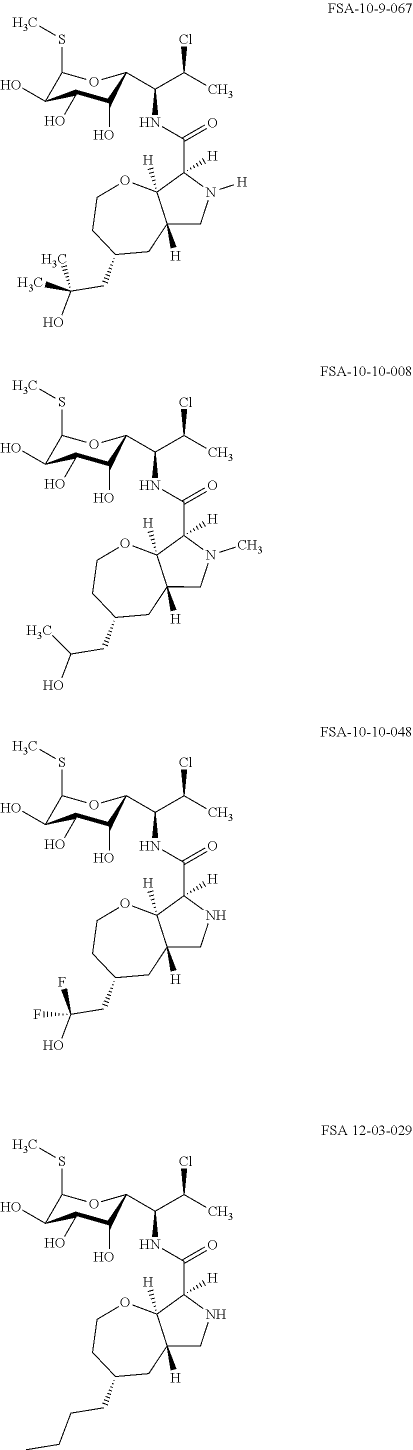 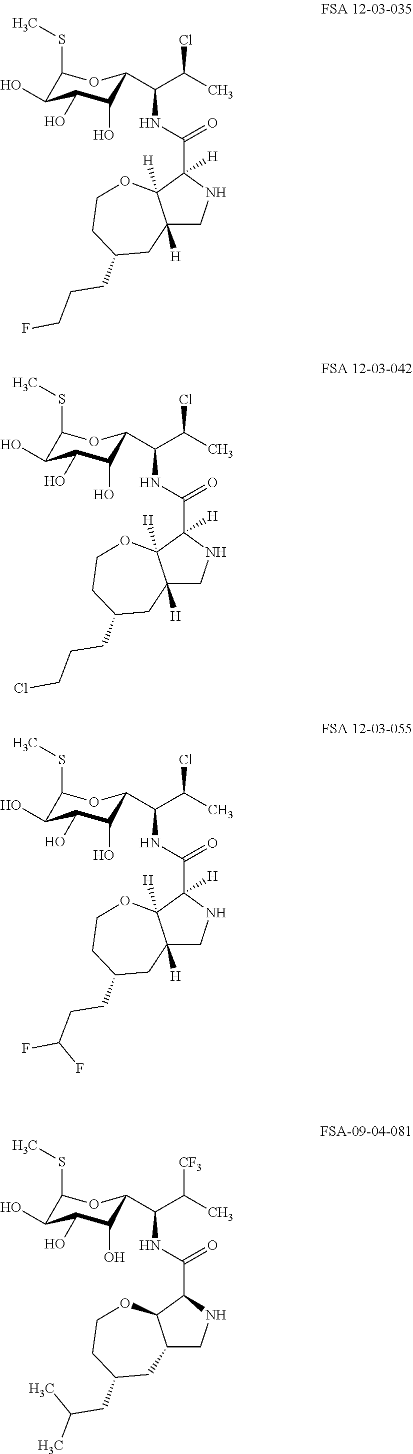 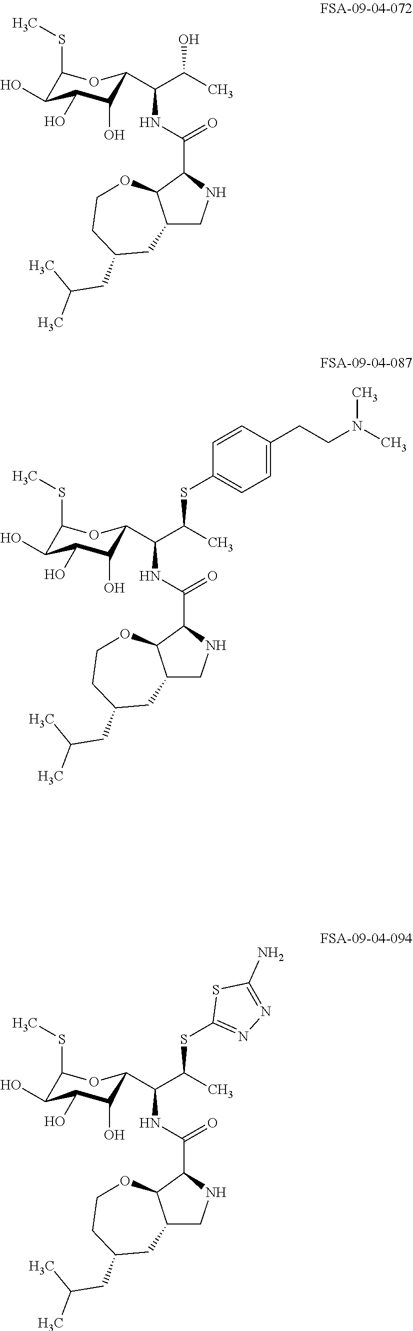 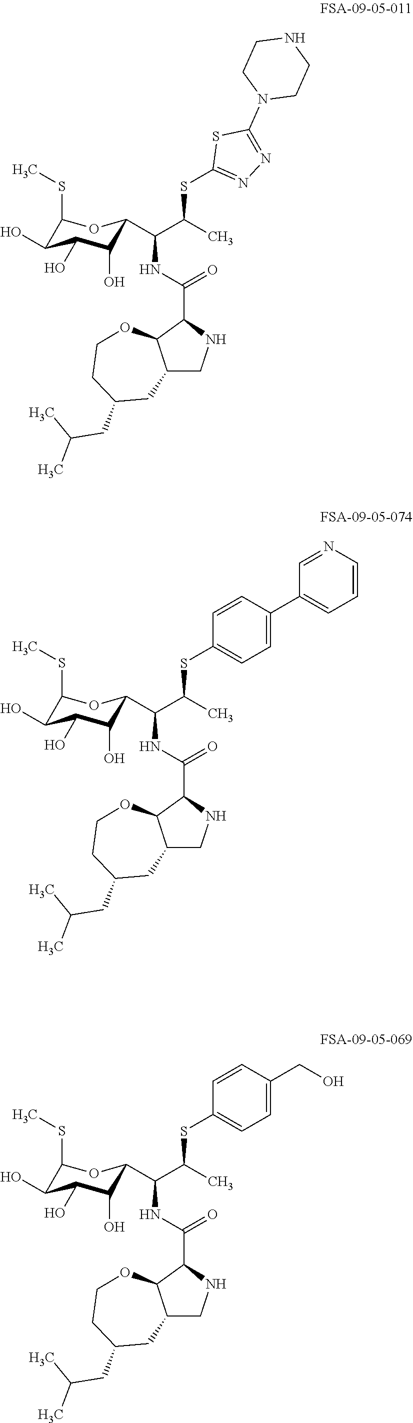 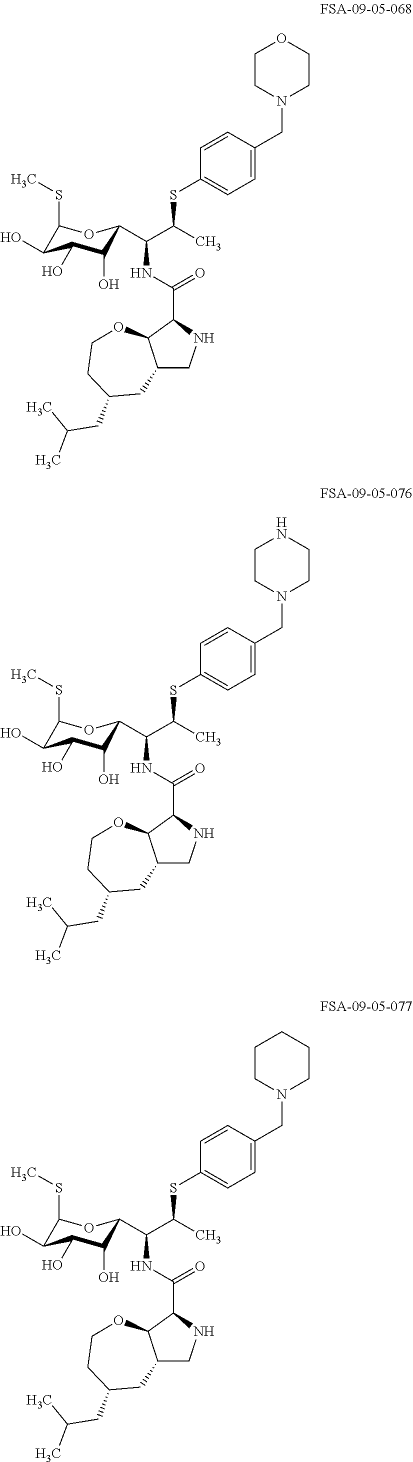 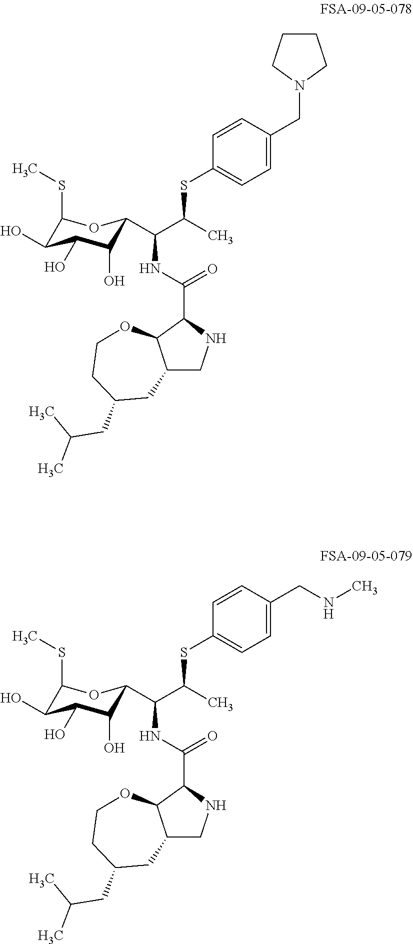
or a pharmaceutically acceptable salt thereof.

67. The compound of claim 1, wherein the compound is: 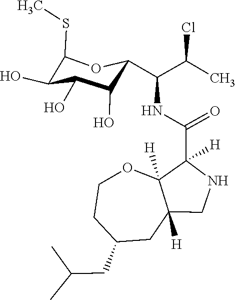
or a pharmaceutically acceptable salt thereof.

68. A pharmaceutical composition comprising a compound of any of claims 1-67, or a pharmaceutically acceptable salt thereof, and a pharmaceutically acceptable excipient.

69. A kit comprising a compound of any of claims 1-67, or a pharmaceutically acceptable salt thereof, or a pharmaceutical composition of claim 68, and instructions for administering the compound or pharmaceutical composition to a subject in need thereof.

70. A method of preventing an infectious disease comprising administering an effective amount of a compound of any of claims 1-67, or pharmaceutically acceptable salt thereof, or a pharmaceutical composition of claim 68, to a subject in need thereof.

71. A method of treating an infectious disease comprising administering an effective amount of a compound of any of claims 1-67, or pharmaceutically acceptable salt thereof, or a pharmaceutical composition of claim 68, to a subject in need thereof.

72. The method of claim 70 or 71, wherein the infectious disease is a bacterial infection.

73. The method of claim 72, wherein the bacterial infection is an infection caused by a Gram positive bacteria.

74. The method of claim 72, wherein the bacterial infection is an infection caused by a Gram negative bacteria.

75. The method of claim 72, wherein the bacterial infection is a Staphylococcus infection, a Streptococcus infection, an Enterococcus infection, an Acetinobacter infection, a Clostridium infection, a Bacteroides infection, an Escherichia infection, a Pseudomonas infection, a Neisseria infection, a Klebsiella infection, or a Haemophilus infection.

76. The method of claim 72, wherein the bacterial infection is a C. difficile infection or a B. fragilis infection.

77. The method of claim 70 or 71, wherein the infectious disease is a parasitic infection.

78. A method of killing a microorganism comprising contacting the microorganism with an effective amount of a compound of any of claims 1-67, or pharmaceutically acceptable salt thereof, or a pharmaceutical composition of claim 68.

79. A method of inhibiting the growth of a microorganism comprising contacting the microorganism with an effective amount of a compound of any of claims 1-67, or pharmaceutically acceptable salt thereof, or a pharmaceutical composition of claim 68.

80. A method of preparing a compound of Formula (I): 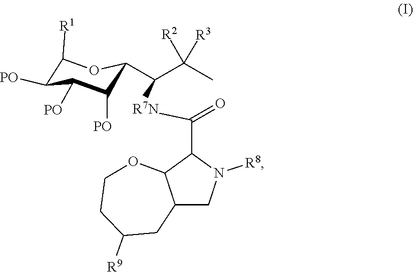
or a salt thereof, the method comprising coupling a compound of Formula (A): 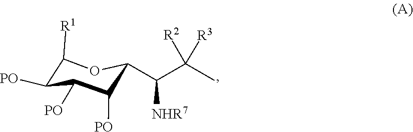
or salt thereof, with a compound of Formula (B): 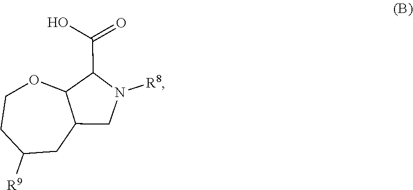
to yield a compound of Formula (I), wherein P, R¹, R², R³, R⁷, R⁸, and R⁹ are as defined in claim 1.

81. The method of claim 80 further comprising adding an amide coupling reagent.

82. The method of claim 81, wherein the amide coupling reagent is 3-(Diethoxyphosphoryloxy)-1,2,3-benzotriazin-4(3H)-one (DEPBT), 1-[bis(dimethylamino)methylene]-1H-1,2,3-triazolo[4,5-b]pyridinium 3-oxid hexafluorophosphate (HATU), 1-ethyl-3-(3-dimethylaminopropyl)carbodiimide (EDC), hydroxybenzotriazole (HOBt), or a combination thereof.

83. The method of claim 80 further comprising forming a compound of Formula (C): 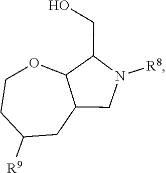
or a salt thereof, the method comprising cyclizing a compound of Formula (D): 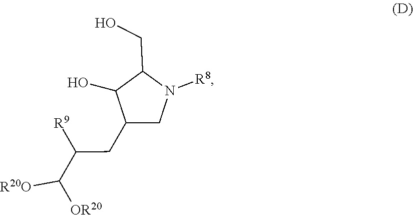
or a salt thereof; wherein:
each R²⁰ is independently hydrogen, substituted or unsubstitued alkyl, or both instances of R²⁰ join together with the atoms to which they are attached form a substituted or unsubstitued heterocycle.

84. The method of claim 83 further comprising oxidizing a compound of Formula (E): 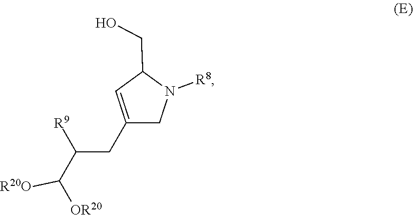
or salt thereof, to provide the compound of Formula (D).

85. The method of claim 84 further comprising coupling a compound of Formula (F): 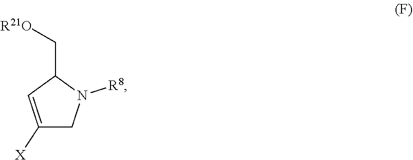
or a salt thereof, with a compound of Formula (G): 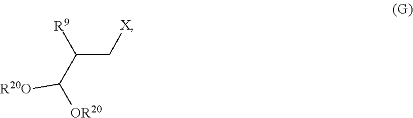
or a salt thereof, wherein
R²¹ is an oxygen protecting group or hydrogen; and
each X is independently a leaving group.
